SEQUENCE LISTING (221 sequences)

SEQ ID NO: 1 - DNA, 1761 bp, Homo sapiens
atgccgaaaccaatcaatgtccgagttaccaccatggatgcagagctggagtttgcaatccagccaaatacaactggaaaacagctttttgatcaggtggtaaagactatcggcctccgggaagtgtggtactttggcctccactatgtggataataaaggatttcctacctggctgaagctggataagaaggtgtctgcccaggaggtcaggaaggagaatcccctccagttcaagttccgggccaagttctaccctgaagatgtggctgaggagctcatccaggacatcacccagaaacttttcttcctccaagtgaaggaaggaatccttagcgatgagatctactgcccccctgagactgccgtgctcttggggtcctacgctgtgcaggccaagtttggggactacaacaaagaagtgcacaagtctgggtacctcagctctgagcggctgatccctcaaagagtgatggaccagcacaaacttaccagggaccagtgggaggaccggatccaggtgtggcatgcggaacaccgtgggatgctcaaagataatgctatgttggaatacctgaagattgctcaggacctggaaatgtatggaatcaactatttcgagataaaaaacaagaaaggaacagacctttggcttggagttgatgcccttggactgaatatttatgagaaagatgataagttaaccccaaagattggctttccttggagtgaaatcaggaacatctctttcaatgacaaaaagtttgtcattaaacccatcgacaagaaggcacctgactttgtgttttatgccccacgtctgagaatcaacaagcggatcctgcagctctgcatgggcaaccatgagttgtatatgcgccgcaggaagcctgacaccatcgaggtgcagcagatgaaggcccaggcccgggaggagaagcatcagaagcagctggagcggcaacagctggaaacagagaagaaaaggagagaaaccgtggagagagagaaagagcagatgatgcgcgagaaggaggagttgatgctgcggctgcaggactatgaggagaagacaaagaaggcagagagagagctctcggagcagattcagagggccctgcagctggaggaggagaggaagcgggcacaggaggaggccgagcgcctagaggctgaccgtatggctgcactgcgggctaaggaggagctggagagacaggcggtggatcagataaagagccaggagcagctggctgcggagcttgcagaatacacagccaagattgccctcctggaagaggcgcggaggcgcaaggaggatgaagttgaagagtggcagcacagggccaaagaagcccaggatgacctggtgaagaccaaggaggagctgcacctggtgatgacagcacccccgcccccaccaccccccgtgtacgagccggtgagctaccatgtccaggagagcttgcaggatgagggcgcagagcccacgggctacagcgcggagctgtctagtgagggcatccgggatgaccgcaatgaggagaagcgcatcactgaggcagagaagaacgagcgtgtgcagcggcagctcgtgacgctgagcagcgagctgtcccaggcccgagatgagaataagaggacccacaatgacatcatccacaacgagaacatgaggcaaggccgggacaagtacaagacgctgcggcagatccggcagggcaacaccaagcagcgcatcgacgagttcgaggccctgtaa

SEQ ID NO: 2 - protein, 586 aa, Homo sapiens
MPKPINVRVTTMDAELEFAIQPNTTGKQLFDQVVKTIGLREVWYFGLHYVDNKGFPTWLKLDKKVSAQEVRKENPLQFKFRAKFYPEDVAEELIQDITQKLFFLQVKEGILSDEIYCPPETAVLLGSYAVQAKFGDYNKEVHKSGYLSSERLIPQRVMDQHKLTRDQWEDRIQVWHAEHRGMLKDNAMLEYLKIAQDLEMYGINYFEIKNKKGTDLWLGVDALGLNIYEKDDKLTPKIGFPWSEIRNISFNDKKFVIKPIDKKAPDFVFYAPRLRINKRILQLCMGNHELYMRRRKPDTIEVQQMKAQAREEKHQKQLERQQLETEKKRRETVEREKEQMMREKEELMLRLQDYEEKTKKAERELSEQIQRALQLEEERKRAQEEAERLEADRMAALRAKEELERQAVDQIKSQEQLAAELAEYTAKIALLEEARRRKEDEVEEWQHRAKEAQDDLVKTKEELHLVMTAPPPPPPPVYEPVSYHVQESLQDEGAEPTGYSAELSSEGIRDDRNEEKRITEAEKNERVQRQLVTLSSELSQARDENKRTHNDIIHNENMRQGRDKYKTLRQIRQGNTKQRIDEFEAL

SEQ ID NO: 3 - DNA, 1752 bp, Homo sapiens
atgccgaaaccaatcaacgtaagagtaactacaatggatgctgagctggaatttgccattcagcccaatacaactggcaaacaactttttgaccaggtggtgaaaacagttggtttgcgtgaggtctggttttttgggctgcagtatgtagacagcaaaggttattctacatggcttaaactaaataaaaaggtaacacagcaggatgttaaaaaagagaatcctttacagttcaagtttagagctaaattctttcctgaagatgtttctgaggaattaattcaagaaataacccagagactcttcttcttgcaagttaaagaagccatcttaaatgatgagatatattgcccgccagaaactgcagttcttttggcttcctatgctgtccaagccaagtatggagattacaataaagagattcataagccaggctacctggctaatgatagactcctaccccagcgtgtattggaacaacacaaactaacaaaagaacagtgggaagaaagaatacagaactggcatgaagaacatagaggaatgttaagggaggattctatgatggaatacctgaagattgcacaagatctagaaatgtatggagtcaactattttgaaataaaaaataaaaaaggaactgaattgtggctaggtgttgatgctttgggtctgaatatttatgagcatgacgacaagttaacacctaaaattggttttccctggagtgaaatcagaaatatttcatttaatgacaaaaaatttgttataaagccaatcgacaaaaaggcacctgattttgtgttttatgcacctcgtctgagaatcaataagcggattttggccttatgtatgggaaaccatgaactatacatgcgaagaaggaagcctgatactattgaagtacaacagatgaaggctcaggctagggaggagaaacatcagaagcagttggaaagggcacaattagagaatgaaaagaagaaaagagaaatagcagaaaaggaaaaggaaagaatagaacgtgaaaaggaagagctaatggaacgtctaaaacaaattgaagagcagacaattaaagctcagaaagaactagaagaacagactcgaaaagctctagaactggatcaagaacgaaaacgagcaaaagaagaagcagaacgacttgaaaaggagcgtcgagctgctgaagaggcaaagtctgccatagcaaaacaagctgccgaccagatgaagaatcaggagcagctagcagcagaacttgctgaattcactgccaagattgcacttctagaggaagccaagaagaaaaaggaagaggaagcaactgagtggcaacacaaagcttttgcagcccaggaagacttggaaaagaccaaagaagagttaaaaactgtgatgtctgccccccctccacctccaccaccaccagtcattcctccaacagaaaacgaacatgatgaacacgatgagaataatgctgaagctagtgctgaattatcaaatgaaggggtaatgaaccatagaagcgaggaagaacgtgtaaccgaaacacagaaaaatgagcgtgttaagaagcaacttcaggcattaagttcagaattagcccaagccagagatgaaaccaagaaaacacaaaatgatgttcttcatgctgagaatgttaaagcaggccgtgataagtacaagactctgcgacagattcgacaaggcaatacaaagcagcgtatcgatgagtttgaagcaatgtga

SEQ ID NO: 4 - protein, 583 aa, Homo sapiens
MPKPINVRVTTMDAELEFAIQPNTTGKQLFDQVVKTVGLREVWFFGLQYVDSKGYSTWLKLNKKVTQQDVKKENPLQFKFRAKFFPEDVSEELIQEITQRLFFLQVKEAILNDEIYCPPETAVLLASYAVQAKYGDYNKEIHKPGYLANDRLLPQRVLEQHKLTKEQWEERIQNWHEEHRGMLREDSMMEYLKIAQDLEMYGVNYFEIKNKKGTELWLGVDALGLNIYEHDDKLTPKIGFPWSEIRNISFNDKKFVIKPIDKKAPDFVFYAPRLRINKRILALCMGNHELYMRRRKPDTIEVQQMKAQAREEKHQKQLERAQLENEKKKREIAEKEKERIEREKEELMERLKQIEEQTIKAQKELEEQTRKALELDQERKRAKEEAERLEKERRAAEEAKSAIAKQAADQMKNQEQLAAELAEFTAKIALLEEAKKKKEEEATEWQHKAFAAQEDLEKTKEELKTVMSAPPPPPPPPVIPPTENEHDEHDENNAEASAELSNEGVMNHRSEEERVTETQKNERVKKQLQALSSELAQARDETKKTQNDVLHAENVKAGRDKYKTLRQIRQGNTKQRIDEFEAM

SEQ ID NO: 5 - DNA, 1734 bp, Homo sapiens
atgcccaaaacgatcagtgtgcgtgtgaccaccatggatgcagagctggagtttgccatccagcccaacaccaccgggaagcagctatttgaccaggtggtgaaaactattggcttgagggaagtttggttctttggtctgcagtaccaggacactaaaggtttctccacctggctgaaactcaataagaaggtgactgcccaggatgtgcggaaggaaagccccctgctctttaagttccgtgccaagttctaccctgaggatgtgtccgaggaattgattcaggacatcactcagcgcctgttctttctgcaagtgaaagagggcattctcaatgatgatatttactgcccgcctgagaccgctgtgctgctggcctcgtatgctgtccagtctaagtatggcgacttcaataaggaagtgcataagtctggctacctggccggagacaagttgctcccgcagagagtcctggaacagcacaaactcaacaaggaccagtgggaggagcggatccaggtgtggcatgaggaacaccgtggcatgctcagggaggatgctgtcctggaatatctgaagattgctcaagatctggagatgtatggtgtgaactacttcagcatcaagaacaagaaaggctcagagctgtggctgggggtggatgccctgggtctcaacatctatgagcagaatgacagactaactcccaagataggcttcccctggagtgaaatcaggaacatctctttcaatgataagaaatttgtcatcaagcccattgacaaaaaagccccggacttcgtcttctatgctccccggctgcggattaacaagcggatcttggccttgtgcatggggaaccatgaactatacatgcgccgtcgcaagcctgataccattgaggtgcagcagatgaaggcacaggcccgggaggagaagcaccagaagcagatggagcgtgctatgctggaaaatgagaagaagaagcgtgaaatggcagagaaggagaaagagaagattgaacgggagaaggaggagctgatggagaggctgaagcagatcgaggaacagactaagaaggctcagcaagaactggaagaacagacccgtagggctctggaacttgagcaggaacggaagcgtgcccagagcgaggctgaaaagctggccaaggagcgtcaagaagctgaagaggccaaggaggccttgctgcaggcctcccgggaccagaaaaagactcaggaacagctggccttggaaatggcagagctgacagctcgaatctcccagctggagatggcccgacagaagaaggagagtgaggctgtggagtggcagcagaaggcccagatggtacaggaagacttggagaagacccgtgctgagctgaagactgccatgagtacacctcatgtggcagagcctgctgagaatgagcaggatgagcaggatgagaatggggcagaggctagtgctgacctacgggctgatgctatggccaaggaccgcagtgaggaggaacgtaccactgaggcagagaagaatgagcgtgtgcagaagcacctgaaggccctcacttcggagctggccaatgccagagatgagtccaagaagactgccaatgacatgatccatgctgagaacatgcgactgggccgagacaaatacaagaccctgcgccagatccggcagggcaacaccaagcagcgcattgacgaatttgagtctatgtaa

SEQ ID NO: 6 - protein, 577 aa, Homo sapiens
MPKTISVRVTTMDAELEFAIQPNTTGKQLFDQVVKTIGLREVWFFGLQYQDTKGFSTWLKLNKKVTAQDVRKESPLLFKFRAKFYPEDVSEELIQDITQRLFFLQVKEGILNDDIYCPPETAVLLASYAVQSKYGDFNKEVHKSGYLAGDKLLPQRVLEQHKLNKDQWEERIQVWHEEHRGMLREDAVLEYLKIAQDLEMYGVNYFSIKNKKGSELWLGVDALGLNIYEQNDRLTPKIGFPWSEIRNISFNDKKFVIKPIDKKAPDFVFYAPRLRINKRILALCMGNHELYMRRRKPDTIEVQQMKAQAREEKHQKQMERAMLENEKKKREMAEKEKEKIEREKEELMERLKQIEEQTKKAQQELEEQTRRALELEQERKRAQSEAEKLAKERQEAEEAKEALLQASRDQKKTQEQLALEMAELTARISQLEMARQKKESEAVEWQQKAQMVQEDLEKTRAELKTAMSTPHVAEPAENEQDEQDENGAEASADLRADAMAKDRSEEERTTEAEKNERVQKHLKALTSELANARDESKKTANDMIHAENMRLGRDKYKTLRQIRQGNTKQRIDEFESM

SEQ ID NO: 7 - DNA, 582 bp, Homo sapiens
atggctgccatccggaagaaactggtgattgttggtgatggagcctgtggaaagacatgcttgctcatagtcttcagcaaggaccagttcccagaggtgtatgtgcccacagtgtttgagaactatgtggcagatatcgaggtggatggaaagcaggtagagttggctttgtgggacacagctgggcaggaagattatgatcgcctgaggcccctctcctacccagataccgatgttatactgatgtgtttttccatcgacagccctgatagtttagaaaacatcccagaaaagtggaccccagaagtcaagcatttctgtcccaacgtgcccatcatcctggttgggaataagaaggatcttcggaatgatgagcacacaaggcgggagctagccaagatgaagcaggagccggtgaaacctgaagaaggcagagatatggcaaacaggattggcgcttttgggtacatggagtgttcagcaaagaccaaagatggagtgagagaggtttttgaaatggctacgagagctgctctgcaagctagacgtgggaagaaaaaatctggttgccttgtcttgtga

SEQ ID NO: 8 - protein, 193 aa, Homo sapiens
MAAIRKKLVIVGDGACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 9 - DNA, 1815 bp, Artificial Sequence
  SYNTHETIC DNA
atgggatgtattaaatcaaaaaggaaagacggatccctggtcgacgttgaattcaccatgccgaaaccaatcaatgtccgagttaccaccatggatgcagagctggagtttgcaatccagccaaatacaactggaaaacagctttttgatcaggtggtaaagactatcggcctccgggaagtgtggtactttggcctccactatgtggataataaaggatttcctacctggctgaagctggataagaaggtgtctgcccaggaggtcaggaaggagaatcccctccagttcaagttccgggccaagttctaccctgaagatgtggctgaggagctcatccaggacatcacccagaaacttttcttcctccaagtgaaggaaggaatccttagcgatgagatctactgcccccctgagactgccgtgctcttggggtcctacgctgtgcaggccaagtttggggactacaacaaagaagtgcacaagtctgggtacctcagctctgagcggctgatccctcaaagagtgatggaccagcacaaacttaccagggaccagtgggaggaccggatccaggtgtggcatgcggaacaccgtgggatgctcaaagataatgctatgttggaatacctgaagattgctcaggacctggaaatgtatggaatcaactatttcgagataaaaaacaagaaaggaacagacctttggcttggagttgatgcccttggactgaatatttatgagaaagatgataagttaaccccaaagattggctttccttggagtgaaatcaggaacatctctttcaatgacaaaaagtttgtcattaaacccatcgacaagaaggcacctgactttgtgttttatgccccacgtctgagaatcaacaagcggatcctgcagctctgcatgggcaaccatgagttgtatatgcgccgcaggaagcctgacaccatcgaggtgcagcagatgaaggcccaggcccgggaggagaagcatcagaagcagctggagcggcaacagctggaaacagagaagaaaaggagagaaaccgtggagagagagaaagagcagatgatgcgcgagaaggaggagttgatgctgcggctgcaggactatgaggagaagacaaagaaggcagagagagagctctcggagcagattcagagggccctgcagctggaggaggagaggaagcgggcacaggaggaggccgagcgcctagaggctgaccgtatggctgcactgcgggctaaggaggagctggagagacaggcggtggatcagataaagagccaggagcagctggctgcggagcttgcagaatacacagccaagattgccctcctggaagaggcgcggaggcgcaaggaggatgaagttgaagagtggcagcacagggccaaagaagcccaggatgacctggtgaagaccaaggaggagctgcacctggtgatgacagcacccccgcccccaccaccccccgtgtacgagccggtgagctaccatgtccaggagagcttgcaggatgagggcgcagagcccacgggctacagcgcggagctgtctagtgagggcatccgggatgaccgcaatgaggagaagcgcatcactgaggcagagaagaacgagcgtgtgcagcggcagctgctgacgctgagcagcgagctgtcccaggcccgagatgagaataagaggacccacaatgacatcatccacaacgagaacatgaggcaaggccgggacaagtacaaggagctgcggcagatccggcagggcaacaccaagcagcgcattgacgagttcgaggccctg

SEQ ID NO: 10 - protein, 596 aa, Artificial Sequence
  SYNTHETIC PROTEIN - FUSION PROTEIN
MGCIKSKRKDMPKPINVRVTTMDAELEFAIQPNTTGKQLFDQVVKTIGLREVWYFGLHYVDNKGFPTWLKLDKKVSAQEVRKENPLQFKFRAKFYPEDVAEELIQDITQKLFFLQVKEGILSDEIYCPPETAVLLGSYAVQAKFGDYNKEVHKSGYLSSERLIPQRVMDQHKLTRDQWEDRIQVWHAEHRGMLKDNAMLEYLKIAQDLEMYGINYFEIKNKKGTDLWLGVDALGLNIYEKDDKLTPKIGFPWSEIRNISFNDKKFVIKPIDKKAPDFVFYAPRLRINKRILQLCMGNHELYMRRRKPDTIEVQQMKAQAREEKHQKQLERQQLETEKKRRETVEREKEQMMREKEELMLRLQDYEEKTKKAERELSEQIQRALQLEEERKRAQEEAERLEADRMAALRAKEELERQAVDQIKSQEQLAAELAEYTAKIALLEEARRRKEDEVEEWQHRAKEAQDDLVKTKEELHLVMTAPPPPPPPVYEPVSYHVQESLQDEGAEPTGYSAELSSEGIRDDRNEEKRITEAEKNERVQRQLLTLSSELSQARDENKRTHNDIIHNENMRQGRDKYKELRQIRQGNTKQRIDEFEAL

SEQ ID NO: 11 - DNA, 2244 bp, Homo sapiens
atggagacggcggagaaggagtgcggcgccctgggcgggctcttccaggccatagtcaacgacatgaagagctcctaccctatctgggaggacttcaactccaaggccacgaagctgcattcccagctgaggaccaccgtgctggctgctgtggccttcctggatgccttccagaaagtggctgacatggctaccaacacccgaggggccacgagggacatcggctcggcgctcacacgcatgtgcatgcgccaccgcagcatcgagaccaagctgcggcagttcaccaacgcactgctggagagcctcatcaacccgctgcaggagcgcatcgaggactggaagaaggcggccaaccagctggacaaggaccacgcgaaagagtacaaacgagcccggcatgagatcaaaaagaagtcgtcggacacgctgaagctgcagaagaaggcgcgcaaagagctacttgggaaaggagacctgcagccccagctggacagtgccctgcaggacgtcaacgacatgtacctgctgctggaggagacggagaagcaggccgtgcgccgggcgctgatcgaggagcggggccgcttctgcaccttcatcaccttcctgcagcctgtggtgaatggagagctgaccatgctgggagagatcacccacctgcagggcatcatcgacgacttggtggtgctgacagcagaaccccacaaactgcctcccgccagcgagcaggtaatcaaagacctaaagggctcggactacagctggtcctaccagaccccaccctcatcacccagcagctccagctcccggaagtccagcatgtgcagtgcccccagcagcagtagcagtgccaagggtggcggagccccatggcctgggggtgcccaaacatactcacccagttccacctgtcgctaccgcagcctggcgcagccagccaccaccaccgctcgcctctccagcgtttcctcccatgactctggcttcgtctcccaggacgccacctactccaagcccccctcgcctatgccttcagacatcaccagccagaagtcctccagctctgcatcttccgaggcctcggaaacctgccagtccgttagcgagtgcagctcccccacctcggactggtccaaggtcggctcccatgagcagccctcaggcgccactctgcagcggaggaaggaccgagtggagctcctgcgagacacagagccaggccctgccagtgggggcaccctgggccccagcggggaagaggcaccgcgaccccggatgtcccctgccaccatcgcagccaagcacggtgaggaggtgtcccccgccgccagtgacctggccatggtgctgacgcggggcctgagcctggagcaccagaagagcagccgggactcgctgcagtactccagcggctacagcacgcagaccaccacgccctcctgctctgaggacaccatcccctcccaaggctccgactacgactgctactccgtgaatggggatgcggacagcgagggcccgcccgagtttgacaagtcatccaccatcccgcgcaacagcaacatcgcccagaactaccgccgcctgatccagaccaagcgcccagcctccactgctgggctgcccaccgcggggctgcccacggccactggcctgccctcgggcgcaccccccggcgtggccaccatccgccgcacaccctccaccaagcccaccgtgcgccgcgccctgtccagcgctggccccatccccatccggccgcccatcgtccctgtgaagacgcccacggtgcctgactcccccggctacatggggcccacacgggcgggcagtgaggagtgcgtcttctataccgacgagaccgcctcacccctggcaccggacctcgccaaggcctccccaaagaggctcagcctgcccaacacagcctggggcagcccatccccagaggcagccgggtaccccggggcaggggccgaggacgagcagcagcagctggcggccaaccggcacagcctggtggagaagctgggggagctggtggcgggtgcccacgcactgggtgagggccagttccccttccccactgctctgtcggccacccccacggaggagacgcccaccccacccccagccgccaccagcgaccccccggccgaagacatgctggtggccatccggcgtggggtccggctccgcaggaccgtcaccaacgacaggtcggcgccccgcatcttatga

SEQ ID NO: 12 - protein, 747 aa, Homo sapiens
METAEKECGALGGLFQAIVNDMKSSYPIWEDFNSKATKLHSQLRTTVLAAVAFLDAFQKVADMATNTRGATRDIGSALTRMCMRHRSIETKLRQFTNALLESLINPLQERIEDWKKAANQLDKDHAKEYKRARHEIKKKSSDTLKLQKKARKELLGKGDLQPQLDSALQDVNDMYLLLEETEKQAVRRALIEERGRFCTFITFLQPVVNGELTMLGEITHLQGIIDDLVVLTAEPHKLPPASEQVIKDLKGSDYSWSYQTPPSSPSSSSSRKSSMCSAPSSSSSAKGGGAPWPGGAQTYSPSSTCRYRSLAQPATTTARLSSVSSHDSGFVSQDATYSKPPSPMPSDITSQKSSSSASSEASETCQSVSECSSPTSDWSKVGSHEQPSGATLQRRKDRVELLRDTEPGPASGGTLGPSGEEAPRPRMSPATIAAKHGEEVSPAASDLAMVLTRGLSLEHQKSSRDSLQYSSGYSTQTTTPSCSEDTIPSQGSDYDCYSVNGDADSEGPPEFDKSSTIPRNSNIAQNYRRLIQTKRPASTAGLPTAGLPTATGLPSGAPPGVATIRRTPSTKPTVRRALSSAGPIPIRPPIVPVKTPTVPDSPGYMGPTRAGSEECVFYTDETASPLAPDLAKASPKRLSLPNTAWGSPSPEAAGYPGAGAEDEQQQLAANRHSLVEKLGELVAGAHALGEGQFPFPTALSATPTEETPTPPPAATSDPPAEDMLVAIRRGVRLRRTVTNDRSAPRIL

SEQ ID NO: 13 - DNA, 1854 bp, Homo sapiens
atgagctggggcaccgagctctgggatcagtttgacaacttagaaaaacacacacagtggggaattgatattcttgagaaatatatcaagtttgtgaaagaaaggacagagattgaactcagctatgcaaagcaactcaggaatctttcaaagaagtaccaacctaaaaagaactcgaaggaggaagaagaatacaagtatacgtcatgtaaagctttcatttccaacctgaacgaaatgaatgattacgcagggcagcatgaagttatctccgagaacatggcatcacagatcattgtggacttggcacgctatgttcaggaactgaaacaggagaggaaatcaaactttcacgatggccgtaaagcacagcagcacatcgagacttgctggaagcagcttgaatctagtaaaaggcgatttgaacgcgattgcaaagaggcggacagggcgcagcagtactttgagaaaatggacgctgacatcaatgtcacaaaagcggatgttgaaaaggcccgacaacaagctcaaatacgtcaccaaatggcagaggacagcaaagcagattactcatccattctccagaaattcaaccatgagcagcatgaatattaccatactcacatccccaacatcttccagaaaatacaagagatggaggaaaggaggattgtgagaatgggagagtccatgaagacatatgcagaggttgatcggcaggtgatcccaatcattgggaagtgcctggatggaatagtaaaagcagccgaatcaattgatcagaaaaatgattcacagctggtaatagaagcttataaatcagggtttgagcctcctggagacattgaatttgaggattacactcagccaatgaagcgcactgtgtcagataacagcctttcaaattccagaggagaaggcaaaccagacctcaaatttggtggcaaatccaaaggaaagttatggccgttcatcaaaaaaaataagcttatgtcccttttaacatccccccatcagcctccccctccccctcctgcctctgcctcaccctctgctgttcccaacggcccccagtctcccaagcagcaaaaggaacccctctcccaccgcttcaacgagttcatgacctccaaacccaaaatccactgcttcaggagcctaaagcgtgggctttctctcaagctgggtgcaacaccggaggatttcagcaacctcccacctgaacaaagaaggaaaaagctgcagcagaaagtcgatgagttaaataaagaaattcagaaggagatggatcaaagagatgccataacaaaaatgaaagatgtctacctaaagaatcctcagatgggagacccagccagtttggatcacaaattagcagaagtcagccaaaatatagagaaactgcgagtagagacccagaaatttgaggcctggctggctgaggttgaaggccggctcccagcacgcagcgagcaggcgcgccggcagagcggactgtacgacagccagaacccacccacagtcaacaactgcgcccaggaccgtgagagcccagatggcagttacacagaggagcagagtcaggagagtgagatgaaggtgctggccacggattttgacgacgagtttgatgatgaggagcccctccctgccatagggacgtgcaaagctctctacacatttgaaggtcagaatgaaggaacgatttccgtagttgaaggagaaacattgtatgtcatagaggaagacaaaggcgatggctggacccgcattcggagaaatgaagatgaagagggttatgtccccacttcatatgtcgaagtctgtttggacaaaaatgccaaagattcctag

SEQ ID NO: 14 - protein, 617 aa, Homo sapiens
MSWGTELWDQFDNLEKHTQWGIDILEKYIKFVKERTEIELSYAKQLRNLSKKYQPKKNSKEEEEYKYTSCKAFISNLNEMNDYAGQHEVISENMASQIIVDLARYVQELKQERKSNFHDGRKAQQHIETCWKQLESSKRRFERDCKEADRAQQYFEKMDADINVTKADVEKARQQAQIRHQMAEDSKADYSSILQKFNHEQHEYYHTHIPNIFQKIQEMEERRIVRMGESMKTYAEVDRQVIPIIGKCLDGIVKAAESIDQKNDSQLVIEAYKSGFEPPGDIEFEDYTQPMKRTVSDNSLSNSRGEGKPDLKFGGKSKGKLWPFIKKNKLMSLLTSPHQPPPPPPASASPSAVPNGPQSPKQQKEPLSHRFNEFMTSKPKIHCFRSLKRGLSLKLGATPEDFSNLPPEQRRKKLQQKVDELNKEIQKEMDQRDAITKMKDVYLKNPQMGDPASLDHKLAEVSQNIEKLRVETQKFEAWLAEVEGRLPARSEQARRQSGLYDSQNPPTVNNCAQDRESPDGSYTEEQSQESEMKVLATDFDDEFDDEEPLPAIGTCKALYTFEGQNEGTISVVEGETLYVIEEDKGDGWTRIRRNEDEEGYVPTSYVEVCLDKNAKDS

SEQ ID NO: 15 - DNA, 1806 bp, Homo sapiens
atggattggggcactgagctgtgggatcagttcgaggtgctcgagcgccacacgcagtgggggctggacctgttggacagatatgtaaagttcgtgaaagaacgcaccgaagtggaacaggcttacgccaaacaactgcggagcctggtgaaaaaatatctgcccaagagacctgccaaggatgatcctgagtccaaattcagccagcaacagtccttcgtacagattctccaggaggtgaatgactttgcaggccagcgggagctggtggctgagaacctcagtgtccgtgtatgtcttgagctgaccaagtactcacaagagatgaaacaggagaggaagatgcacttccaagaagggcggcgggcccagcagcagctggaaaatggctttaaacagctggagaatagtaagcgtaaatttgagcgggactgccgggaggcagagaaggcagcccagactgctgaacggctagaccaggatatcaacgccaccaaggctgatgtggagaaggccaagcagcaagcccaccttcggagtcacatggccgaagaaagcaaaaacgaatatgcggctcaactgcagcgcttcaaccgagaccaagcccacttctatttttcacagatgccccagatattcgataagctccaagacatggatgaacgcagggccacccgcctgggtgccgggtatgggctcctgtcggaggccgagctggaggtggtgcccataatagccaagtgcttggagggcatgaaggtggctgcaaatgctgtggatcccaagaacgactcccacgtccttatagagctgcacaagtcaggttttgcccgcccgggcgacgtggaattcgaggacttcagccagcccatgaaccgtgcaccctccgacagcagtctgggcaccccctcggatggacggcctgaactccgaggcccgggtcgcagccgcaccaagcgctggccttttggcaagaagaacaagcctcgccccccacccctctcccccctggggggccccgtaccctcggcattgcctaacggacccccgtccccccgctccggccgtgaccccttggccatactgagcgagatcagtaagtcggtcaaaccgaggctagcatccttccgcagccttcgaggcagccgtgggacagtggtgaccgaggattttagccacttgcccccagagcagcagcgaaaacggcttcaacagcagttggaagaacgcagtcgtgaacttcagaaggaggttgaccagagggaagccctaaagaaaatgaaggatgtctatgagaagacacctcagatgggggaccccgccagcttggagccccagatcgctgaaaccctgagcaacattgaacggctgaaattggaagtgcagaagtatgaggcgtggctggcagaagctgaaagtcgagtccttagcaaccggggagacagcctgagccggcacgcccggcctcccgacccccccgctagcgccccgccagacagcagcagcaacagcgcatcacaggacaccaaggagagctctgaagagcctccctcagaagagagccaggacacccccatttacacggagtttgatgaggatttcgaggaggaacccacatcccccataggtcactgtgtggccatctaccactttgaagggtccagcgagggcactatctctatggccgagggtgaagacctcagtcttatggaagaagacaaaggggacggctggacccgggtcaggcggaaagagggaggcgagggctacgtgcccacctcctacctccgagtcacgctcaattga

SEQ ID NO: 16 - protein, 601 aa, Homo sapiens
MDWGTELWDQFEVLERHTQWGLDLLDRYVKFVKERTEVEQAYAKQLRSLVKKYLPKRPAKDDPESKFSQQQSFVQILQEVNDFAGQRELVAENLSVRVCLELTKYSQEMKQERKMHFQEGRRAQQQLENGFKQLENSKRKFERDCREAEKAAQTAERLDQDINATKADVEKAKQQAHLRSHMAEESKNEYAAQLQRFNRDQAHFYFSQMPQIFDKLQDMDERRATRLGAGYGLLSEAELEVVPIIAKCLEGMKVAANAVDPKNDSHVLIELHKSGFARPGDVEFEDFSQPMNRAPSDSSLGTPSDGRPELRGPGRSRTKRWPFGKKNKPRPPPLSPLGGPVPSALPNGPPSPRSGRDPLAILSEISKSVKPRLASFRSLRGSRGTVVTEDFSHLPPEQQRKRLQQQLEERSRELQKEVDQREALKKMKDVYEKTPQMGDPASLEPQIAETLSNIERLKLEVQKYEAWLAEAESRVLSNRGDSLSRHARPPDPPASAPPDSSSNSASQDTKESSEEPPSEESQDTPIYTEFDEDFEEEPTSPIGHCVAIYHFEGSSEGTISMAEGEDLSLMEEDKGDGWTRVRRKEGGEGYVPTSYLRVTLN

SEQ ID NO: 17 - DNA, 2361 bp, Homo sapiens
atggggctgcagcccctggagttcagcgactgctacctcgacagcccgtggttccgggagaggatccgcgctcacgaagcggaactcgagaggaccaacaagttcatcaaagagctcattaaggacgggaagaacctcatcgctgcgacgaaaagtctgtcagtggcccagcggaagtttgctcattcactcagagactttaagtttgagtttatcggtgatgctgtgacagatgatgaacgatgcatagatgcttccttacgtgaattttcaaattttttgaagaatctggaggaacagagagaaattatggcattaagtgtaactgaaaccctgattaaacccttggaaaaattcagaaaagagcaacttggagctgtaaaggaagaaaaaaagaagtttgacaaagagacagaaaagaattatagtctaattgataaacatttgaatttatcagcaaaaaagaaagactcacatttacaagaggcagatatccaagtagagcagaaccggcaacacttctatgaactgtctctcgagtatgtgtgtaagctgcaggaaatccaagaaagaaagaagtttgagtttgtggaacctatgctgtcattttttcaggggatgtttaccttctatcatcagggccatgaacttgccaaagacttcaatcactacaaaatggaactacagatcaacattcagaatacacggaatcgatttgaaggaacaaggtcagaagtggaagagctcatgaacaaaatcagacagaatcccaaggaccacaaacgagcaagtcagtttacagccgaaggctacctgtatgtccaggaaaaaaggcctgctccgtttggttccagttgggtcaaacactattgcatgtatcgaaaagcagcaaagaagttcaacatgatcccatttgagcacagatctggagggaaacttggggacggagaggtgttctttttgaaagaatgtaccaagaggcatactgactccattgacagaaggttttgttttgacatagaagctgctgatcggcctggcgtttccttgaccatgcaggcattttccgaagaggaaaggaagcagtggttggaagctctgggtggaaaggaagctctgtcccatagttttaatacagccatcatcccaagaccagaaggaaatgcacagttggataagatggggttcacaattatcagaaaatgcatcagtgccgttgaaacacgaggtataaatgaccaaggattgtacagagttgtgggggtgagttcaaaggtccagagacttctgagtatgttgatggatgtaaaaacatgcaatgaggtggacctggagaattctgcagattgggaagtgaagacaataacaagtgccttgaaacagtatttgaggagtcttccagagcctctcatgacctatgagttacatggagatttcattgttccagccaaaagcggcagtccagaatctcgtgttaatgcgatccatttcttggtacacaaactgccagagaagaataaagagatgttggatattttggtgaaacacttaacaaatgtttcaaatcactccaagcagaacctgatgactgtggcaaacttaggagtggtgtttggaccaactctgatgaggccacaggaagaaactgtcgctgccctcatggacttgaagtttcagaatattgttgtggaaatcttaattgaaaaccatgaaaagatttttcggacgccgcccgatactacattccctgagcccacctgcctgtcagcatcacccccaaatgcgccaccaaggcagtcgaagagacaaggccagagaaccaagaggcccgtggccgtctacaatctttgtctggagctggaagatggtgacaatccttacccttccaaggaggacacccctaccagcagtctggactcactttcctccccgtctcccgtgactacagctgtccctgggcctcctggaccagacaaaaaccaccttctggcagatggagggagctttggagactgggcatccactatcccaggccagacccgatcgtctgtggtccagtggcttaacccacagtctccaaccacaacaagctccaactcagctgtgacacctctttcacccgggtcgtcccctttccccttttctcctcctgctactgtagcggacaagccacctgaaagcatccgcagtcggaaggctcgagccgtgtatccgtgtgaagcagaacacagctcggaattatcttttgatataggagcaatttttgaggatgtacaaacctccagggaacctggctggctagaagggactctgaacggcaagagggggctgattccacagaactacgtcaagctgctgtag

SEQ ID NO: 18 - protein, 786 aa, Homo sapiens [sequence omitted]

SEQ ID NO: 19 - DNA, 2412 bp, Homo sapiens
atgaagaagcagttcaaccgcatgaagcagctggctaaccagaccgtgggcagagctgagaaaacagaagtccttagtgaagatctattacagattgagagacgcctggacacggtgcggtcaatatgccaccattcccataagcgcttggtggcatgtttccagggccagcatggcaccgatgccgagaggagacacaaaaaactgcctctgacagctcttgctcaaaatatgcaagaagcatcgactcagctggaagactctctcctggggaagatgctggagacgtgtggagatgctgagaatcagctggctctcgagctctcccagcacgaagtctttgttgagaaggagatcgtggaccctctgtacggcatagctgaggtggagattcccaacatccagaagcagaggaagcagcttgcaagattggtgttagactgggattcagtcagagccaggtggaaccaagctcacaaatcctcaggaaccaactttcgggggcttccatcaaaaatagatactctaaaggaagagatggatgaagctggaaatagagtagaacagtgcaaggatcaacttgcagcagacatgtacaactttatggccaaagaaggggagtatggcaaattctttgttacgttattagaagcccaagcagattaccatagaaaagcattagcagtcttagaaaagaccctccccgaaatgcgagcccatcaagataagtgggcggaaaaaccagcctttgggactcccctagaagaacacctgaagaggagcgggcgcgagattgcgctgcccattgaagcctgcgtcatgctgcttctggagacaggcatgaaggaggagggccttttccgaattggggctggggcctccaagttaaagaagctgaaagctgctttggactgttctacttctcacctggatgagttctattcagacccccatgctgtagcaggtgctttaaaatcctatttacgggaattgcctgaacctttgatgacttttaatctgtatgaagaatggacacaagttgcaagtgtgcaggatcaagacaaaaaacttcaagacttgtggagaacatgtcagaagttgccaccacaaaattttgttaactttagatatttgatcaagttccttgcaaagcttgctcagaccagcgatgtgaataaaatgactcccagcaacattgcgattgtgttaggccctaacttgttatgggccagaaatgaaggaacacttgctgaaatggcagcagccacatccgtccatgtggttgcagtgattgaacccatcattcagcatgccgactggttcttccctgaagaggtggaatttaatgtatcagaagcatttgtacctctcaccaccccgagttctaatcactcattccacactggaaacgactctgactcggggaccctggagaggaagcggcctgctagcatggcggtgatggaaggagacttggtgaagaaggaaagtcctcccaaaccgaaggaccctgtatctgcagctgtgccagcaccagggagaaacaacagtcagatagcatctggccaaaatcagccccaggcagctgctggctcccaccagctctccatgggccaacctcacaatgctgcagggcccagcccgcatacactgcgccgagctgttaaaaaacccgctccagcacccccgaaaccgggcaacccacctcctggccaccccgggggccagagttcttcaggaacatctcagcatccacccagtctgtcaccaaagccacccacccgaagcccctctcctcccacccagcacacgggccagcctccaggccagccctccgccccctcccagctctcagcaccccggaggtactccagcagcttgtctccaatccaagctcccaatcacccaccgccgcagccccctacgcaggccacgccactgatgcacaccaaacccaatagccagggccctcccaaccccatggcattgcccagtgagcatggacttgagcagccatctcacacccctccccagactccaacgccccccagtactccgcccctaggaaaacagaaccccagtctgccagctcctcagaccctggcagggggtaaccctgaaactgcacagccacatgctggaaccttaccgagaccgagaccagtaccaaagccaaggaaccggcccagcgtgcccccacccccccaacctcctggtgtccactcagctggggacagcagcctcaccaacacagcaccaacagcttccaagatagtaacagactccaattccagggtttcagaaccgcatcgcagcatctttcctgaaatgcactcagactcagccagcaaagacgtgcctggccgcatcctgctggatatagacaatgataccgagagcactgccctgtga

SEQ ID NO: 20 - protein, 803 aa, Homo sapiens
MKKQFNRMKQLANQTVGRAEKTEVLSEDLLQIERRLDTVRSICHHSHKRLVACFQGQHGTDAERRHKKLPLTALAQNMQEASTQLEDSLLGKMLETCGDAENQLALELSQHEVFVEKEIVDPLYGIAEVEIPNIQKQRKQLARLVLDWDSVRARWNQAHKSSGTNFRGLPSKIDTLKEEMDEAGNRVEQCKDQLAADMYNFMAKEGEYGKFFVTLLEAQADYHRKALAVLEKTLPEMRAHQDKWAEKPAFGTPLEEHLKRSGREIALPIEACVMLLLETGMKEEGLFRIGAGASKLKKLKAALDCSTSHLDEFYSDPHAVAGALKSYLRELPEPLMTFNLYEEWTQVASVQDQDKKLQDLWRTCQKLPPQNFVNFRYLIKFLAKLAQTSDVNKMTPSNIAIVLGPNLLWARNEGTLAEMAAATSVHVVAVIEPIIQHADWFFPEEVEFNVSEAFVPLTTPSSNHSFHTGNDSDSGTLERKRPASMAVMEGDLVKKESPPKPKDPVSAAVPAPGRNNSQIASGQNQPQAAAGSHQLSMGQPHNAAGPSPHTLRRAVKKPAPAPPKPGNPPPGHPGGQSSSGTSQHPPSLSPKPPTRSPSPPTQHTGQPPGQPSAPSQLSAPRRYSSSLSPIQAPNHPPPQPPTQATPLMHTKPNSQGPPNPMALPSEHGLEQPSHTPPQTPTPPSTPPLGKQNPSLPAPQTLAGGNPETAQPHAGTLPRPRPVPKPRNRPSVPPPPQPPGVHSAGDSSLTNTAPTASKIVTDSNSRVSEPHRSIFPEMHSDSASKDVPGRILLDIDNDTESTAL

SEQ ID NO: 21 - DNA, 2445 bp, Homo sapiens
atggggctcccagcgctcgagttcagcgactgctgcctcgatagtccgcacttccgagagacgctcaagtcgcacgaagcagagctggacaagaccaacaaattcatcaaggagctcatcaaggacgggaagtcactcataagcgcgctcaagaatttgtcttcagcgaagcggaagtttgcagattccttaaatgaatttaaatttcagtgcataggagatgcagaaacagatgatgagatgtgtatagcaaggtctttgcaggagtttgccactgtcctcaggaatcttgaagatgaacggatacggatgattgagaatgccagcgaggtgctcatcactcccttggagaagtttcgaaaggaacagatcggggctgccaaggaagccaaaaagaaatatgacaaagagacagaaaagtattgtggcatcttagaaaaacacttgaatttgtcttccaaaaagaaagaatctcagcttcaggaggcagacagccaagtggacctggtccggcagcatttctatgaagtatccctggaatatgtcttcaaggtgcaggaagtccaagagagaaagatgtttgagtttgtggagcctctgctggccttcctgcaaggactcttcactttctatcaccatggttacgaactggccaaggatttcggggacttcaagacacagttaaccattagcatacagaacacaagaaatcgctttgaaggcactagatcagaagtggaatcactgatgaaaaagatgaaggagaatccccttgagcacaagaccatcagtccctacaccatggagggatacctctacgtgcaggagaaacgtcactttggaacttcttgggtgaagcactactgtacatatcaacgggattccaaacaaatcaccatggtaccatttgaccaaaagtcaggaggaaaagggggagaagatgaatcagttatcctcaaatcctgcacacggcggaaaacagactccattgagaagaggttttgctttgatgtggaagcagtagacaggccaggggttatcaccatgcaagctttgtcggaagaggaccggaggctctggatggaagccatggatggccgggaacctgtctacaactcgaacaaagacagccagagtgaagggactgcgcagttggacagcattggcttcagcataatcaggaaatgcatccatgctgtggaaaccagagggatcaacgagcaagggctgtatcgaattgtgggggtcaactccagagtgcagaagttgctgagtgtcctgatggaccccaagactgcttctgagacagaaacagatatctgtgctgaatgggagataaagaccatcactagtgctctgaagacctacctaagaatgcttccaggaccactcatgatgtaccagtttcaaagaagtttcatcaaagcagcaaaactggagaaccaggagtctcgggtctctgaaatccacagccttgttcatcggctcccagagaaaaatcggcagatgttacagctgctcatgaaccacttggcaaatgttgctaacaaccacaagcagaatttgatgacggtggcaaaccttggtgtggtgtttggacccactctgctgaggcctcaggaagaaacagtagcagccatcatggacatcaaatttcagaacattgtcattgagatcctaatagaaaaccacgaaaagatatttaacaccgtgcccgatatgcctctcaccaatgcccagctgcacctgtctcggaagaagagcagtgactccaagcccccgtcctgcagcgagaggcccctgacgctcttccacaccgttcagtcaacagagaaacaggaacaaaggaacagcatcatcaactccagtttggaatctgtctcatcaaatccaaacagcatccttaattccagcagcagcttacagcccaacatgaactccagtgacccagacctggctgtggtcaaacccacccggcccaactcactccccccgaatccaagcccaacttcacccctctcgccatcttggcccatgttctcggcgccatccagccctatgcccacctcatccacgtccagcgactcatcccccgtcaggtctgttgcagggtttgtttggttttctgttgctgccgttgttctctcattggctcggtcctctcttcatgcagtgttcagcctcctcgtcaactttgttccctgccatccaaacctgcacttgctttttgacaggccagaagaagcggtacatgaagactccagcacaccgttccggaaggcaaaagccttgtatgcctgcaaagctgaacatgactcagaactttcgttcacagcaggcacggtcttcgataatgttcacccatctcaggagcctggctggttggaggggactctgaacggaaagactggcctcatccctgagaattacgtggagttcctctaa

SEQ ID NO: 22 - protein, 814 aa, Homo sapiens
MGLPALEFSDCCLDSPHFRETLKSHEAELDKTNKFIKELIKDGKSLISALKNLSSAKRKFADSLNEFKFQCIGDAETDDEMCIARSLQEFATVLRNLEDERIRMIENASEVLITPLEKFRKEQIGAAKEAKKKYDKETEKYCGILEKHLNLSSKKKESQLQEADSQVDLVRQHFYEVSLEYVFKVQEVQERKMFEFVEPLLAFLQGLFTFYHHGYELAKDFGDFKTQLTISIQNTRNRFEGTRSEVESLMKKMKENPLEHKTISPYTMEGYLYVQEKRHFGTSWVKHYCTYQRDSKQITMVPFDQKSGGKGGEDESVILKSCTRRKTDSIEKRFCFDVEAVDRPGVITMQALSEEDRRLWMEAMDGREPVYNSNKDSQSEGTAQLDSIGFSIIRKCIHAVETRGINEQGLYRIVGVNSRVQKLLSVLMDPKTASETETDICAEWEIKTITSALKTYLRMLPGPLMMYQFQRSFIKAAKLENQESRVSEIHSLVHRLPEKNRQMLQLLMNHLANVANNHKQNLMTVANLGVVFGPTLLRPQEETVAAIMDIKFQNIVIEILIENHEKIFNTVPDMPLTNAQLHLSRKKSSDSKPPSCSERPLTLFHTVQSTEKQEQRNSIINSSLESVSSNPNSILNSSSSLQPNMNSSDPDLAVVKPTRPNSLPPNPSPTSPLSPSWPMFSAPSSPMPTSSTSSDSSPVRSVAGFVWFSVAAVVLSLARSSLHAVFSLLVNFVPCHPNLHLLFDRPEEAVHEDSSTPFRKAKALYACKAEHDSELSFTAGTVFDNVHPSQEPGWLEGTLNGKTGLIPENYVEFL

SEQ ID NO: 23 - DNA, 3787 bp, Homo sapiens
atgattgctcacaaacagaaaaagacaaagaaaaaacgtgcttgggcatcaggtcaactctctactgatattacaacttctgaaatggggctcaagtccttaagttccaactctatttttgatccggattacatcaaggagttggtgaatgatatcaggaagttctcccacatcttactatatttgaaagaagccatattttcagactgttttaaagaagttattcatatacgtctagaggaactgctccgtgttttaaagtctataatgaataaacatcagaacctcaattctgttgatcttcaaaatgctgcagaaatgctcactgcaaaagtgaaagctgtgaacttcacagaagttaatgaagaaaacaaaaacgatctcttccaggaagtgttttcttctattgaaactttggcatttacctttggaaatatccttacaaacttccttatgggagatgtaggcaatgattcattcttgcgactgcctgtttctcgagaaactaagtcgtttgaaaatgtttctgtggaatcagtggactcatccagtgaaaaaggaaatttttcccctttagaactagacaacgtgctgttaaagaacactgactctatcgagctggctttgtcatatgctaaaacttggtcaaaatatactaagaacatagtttcatgggttgaaaaaaagcttaacttggaattggagtccactagaaatatggtcaagttggcagaggcaactagaactaacattggaattcaggagttcatgccactgcagtctctgtttactaatgctcttcttaatgatatagaaagcagtcaccttttacaacaaacaattgcagctctccaggctaacaaatttgtgcagcctctacttggaaggaaaaatgaaatggaaaaacaaaggaaagaaataaaagagctttggaaacaggagcaaaataaaatgcttgaagcagagaatgctctcaaaaaggcaaaattattatgcatgcaacgtcaagatgaatatgagaaagcaaagtcttccatgtttcgtgcagaagaggagcatctgtcttcaagtggcggattagcaaaaaatctcaacaagcaactagaaaaaaagcgaaggttggaagaggaggctctccaaaaagtagaagaagcagatgaactttacaaagtttgtgtgacaaatgttgaagaaagaagaaatgatgtagaaaataccaaaagagaaattttagcacaactccggacacttgttttccagtgtgatcttacccttaaagcggtaacagttaacctcttccacatgcagcatctgcaggctgcttcccttgcagacagattacagtctctctgtggtagtgccaaactctatgacccaggccaagagtacagtgaatttgtcaaggccacaaattcaactgaagaagaaaaagttgatggaaatgtaaataaacatttaaatagttcccaaccttcaggatttggacctgccaactctttagaggatgttgtacgccttcctgacagttctaataaaattgaagaggacagatgctctaacagtgcagatataacaggtccttcctttataagatcatggacatttgggatgtttagtgattctgagagcactggagggagcagcgaatctagatctctggattcagaatctataagtccaggagactttcatcgaaaacttccacgaacaccatccagtggaactatgtcctctgcagatgatctagatgaaagagagccaccttccccttcagaaactggacccaattcccttggaacatttaagaaaacattgatgtcaaaggcagctctcacacacaagtttcgcaaattgagatcccccacgaaatgtagggattgtgaaggcattgtagtgttccaaggtgttgaatgtgaagagtgtctccttgtttgtcatcgaaagtgtttggaaaatttagtcattatttgtggtcatcagaaacttccaggaaaaatacacttatttggagcagaattcacactagttgcaaaaaaggaaccagatggtatcccttttatactcaaaatatgtgcctcagagattgaaaatagagctttgtgtctacagggaatttatcgtgtgtgtggaaacaaaataaaaactgaaaaattgtgtctagctttggaaaatggtatgcacttggtagatatttcagaatttagttcacatgatatctgtgacgtcttgaaattataccttcggcagctcccagaaccatttattttatttcgattgtacaaggaatttatagaccttgcaaaagagatccaacatgtaaatgaagaacaagagacaaaaaagaatagtcttgaagacaaaaaatggccaaatatgtgtatagaaataaaccgaattcttctaaaaagcaaagaccttctaagacaattgccagcatcaaattttaacagtcttcatttccttatagtacatctaaagcgggtagtagatcatgcagaagaaaacaagatgaactccaaaaacttgggggtgatatttggaccaagtctcattaggccaaggccacaaactgctcctatcaccatctcctcccttgcagagtattcaaatcaagcacgcttggtagagtttctcattacttactcacagaagatcttcgatgggtccctacaaccacaagatgttatgtgtagcataggtgttgttgatcaaggctgttttccaaagcctctgttatcaccagaagaaagagacattgaacgttccatgaagtcactatttttttcttcaaaggaagatatccatacttcagagagtgaaagcaaaatttttgaacgagctacatcatttgaggaatcagaacgcaagcaaaatgcgttaggaaaatgtgatgcatgtctcagtgacaaagcacagttgcttctagaccaagaggctgaatcagcatcccaaaagatagaagatggtaaagcccctaagccactttctctgaaatctgataggtcaacaaacaatgtggagaggcatactccaaggaccaagattagacctgtaagtttgcctgtagatagactacttcttgcaagtcctcctaatgagagaaatggcagaaatatgggaaatgtaaatttagacaagttttgcaagaatcctgcctttgaaggagttaatagaaaagacgctgctactactgtttgttccaaatttaatggctttgaccagcaaactctacagaaaattcaggacaaacagtatgaacaaaacagcctaactgccaagactacaatgatcatgcccagtgcactccaggaaaaaggagtgacaacaagcctccagattagtggggaccattctatcaatgccactcaacccagtaagccatatgcagagccagtcaggtcagtgagagaggcatctgagagacggtcttcagattcctaccctctcgctcctgtcagagcacccagaacactgcagcctcaacattggacaacattttataaaccacatgctcccatcatcagtatcagggggaatgaggagaagccagcttcaccctcagcagcatgccctcctggcacagatcacgatccccacggtctcgtggtgaagtcaatgccagacccagacaaagcatcagcttgtcctgggcaagcaactggtcaacctaaagaagactctgaggagcttggcttgcctgatgtgaatccaatgtgtcagagaccaaggctaaaacgaatgcaacagtttgaagacctcgaagatgaaattccacaatttgtgtagg

SEQ ID NO: 24 - protein, 1261 aa, Homo sapiens
MIAHKQKKTKKKRAWASGQLSTDITTSEMGLKSLSSNSIFDPDYIKELVNDIRKFSHILLYLKEAIFSDCFKEVIHIRLEELLRVLKSIMNKHQNLNSVDLQNAAEMLTAKVKAVNFTEVNEENKNDLFQEVFSSIETLAFTFGNILTNFLMGDVGNDSFLRLPVSRETKSFENVSVESVDSSSEKGNFSPLELDNVLLKNTDSIELALSYAKTWSKYTKNIVSWVEKKLNLELESTRNMVKLAEATRTNIGIQEFMPLQSLFTNALLNDIESSHLLQQTIAALQANKFVQPLLGRKNEMEKQRKEIKELWKQEQNKMLEAENALKKAKLLCMQRQDEYEKAKSSMFRAEEEHLSSSGGLAKNLNKQLEKKRRLEEEALQKVEEADELYKVCVTNVEERRNDVENTKREILAQLRTLVFQCDLTLKAVTVNLFHMQHLQAASLADRLQSLCGSAKLYDPGQEYSEFVKATNSTEEEKVDGNVNKHLNSSQPSGFGPANSLEDVVRLPDSSNKIEEDRCSNSADITGPSFIRSWTFGMFSDSESTGGSSESRSLDSESISPGDFHRKLPRTPSSGTMSSADDLDEREPPSPSETGPNSLGTFKKTLMSKAALTHKFRKLRSPTKCRDCEGIVVFQGVECEECLLVCHRKCLENLVIICGHQKLPGKIHLFGAEFTLVAKKEPDGIPFILKICASEIENRALCLQGIYRVCGNKIKTEKLCLALENGMHLVDISEFSSHDICDVLKLYLRQLPEPFILFRLYKEFIDLAKEIQHVNEEQETKKNSLEDKKWPNMCIEINRILLKSKDLLRQLPASNFNSLHFLIVHLKRVVDHAEENKMNSKNLGVIFGPSLIRPRPQTAPITISSLAEYSNQARLVEFLITYSQKIFDGSLQPQDVMCSIGVVDQGCFPKPLLSPEERDIERSMKSLFFSSKEDIHTSESESKIFERATSFEESERKQNALGKCDACLSDKAQLLLDQEAESASQKIEDGKAPKPLSLKSDRSTNNVERHTPRTKIRPVSLPVDRLLLASPPNERNGRNMGNVNLDKFCKNPAFEGVNRKDAATTVCSKFNGFDQQTLQKIQDKQYEQNSLTAKTTMIMPSALQEKGVTTSLQISGDHSINATQPSKPYAEPVRSVREASERRSSDSYPLAPVRAPRTLQPQHWTTFYKPHAPIISIRGNEEKPASPSAACPPGTDHDPHGLVVKSMPDPDKASACPGQATGQPKEDSEELGLPDVNPMCQRPRLKRMQQFEDLEDEIPQFV

SEQ ID NO: 25 - DNA, 2625 bp, Homo sapiens
atggggctgcccactctggagttcagcgattcctacttggacagcccagatttcagggagcgcttgcagtgtcacgagattgagctggagcgaaccaacaagttcatcaaggagctcattaaggacggctctctgctcattggggcgttgaggaatctgtctatggcagtgcagaaattttcccagtcattgcaagatttccagtttgaatgtattggtgatgctgaaacagatgatgaaattagtattgctcagtcactaaaagaatttgcaagactactcattgcagtagaagaagaaaggcgaagactgatccaaaacgctaacgatgtattaattgcaccacttgagaaatttcgaaaagaacagataggtgcagcaaaagatggaaagaagaagtttgacaaagagagtgaaaaatattactctatccttgaaaagcatttaaatttgtccgcaaagaaaaaggagtctcatttacaagaggcagatacacaaattgaccgagaacatcagaacttctatgaagcatcattagaatatgtctttaaaattcaagaggtccaagaaaaaaagaagtttgaatttgttgaaccgcttttgtcatttcttcagggcttatttactttttaccatgagggatatgaacttgcccaggaatttgcaccgtataagcaacagctgcagttcaacttgcagaatacaaggaataattttgaaagtactcgacaagaggtagagcggttgatgcaaaggatgaaatctgctaaccaggactacagaccacccagccagtggacgatggaaggctatctgtatgtccaggagaaacgaccgcttggttttacatggattaaacattattgtacatatgataagggaagtaaaacatttacaatgagtgtttcagaaatgaaatccagtgggaaaatgaatggccttgttactagctcaccggaaatgtttaaattaaaatcttgtatccgacgaaagacagattcaattgacaaacgattctgctttgacatagaagtagttgaaaggcatgggatcatcacgttacaggccttctcagaagctaataggaaactctggcttgaagccatggatgggaaggaaccgatttatactctgcctgccattataagcaagaaagaagaaatgtatttgaatgaagcagggttcaactttgtgagaaaatgcattcaagctgtggaaacaagaggtatcaccattttaggactctaccgaataggaggagtgaactccaaagttcaaaaactcatgaataccacattttctcctaaatcccctcctgatattgatattgatattgaactgtgggacaataagacgataacaagtgggctgaaaaactacctcaggtgccttgcagaaccactgatgacttacaagttgcacaaagattttatcattgctgttaaatctgatgatcaaaactacagggtggaggctgtacatgcattggtgcacaaattgccggagaaaaacagagagatgctggacatcttaataaagcatctggtcaaagtatcactacacagccaacaaaatctcatgactgtctcaaatcttggtgtcatatttggcccaactctaatgagagcacaggaagaaactgtggctgctatgatgaatattaaatttcagaatattgtggtagaaattctgatagagcactatgaaaagatttttcatactgctccagacccaagcattcctcttcctcagcctcagtctcgatctggatcccgaaggacacgagcaatctgcctctctacagggtctaggaagcccagagggaggtatactccatgcctggccgaacctgatagtgactcctatagcagcagcccagacagcacacctatggggagcattgagtcactctcttctcattcctctgaacaaaatagcactacaaagtcagcttcctgccagcccagggagaaatctggagggattccttggattgcaaccccatcatcttccaatggacagaaaagccttggtctgtggacaactagtcctgaatcaagttccagagaagatgcaaccaagacagatgcagaatcagactgccagagtgttgcttcagtcactagcccaggagacgtttccccacccatagacctagtcaagaaagagccttatgggctttcaggactgaaaagagcttctgcttcttctctcagatccatctctgcagctgaaggaaacaagagctacagtggatctattcaaagcttaacttctgtaggttccaaggagacacccaaagcttcaccaaacccagacctgcctccgaaaatgtgcaggagattaagactagacactgcctcaagcaatggctatcagcggcctggctcagtagtggcagcaaaagctcaactgtttgaaaatgttggttcacctaaaccagtttcttctgggcgccaagccaaagccatgtactcctgtaaagcagagcacagtcatgagctttccttcccacaaggagcaatattttctaatgtgtacccatcagtggaaccaggatggttaaaggcaacttatgaaggcaaaacaggactagttccagaaaattatgttgtcttcctctaa

SEQ ID NO: 26 - protein, 874 aa, Homo sapiens
MGLPTLEFSDSYLDSPDFRERLQCHEIELERTNKFIKELIKDGSLLIGALRNLSMAVQKFSQSLQDFQFECIGDAETDDEISIAQSLKEFARLLIAVEEERRRLIQNANDVLIAPLEKFRKEQIGAAKDGKKKFDKESEKYYSILEKHLNLSAKKKESHLQEADTQIDREHQNFYEASLEYVFKIQEVQEKKKFEFVEPLLSFLQGLFTFYHEGYELAQEFAPYKQQLQFNLQNTRNNFESTRQEVERLMQRMKSANQDYRPPSQWTMEGYLYVQEKRPLGFTWIKHYCTYDKGSKTFTMSVSEMKSSGKMNGLVTSSPEMFKLKSCIRRKTDSIDKRFCFDIEVVERHGIITLQAFSEANRKLWLEAMDGKEPIYTLPAIISKKEEMYLNEAGFNFVRKCIQAVETRGITILGLYRIGGVNSKVQKLMNTTFSPKSPPDIDIDIELWDNKTITSGLKNYLRCLAEPLMTYKLHKDFIIAVKSDDQNYRVEAVHALVHKLPEKNREMLDILIKHLVKVSLHSQQNLMTVSNLGVIFGPTLMRAQEETVAAMMNIKFQNIVVEILIEHYEKIFHTAPDPSIPLPQPQSRSGSRRTRAICLSTGSRKPRGRYTPCLAEPDSDSYSSSPDSTPMGSIESLSSHSSEQNSTTKSASCQPREKSGGIPWIATPSSSNGQKSLGLWTTSPESSSREDATKTDAESDCQSVASVTSPGDVSPPIDLVKKEPYGLSGLKRASASSLRSISAAEGNKSYSGSIQSLTSVGSKETPKASPNPDLPPKMCRRLRLDTASSNGYQRPGSVVAAKAQLFENVGSPKPVSSGRQAKAMYSCKAEHSHELSFPQGAIFSNVYPSVEPGWLKATYEGKTGLVPENYVVFL

SEQ ID NO: 27 - DNA, 2457 bp, Homo sapiens
atgaagaagcagttcaatcgcatgcgccagctggccaaccagacggtgggcagggctgaaaagacagaagttttgagtgaagaccttcttcaggtggagaagcgtctggagctggtgaaacaggtgtcccacagcacgcacaagaagctcaccgcatgtctgcagggccagcaaggggcagaggctgacaagcgctccaaaaagttgcctttgacaacactggctcagtgtctgatggaggggtcagctatcctgggagatgacacacttcttgggaagatgctgaaactctgtggagagacggaggacaagctggctcaggagctgatacattttgagttgcaagtagagagagacgtgattgagcccctgtttttgctggcggaggtggaaatcccaaatattcaaaagcagaggaaacacttagccaagttggtgctggacatggattcctcacgaaccaggtggcagcagacttccaagtcttcaggtttgtccagcagcttacagcctgcgggtgccaaggctgatgccctcagggaagaaatggaagaggctgccaacagagtggagatttgcagggaccagctctcagctgatatgtacagttttgtggccaaagaaattgactatgcaaactactttcaaacgctaatagaagtgcaagctgaataccacaggaagtccctgacactattgcaggctgtattgcctcagatcaaagcacaacaggaggcctgggtagagaagccttccttcgggaagccgctggaggagcacctcaccatcagcggccgggagatcgccttccccatcgaggcgtgtgtgaccatgctgcttgagtgtgggatgcaggaggagggactcttccgagtagccccctctgcctccaaactgaagaagctgaaagcggccctggactgctgcgtggtggatgtgcaggagtactcggcagacccccacgcaattgcaggagctttgaaatcttacctccgagagttgccagaacctcttatgacctttgaactctatgatgagtggatccaggcttccaatgtccaggagcaagacaagaagcttcaggctctatggaatgcttgtgaaaagttgcccaaggccaatcacaacaacatccgatacttgataaaatttttatccaagctgtcagaatatcaagatgtaaacaagatgactcccagtaatatggcaattgttttaggacccaacctcctatggccacaagcagaagggaacattacagagatgatgaccacagtgtcgctgcaaattgttgggatcattgaacctatcatccagcatgcagactggttcttccctggggagatagagttcaacattactggcaattatgggagtccagtacacgtgaaccataatgccaactacagctcaatgccctccccagacatggaccctgctgaccggcgccagcccgagcaggcccgccggcccctcagcgtcgccacggataatatgatgctggagttttacaaaaaggatggccttaggaaaatccaaagcatgggtgtgagggtcatggacacaaactgggtggctcgaagaggctcctcggccggtcggaaagtgtcctgcgccccgccctccatgcagcctcccgccccgcccgccgagctggctgcgcccctgccttcgccgctgccggagcagcccctggacagccccgcggcccccgcgctctctccatccggcctgggcctccagcctgggcccgagcgcaccagcacaacaaaaagcaaggaactttctccaggctctgcacagaaaggaagtccaggctccagccagggcacagcctgtgcagggactcaaccaggggctcaacctggagctcagccgggcgccagccccagccccagccagccgcctgcagaccagagtcctcacaccctccggaaagtttcaaagaagctggcaccgattccacccaaggtcccctttggccagccgggggctatggcagaccagtccgctggccagctgtccccagtcagcctgtcccccaccccgcccagcaccccgtcaccctatggactgagctaccctcaggggtactccttggcctcgggccagctctccccagctgcagctcctcccctggcctctccttctgtctttacaagcactttgagcaaatcgcggcccactcctaagccgcgacagagacctactctgccgcctcctcagcctcccacagtaaacctctcggcctctagtccacagtccacggaggcccccatgctagatggcatgtcccctggggaaagcatgtctacagatcttgtccactttgatattccctcgatccacatagagctcgggtcgacgctccgcctgagtcccctggagcacatgcggcgacactcagtaactgacaagagggactcggaggaggagtctgagagcaccgccctctga

SEQ ID NO: 28 - protein, 818 aa, Homo sapiens
MKKQFNRMRQLANQTVGRAEKTEVLSEDLLQVEKRLELVKQVSHSTHKKLTACLQGQQGAEADKRSKKLPLTTLAQCLMEGSAILGDDTLLGKMLKLCGETEDKLAQELIHFELQVERDVIEPLFLLAEVEIPNIQKQRKHLAKLVLDMDSSRTRWQQTSKSSGLSSSLQPAGAKADALREEMEEAANRVEICRDQLSADMYSFVAKEIDYANYFQTLIEVQAEYHRKSLTLLQAVLPQIKAQQEAWVEKPSFGKPLEEHLTISGREIAFPIEACVTMLLECGMQEEGLFRVAPSASKLKKLKAALDCCVVDVQEYSADPHAIAGALKSYLRELPEPLMTFELYDEWIQASNVQEQDKKLQALWNACEKLPKANHNNIRYLIKFLSKLSEYQDVNKMTPSNMAIVLGPNLLWPQAEGNITEMMTTVSLQIVGIIEPIIQHADWFFPGEIEFNITGNYGSPVHVNHNANYSSMPSPDMDPADRRQPEQARRPLSVATDNMMLEFYKKDGLRKIQSMGVRVMDTNWVARRGSSAGRKVSCAPPSMQPPAPPAELAAPLPSPLPEQPLDSPAAPALSPSGLGLQPGPERTSTTKSKELSPGSAQKGSPGSSQGTACAGTQPGAQPGAQPGASPSPSQPPADQSPHTLRKVSKKLAPIPPKVPFGQPGAMADQSAGQLSPVSLSPTPPSTPSPYGLSYPQGYSLASGQLSPAAAPPLASPSVFTSTLSKSRPTPKPRQRPTLPPPQPPTVNLSASSPQSTEAPMLDGMSPGESMSTDLVHFDIPSIHIELGSTLRLSPLEHMRRHSVTDKRDSEEESESTAL

SEQ ID NO: 29 - DNA, 3411 bp, Homo sapiens
atgttctccaggaagaaacgagagctcatgaaaaccccttccatctcgaaaaagaaccgcgcgggaagccccagcccgcagccctcgggggagctgcccaggaaggatggggctgacgcggtgttccccggaccaagcctggagccgcccgctgggtcctccggcgtcaaggccacagggaccctcaagcggcccaccagcctgagccgccacgccagcgcggctggcttccccctgtcgggtgctgcctcctggacactgggccggagccaccggagcccactgacagccgccagcccgggcgagctgcccaccgagggtgccggcccggacgtcgtcgaggacatctcccatctgctggcggacgtggcccgcttcgctgagggccttgagaaacttaaggagtgtgtgttgcgtgacgacctccttgaggcccgccgcccgcgggcccacgagtgcctgggtgaggctctgcgtgtcatgcatcagatcatctccaagtacccgctgctgaacaccgtggagacgctcaccgcagccggcaccctcattgccaaggtcaaagccttccattatgagagcaacaatgatctggagaaacaggagttcgagaaggccctggagacgattgctgtggccttcagtagcacagtgtccgagttcctcatgggtgaagtggacagcagcaccctcctagcagtgcctcctggggactcgagccagtccatggaaagcctgtatggaccgggcagtgagggcacgcctcccagcctggaagactgtgacgccggctgcctgcccgccgaggaggtggacgtgctgctacagcgctgtgaggggggcgtggatgccgcactgctgtatgccaagaacatggccaagtacatgaaggacctcatcagctacctggagaagcggacgacgctggagatggagtttgccaagggcctgcagaagatcgctcacaactgcagacagagcgtcatgcaggagccccacatgccgctcctgtccatctactcgctggccctggagcaggacctggagttcggccacagcatggtgcaggcggtgggcaccttgcagacccagaccttcatgcagcccctgaccctgcggcggcttgaacacgagaagcgcaggaaggagatcaaggaggcctggcaccgtgcccagaggaagctgcaagaggcggagtccaacctgcgcaaggccaagcagggttacgtgcagcgctgcgaggaccacgacaaggctcgcttcctcgtggccaaggcggaggaggagcaggctggcagcgcgccgggagcaggcagcacggccaccaagaccctggacaagcggcggcggctggaggaggaggccaagaacaaggcggaggaagctatggccacctaccgcacctgcgtggccgacgcgaagacgcagaagcaggagctggaggataccaaggtgacggcgctgcggcagatccaggaggtcatccggcagagcgaccaaaccatcaagtcggccacgatctcctactaccagatgatgcatatgcagacggcgccgctgcccgtgcacttccagatgctgtgtgagagcagcaagctgtatgacccaggccagcagtacgcctcccacgtgcgccagctgcagcgggaccaggagcccgatgtgcactacgactttgagccccacgtctccgccaacgcctggtcccccgtcatgcgtgcccggaagagcagcttcaacgtgagtgatgtggcgcggccggaggctgccgggagccccccagaagaaggcgggtgcactgagggcacacctgccaaggaccacagggccgggcgaggacaccaggttcacaagtcatggccgctctcgatctcagactcggacagtgggctggaccccggccctggcgcaggggactttaagaagttcgagcggacgtcatccagtggtaccatgtcgtccacggaggagctggtggacccagacggtggagccggggcttcagcctttgagcaggctgacctcaacggcatgacccccgagctgccggtggccgtgcccagtggaccgttccgccacgaggggctgtccaaggcggcccgtactcaccggctccggaagctccgcacgcccgccaagtgccgcgagtgcaacagctacgtctacttccagggtgctgagtgtgaagagtgctgcctggcctgccacaagaaatgtctggagacgctggccatacagtgcgggcacaagaagctgcaaggccgcctgcagctgttcggccaggacttcagccacgcggcccgcagcgcccccgacggcgtgcccttcatcgtcaagaagtgcgtctgcgagatcgagcggcgggcgctgcgcaccaagggcatctaccgggtcaatggggtaaagacacgcgtggagaagctgtgccaggccttcgagaacggcaaggagctggtcgagctgtcgcaggcctcgccccacgacatcagcaacgtcctcaagctctacctgcgtcagcttcccgagccgctcatctccttccgcctctaccacgagctcgtagggctggccaaggacagcctgaaggcagaggccgaggccaaggcggcgtcccggggccggcaggacggctcggagagcgaggcagtggcggtggccctggcaggtcggctgcgggagctcctgcgggacctgccgcctgagaaccgggcctcgctgcagtacctgctgcgtcacctacgcaggatcgtggaggtggagcaggacaacaagatgacccccgggaacctgggcatcgtgttcgggcccacgctgcttcggccacggcccaccgaggccaccgtgtccctctcctccctggtggattatccccatcaggcccgcgtcatcgagactctcatcgtccactacggcctggtcttcgaggaggagccggaggagacccccgggggccaggacgagtcatccaaccagcgagctgaggtagtcgtccaggtgccgtacctggaggcgggcgaggcggtggtctacccgctgcaggaggcggcggcggacgggtgcagagaatcccgagttgtgtccaacgattcggactcggacctagaggaggcctccgagctgctgtcctcatcggaggccagtgccctgggccacctcagcttcctggagcagcagcagagcgaggccagcctagaggtggcttctggcagccacagcggcagtgaggagcagctggaggccacagcccgggaggacggggacggggacgaggacggcccggcccagcagctctcaggattcaacaccaaccagtccaacaacgtgctgcaggccccactgccccccatgaggctccgtggcgggcggatgacactgggctcctgcagggaaaggcagccggaattcgtgtga

SEQ ID NO: 30 - protein, 1137 aa, Homo sapiens
MFSRKKRELMKTPSISKKNRAGSPSPQPSGELPRKDGADAVFPGPSLEPPAGSSGVKATGTLKRPTSLSRHASAAGFPLSGAASWTLGRSHRSPLTAASPGELPTEGAGPDVVEDISHLLADVARFAEGLEKLKECVLRDDLLEARRPRAHECLGEALRVMHQIISKYPLLNTVETLTAAGTLIAKVKAFHYESNNDLEKQEFEKALETIAVAFSSTVSEFLMGEVDSSTLLAVPPGDSSQSMESLYGPGSEGTPPSLEDCDAGCLPAEEVDVLLQRCEGGVDAALLYAKNMAKYMKDLISYLEKRTTLEMEFAKGLQKIAHNCRQSVMQEPHMPLLSIYSLALEQDLEFGHSMVQAVGTLQTQTFMQPLTLRRLEHEKRRKEIKEAWHRAQRKLQEAESNLRKAKQGYVQRCEDHDKARFLVAKAEEEQAGSAPGAGSTATKTLDKRRRLEEEAKNKAEEAMATYRTCVADAKTQKQELEDTKVTALRQIQEVIRQSDQTIKSATISYYQMMHMQTAPLPVHFQMLCESSKLYDPGQQYASHVRQLQRDQEPDVHYDFEPHVSANAWSPVMRARKSSFNVSDVARPEAAGSPPEEGGCTEGTPAKDHRAGRGHQVHKSWPLSISDSDSGLDPGPGAGDFKKFERTSSSGTMSSTEELVDPDGGAGASAFEQADLNGMTPELPVAVPSGPFRHEGLSKAARTHRLRKLRTPAKCRECNSYVYFQGAECEECCLACHKKCLETLAIQCGHKKLQGRLQLFGQDFSHAARSAPDGVPFIVKKCVCEIERRALRTKGIYRVNGVKTRVEKLCQAFENGKELVELSQASPHDISNVLKLYLRQLPEPLISFRLYHELVGLAKDSLKAEAEAKAASRGRQDGSESEAVAVALAGRLRELLRDLPPENRASLQYLLRHLRRIVEVEQDNKMTPGNLGIVFGPTLLRPRPTEATVSLSSLVDYPHQARVIETLIVHYGLVFEEEPEETPGGQDESSNQRAEVVVQVPYLEAGEAVVYPLQEAAADGCRESRVVSNDSDSDLEEASELLSSSEASALGHLSFLEQQQSEASLEVASGSHSGSEEQLEATAREDGDGDEDGPAQQLSGFNTNQSNNVLQAPLPPMRLRGGRMTLGSCRERQPEFVA

SEQ ID NO: 31 - DNA, 2028 bp, Homo sapiens
atggccaagcatggagccgacgagccatcctccaggtcagggagtccggacagggaaggtagggcctctgaggacagatcgctgcttcatcagaggctggctgtccgggagctcatcgacactgaggtctcctacttgcacatgctccagctctgtgcctctgacatcaggagccgcctccagcagttgccgcagggagatctggatgtcctgttctcaaacattgatgatatcatcaaagtgaacagcagattcctccatgatctgcaggagacagcctccaaggaagaggaacaagtgcagctagttggtaacatatttctggaattccaagaggagttggagcaagtctataaggtctactgtgccagctacgaccaggccttgctactggtggacacgtaccggaaggagccggagctgcagcggcacatccagggcatcgttgaggcggtggtgccgcaagctggatcttcaggcctcagtttcttgctggtaattcctctgcagaggatcaccaggtacccactgctgctgcagaaaatcctggagaacacagtccctgatgccagtgcctatcctgtccttcagagggctgtctctgccctccaggacgtgaacaccaatatcaatgagtacaagatgcgcaaggaagtggcctccaagtacaccaaggtagagcagctgaccctccgggagcggctggcccgcatcaacacacacaccctctccaagaagaccacccggctgagccagctgctgaagcaggaggcggggctgatccccaggacagaagacaaggaatttgatgatttagaagagaggttccagtgggtgtctctgtgtgtgactgagctgaagaacaacgtggctgcttacctggacaatctgcaggctttcctctacttcaggccgcacgaatacaatctggacatccccgaggggcctgcagtgcagtattgcaatttggcaagagaccttcaccttgaggccttcctgaaatttaagcaacggctagaaggcctggtgtggcagccactgtgcagcctggccaaagccctgcttggccctcagaacctgatcaagaagcgtctggacaagctactggactttgagcgggtggaagagaagctgctggaggtgggcagtgtgacctaccaggaggaggccgcccggcacacataccaggcactcaactcgctgctagtggctgagctcccacagtttaaccagctggtcatgcagtggctgggccagatcmtgtgcacattcgtgaccctccagagggaccttgcaaagcaagtgctgcagagggcagagggaagcatggcccagctgccccaccaccacgtcccagagcctgccttcaggaagctggtggaggacgcactgggccggacgagtaaccagcttcgctcctttcaagagacctttgagaaagtgcagccacctcccaccacacaacygctccttccagggtctgaacgccaggtgcaggctctcctgagcaggtatggccctgggaagctgtaccaggtgacaagcaacatcagtgggactgggactctggacctgactctgcctcggggccaaatcgtggccatccttcaaaacaaggacaccaaaggcaacagcggccgctggctggtggacaccgggggacatcgtgggtatgtgccggctgggaaactrcagctgtaccatgtggtccccagtgcagaggagctcagaaggcaggcggggctgaacaaagacccccgatgtctaacaccggagcccagcccagctctagtgccctctattcccaccrtgaaccaggtcatagccgcgtacccttttgtggccagaagcagccatgaagtgagcctgcaggcaggccagcctgtgaccatcctggaggcccaggacaagaaggggaaccctgagtggagcctggtggaagtgaatggacagaggggttatgtgccttctggcttcttggccagggctcggagcccagttctgtggggctggagtctgccctcttag

SEQ ID NO: 32 - protein, 675 aa, Homo sapiens
MAKHGADEPSSRSGSPDREGRASEDRSLLHQRLAVRELIDTEVSYLHMLQLCASDIRSRLQQLPQGDLDVLFSNIDDIIKVNSRFLHDLQETASKEEEQVQLVGNIFLEFQEELEQVYKVYCASYDQALLLVDTYRKEPELQRHIQGIVEAVVPQAGSSGLSFLLVIPLQRITRYPLLLQKILENTVPDASAYPVLQRAVSALQDVNTNINEYKMRKEVASKYTKVEQLTLRERLARINTHTLSKKTTRLSQLLKQEAGLIPRTEDKEFDDLEERFQWVSLCVTELKNNVAAYLDNLQAFLYFRPHEYNLDIPEGPAVQYCNLARDLHLEAFLKFKQRLEGLVWQPLCSLAKALLGPQNLIKKRLDKLLDFERVEEKLLEVGSVTYQEEAARHTYQALNSLLVAELPQFNQLVMQWLGQIMCTFVTLQRDLAKQVLQRAEGSMAQLPHHHVPEPAFRKLVEDALGRTSNQLRSFQETFEKVQPPPTTQPLLPGSERQVQALLSRYGPGKLYQVTSNISGTGTLDLTLPRGQIVAILQNKDTKGNSGRWLVDTGGHRGYVPAGKLQLYHVVPSAEELRRQAGLNKDPRCLTPEPSPALVPSIPTMNQVIAAYPFVARSSHEVSLQAGQPVTILEAQDKKGNPEWSLVEVNGQRGYVPSGFLARARSPVLWGWSLPS

SEQ ID NO: 33 - DNA, 2334 bp, Homo sapiens
atggagcccaaagaagccactgggaaagaaaacatggtcaccaagaaaaagaatctggccttcttgaggtctagactctatatgctggagagaaggaagactgacactgtggttgagagcagtgtttctggggaccactctggcaccttgaggaggagccaatctgacaggaccgaatacaaccagaaattacaagaaaagatgactccacagggtgagtgttctgtagctgagaccttaaccccagaggaagagcatcatatgaagaggatgatggcaaagcgggaaaagatcattaaggagctgatacagacagaaaaggattatctcaatgatctagagctgtgtgttagggaagtggttcagcccctgagaaataaaaagactgataggctggatgtggatagcttgtttagcaacattgagtccgtgcatcagatatcagccaagctgctgtcattgttggaagaggccacaacagacgtggaaccggccatgcaagtaattggagaagtattcttgcagattaaagggccactggaagatatttataaaatctactgctatcaccatgatgaagcacatagtatactggagtcctatgaaaaggaagaagagctgaaggaacatttgagccactgtatccagtccttaaagaaaatatacatgcaagaaggcaaaccaaacttattggacatgggctctttgatgatcaaaccaattcaacgtgtgatgaaataccccctattactgtgcgaacttcggaattccacccctccctctcacccagattacagagcactggacgatgcctttgctgctgtgaaggacattaatgttaacatcaatgaacttaaaagaaggaaagatttagttctaaaatacaagaagaatgacgaggatgaatcacttaaagacaaattgtctaaactaaatattcattcaattagcaagaaatcaaaaagagtgacaaatcatctgaagattctgaccagaggagaatcacaggttaaagacaatacctttaacagagaagaaaagctgtttagagctttagaaaagactgtgaggctttgtgtgaagaacatttcactctgtcttcaacacatacaggatgccatgcccctggctctgcagagtgtgatggaccttcaggagatttcatacaacaaagacgatgagatggactattctgagaccctaagtaatgccttaaattcgtgtcatgactttgcatctcacttacagagactcatcctgacccccttgtcagccctgctgtccttattcccagggcctcacaagctcatccagaaacgctatgacaaactgctggattgcaacagctacctgcagcgatcaacgggagaggagtcagacttggccaaaaaggagtatgaggccctcaacgcccagcttgtggaggagctccaggcattcaaccaggctgctcggaagattctgttgaactgtctatgcagcttcattaccctccttagggacctgatgctcgtggcacagcaggcttactccacacttgtgccgatgccactgttggtttcaagcatttctgagattcagaatcaagtactagaagagatccaaaatttgaattgtgtgaaagaaaacagtgccacctttattgagaggaaactcagttttgaaaagaagaaacctgtgcagattctgccagaaatgccacatcaaactgacattcatcgctccaaacttctatccacatatagtgcagaggaactctatcaagctaagcgcaagtgcaatgctacacaagaatatgacatcaatcttctggaaggagacttggtggctgtgatagaacagaaagatccactggggagtacaagcaggtggcttgtggacacaggaaatgtgaaaggatatgtttattcctccttcctaaaaccctacaatccagcaaaaatgcagaaagtggatgctgagaacaggttctgtgacgatgattttgagaacatcagcctcttcgtgtcttcacggccagctagtgacagtgtcacaggcacctcagaaagcagcattggtgatagcagctcatctcttagtggcacatgtggaaagtttgaaacaaatggtactgatgttgacagttttcaagaagtagacgaacagattttctatgcagttcatgcttttcaagcacggagtgaccatgaactcagccttcaggaataccagagagttcatatactcaggttttgtgacctaagtggcaataaagagtggtggttagctgaagctcaagggcagaaaggatacgtgccagctaactaccttggaaagatgacttatgcttaa

SEQ ID NO: 34 - protein, 777 aa, Homo sapiens
MEPKEATGKENMVTKKKNLAFLRSRLYMLERRKTDTVVESSVSGDHSGTLRRSQSDRTEYNQKLQEKMTPQGECSVAETLTPEEEHHMKRMMAKREKIIKELIQTEKDYLNDLELCVREVVQPLRNKKTDRLDVDSLFSNIESVHQISAKLLSLLEEATTDVEPAMQVIGEVFLQIKGPLEDIYKIYCYHHDEAHSILESYEKEEELKEHLSHCIQSLKKIYMQEGKPNLLDMGSLMIKPIQRVMKYPLLLCELRNSTPPSHPDYRALDDAFAAVKDINVNINELKRRKDLVLKYKKNDEDESLKDKLSKLNIHSISKKSKRVTNHLKILTRGESQVKDNTFNREEKLFRALEKTVRLCVKNISLCLQHIQDAMPLALQSVMDLQEISYNKDDEMDYSETLSNALNSCHDFASHLQRLILTPLSALLSLFPGPHKLIQKRYDKLLDCNSYLQRSTGEESDLAKKEYEALNAQLVEELQAFNQAARKILLNCLCSFITLLRDLMLVAQQAYSTLVPMPLLVSSISEIQNQVLEEIQNLNCVKENSATFIERKLSFEKKKPVQILPEMPHQTDIHRSKLLSTYSAEELYQAKRKCNATQEYDINLLEGDLVAVIEQKDPLGSTSRWLVDTGNVKGYVYSSFLKPYNPAKMQKVDAENRFCDDDFENISLFVSSRPASDSVTGTSESSIGDSSSSLSGTCGKFETNGTDVDSFQEVDEQIFYAVHAFQARSDHELSLQEYQRVHILRFCDLSGNKEWWLAEAQGQKGYVPANYLGKMTYA

SEQ ID NO: 35 - DNA, 1566 bp, Homo sapiens
atgtctctgtctcgctcagaggagatgcaccggctcacggaaaatgtctataagaccatcatggagcagttcaaccctagcctccggaacttcatcgccatggggaagaattacgagaaggcactggcaggtgtgacgtatgcagccaaaggctactttgacgccctggtgaagatgggggagctggccagcgagagccagggctccaaagaactcggagacgttctcttccagatggctgaagtccacaggcagatccagaatcagctggaagaaatgctgaagtcttttcacaacgagctgcttacgcagctggagcagaaggtggagctggactccaggtatctgagtgctgcgctgaagaaataccagactgagcaaaggagcaaaggcgacgccctggacaagtgtcaggctgagctgaagaagcttcggaagaagagccagggcagcaagaatcctcagaagtactcggacaaggagctgcagtacatcgacgccatcagcaacaagcagggcgagctggagaattacgtgtccgacggctacaagaccgcactgacagaggagcgcaggcgcttctgcttcctggtggagaagcagtgcgccgtggccaagaactccgcggcctaccactccaagggcaaggagctgctggcgcagaagctgccgctgtggcaacaggcctgtgccgaccccagcaagatcccggagcgcgcggtgcagctcatgcagcaggtggccagcaacggcgccaccctccccagcgccctgtcggcctccaagtccaacctggtcatttccgaccccattccgggggccaagcccctgccggtgccccccgagctggcaccgttcgtggggcggatgtctgcccaggagagcacacccatcatgaacggcgtcacaggcccggatggcgaggactacagcccgtgggctgaccgcaaggctgcccagcccaaatccctgtctcctccgcagtctcagagcaagctcagcgactcctactccaacacactccccgtgcgcaagagcgtgaccccaaaaaacagctatgccaccaccgagaacaagactctgcctcgctcgagctccatggcagccggcctggagcgcaatggccgtatgcgggtgaaggccatcttctcccacgctgctggggacaacagcaccctcctgagcttcaaggagggtgacctcattaccctgctggtgcctgaggcccgcgatggctggcactacggagagagtgagaagaccaagatgcggggctggtttcccttctcctacacccgggtcttggacagcgatggcagtgacaggctgcacatgagcctgcagcaagggaagagcagcagcacgggcaacctcctggacaaggacgacctggccatcccaccccccgattacggcgccgcctcccgggccttccccgcccagacggccagcggcttcaagcagaggccctacagtgtggccgtgcccgccttctcccagggcctggatgactatggagcgcggtccatgagcagtggcagcggcacgctggtgtccacagtgtga

SEQ ID NO: 36 - protein, 521 aa, Homo sapiens
MSLSRSEEMHRLTENVYKTIMEQFNPSLRNFIAMGKNYEKALAGVTYAAKGYFDALVKMGELASESQGSKELGDVLFQMAEVHRQIQNQLEEMLKSFHNELLTQLEQKVELDSRYLSAALKKYQTEQRSKGDALDKCQAELKKLRKKSQGSKNPQKYSDKELQYIDAISNKQGELENYVSDGYKTALTEERRRFCFLVEKQCAVAKNSAAYHSKGKELLAQKLPLWQQACADPSKIPERAVQLMQQVASNGATLPSALSASKSNLVISDPIPGAKPLPVPPELAPFVGRMSAQESTPIMNGVTGPDGEDYSPWADRKAAQPKSLSPPQSQSKLSDSYSNTLPVRKSVTPKNSYATTENKTLPRSSSMAAGLERNGRMRVKAIFSHAAGDNSTLLSFKEGDLITLLVPEARDGWHYGESEKTKMRGWFPFSYTRVLDSDGSDRLHMSLQQGKSSSTGNLLDKDDLAIPPPDYGAASRAFPAQTASGFKQRPYSVAVPAFSQGLDDYGARSMSSGSGTLVSTV

SEQ ID NO: 37 - DNA, 1536 bp, Homo sapiens
atgtcccgggggcccgaggaggtgaaccggctcacggagagcacctaccggaatgttatggaacagttcaatcctgggctgcgaaatttaataaacctggggaaaaattatgagaaagctgtaaacgctatgatcctggcaggaaaagcctactacgatggagtggccaagatcggtgagattgccactgggtcccccgtgtcaactgaactgggacatgtcctcatagagatttcaagtacccacaagaaactcaacgagagtcttgatgaaaattttaaaaaattccacaaagagattatccatgagctggagaagaagatagaacttgacgtgaaatatatgaacgcaactctaaaaagataccaaacagaacacaagaataaattagagtctttggagaaatcccaagctgagttgaagaagatcagaaggaaaagccaaggaagccgaaacgcactcaaatatgaacacaaagaaattgagtatgtggagaccgttacttctcgtcagagtgaaatccagaaattcattgcagatggttgcaaagaggctctgcttgaagagaagaggcgcttctgctttctggttgataagcactgtggctttgcaaaccacatacattattatcacttacagtctgcagaactactgaattccaagctgcctcggtggcaggagacctgtgttgatgccatcaaagtgccagagaaaatcatgaatatgatcgaagaaataaagaccccagcctctacccccgtgtctggaactcctcaggcttcacccatgatcgagagaagcaatgtggttaggaaagattacgacaccctttctaaatgctcaccaaagatgccccccgctccttcaggcagagcatataccagtcccttgatcgatatgtttaataacccagccacggctgccccgaattcacaaagggtaaataattcaacaggtacttccgaagatcccagtttacagcgatcagtttcggttgcaacgggactgaacatgatgaagaagcagaaagtgaagaccatcttcccgcacactgcgggctccaacaagaccttactcagctttgcacagggagatgtcatcacgctgctcatccccgaggagaaggatggctggctctatggagaacacgacgtgtccaaggcgaggggttggttcccgtcgtcgtacacgaagttgctggaagaaaatgagacagaagcagtgaccgtgcccacgccaagccccacaccagtgagaagcatcagcaccgtgaacttgtctgagaatagcagtgttgtcatccccccacccgactacttggaatgcctatccatgggggcagctgccgacaggagagcagattcggccaggacgacatccacctttaaggccccagcgtccaagcccgagaccgcggctcctaacgatgccaacgggactgcaaagccgccttttctcagcggagaaaacccctttgccactgtgaaactccgcccgactgtgacgaatgatcgctcagcacccatcattcgatga

SEQ ID NO: 38 - protein, 511 aa, Homo sapiens
MSRGPEEVNRLTESTYRNVMEQFNPGLRNLINLGKNYEKAVNAMILAGKAYYDGVAKIGEIATGSPVSTELGHVLIEISSTHKKLNESLDENFKKFHKEIIHELEKKIELDVKYMNATLKRYQTEHKNKLESLEKSQAELKKIRRKSQGSRNALKYEHKEIEYVETVTSRQSEIQKFIADGCKEALLEEKRRFCFLVDKHCGFANHIHYYHLQSAELLNSKLPRWQETCVDAIKVPEKIMNMIEEIKTPASTPVSGTPQASPMIERSNVVRKDYDTLSKCSPKMPPAPSGRAYTSPLIDMFNNPATAAPNSQRVNNSTGTSEDPSLQRSVSVATGLNMMKKQKVKTIFPHTAGSNKTLLSFAQGDVITLLIPEEKDGWLYGEHDVSKARGWFPSSYTKLLEENETEAVTVPTPSPTPVRSISTVNLSENSSVVIPPPDYLECLSMGAAADRRADSARTTSTFKAPASKPETAAPNDANGTAKPPFLSGENPFATVKLRPTVTNDRSAPIIR

SEQ ID NO: 39 - DNA, 4734 bp, Homo sapiens
atggaggctggctcagtggttcgagccatttttgacttctgccctagcgtatcagaagaactgccgctctttgtgggagatattattgaggtgctggcagtggtggatgaattttggcttctaggaaagaaggaggatgttacaggacaattccccagcagttttgtggaaattgtgaccattcccagtctaaaagagggagagaggctgtttgtgtgcatttgtgaattcacatcccaagagttggataatcttcccctccatcgaggtgacctggtgattctcgatggcattcccactgcaggctggctgcagggccgaagctgctggggcgcacggggcttcttcccatcttcatgtgtccgcgagctctgcctctcctcacagagccggcagtggcactcccagagcgccctgtttcagattccggaatattccatgggacaagcccgggccctaatggggctttctgctcagctggatgaagagctggacttcagggaaggggatgtgatcaccattattggagttcctgaaccaggctggtttgaaggggagttagagggccgaagaggcatttttccagaaggttttgtagagctgttggggcccctgaggactgtggatgagtcagtaagttctggaaatcaagatgactgcattgttaatggtgaagtagatacccccgtaggagaagaagagatagggccggatgaggatgaggaggagccagggacctatggggtcgccctgtacagattccaagccctggagccaaatgagctggatttcgaggtcggggataaaatccgaattctggcgaccttggaagatggctggctggaaggatccctgaagggcaggacaggcatctttccttaccggtttgtgaaattatgtcctgacacacgggtggaggaaaccatggctctgccccaggaaggcagccttgccaggatcccggaaacttctttggattgtttggagaacaccttaggagtagaggaacaaagacatgaaaccagtgaccatgaggccgaggagcctgactgcattatttctgaagcacccacttctcccctcggtcatctgacttcagagtatgacacagacagaaactcttatcaggacgaggacaccgcaggagggcccccgagaagcccaggcgtggagtgggaaatgcctcttgccacagactctcccacatctgaccctacagaagtagtcaatggtatttcctcccaacctcaggtcccttttcatcccaacttgcagaaaagccagtattattctacagtgggaggaagccacccgcactcagaacagtaccccgaccttcttcccctagaagcaaggactagagactatgccagcctacctcccaaaagaatgtattcccagctaaaaactcttcagaagccagtgctccctctttacaggggctcttctgtttcagcttcaagggtagtcaaacccagacaatcaagtcctcagctccacaacctagcaagttatactaaaaagcaccacacgtccagtgtttactccatctcagagagattggagatgaagcctggtccgcaagcccaagggcttgttatggaagcagcaacacattcacagggagacggcagcactgacctggactcgaagctgacacaacagctgatcgagtttgagaagagcttggcagggcccggcacagagccagataaaattttacgccacttttcaatcatggactttaactctgagaaggatattgtccgaggttcctcaaagttaatcaccgagcaggagctgccggaaaggagaaaggccctaaggccaccgccacctcgtccctgtactccggtatccacttctccccatttgctggttgaccagaacctaaaacctgcaccacccttggtggtgcgaccctctcgcccagctcccctgcctccctcagcacagcagagaacgaatgcggtatcccccaagctcctatctcgacaccgtcctacctgtgagaccttagaaaaggagggccctggtcatatgggaaggagtctggaccagacctccccatgccccttagtgctggtgaggattgaggaaatggagcgggacttggatatgtacagtagagctcaagaagagctaaacctcatgctggaggagaagcaggatgaatcatcaagagcagagaccctcgaggatctcaagttctgtgaaagtaacattgaaagtttgaatatggaactccagcaactaagagaaatgacgctcctctcctcccagtcttcatcactggtggccccttctgggtctgtgtctgccgaaaatccagagcagaggatgctggagaagagagccaaggtcatagaagaacttcttcagacagaaagagactacattcgggatctggaaatgtgtattgagcggatcatggtacccatgcagcaggcacaggtaccaaacattgattttgagggactttttggaaatatgcagatggtgattaaggtctcgaagcaattattggctgctctggaaatcagcgatgctgtaggacctgtgtttcttggtcaccgggatgagcttgagggaacatacaagatttactgccagaatcatgatgaggccattgcgctgcttgaaatctacgagaaggatgagaagatccagaagcatcttcaggactccttggcagatctgaagagcctatacaacgaatggggatgcacaaattatattaacctgggctccttcctcatcaaaccagtacagagagtaatgcgttacccgctgttgctaatggagttgctgaattccaccccagaatcccacccagataaagtgcctttaaccaatgcagtccttgcggtcaaggaaatcaacgttaacattaatgaatataaacggcgaaaggacctggtcctcaagtaccgtaagggtgatgaagatagccttatggagaaaatttccaaactgaacatccactccatcatcaagaaatccaaccgagttagcagtcacctgaagcatctcactggctttgctcctcagataaaagatgaagtatttgaagaaacagaaaaaaacttccgaatgcaagaaagattgattaagtcttttatccgagacctgtctctctacctccagcacatccgggagtccgcatgtgtgaaagtggtggctgctgtgagcatgtgggatgtgtgcatggagagaggacaccgggacctggagcagtttgagagggtgcatcgctacatcagtgaccagctcttcacaaactttaaggagaggacagagcggcttgtcatctcccccttaaatcagttactgagcatgtttacagggccccataagctggtacagaaacgctttgacaagctcctggacttctataactgtacagaacgggcagaaaagctaaaggacaagaagaccctggaggagctgcagtcggcccggaacaactatgaggccctgaatgcacagctgctggatgagctgcccaagttccaccagtacgcccagggcctcttcaccaactgtgtccacggctatgctgaagcccactgtgactttgtgcaccaggctctggagcaattaaagccactgctttcgttactcaaagtggctggcagagagggaaaccttatcgccatcttccacgaagagcacagcagagttctgcagcaactccaggtttttaccttcttcccggagtctcttccagctaccaagaaaccgtttgagaggaaaaccattgaccgccagtctgctcgaaagccactcctgggcctgccaagttacatgctacagtcagaagaactccgggcctccctcctggccaggtatccccctgaaaaactcttccaggcagaacggaacttcaatgctgcccaagacttggatgtctcacttttggaaggtgacctggtgggtgtgattaagaaaaaagaccccatgggcagccagaaccgctggctgattgacaatggagtcaccaaaggcttcgtgtacagctctttcctaaagccctacaatcctcgccgcagccactccgatgcctccgtgggtagccactcctccacagagtctgagcacggcagctcctcccccaggttcccacgccagaacagcggcagcaccctgaccttcaaccccagcagcatggctgtatcctttacctcggggtcttgccagaagcagcctcaagatgcatctcctccgccaaaagaatgtgaccaaggaactctcagtgcatccctaaatccgagtaattcagagagtagtccttccagatgcccttcagacccagactccacctcccagccaaggtcaggggactctgcagatgtagctagagatgtaaagcagcccactgccacgccgaggagctaccggaacttcaggcatccagaaatagttggctactccgtaccaggacgaaatgggcaaagtcaagacctcgtcaaaggatgtgcaagaacagcccaggctccggaagacagaagtacagagccagatggcagtgaggcagaaggcaaccaggtctattttgctgtctacaccttcaaggcacgaaacccaaatgagctgagcgtgtcagccaatcagaaactcaagatcctcgagtttaaagatgttacaggaaatacagagtggtggttagctgaggttaacgggaagaagggctacgttccctccaattatatccgcaaaaccgagtacacctga

SEQ ID NO: 40 - protein, 1577 aa, Homo sapiens
MEAGSVVRAIFDFCPSVSEELPLFVGDIIEVLAVVDEFWLLGKKEDVTGQFPSSFVEIVTIPSLKEGERLFVCICEFTSQELDNLPLHRGDLVILDGIPTAGWLQGRSCWGARGFFPSSCVRELCLSSQSRQWHSQSALFQIPEYSMGQARALMGLSAQLDEELDFREGDVITIIGVPEPGWFEGELEGRRGIFPEGFVELLGPLRTVDESVSSGNQDDCIVNGEVDTPVGEEEIGPDEDEEEPGTYGVALYRFQALEPNELDFEVGDKIRILATLEDGWLEGSLKGRTGIFPYRFVKLCPDTRVEETMALPQEGSLARIPETSLDCLENTLGVEEQRHETSDHEAEEPDCIISEAPTSPLGHLTSEYDTDRNSYQDEDTAGGPPRSPGVEWEMPLATDSPTSDPTEVVNGISSQPQVPFHPNLQKSQYYSTVGGSHPHSEQYPDLLPLEARTRDYASLPPKRMYSQLKTLQKPVLPLYRGSSVSASRVVKPRQSSPQLHNLASYTKKHHTSSVYSISERLEMKPGPQAQGLVMEAATHSQGDGSTDLDSKLTQQLIEFEKSLAGPGTEPDKILRHFSIMDFNSEKDIVRGSSKLITEQELPERRKALRPPPPRPCTPVSTSPHLLVDQNLKPAPPLVVRPSRPAPLPPSAQQRTNAVSPKLLSRHRPTCETLEKEGPGHMGRSLDQTSPCPLVLVRIEEMERDLDMYSRAQEELNLMLEEKQDESSRAETLEDLKFCESNIESLNMELQQLREMTLLSSQSSSLVAPSGSVSAENPEQRMLEKRAKVIEELLQTERDYIRDLEMCIERIMVPMQQAQVPNIDFEGLFGNMQMVIKVSKQLLAALEISDAVGPVFLGHRDELEGTYKIYCQNHDEAIALLEIYEKDEKIQKHLQDSLADLKSLYNEWGCTNYINLGSFLIKPVQRVMRYPLLLMELLNSTPESHPDKVPLTNAVLAVKEINVNINEYKRRKDLVLKYRKGDEDSLMEKISKLNIHSIIKKSNRVSSHLKHLTGFAPQIKDEVFEETEKNFRMQERLIKSFIRDLSLYLQHIRESACVKVVAAVSMWDVCMERGHRDLEQFERVHRYISDQLFTNFKERTERLVISPLNQLLSMFTGPHKLVQKRFDKLLDFYNCTERAEKLKDKKTLEELQSARNNYEALNAQLLDELPKFHQYAQGLFTNCVHGYAEAHCDFVHQALEQLKPLLSLLKVAGREGNLIAIFHEEHSRVLQQLQVFTFFPESLPATKKPFERKTIDRQSARKPLLGLPSYMLQSEELRASLLARYPPEKLFQAERNFNAAQDLDVSLLEGDLVGVIKKKDPMGSQNRWLIDNGVTKGFVYSSFLKPYNPRRSHSDASVGSHSSTESEHGSSSPRFPRQNSGSTLTFNPSSMAVSFTSGSCQKQPQDASPPPKECDQGTLSASLNPSNSESSPSRCPSDPDSTSQPRSGDSADVARDVKQPTATPRSYRNFRHPEIVGYSVPGRNGQSQDLVKGCARTAQAPEDRSTEPDGSEAEGNQVYFAVYTFKARNPNELSVSANQKLKILEFKDVTGNTEWWLAEVNGKKGYVPSNYIRKTEYT

SEQ ID NO: 41 - DNA, 2224 bp, Homo sapiens
atgcagccgccgccgaggaaggtgaaagttacacaagaactgaaaaacattcaagttgagcagatgacaaaacttcaagccaaacatcaagcagaatgtgatttgcttgaagatatgaggacattcagtcagaagaaggctgctattgaaagagagtatgcacagggtatgcagaagttggctagtcaatacctgaagagagattggcctggagtaaaagctgatgatcggaatgattacaggagcatgtatcccgtttggaaatcttttctcgagggaacaatgcaggtagcccagtctcggatgaatatatgtgaaaactataaaaacttcatttctgagcctgcaaggacagtgagaagcttaaaagaacagcaactaaaaaggtgtgtggaccagttgacaaagatccaaactgaattacaagagacagtgaaagatttagctaaaggcaaaaagaaatactttgagactgaacagatggctcatgcagtacgagagaaagctgacatcgaggcaaaatctaaacttagtctttttcaatcaagaatcagtttacagaaggcaagtgtaaagttaaaagcccggcgatctgagtgtaattccaaagctacccacgcaaggaatgattatcttcttaccctagcggcagcaaatgcacatcaggatcgctactatcaaacagatttagttaacattatgaaggctcttgatggaaatgtgtatgatcatctcaaggattatttaatagccttcagccggactgagctagaaacatgccaagctgtgcagaacacattccagtttttattagaaaactccagcaaggtggtccgggactacaatcttcagctgtttttgcaagaaaacgctgtatttcacaaaccccagcccttccagttccagccttgtgacagtgatactagccgacagttagaatcagaaactgggaccacagaggagcacagtctaaataaggaagctcgaaaatgggccacacgtgtggcacgtgagcataaaaacattgttcaccaacaacgggttctaaatgatctggagtgtcatggagctgctgtatcagaacaaagccgagcagagctagaacagaaaatagatgaagctagagaaaatattcgtaaagcagagataattaaattgaaagctgaagcccggttggacctgctaaagcagattggtgtttctgtggacacatggctaaagagtgccatgaaccaagtaatggaagaactggaaaatgagcgatgggcccgccctcctgcagtgaccagtaatggcactttacactcgcttaatgcagataccgaaagagaagaaggcgaagagtttgaagataacatggatgttttcgatgacagcagttccagcccttctggcaccttaagaaattatccactcacctgcaaagttgtttattcctacaaggcttctcaaccagatgagttgaccattgaggaacatgaggtgttagaagtgattgaagatggagatatggaagactgggtaaaggctcgaaataaagttggccaagtgggttatgtgccagaaaagtacctacagtttcccacctcgaacagcctcctgagcatgctgcagtccctggccgctttggacagtcggtcacacacgtccagcaattccacggaagcagaactcgtttcaggcagcctcaacggagatgccagtgtatgttttgtgaaagcactttatgattatgagggccagacagatgatgagttatcttttcctgagggagcaataatccgtatcttgaacaaagaaaaccaagatgatgatggcttctgggaaggggaattcaatgggcgtattggagttttcccatcggtgctagtggaagaactttcagcctcagaaaatggtgacactccatggatgagagagattcagatctctccttcccccaagccacacgcctccctgcctccactgccgttgtacgaccagcctcccagcagcccctaccccagcccagataagaggagctccctgtactttccccggtctccttcagcaaacgaaaaaagccttcatgctgagtcaccaggattctcacaggcatcaaggcatactcctgagacctcatatggcaaactgcgacctgtccgggcagctccccctccacctacacagaatcaccgaaggccagcagagaagattgaagatgtggaaatcacactggtgtgat

SEQ ID NO: 42 - protein, 740 aa, Homo sapiens
MQPPPRKVKVTQELKNIQVEQMTKLQAKHQAECDLLEDMRTFSQKKAAIEREYAQGMQKLASQYLKRDWPGVKADDRNDYRSMYPVWKSFLEGTMQVAQSRMNICENYKNFISEPARTVRSLKEQQLKRCVDQLTKIQTELQETVKDLAKGKKKYFETEQMAHAVREKADIEAKSKLSLFQSRISLQKASVKLKARRSECNSKATHARNDYLLTLAAANAHQDRYYQTDLVNIMKALDGNVYDHLKDYLIAFSRTELETCQAVQNTFQFLLENSSKVVRDYNLQLFLQENAVFHKPQPFQFQPCDSDTSRQLESETGTTEEHSLNKEARKWATRVAREHKNIVHQQRVLNDLECHGAAVSEQSRAELEQKIDEARENIRKAEIIKLKAEARLDLLKQIGVSVDTWLKSAMNQVMEELENERWARPPAVTSNGTLHSLNADTEREEGEEFEDNMDVFDDSSSSPSGTLRNYPLTCKVVYSYKASQPDELTIEEHEVLEVIEDGDMEDWVKARNKVGQVGYVPEKYLQFPTSNSLLSMLQSLAALDSRSHTSSNSTEAELVSGSLNGDASVCFVKALYDYEGQTDDELSFPEGAIIRILNKENQDDDGFWEGEFNGRIGVFPSVLVEELSASENGDTPWMREIQISPSPKPHASLPPLPLYDQPPSSPYPSPDKRSSLYFPRSPSANEKSLHAESPGFSQASRHTPETSYGKLRPVRAAPPPPTQNHRRPAEKIEDVEITLV

SEQ ID NO: 43 - DNA, 2469 bp, Homo sapiens
atggggtttgggagtgacctgaagaattcacatgaagcagtgttaaaattgcaagactgggaattacggttactggaaacagtaaagaaatttatggccctgagaataaaaagtgataaagaatatgcatctactttacagaacctttgtaatcaagttgataaggaaagtactgtccaaatgaattatgtcagcaacgtatccaagtcttggctacttatgattcagcagacagaacaacttagtaggataatgaagacacatgcagaggacttgaactctggacctttacacaggctcaccatgatgattaaggacaagcagcaggtgaagaaaagttacataggtgttcatcagcagatagaggcagagatgatcaaggttaccaaaacagaattggagaagttaaaatgcagctatagacaattaataaaagaaatgaattctgccaaagagaaatataaagaagctttagctaaagggaaggaaactgaaaaggccaaggaacgatacgacaaagccacaatgaaacttcatatgttgcacaatcagtatgtattggcgttgaaaggggcacagctccatcagaatcagtattatgatatcacacttcccctgcttctggactccttacaaaagatgcaagaagaaatgataaaagcactcaaaggtatatttgatgaatacagccagataaccagtcttgtcacagaggaaatagtgaatgtccataaagagattcaaatgtcggttgaacagatagatcctagtacagaatacaataatttcatagatgttcacagaacaacggctgctaaagaacaagaaatagagtttgatacttccttactggaagaaaatgaaaatcttcaggcaaatgagatcatgtggaataacttaacagcagaaagtttgcaagtaatgttgaaaacgttagcggaagaacttatgcaaacacagcagatgcttttaaacaaggaggaggctgttttggagttagagaagagaattgaagaatcttctgaaacttgtgagaagaagtctgatattgtgcttctgctaagccaaaaacaggcacttgaagaactgaaacagtcagtccagcagctgagatgcactgaagcaaagttttcagcacagaaggaattactagagcaaaaagtgcaagaaaatgatgggaaagagccacctccagtagtaaattatgaagaagatgcacgatcagttacatctatggaaagaaaggagaggctatccaaatttgaatctattcgtcattcaattgctggaataattaggtctccaaaatctgcagtgggctcttcagcactttctgatatgatctccatcagtgagaagcctttggcagaacaggactggtaccatggtgcaattcccagaatagaagctcaagaactgttaaaaaaacaaggagactttttggtgcgagagagtcatgggaaacctggtgaatatgtcctttctgtatattctgatggacagaggagacattttatcatacaatatgttgataacatgtatcgattcgagggcactgggttttcaaacattcctcaacttatagatcatcactatacaacaaaacaggtcatcactaagaaatcaggtgtagttctgctgaatcctattcctaaggacaagaaatggattctcagtcatgaagatgtcatattgggagaattactgggcaagggaaattttggtgaagtatataagggcacattaaaggataaaacttctgttgctgttaaaacatgtaaagaagatcttcctcaggaattgaaaataaaatttttacaagaagccaaaattctcaagcaatatgatcatcccaatattgtcaaacttataggagtttgcacacaaagacagcctgtctacatcattatggaactggtttcaggaggtgatttcctcacctttctgagaaggaagaaggatgaactaaaactcaaacagttagtgaaattttcattagacgctgctgctggtatgttgtatctcgagagtaaaaactgtatacacagggaccttgctgcaagaaactgcctggtaggtgaaaataatgttctgaaaatcagtgactttggaatgtctcgtcaagaggatggtggagtgtattcatcttctggcttaaagcagattcccattaaatggacagcaccggaagctcttaattatgggagatacagttcagagagtgacgtgtggagctttggcatccttctctgggagaccttcagcttaggggtttgtccgtaccctggaatgacaaatcagcaagcaagagagcaagtagaaagaggataccggatgtcagctccccagcactgtccagaggatatttccaaaatcatgatgaagtgttgggattataaacctgaaaatcgccctaagttcagtgaacttcagaaagagctcactatcatcaagagaaaactcacatag

SEQ ID NO: 44 - protein, 822 aa, Homo sapiens
MGFGSDLKNSHEAVLKLQDWELRLLETVKKFMALRIKSDKEYASTLQNLCNQVDKESTVQMNYVSNVSKSWLLMIQQTEQLSRIMKTHAEDLNSGPLHRLTMMIKDKQQVKKSYIGVHQQIEAEMIKVTKTELEKLKCSYRQLIKEMNSAKEKYKEALAKGKETEKAKERYDKATMKLHMLHNQYVLALKGAQLHQNQYYDITLPLLLDSLQKMQEEMIKALKGIFDEYSQITSLVTEEIVNVHKEIQMSVEQIDPSTEYNNFIDVHRTTAAKEQEIEFDTSLLEENENLQANEIMWNNLTAESLQVMLKTLAEELMQTQQMLLNKEEAVLELEKRIEESSETCEKKSDIVLLLSQKQALEELKQSVQQLRCTEAKFSAQKELLEQKVQENDGKEPPPVVNYEEDARSVTSMERKERLSKFESIRHSIAGIIRSPKSAVGSSALSDMISISEKPLAEQDWYHGAIPRIEAQELLKKQGDFLVRESHGKPGEYVLSVYSDGQRRHFIIQYVDNMYRFEGTGFSNIPQLIDHHYTTKQVITKKSGVVLLNPIPKDKKWILSHEDVILGELLGKGNFGEVYKGTLKDKTSVAVKTCKEDLPQELKIKFLQEAKILKQYDHPNIVKLIGVCTQRQPVYIIMELVSGGDFLTFLRRKKDELKLKQLVKFSLDAAAGMLYLESKNCIHRDLAARNCLVGENNVLKISDFGMSRQEDGGVYSSSGLKQIPIKWTAPEALNYGRYSSESDVWSFGILLWETFSLGVCPYPGMTNQQAREQVERGYRMSAPQHCPEDISKIMMKCWDYKPENRPKFSELQKELTIIKRKLT

SEQ ID NO: 45 - DNA, 2469 bp, Homo sapiens
atgggcttctcttccgagctgtgcagcccccagggccacggggtcctgcagcaaatgcaggaggccgagcttcgtctactggagggcatgagaaagtggatggcccagcgggtcaagagtgacagggagtatgcaggactgcttcaccacatgtccctgcaggacagtgggggccagagccgggccatcagccctgacagccccatcagtcagtcctgggctgagatcaccagccaaactgagggcctgagccgcttgctgcggcagcacgcagaggatctgaactcagggcccctgagcaagctgagcctgctcatccgggaacggcagcagcttcgcaagacctacagcgagcagtggcagcagctgcagcaggagctcaccaagacccacagccaggacattgagaagctgaagagccagtaccgagctctggcacgggacagtgcccaagccaagcgcaagtaccaggaggccagcaaagacaaggaccgtgacaaggctaaggacaagtatgtgcgcagcctgtggaagctctttgctcaccacaaccgctatgtgctgggcgtgcgggctgcgcagctacaccaccagcaccaccaccagctcctgctgcccggcctgctgcggtcactgcaggacctgcacgaggagatggcttgcatcctgaaggagatcctgcaggaatacctggagattagcagcctggtgcaggatgaggtggtggccattcaccgggagatggctgcagctgctgcccgcatccagcctgaggctgagtaccaaggcttcctgcgacagtatgggtccgcacctgacgtcccaccctgtgtcacgttcgatgagtcactgcttgaggagggtgaaccgctggagcctggggagctccagctgaacgagctgactgtggagagcgtgcagcacacgctgacctcagtgacagatgagctggctgtggccaccgagatggtgttcaggcggcaggagatggttacgcagctgcaacaggagctccggaatgaagaggagaacacccacccccgggagcgggtgcagctgctgggcaagaggcaagtgctgcaagaagcactgcaggggctgcaggtagcgctgtgcagccaggccaagctgcaggcccagcaggagttgctgcagaccaagctggagcacctgggccccggcgagcccccgcctgtgctgctcctgcaggatgaccgccactccacgtcgtcctcggagcaggagcgagaggggggaaggacacccacgctggagatccttaagagccacatctcaggaatcttccgccccaagttctcgctccctccaccgctgcagctcattccggaggtgcagaagcccctgcatgagcagctgtggtaccacggggccatcccgagggcagaggtggctgagctgctggtgcactctggggacttcctggtgcgggagagccagggcaagcaggagtacgtgctgtcggtgctgtgggatggtctgccccggcacttcatcatccagtccttggataacctgtaccgactggaaggggaaggctttcctagcattcctttgctcatcgaccacctactgagcacccagcagcccctcaccaagaagagtggtgttgtcctgcacagggctgtgcccaaggacaagtgggtgctgaaccatgaggacctggtgttgggtgagcagattggacgggggaactttggcgaagtgttcagcggacgcctgcgagccgacaacaccctggtggcggtgaagtcttgtcgagagacgctcccacctgacctcaaggccaagtttctacaggaagcgaggatcctgaagcagtacagccaccccaacatcgtgcgtctcattggtgtctgcacccagaagcagcccatctacatcgtcatggagcttgtgcaggggggcgacttcctgaccttcctccgcacggagggggcccgcctgcgggtgaagactctgctgcagatggtgggggatgcagctgctggcatggagtacctggagagcaagtgctgcatccaccgggacctggctgctcggaactgcctggtgacagagaagaatgtcctgaagatcagtgactttgggatgtcccgagaggaagccgatggggtctatgcagcctcagggggctccagacaagtccccgtgaagtggaccgcacctgaggcccttaactacggccgctactcctccgaaagcgacgtgtggagctttggcatcttgctctgggagaccttcagcctgggggcctccccctatcccaacctcagcaatcagcagacacgggagtttgtggagaaggggggccgtctgccctgcccagagctgtgtcctgatgccgtgttcaggctcatggagcagtgctgggcctatgagcctgggcagcggcccagcttcagcaccatctaccaggagctgcagagcatccgaaagcggcatcggtga

SEQ ID NO: 46 - protein, 822 aa, Homo sapiens [sequence omitted]

SEQ ID NO: 47 - DNA, 2073 bp, Homo sapiens
atgcagccgccgccccgaaaagtgaagccggcccaggaggtgaagcttcgcttcctggaacagctgagcatccttcagacctggcagcagagggaggcggatctgctggaggacatcagatcctacagcaagcagagggcagccattgaacgggagtatgggcaggcactccagaaactggctggcccattcctgaagagggaagggcaccggagcggtgagatggacagcaggggcaggacagtgttcggtgcctggcgctgcctgctggatgccaccgtggctgggggccaaacccgactccaggcgtctgaccgataccgtgacctagcagggggtacagggcggagcgccaaggagcaggtgcttaggaagggaacagagaacctccagagggcgcaggctgaggtgctgcagtctgtccgggagctgagccgaagtcggaagctgtatgggcagcgggaacgtgtgtgggccttggcacaggagaaggcggctgatgtccaggccaggctaaaccgaagtgaccatgggatcttccactctcggaccagtctccagaaactgagcaccaagctgtccgcccagtcagcccagtactcccagcagctgcaagcagcccgcaatgagtacctgcttaacttggtggctaccaatgcccacctcgaccattactaccaggaggaactgccagctctgctcaaggccctggtcagtgagctgtcagagcacttgagggaccccctgacctccctgagccacactgagctggaagccgcagaggtcatcctggagcatgcccaccgcggggagcagacaacctcccaggtaagctgggagcaagacctgaagctgtttcttcaggagcctggtgtattttcccccaccccacctcagcagtttcagccagcagggactgatcaggtgtgtgtcctggagtggggagcagaaggcgtggctggcaagagtggcctggagaaagaggttcagcgcttgaccagccgagctgcccgtgactacaagatccagaaccatgggcatcgggtactgcaacgactggagcagaggcggcagcaggcttcagagcgggaggctccaagcatagaacagaggttacaggaagtgcgagagagcatccgccgggcacaggtgagccaggtgaagggggctgcccggctggccctgctgcagggggctggcttagatgtggagcgctggctgaagccagccatgacccaggcccaggatgaggtggagcaggagcggcggctcagtgaggctcggctgtcccagagggacctctctccaaccgctgaggatgctgagctttctgactttgaggaatgtgaggagacgggagagctctttgaggagcctgccccccaagccctggccacgagggccctcccctgccctgcacacgtggtatttcgctatcaggcagggcgtgaggatgagctgacaatcacggagggtgagtggctggaggtcatagaggagggagatgctgacgaatgggtcaaggctcggaaccagcacggcgaggtaggctttgtccctgagcgatatctcaacttcccggacctctccctcccagagagcagccaagacagtgacaatccctgcggggcagagcccacagcattcctggcacaggccctgtacagctacaccggacagagtgcagaggagctgagcttccctgagggggcactcatccgtctgctgccccgggcccaagatggagtagatgacggcttctggaggggagaatttgggggccgtgttggggtcttcccctccctgctggtggaagagctgcttggccccccagggccacctgaactctctgaccctgaacagatgctgccgtccccttctcctcccagcttctccccacctgcacctacctctgtgttggatgggccccctgcacctgtcctgcctggggacaaagccctggacttccctgggttcctggacatgatggcacctcgactcaggccgatgcgtccaccacctcccccgccggctaaagccccggatcctggccacccagatcccctcacctga

SEQ ID NO: 48 - protein, 690 aa, Homo sapiens
MQPPPRKVKPAQEVKLRFLEQLSILQTWQQREADLLEDIRSYSKQRAAIEREYGQALQKLAGPFLKREGHRSGEMDSRGRTVFGAWRCLLDATVAGGQTRLQASDRYRDLAGGTGRSAKEQVLRKGTENLQRAQAEVLQSVRELSRSRKLYGQRERVWALAQEKAADVQARLNRSDHGIFHSRTSLQKLSTKLSAQSAQYSQQLQAARNEYLLNLVATNAHLDHYYQEELPALLKALVSELSEHLRDPLTSLSHTELEAAEVILEHAHRGEQTTSQVSWEQDLKLFLQEPGVFSPTPPQQFQPAGTDQVCVLEWGAEGVAGKSGLEKEVQRLTSRAARDYKIQNHGHRVLQRLEQRRQQASEREAPSIEQRLQEVRESIRRAQVSQVKGAARLALLQGAGLDVERWLKPAMTQAQDEVEQERRLSEARLSQRDLSPTAEDAELSDFEECEETGELFEEPAPQALATRALPCPAHVVFRYQAGREDELTITEGEWLEVIEEGDADEWVKARNQHGEVGFVPERYLNFPDLSLPESSQDSDNPCGAEPTAFLAQALYSYTGQSAEELSFPEGALIRLLPRAQDGVDDGFWRGEFGGRVGVFPSLLVEELLGPPGPPELSDPEQMLPSPSPPSFSPPAPTSVLDGPPAPVLPGDKALDFPGFLDMMAPRLRPMRPPPPPPAKAPDPGHPDPLT

SEQ ID NO: 49 - DNA, 1431 bp, Homo sapiens
atgtccggcgctcgctgccggaccctgtaccccttctccggggagcggcacggccaggggctgcgcttcgccgcgggcgagctgatcacgctgctgcaggtcccggacggcggctggtgggaaggcgagaaggaggacgggctccgtggctggttcccggcgagctacgtgcagttgctggagaagcctggaatggtcccccctccgccgggagaagaaagccagacggtcatccttccacctggctggcagagctacctgtcgcctcagggccggcggtactatgtcaacacgaccaccaatgagaccacctgggaacgtcccagcagttctcctgggattccagccagccctggctctcacaggagctctctgcctccaacagtgaatggataccacgcatcagggaccccagcgcaccctccagagactgcccacatgagtgtccgaaaatccaccggtgattcccagaacctgggatcctcatcgccaagcaaaaagcagagcaaggaaaacaccatcacaataaactgtgtgacgttccctcacccagacacgatgccggaacagcagctgctgaaaccaaccgagtggagctactgcgactacttctgggctgataagaaggacccccaaggcaacggcaccgtggctgggtttgaactactgctccagaaacagctgaagggcaaacaaatgcagaaggaaatgtcagaattcatccgggaaaggataaagattgaagaagactatgcgaagaacttagctaagctctctcagaactccttggcttcacaggaggaaggctccttgggagaggcgtgggcccaggtgaagaagagcctggcggacgaagcagaagttcacctcaagttctctgccaagcttcacagcgaggtggagaagcccctgatgaacttccgtgagaacttcaagaaagacatgaagaagtgcgaccaccacattgccgaccttcgcaagcagctcgccagccgctatgcctcggtggagaaggcccggaaagccctcacagagcggcagagagacctggagatgaagacccagcagctggagatcaagctgagcaacaagacagaggaggacatcaagaaggcgcggagaaagtccacacaggctggagacgacctcatgcgctgtgtggatctctacaaccaggcccagtccaaatggtttgaagagatggtgaccaccacattggagctagagcggctggaggtggagagggtagagatgatccggcagcacctgtgccagtacacgcagctgcggcatgaaacagacatgttcaaccaaagcacagtcgagcccgtggatcagctgcttcgaaaagtggacccggccaaagacagggagctgtgggtcagagagcacaagacgggcaacatccgccctgtggacatggagatctag

SEQ ID NO: 50 - protein, 476 aa, Homo sapiens
MSGARCRTLYPFSGERHGQGLRFAAGELITLLQVPDGGWWEGEKEDGLRGWFPASYVQLLEKPGMVPPPPGEESQTVILPPGWQSYLSPQGRRYYVNTTTNETTWERPSSSPGIPASPGSHRSSLPPTVNGYHASGTPAHPPETAHMSVRKSTGDSQNLGSSSPSKKQSKENTITINCVTFPHPDTMPEQQLLKPTEWSYCDYFWADKKDPQGNGTVAGFELLLQKQLKGKQMQKEMSEFIRERIKIEEDYAKNLAKLSQNSLASQEEGSLGEAWAQVKKSLADEAEVHLKFSAKLHSEVEKPLMNFRENFKKDMKKCDHHIADLRKQLASRYASVEKARKALTERQRDLEMKTQQLEIKLSNKTEEDIKKARRKSTQAGDDLMRCVDLYNQAQSKWFEEMVTTTLELERLEVERVEMIRQHLCQYTQLRHETDMFNQSTVEPVDQLLRKVDPAKDRELWVREHKTGNIRPVDMEI

SEQ ID NO: 51 - DNA, 2913 bp, Homo sapiens
atggacgcagcagagccgggactccccccaggtcctgagggcaggaagaggtacagtgacatcttccggagcctggacaacctcgaaatctcactggggaacgtgacccttgagatgctggctggagaccctctactctcagaagacccagaacctgacaagacccctacagccactgttaccaacgaagccagctgttggagcggcccctccccagagggtcctgtacccctcacaggggaggaactggacttgcggctcattcggacaaaggggggtgtggacgcagccctggaatatgccaagacctggagccgctatgccaaggaactgcttgcctggactgaaaagagagccagctatgagctggagtttgctaagagcaccatgaagatcgctgaagctggcaaggtgtccattcaacagcagagccacatgcctctgcagtacatctacaccctgtttctggagcacgatctcagcctgggaaccctggccatggagacagtggcccagcagaaaagagactactaccagcccctcgccgccaaacggactgagattgagaagtggcggaaggagttcaaggagcagtggatgaaggagcagaagcggatgaatgaggcggtgcaggcactgcggcgcgcccagctgcagtatgtgcaacgcagcgaggacctgcgggcacgctcccaggggtcccctgaggactcggccccccaggcctcgccgggacctagcaagcagcaggagcggcggcggcgctcgcgagaggaggcccaggccaaggcgcaggaggccgaggcgctgtaccaggcctgtgtccgcgaggccaacgcgcggcagcaggacctggagatcgccaagcagcgaatcgtgtcgcacgtgcgcaagctggtgtttcagggggatgaagtgctgaggcgggtgacgctgagtctcttcgggctgcggggggcgcaggcagagcgtggcccccgcgccttcgccgccctggccgagtgctgtgcgccctttgagccgggccagcgctaccaggagtttgtacgggcgctgcggcccgaggccccgccgcccccgccgcccgccttctccttccaggagttccttccctccttgaacagctcccctctggacatcagaaagaagctctctgggcctcttcctccaaggctggatgagaattcagctgagccaggcccttgggaggatccgggcacaggctggcgctggcaagggactccaggccccactccgggcagcgatgtggacagcgtgggtggcggcagcgagtctcggtccctggactcacccacttccagcccaggcgctggcacgaggcagctggtgaaggcttcgtccacaggcactgagtcctcagatgactttgaggagcgagaccctgacctgggagacgggctggagaatgggctgggcagccccttcgggaagtggacactgtccagcgcggctcagacccaccagctgcggcgactgcggggcccagccaagtgccgcgagtgcgaagccttcatggtcagcgggacggagtgtgaggagtgctttctgacctgccacaagcgctgcctggagactctcctgatcctctgtggacacaggcggctcccagcccggacacccctttttggggttgacttcctgcagctacccagggacttcccggaggaggtaccctttgtggtcacgaagtgcacggctgagatagaacaccgtgccctggatgtgcagggcatttaccgggtcagcgggtcccgggtccgtgtggagcggctgtgccaggctttcgagaatggccgagcgttggtggagctgtcggggaactcgcctcatgacgtctcgagtgtcctcaagcgatttcttcaggagctcaccgagcccgtgatccccttccacctctacgacgccttcatctctctggctaagaccttgcatgcagaccctggggacgaccctgggacccccagccccagccctgatgttatccgctcgctgaagaccctcttggtacagctgcctgactctaactacaacaccctgcggcacctggtggcccatctgttcagggtggctgcacgatttatggaaaacaagatgtctgccaacaacctgggcattgtgtttgggccgacactgctgcggccgccggacggcccgcgggcagccagcgccatccctgtcacctgcctgctggactctgggcatcaggcccagcttgtggagttcctcatcgtgcactacgagcagatctttgggatggatgagctcccccaggccactgagcccccgccccaagacttcagcccagcccctgggcccctcacaaccagctcccaaccgccacccccgcaccttgacccagactcccagcccccagtcctagcctcagaccccggcccagacccccagcaccacagtaccctggagcagcatcccacggccacacctaccgagattccaactccacagagtgaccagagagaggacgtggctgaagacaccaaagatgggggaggggaagtgtccagccaaggcccagaggactcactcctggggacacagtctcgtggccacttcagccgccagccatgtaagtatccccggggcggtgtgaggcctgtaacccaccagctgtccagtctggccctggtggcttccaagctgtgcgaggagacccccatcacatcagtgcccagagggagtttgcgggggcgggggcccagccctgcagctgcctcccctgagggcagccccctgcgccgcaccccgctgcccaagcattttgagattacccaggagacagcccggctactctcgaaattggacagcgaggctgtgcccagggccacctgctgcccggacgtccagcctgaggaagccgaggaccatctctga

SEQ ID NO: 52 - protein, 970 aa, Homo sapiens
MDAAEPGLPPGPEGRKRYSDIFRSLDNLEISLGNVTLEMLAGDPLLSEDPEPDKTPTATVTNEASCWSGPSPEGPVPLTGEELDLRLIRTKGGVDAALEYAKTWSRYAKELLAWTEKRASYELEFAKSTMKIAEAGKVSIQQQSHMPLQYIYTLFLEHDLSLGTLAMETVAQQKRDYYQPLAAKRTEIEKWRKEFKEQWMKEQKRMNEAVQALRRAQLQYVQRSEDLRARSQGSPEDSAPQASPGPSKQQERRRRSREEAQAKAQEAEALYQACVREANARQQDLEIAKQRIVSHVRKLVFQGDEVLRRVTLSLFGLRGAQAERGPRAFAALAECCAPFEPGQRYQEFVRALRPEAPPPPPPAFSFQEFLPSLNSSPLDIRKKLSGPLPPRLDENSAEPGPWEDPGTGWRWQGTPGPTPGSDVDSVGGGSESRSLDSPTSSPGAGTRQLVKASSTGTESSDDFEERDPDLGDGLENGLGSPFGKWTLSSAAQTHQLRRLRGPAKCRECEAFMVSGTECEECFLTCHKRCLETLLILCGHRRLPARTPLFGVDFLQLPRDFPEEVPFVVTKCTAEIEHRALDVQGIYRVSGSRVRVERLCQAFENGRALVELSGNSPHDVSSVLKRFLQELTEPVIPFHLYDAFISLAKTLHADPGDDPGTPSPSPDVIRSLKTLLVQLPDSNYNTLRHLVAHLFRVAARFMENKMSANNLGIVFGPTLLRPPDGPRAASAIPVTCLLDSGHQAQLVEFLIVHYEQIFGMDELPQATEPPPQDFSPAPGPLTTSSQPPPPHLDPDSQPPVLASDPGPDPQHHSTLEQHPTATPTEIPTPQSDQREDVAEDTKDGGGEVSSQGPEDSLLGTQSRGHFSRQPCKYPRGGVRPVTHQLSSLALVASKLCEETPITSVPRGSLRGRGPSPAAASPEGSPLRRTPLPKHFEITQETARLLSKLDSEAVPRATCCPDVQPEEAEDHL

SEQ ID NO: 53 - DNA, 2268 bp, Homo sapiens
atggaggctgtgattgagaaggaatgcagcgcgctcggaggcctcttccagaccatcatcagcgacatgaaggggagctatccagtttgggaagatttcataaacaaagcaggaaagctgcagtcccagcttcggacaacagtagtagcagcagctgccttcttggacgcctttcagaaagtggctgacatggccaccaacacacgtggtgggaccagggagattggatctgctctcaccaggatgtgcatgaggcacagaagcattgaagccaagctgaggcagttttcgagcgctttaattgattgtctgataaacccacttcaagaacagatggaagaatggaagaaagtggccaaccagctggataaagaccacgcaaaagaatataagaaagcccgccaagagataaaaaagaagtcctcggatacgctgaaactgcagaagaaagcaaaaaaagggagaggtgatatccagcctcagttggacagtgctctccaagatgtcaatgataagtatctcttattggaagaaacagaaaagcaggctgtccggaaggctttgattgaagaacgtggccgattctgtaccttcatctctatgctgcggccagtgattgaagaagaaatctcaatgctaggggaaataacccaccttcagaccatctcggaagatctaaaaagcctgaccatggaccctcacaaactgccctcctcaagtgaacaggtgattctggacttgaaaggttctgattacagctggtcgtatcagacgccaccctcttcccccagcaccaccatgtccagaaagtccagtgtctgcagcagcctgaacagtgtcaacagcagtgactcccggtccagcggctcccactcgcattcccccagctcacattaccgctaccgcagctccaacctggcccagcaggctcctgtgaggctgtccagcgtgtcctcccatgactcaggattcatatcccaggatgccttccagtccaagtcaccatcccccatgccgccagaggcccccaaccagttgtctaacgggttttctcactatagtttatcaagtgagtcccacgtggggcccacgggtgcaggccttttccctcattgcctgcctgcctcccgcctgctccctcgggtcacctctgtccaccttccagactacgctcattattacaccattgggcccggcatgttcccgtcatctcagatccctagctggaaggactgggctaagcctgggccctatgaccagcctctggtgaacaccctgcagcgccgcaaagagaagcgagaaccggaccccaacgggggaggacccactaccgccagcggcccacctgcagcagctgaggaggctcagagaccacggagcatgactgtatcggctgccaccaggcctggtgaggagatggaggcttgtgaggagctggccctggccctgtctcggggcctgcagctggacacccagaggagcagccgggactcgcttcagtgctccagcggctacagcacccagacaaccaccccctgctgctctgaggacaccatcccttcccaagtttcagattatgattatttctctgtaagtggtgaccaggaggcagatcagcaggagttcgacaagtcctccaccattccaagaaacagcgacatcagccagtcctaccgacggatgttccaagccaagcgtccagcctcaactgctggcctccccaccaccctgggacctgctatggtcactccaggggttgcaactatccgacggaccccttccaccaagccttctgtccgccggggaaccattggagctggtcccatccccatcaagacacccgtgatccctgtcaagaccccaaccgtcccagacctcccaggggtgatgccagcccctccagatgggccagaagagcggggggagcacagccctgagtcgccatctgtgggtgagggcccccaaggtgtcaccagcatgccctcctcaatgtggagcggccaagcttccgttaaccctccacttccaggcccgaagcccagtatccctgaggagcacagacaggcaattccagaaagtgaagctgaagaccaggaacgggaacccccaagtgccactgtctccccaggccagattccagagagtgaccctgcagacctgagcccaagggatactccacaaggagaagacatgctgaacgccatccgaaggggcgtgaaactgaagaagaccacgacaaacgatcgctcagcccctcgcttttcttag

SEQ ID NO: 54 - protein, 755 aa, Homo sapiens
MEAVIEKECSALGGLFQTIISDMKGSYPVWEDFINKAGKLQSQLRTTVVAAAAFLDAFQKVADMATNTRGGTREIGSALTRMCMRHRSIEAKLRQFSSALIDCLINPLQEQMEEWKKVANQLDKDHAKEYKKARQEIKKKSSDTLKLQKKAKKGRGDIQPQLDSALQDVNDKYLLLEETEKQAVRKALIEERGRFCTFISMLRPVIEEEISMLGEITHLQTISEDLKSLTMDPHKLPSSSEQVILDLKGSDYSWSYQTPPSSPSTTMSRKSSVCSSLNSVNSSDSRSSGSHSHSPSSHYRYRSSNLAQQAPVRLSSVSSHDSGFISQDAFQSKSPSPMPPEAPNQLSNGFSHYSLSSESHVGPTGAGLFPHCLPASRLLPRVTSVHLPDYAHYYTIGPGMFPSSQIPSWKDWAKPGPYDQPLVNTLQRRKEKREPDPNGGGPTTASGPPAAAEEAQRPRSMTVSAATRPGEEMEACEELALALSRGLQLDTQRSSRDSLQCSSGYSTQTTTPCCSEDTIPSQVSDYDYFSVSGDQEADQQEFDKSSTIPRNSDISQSYRRMFQAKRPASTAGLPTTLGPAMVTPGVATIRRTPSTKPSVRRGTIGAGPIPIKTPVIPVKTPTVPDLPGVMPAPPDGPEERGEHSPESPSVGEGPQGVTSMPSSMWSGQASVNPPLPGPKPSIPEEHRQAIPESEAEDQEREPPSATVSPGQIPESDPADLSPRDTPQGEDMLNAIRRGVKLKKTTTNDRSAPRFS

SEQ ID NO: 55 - DNA, 2409 bp, Homo sapiens
atgggtcatcccccgctggagttcagcgactgctacctggacagccccgatttccgcgagaggctcaagtgttatgagcaggaactggagaggaccaacaaattcatcaaagacgtaatcaaagacggcaacgcgcttatcagcgctatgagaaattattcttctgctgttcagaaattttcccagacgctgcagtcatttcagtttgatttcattggagacactctgactgatgatgaaattaacatcgctgaatccttcaaggaatttgctgaattgctcaacgaggtagaaaatgagaggatgatgatggtacacaatgctagtgatttgctgattaaacccttggaaaatttccggaaggaacaaataggcttcaccaaggagcggaaaaagaaatttgaaaaggatggtgagaggttttattctttactggatcggcacttacacctgtcttcaaaaaagaaagaatctcagttacaagaggcagacctacaggtggacaaggagaggcacaattttttcgagtcctctcttgattatgtttatcaaatccaggaagttcaggagtccaagaagttcaatattgtggagcctgtcttggcctttcttcatagtctgttcatttctaacagcctgactgtggagctcacacaggatttcctcccatacaaacaacagctccaactcagtttacagaatacaagaaatcatttctccagtacccgggaagagatggaagaacttaagaaaaggatgaaagaagctccccagacatgcaaacttccaggacagccaactattgaaggctatctctatacacaagagaaatgggctttaggaatatcctgggtgaaatactattgccagtatgagaaagagaccaaaacactgaccatgacgcctatggagcagaagccaggtgctaagcaggggcccttggacttaacactgaagtactgtgtgagaaggaagacggagtctatcgacaagaggttctgttttgacatagaaactaatgaaaggccaggaaccatcactctgcaggccctttcagaagctaacagaaggctatggatggaagccatggatgggaaagaacctatctaccacagccctataacaaaacagcaagaaatggagctaaatgaagtgggcttcaagtttgtcaggaagtgcatcaatattattgagaccaaagggatcaagacagaagggttgtaccgcactgtgggcagcaatattcaggttcagaagctgctgaatgccttttttgatcctaaatgcccaggagatgttgattttcataatagtgactgggacattaagacaatcaccagctccttgaaattctacctcaggaatctttctgaacctgtcatgacctatagacttcacaaagagctggtctctgctgccaagtctgacaacctggattaccgcctaggagctattcactccctggtatataagctaccagaaaagaaccgagagatgctggaacttctgataagacacttggtcaatgtgtgtgagcacagcaaagagaatcttatgaccccctccaacatgggagtaatctttgggcccaccctgatgagagctcaggaggacactgtggccgccatgatgaacatcaaattccagaacatagtggtggaaatactaatcgagcactttggcaagatctatttaggtccacctgaggaaagcgctgcaccgccagtgcctccgcctcgggtgacagcaagaaggcacaaaccaatcacgatttcaaagcgcttgctgcgagaaaggacggttttctatacttcttccctggatgaaagcgaagatgaaatccaacatcaaacaccgaatggtactatcaccagcagcatagaaccccccaagccaccacaacaccccaaactacctattcagaggagtggggaaactgatcctgggaggaagtccccaagcaggcctattttggatggcaagttggagccctgcccagaggtggacgtggggaagttggtgtctaggctgcaggatggagggaccaagatcaccccaaaggccaccaatggacccatgccaggctctgggcccaccaagaccccctctttccacataaagagaccagctccccggcccctggcccaccacaaggagggggatgctgacagtttcagcaaagtgcggcctccaggagaaaagccaaccatcatccgccccccagtgaggcccccagatcctccctgccgggcagctactccccaaaagccagaaccaaagccagatattgtggctggcaatgcgggggaaatcacatcatctgtggtggcttccaggaccaggttttttgaaacagcttcccggaaaacaggaagttctcaaggcagacttcctggagatgaaagttga

SEQ ID NO: 56 - protein, 802 aa, Homo sapiens
MGHPPLEFSDCYLDSPDFRERLKCYEQELERTNKFIKDVIKDGNALISAMRNYSSAVQKFSQTLQSFQFDFIGDTLTDDEINIAESFKEFAELLNEVENERMMMVHNASDLLIKPLENFRKEQIGFTKERKKKFEKDGERFYSLLDRHLHLSSKKKESQLQEADLQVDKERHNFFESSLDYVYQIQEVQESKKFNIVEPVLAFLHSLFISNSLTVELTQDFLPYKQQLQLSLQNTRNHFSSTREEMEELKKRMKEAPQTCKLPGQPTIEGYLYTQEKWALGISWVKYYCQYEKETKTLTMTPMEQKPGAKQGPLDLTLKYCVRRKTESIDKRFCFDIETNERPGTITLQALSEANRRLWMEAMDGKEPIYHSPITKQQEMELNEVGFKFVRKCINIIETKGIKTEGLYRTVGSNIQVQKLLNAFFDPKCPGDVDFHNSDWDIKTITSSLKFYLRNLSEPVMTYRLHKELVSAAKSDNLDYRLGAIHSLVYKLPEKNREMLELLIRHLVNVCEHSKENLMTPSNMGVIFGPTLMRAQEDTVAAMMNIKFQNIVVEILIEHFGKIYLGPPEESAAPPVPPPRVTARRHKPITISKRLLRERTVFYTSSLDESEDEIQHQTPNGTITSSIEPPKPPQHPKLPIQRSGETDPGRKSPSRPILDGKLEPCPEVDVGKLVSRLQDGGTKITPKATNGPMPGSGPTKTPSFHIKRPAPRPLAHHKEGDADSFSKVRPPGEKPTIIRPPVRPPDPPCRAATPQKPEPKPDIVAGNAGEITSSVVASRTRFFETASRKTGSSQGRLPGDES

SEQ ID NO: 57 - DNA, 1335 bp, Homo sapiens
atgtccagctcctacgatgaggcctcactggcgccagaggagaccaccgacagcttctgggaggtggggaactacaagcggaccgtgaagcgcatcgatgacggccaccgtctatgcaacgacctgatgaactgcgtgcaggagcgcgccaagatcgagaaggcgtacgggcagcagctcaccgactgggccaagcgttggcgccagctcatcgagaaaggcccacagtatggcagcctggagcgggcctggggtgccataatgacagaggcagacaaggtgagcgagctgcaccaggaggtgaagaacaatctgctgaatgaggacctggagaaggtgaagaactggcagaaggacgcctatcacaagcagatcatgggtggcttcaaggagacgaaggaggctgaagatggcttccgcaaggcccagaagccttgggccaagaagatgaaggagctggaggcagccaagaaggcctaccatttggcttgcaaagaggaaaagctggccatgacacgggagatgaacagcaagacggagcaatcggtcacacctgagcagcaaaagaagctgcaggacaaagtggacaagtgcaagcaggatgtgcagaagacacaggagaagtatgagaaagtgctggaagatgtgggcaagaccacaccccagtacatggagaacatggagcaggtgtttgagcaatgccagcaatttgaggaaaagcggctggtcttcctcaaggaggtgctgctggacatcaaacggcacctcaacctggctgagaacagcagctacatccatgtgtaccgtgagctggagcaggccatccggggggctgatgcccaggaagacctcagatggttccgcagcaccagtggccccggcatgcccatgaactggccccagtttgaggagtggaacccagaccttcctcacaccaccaccaagaaggagaaacagcctaagaaggcagagggagtggcgctgaccaatgccactggggcggtagagtccacatcccaggctggggaccgcggcagtgttagcagctacgacagaggccagccctacgccaccgagtggtcagacgacgagagtgggaacccctttgggggcagtgagaccaacgggggcgccaacccctttgaggacgactccaagggagtgcgcgtgcgggcactctacgactatgacggccaggagcaggacgagctcagctttaaggccggagacgaactcaccaagctgggcgaggaggatgagcagggctggtgccgtgggcggctggacagcgggcagctgggcctctaccctgccaactacgtggaggctatctag

SEQ ID NO: 58 - protein, 444 aa, Homo sapiens
MSSSYDEASLAPEETTDSFWEVGNYKRTVKRIDDGHRLCNDLMNCVQERAKIEKAYGQQLTDWAKRWRQLIEKGPQYGSLERAWGAIMTEADKVSELHQEVKNNLLNEDLEKVKNWQKDAYHKQIMGGFKETKEAEDGFRKAQKPWAKKMKELEAAKKAYHLACKEEKLAMTREMNSKTEQSVTPEQQKKLQDKVDKCKQDVQKTQEKYEKVLEDVGKTTPQYMENMEQVFEQCQQFEEKRLVFLKEVLLDIKRHLNLAENSSYIHVYRELEQAIRGADAQEDLRWFRSTSGPGMPMNWPQFEEWNPDLPHTTTKKEKQPKKAEGVALTNATGAVESTSQAGDRGSVSSYDRGQPYATEWSDDESGNPFGGSETNGGANPFEDDSKGVRVRALYDYDGQEQDELSFKAGDELTKLGEEDEQGWCRGRLDSGQLGLYPANYVEAI

SEQ ID NO: 59 - DNA, 1462 bp, Homo sapiens
atgtctgtcacatatgatgattccgttggagtagaagtgtccagcgacagcttctgggaggtcgggaactacaagcggactgtgaagcggatcgacgatggccaccgcctgtgcagcgacctcatgaactgcctgcatgagcgggcgcgcatcgagaaggcgtatgcgcagcagctcactgagtgggcccggcgctggaggcagcttgtggagaaagggccccagtacgggaccgtggagaaggcctggatggccttcatgtccgaggcagagagggtgagcgagctgcacctcgaggtgaaggcctcactgatgaacgatgacttcgagaagatcaagaactggcagaaggaagcctttcacaagcagatgatgggcggcttcaaggagaccaaggaagctgaggacggctttcggaaggcacagaagccctgggccaagaagctgaaagaggtagaagcagcaaagaaagcccaccatgcagcgtgcaaagaggagaagctggctatctcacgagaagccaacagcaaggcagacccatccttcaaccctgaacagctcaagaaattgcaagacaaaatagaaaagtgcaagcaagatgttcttaagaccaaagagaagtatgagaagtccctcaaggaactcgaccagggcacaccccagtacatggagaacatggagcaggtgtttgagcagtgccagcagttcgaggagaaacgccttcgcttcttccgggaggttctgctggaggttcagaagcacctaaacctgtccaatgtggctggttacaaagccatttaccatgacctggagcagagcatcagagcagctgatgcagtggaggacctgaggtggttccgagccaatcacgggccaggcatggccatgaactggccgcagtttgaggagtggtccgcagacctgattcgaaccctcagccggagagagaagaagaaggccactgacggcttcaccctgacgggcatcaaccagacaggcgaccagtttttgccgagtaagcccagcagcacccttaatgtcccgagcaaccccgcccagtctgcgcagtcacagtccagctacaaccccttcgaggatgaggacgacacgggcagcaccgtcagtgagaaggaggacattaaggccaaaaatgtgagcagctacgagaagacccagagctatcccaccgactggtcagacgatgagtctaacaaccccttctcctccacggatgccaatggggactcgaatccattcgacgacgacgccacctcggggacggaagtgcgagtccgggccctgtatgactatgaggggcaggagcatgatgagctgagcttcaaggctggggatgagctgaccaagatggaggacgaggatgagcagggctggtgcaagggacgcttggacaacgggcaagttggcctatacccggcaaattatgtggaggcgatccagtgat

SEQ ID NO: 60 - protein, 486 aa, Homo sapiens
MSVTYDDSVGVEVSSDSFWEVGNYKRTVKRIDDGHRLCSDLMNCLHERARIEKAYAQQLTEWARRWRQLVEKGPQYGTVEKAWMAFMSEAERVSELHLEVKASLMNDDFEKIKNWQKEAFHKQMMGGFKETKEAEDGFRKAQKPWAKKLKEVEAAKKAHHAACKEEKLAISREANSKADPSFNPEQLKKLQDKIEKCKQDVLKTKEKYEKSLKELDQGTPQYMENMEQVFEQCQQFEEKRLRFFREVLLEVQKHLNLSNVAGYKAIYHDLEQSIRAADAVEDLRWFRANHGPGMAMNWPQFEEWSADLIRTLSRREKKKATDGFTLTGINQTGDQFLPSKPSSTLNVPSNPAQSAQSQSSYNPFEDEDDTGSTVSEKEDIKAKNVSSYEKTQSYPTDWSDDESNNPFSSTDANGDSNPFDDDATSGTEVRVRALYDYEGQEHDELSFKAGDELTKMEDEDEQGWCKGRLDNGQVGLYPANYVEAIQ

SEQ ID NO: 61 - DNA, 1275 bp, Homo sapiens
atggctccagaagaggacgctggaggggaggccttagggggcagtttctgggaggctggcaactacaggcgcacggtacagcgggtggaggacgggcaccggctgtgcggggacctggtcagctgcttccaggagcgcgcccgcatcgagaaggcttatgcccagcagttggctgactgggtccgaaagtggagggggaccgtggagaagggcccccagtatggcacactggagaaggcctggcatgcctttttcacggcggctgagcggctgagcgcgctgcacctggaggtgcgggagaagctgcaagggcaggacagtgagcgggtgcgcgcctggcagcggggggctttccaccggcctgtgctgggcggcttccgcgagagccgggcggccgaggacggcttccgcaaggcccagaagccctggctgaagaggctgaaggaggttgaggcttccaagaaaagctaccacgcagcccggaaggatgagaagaccgcccagacgagggagagccacgcaaaggcagacagcgccgtctcccaggagcagctgcgcaaactgcaggaacgggtggaacgctgtgccaaggaggccgagaagacaaaagctcagtatgagcagacgctggcagagctgcatcgctacactccacgctacatggaggacatggaacaggcctttgagacctgccaggccgccgagcgccagcggcttcttttcttcaaggatatgctgctcaccttacaccagcacctggacctttccagcagtgagaagttccatgaactccaccgtgacttgcaccagggcattgaggcagccagtgacgaagaggatctgcgctggtggcgcagcacccacgggccaggcatggccatgaactggccacagttcgaggagtggtccttggacacacagaggacaatcagccggaaagagaagggtggccggagccctgatgaggttaccctgaccagcattgtgcctacaagagatggcaccgcacccccaccccagtccccggggtccccaggcacggggcaggatgaggagtggtcagatgaagagagtccccggaaggctgccaccggggttcgggtgagggcactctatgactacgctggccaggaagctgatgagctgagcttccgagcaggggaggagctgctgaagatgagtgaggaggacgagcagggctggtgccaaggccagttgcagagtggccgcattggcctgtaccctgccaactacgtggagtgtgtgggcgcctga

SEQ ID NO: 62 - protein, 424 aa, Homo sapiens [sequence omitted]

SEQ ID NO: 63 - DNA, 2106 bp, Homo sapiens
atgatgaagaggcagctgcaccgcatgcggcagctggcccagacgggcagcttgggacgcaccccggagaccgctgagttcctgggtgaggacctgctgcaggtagaacagcggctggagccggccaagcgggcagcccacaacatccacaagcggctgcaggcctgtctgcagggccagagcggggcagacatggacaagcgggtgaagaagcttcccctcatggctctgtccaccacgatggctgagagcttcaaggagctggaccctgattccagcatggggaaggccttggagatgagctgtgccatccagaatcagctggcccgcatcctggccgagtttgagatgaccctggagagggacgtcctgcagccactcagcaggctgagtgaggaggagctgccagccatcctcaaacacaagaaaagcctccagaagctcgtgtccgactggaacacactcaagagcaggctcagtcaggcaaccaagaattcaggcagcagtcaaggcctaggaggcagcccgggtagtcacagccatacgaccatggccaacaaggtggagacgctgaaggaggaggaggaggagctgaagaggaaagtggagcaatgcagggacgagtacttggctgacctgtaccactttgttaccaaggaggactcctatgccaactacttcattcgtctcctggagattcaggccgattaccatcgcaggtcactgagctcgctggacacagccctggctgagctgagggagaaccacggccaagcagaccactccccttcgatgacagccacccacttccccagggtgtatggggtgtcgctggcaacccacctgcaagagctgggccgggagattgccctgcccatcgaggcctgcgtcatgatgctgctttctgagggcatgaaggaagagggtctcttccgtctggctgctggggcctcggtgctgaagcgtctcaagcagacaatggcctcggacccccacagcctggaggagttctgctccgacccgcacgctgtggcaggtgccctcaagtcctatctgcgggagctgccagagcctctgatgaccttcgacctctatgatgactggatgagggcagccagcctgaaggagccaggggcccggctgcaggccctccaagaggtgtgcagccgcctaccccccgagaacctcagcaacctcaggtacctgatgaagttcctggcacggctggccgaggagcaggaggtgaacaagatgacacccagcaacatcgccatagtcctgggacccaacttgctgtggccacctgagaaagaaggggaccaggcccagctggatgcagcctccgtgtcttccatccaggtggtgggcgtcgtcgaggcgctgatccagagcgcagacaccctcttccctggagacatcaacttcaacgtgtcaggcctcttctcagctgttaccctccaggacacagtcagtgacaggctggcctctgaggaacttccgtccactgccgtgcccaccccagccaccaccccggctccggctccggctccagctccagctccggccccagccttggcttcagcagctaccaaggaaaggacagagtctgaggtgcctcccagaccagcctcccccaaggtcaccaggagtcccccggagacagctgccccagtggaggacatggctcggaggaccaagcgcccggcgccagcccggcccaccatgccgcccccccaggtctccggctcccgctcctcccctccagccccgcccttgccccctggctctggcagccctgggaccccccaagccctgccccgacgtctggttggcagcagcctccgagcccccacagtgccacccccgttaccccccacaccccctcagcctgcccggcgccaaagccggcgttcaccagcctcccccagcccggcctccccaggtccagcctcccccagcccagtctctttgagtaaccctgcacaggtggacctgggggctgccacagcagagggaggagcccctgaggctatcagtggggtccccactcccccagctatccccccgcagccccgccccaggagccttgcctcagagaccaactga

SEQ ID NO: 64 - protein, 701 aa, Homo sapiens
MMKRQLHRMRQLAQTGSLGRTPETAEFLGEDLLQVEQRLEPAKRAAHNIHKRLQACLQGQSGADMDKRVKKLPLMALSTTMAESFKELDPDSSMGKALEMSCAIQNQLARILAEFEMTLERDVLQPLSRLSEEELPAILKHKKSLQKLVSDWNTLKSRLSQATKNSGSSQGLGGSPGSHSHTTMANKVETLKEEEEELKRKVEQCRDEYLADLYHFVTKEDSYANYFIRLLEIQADYHRRSLSSLDTALAELRENHGQADHSPSMTATHFPRVYGVSLATHLQELGREIALPIEACVMMLLSEGMKEEGLFRLAAGASVLKRLKQTMASDPHSLEEFCSDPHAVAGALKSYLRELPEPLMTFDLYDDWMRAASLKEPGARLQALQEVCSRLPPENLSNLRYLMKFLARLAEEQEVNKMTPSNIAIVLGPNLLWPPEKEGDQAQLDAASVSSIQVVGVVEALIQSADTLFPGDINFNVSGLFSAVTLQDTVSDRLASEELPSTAVPTPATTPAPAPAPAPAPAPALASAATKERTESEVPPRPASPKVTRSPPETAAPVEDMARRTKRPAPARPTMPPPQVSGSRSSPPAPPLPPGSGSPGTPQALPRRLVGSSLRAPTVPPPLPPTPPQPARRQSRRSPASPSPASPGPASPSPVSLSNPAQVDLGAATAEGGAPEAISGVPTPPAIPPQPRPRSLASETN

SEQ ID NO: 65 - DNA, 3258 bp, Homo sapiens
atgtccaccccgagccgattcaagaaggacaaagagatcatagccgagtatgaaagtcaagtcaaagaaattcgagctcaactggtagaacaacaaaaatgcctggagcagcaaacggagatgcgagttcagcttctccaggatctgcaagatttcttccgaaaaaaagctgaaattgagacggaatattcccggaatctagagaagttagcagaaaggttcatggcaaaaacaagaagcactaaggatcatcaacaatacaagaaagaccagaacctgttgtctccagtgaactgctggtatttgctcctgaaccaagtaaggagagaaagcaaagaccatgcaaccttgagtgacatctatctgaacaatgtgattatgcggttcatgcagataagtgaggattctaccaggatgtttaaaaagagcaaagagattgcattccaacttcatgaggatttaatgaaggttcttaatgagctttatacggtgatgaaaacataccatatgtatcatgcagagagcatcagtgcagagagcaagctgaaagaggccgaaaaacaagaggaaaagcaaattgggagatctggtgatccagtcttccatattcgactagaggagagacatcaacggcgaagctctgtaaagaaaattgaaaaaatgaaagaaaaaagacaagcaaaatattcagaaaataagctaaaatcaattaaggcacggaacgaatatctcctaacacttgaagccaccaatgcctcagttttcaagtactatattcatgatctttctgatttaattgattgctgtgatcttggctaccatgcaagtctgaacagagccctaagaacatatctgtctgcggagtacaaccttgaaacctccagacatgagggcttagacattattgagaatgcagttgataatttagagcccaggagcgataagcagagattcatggagatgtaccctgctgcgttctgtccaccaatgaagtttgagtttcagtctcacatgggtgatgaggtgtgccaggtcagtgcccagcagccagtccaggcagagctcatgctcaggtaccaacagttgcagtcccgccttgccacgctcaaaatcgagaatgaagaggttaagaaaacgactgaagccaccttgcagacgatacaagatatggtcaccatcgaggactatgatgtttctgaatgcttccagcacagtcgttccacagaatctgtgaagtccactgtctctgaaacctacctgagtaaacccagcatcgccaagagaagagccaaccagcaggaaactgaacagttctacttcatgaaactcagagaatatttggaaggcagtaatctcatcacaaaacttcaagccaaacatgacttgctgcagaggaccctgggagaaggtcatagagctgaatatatgactacaaggcctccaaatgttccccctaagccccagaaacacaggaagtccaggccccgctcacagtataatactaagttgtttaatggggatttggaaacattcgtcaaggactcaggacaggttattcccctcattgtggaaagctgtattcggttcatcaatctctatggtcttcagcatcaggggattttcagagtgtctggttcccaggtggaagtcaatgatattaaaaattcatttgagagaggtgaaaatcctttggctgatgaccagagtaaccatgatattaactcagttgctggcgttctgaagctctatttccgtgggctggaaaaccccctctttcctaaggaaagatttaacgatctgatttcttgtatcagaatagataatctctatgagagggcgcttcacatccgcaaactcctcctgactttgcccaggtcggtccttatagtgatgaggtacctctttgccttcctcaatcatctatcacagtacagcgatgagaatatgatggacccttataacctggccatttgctttggcccaacattgatgcctgtcccagaaatacaggatcaagtgtcttgccaggcacatgtgaatgaaattatcaaaaccatcatcatccaccatgagactattttcccagatgctaaagagctggatggccctgtttatgagaaatgtatggctggagatgactattgcgacagcccatacagtgagcacggtacattggaggaagtggaccaagatgctggtacagagccccacacaagtgaagatgaatgtgagccaatagaagcaatagccaagtttgactatgttgggcggtccgccagagaactatccttcaagaagggtgcctccctgctgctgtatcaccgtgcatctgaggactggtgggaaggcaggcacaacgggattgacgggctggtgcctcaccagtatatagtggtgcaggatatggatgatacgttttcagacactctgagccaaaaagccgacagtgaggccagcagtgggccagtcacggaagacaagtcctcatccaaggacatgaactccccgacagaccgtcatcctgacggctatttagccaggcaacgaaaaagaggagagccaccccctccagtaaggcgtcctggcaggaccagtgatggccattgcccgctccaccctccacatgccctttctaactcctcagttgacctagggtccccaagccttgccagtcacccccggggcctgctgcagaaccgtggcctcaacaatgacagtcctgagcggaggcgcaggcctggccatggcagcctgaccaacatcagccggcacgactccctcaagaagatcgacagccctcccattagaaggtccacgtcatcagggcaatacacgggcttcaatgaccacaagccactggacccagagacaattgctcaggatattgaagaaacgatgaacacagctttgaatgaactccgagaactggagagacagagcacagcaaagcatgcccctgatgtggtgctggataccctggagcaagtgaaaaactctcccacccctgccacttccacggaatctctcagccctttgcacaacgttgccctcaggagctccgagcctcagattcgacgtagcacgagctcctccagtgacacaatgagtactttcaagcctatggtggcacccagaatgggcgtgcagctgaagcctccagcccttaggccaaaacctgctgttcttccaaaaacaaatcctaccataggacctgccccacctccccagggtccaacagacaagtcatgcacaatgtaa

SEQ ID NO: 66 - protein, 1085 aa, Homo sapiens
MSTPSRFKKDKEIIAEYESQVKEIRAQLVEQQKCLEQQTEMRVQLLQDLQDFFRKKAEIETEYSRNLEKLAERFMAKTRSTKDHQQYKKDQNLLSPVNCWYLLLNQVRRESKDHATLSDIYLNNVIMRFMQISEDSTRMFKKSKEIAFQLHEDLMKVLNELYTVMKTYHMYHAESISAESKLKEAEKQEEKQIGRSGDPVFHIRLEERHQRRSSVKKIEKMKEKRQAKYSENKLKSIKARNEYLLTLEATNASVFKYYIHDLSDLIDCCDLGYHASLNRALRTYLSAEYNLETSRHEGLDIIENAVDNLEPRSDKQRFMEMYPAAFCPPMKFEFQSHMGDEVCQVSAQQPVQAELMLRYQQLQSRLATLKIENEEVKKTTEATLQTIQDMVTIEDYDVSECFQHSRSTESVKSTVSETYLSKPSIAKRRANQQETEQFYFMKLREYLEGSNLITKLQAKHDLLQRTLGEGHRAEYMTTRPPNVPPKPQKHRKSRPRSQYNTKLFNGDLETFVKDSGQVIPLIVESCIRFINLYGLQHQGIFRVSGSQVEVNDIKNSFERGENPLADDQSNHDINSVAGVLKLYFRGLENPLFPKERFNDLISCIRIDNLYERALHIRKLLLTLPRSVLIVMRYLFAFLNHLSQYSDENMMDPYNLAICFGPTLMPVPEIQDQVSCQAHVNEIIKTIIIHHETIFPDAKELDGPVYEKCMAGDDYCDSPYSEHGTLEEVDQDAGTEPHTSEDECEPIEAIAKFDYVGRSARELSFKKGASLLLYHRASEDWWEGRHNGIDGLVPHQYIVVQDMDDTFSDTLSQKADSEASSGPVTEDKSSSKDMNSPTDRHPDGYLARQRKRGEPPPPVRRPGRTSDGHCPLHPPHALSNSSVDLGSPSLASHPRGLLQNRGLNNDSPERRRRPGHGSLTNISRHDSLKKIDSPPIRRSTSSGQYTGFNDHKPLDPETIAQDIEETMNTALNELRELERQSTAKHAPDVVLDTLEQVKNSPTPATSTESLSPLHNVALRSSEPQIRRSTSSSSDTMSTFKPMVAPRMGVQLKPPALRPKPAVLPKTNPTIGPAPPPQGPTDKSCTM

SEQ ID NO: 67 - DNA, 3216 bp, Homo sapiens
atgacgtctccagccaaattcaaaaaggataaggagatcatagcagagtacgatactcaggtcaaagagatccgtgctcagctcacagagcagatgaaatgcctggaccagcagtgtgagcttcgggtgcaactgttgcaggacctccaggacttcttccgaaagaaggcagagattgagatggactactcccgcaacctggagaagctggcagaacgcttcctggccaagacacgcagcaccaaggaccagcaattcaagaaggatcagaatgttctctctccagtcaactgctggaatctcctcttaaaccaggtgaagcgggaaagcagggaccataccaccctgagtgacatctacctgaataatatcattcctcgatttgtacaagtcagcgaggactcaggaagactctttaaaaagagtaaagaagtcggccagcagctccaagatgatttgatgaaggtcctgaacgagctctactcggtgatgaagacatatcacatgtacaatgccgacagcatcagtgctcagagcaaactaaaggaggcggagaagcaggaggagaagcaaattggtaaatcggtaaagcaggaggaccggcagaccccacgctcccctgactccacggccaacgttcgcattgaggagaaacatgtccggaggagctcagtgaagaagattgagaagatgaaggagaagcgtcaagccaagtacacggagaataagctgaaggccatcaaagcccggaatgagtacttgctggctttggaggcaaccaatgcatctgtcttcaagtactacatccatgacctatctgaccttattgatcagtgttgtgacttaggctaccatgcaagtctgaaccgggctctacgcaccttcctctctgctgagttaaacctggaacagtcgaagcatgagggtctggatgccatcgagaatgcagtagaaaacctggatgccaccagtgacaagcagcgcctcatggagatgtacaacaacgtcttctgcccccctatgaagtttgagtttcagccccacatgggggatatggcttcccagctctgtgcccagcagcctgtccagagtgagctggtacagagatgccaacaactgcagtctcgcttatccactctaaagattgaaaacgaagaggtaaagaagacaatggaggccaccctgcaaaccatccaggacattgtgactgtcgaggactttgatgtgtctgactgcttccagtacagcaactccatggagtccgtcaagtccacggtctctgaaaccttcatgagcaagcccagcattgctaagaggagagccaaccagcaagagacagagcagttttatttcacaaaaatgaaagagtacctggagggcaggaacctcatcaccaagttacaagccaagcatgaccttctgcagaaaaccctgggagaaagtcagcggacagattgcagtctagccaggcgcagctcaactgtgaggaaacaggactccagccaggcaattcctctggtggtggaaagctgtatccggtttatcagcagacacggactacagcatgaaggaattttccgggtgtcaggatcccaggtggaagtgaatgacatcaaaaatgcctttgagagaggagaggaccccctggctggggaccagaacgaccatgacatggattccatagctggtgtcctgaagctttacttccgggggctggaacaccctctcttccccaaggacatctttcatgacctgatggcctgcgtcacaatggacaacctgcaggagagagctctgcacatccggaaagtcctcctagtcctgcccaaaaccactctgattatcatgagatacctctttgccttcctcaatcatttatcacagttcagtgaagagaacatgatggacccctacaacctcgccatctgcttcgggccctcgctaatgtcagtgccagagggccacgaccaggtgtcctgccaagcccacgtgaatgagctgatcaaaaccatcatcatccagcatgagaacatcttcccaagccccagggagctggagggccctgtctacagcagaggaggaagcatggaggattactgtgatagccctcatggagagactaccccggttgaagactcaacccaggatgtgaccgcagagcaccacacgagcgatgacgaatgtgagcccatcgaggccattgccaagtttgactacgtgggccggacagcccgagagctgtcctttaagaagggagcatccctgctgctttaccagcgggcttccgacgactggtgggaaggccggcacaatggcatcgacggactcatcccccatcagtacatcgtggtccaagacaccgaggacggtgtcgtggagaggtccagccccaagtctgagattgaggtcatttctgagccacctgaagaaaaggtgacagccagagcgggggccagctgtcccagtgggggtcatgtagccgatatttatcttgcaaacatcaacaagcaaaggaagcgtccagaatctgggagcatccggaaaacttttcggagtgacagccatgggctgagcagttccctgactgactcctcctccccaggggtgggggctagctgccgcccatcctcccagcccatcatgagccagagcctccccaaagaagggccagataagtgttccatcagtgggcacgggagcctcaactccatcagccgccactcatccctgaagaatcggctggatagtccacagatccggaagactgccacagcgggaaggtcaaaaagcttcaataaccatcggcccatggaccctgaggtcattgctcaggatattgaggcaacaatgaactcggccctgaatgagctacgggaactagaacggcagagcagtgtcaaacacacccctgacgtggttctggacaccttggagcccctcaaaacctccccagtggtggcccccacgtcagagccctccagccctctgcacacccagctcctcaaggaccccgagcccgccttccagcgcagcgccagtactgctggggacatcgcctgcgccttccggcctgtcaagtctgtcaagatggctgccccggtcaaaccaccagccacacggcccaagcccactgtcttccccaaaacaaatgccactagccctggtgtcaactcatcaacttccccacagtctactgacaagtcttgtactgtctga

SEQ ID NO: 68 - protein, 1071 aa, Homo sapiens
MTSPAKFKKDKEIIAEYDTQVKEIRAQLTEQMKCLDQQCELRVQLLQDLQDFFRKKAEIEMDYSRNLEKLAERFLAKTRSTKDQQFKKDQNVLSPVNCWNLLLNQVKRESRDHTTLSDIYLNNIIPRFVQVSEDSGRLFKKSKEVGQQLQDDLMKVLNELYSVMKTYHMYNADSISAQSKLKEAEKQEEKQIGKSVKQEDRQTPRSPDSTANVRIEEKHVRRSSVKKIEKMKEKRQAKYTENKLKAIKARNEYLLALEATNASVFKYYIHDLSDLIDQCCDLGYHASLNRALRTFLSAELNLEQSKHEGLDAIENAVENLDATSDKQRLMEMYNNVFCPPMKFEFQPHMGDMASQLCAQQPVQSELVQRCQQLQSRLSTLKIENEEVKKTMEATLQTIQDIVTVEDFDVSDCFQYSNSMESVKSTVSETFMSKPSIAKRRANQQETEQFYFTKMKEYLEGRNLITKLQAKHDLLQKTLGESQRTDCSLARRSSTVRKQDSSQAIPLVVESCIRFISRHGLQHEGIFRVSGSQVEVNDIKNAFERGEDPLAGDQNDHDMDSIAGVLKLYFRGLEHPLFPKDIFHDLMACVTMDNLQERALHIRKVLLVLPKTTLIIMRYLFAFLNHLSQFSEENMMDPYNLAICFGPSLMSVPEGHDQVSCQAHVNELIKTIIIQHENIFPSPRELEGPVYSRGGSMEDYCDSPHGETTPVEDSTQDVTAEHHTSDDECEPIEAIAKFDYVGRTARELSFKKGASLLLYQRASDDWWEGRHNGIDGLIPHQYIVVQDTEDGVVERSSPKSEIEVISEPPEEKVTARAGASCPSGGHVADIYLANINKQRKRPESGSIRKTFRSDSHGLSSSLTDSSSPGVGASCRPSSQPIMSQSLPKEGPDKCSISGHGSLNSISRHSSLKNRLDSPQIRKTATAGRSKSFNNHRPMDPEVIAQDIEATMNSALNELRELERQSSVKHTPDVVLDTLEPLKTSPVVAPTSEPSSPLHTQLLKDPEPAFQRSASTAGDIACAFRPVKSVKMAAPVKPPATRPKPTVFPKTNATSPGVNSSTSPQSTDKSCTV

SEQ ID NO: 69 - DNA, 3228 bp, Homo sapiens
atgtcatctcaaactaagttcaagaaagacaaagagatcattgctgaatatgaagcccaaataaaagagatccgcacgcagctggtggagcagttcaaatgtctggagcagcaatcagagtcgcgactgcagctgcttcaagacctccaggagtttttccgccggaaagctgagattgagctcgagtactcccgcagcctggagaagctggctgagcgcttctcctccaaaatccgcagctcccgggagcaccagttcaagaaggaccagtacctcctctcgcctgtgaactgttggtatctggttctgcatcagacccggcgggagagccgagaccatgccaccctcaatgacatcttcatgaacaatgtcatcgtccgcctctcccagatcagtgaggatgtcatcagactcttcaaaaagagcaaggagattggcctgcagatgcacgaggagctcctgaaggtgaccaatgagctctacacagtcatgaaaacctaccacatgtaccatgcagagagcatcagtgcggaaagcaagctgaaggaggctgagaagcaggaggagaagcagttcaataagtcaggagacctcagcatgaacctgctccggcacgaggaccggccccagcgccgcagctctgtgaagaagattgagaagatgaaggagaagaggcaggccaagtactctgagaacaagctgaaatgcacaaaggcccggaatgactacttgctcaatctggcagccaccaacgcagctataagcaaatactacatccatgatgtctctgatctgatcgattgctgtgatttgggcttccatgccagcctggcccgcaccttccggacctatctctcagctgaatacaacctggagacctctcgccacgaagggctggatgtcattgagaatgcagtggacaacctggattcccgaagtgacaagcacacagtcatggacatgtgcaatcaagtcttctgccctccactcaagttcgagttccagccccacatgggggatgaggtctgccaggtcagcgctcagcagcccgtccagacagaactgctcatgcgttatcaccagctgcagtccagactggccaccctcaagatagagaatgaggaggttaggaaaaccctggatgccaccatgcagacattacaggacatgctgactgtggaggactttgatgtctccgatgccttccaacacagtcgatcgacagagtccgtcaagtcggctgcctctgagacctacatgagcaagatcaacattgccaagaggagagccaaccagcaggaaacagaaatgttttattttacaaaatttaaagagtatgtgaatggcagtaacctcatcaccaagctgcaggccaagcacgatttactcaagcagaccctgggcgaaggggaaagagcagaatgcggcaccaccagaggaagaagaaatgctcgaaccaggaaccaggattcaggacaagctataccgcttgtagtcgagagctgcatccgttacatcaatttatatggactccagcagcaggggatcttcagagtgccaggatctcaggtggaagtcaatgacatcaaaaattcctttgagagaggtgaagacccccttgtggacgatcaaaatgaacgagatatcaattcagtcgctggtgttttaaaactgtatttccgaggactggaaaacccactctttcctaaggaaaggtttcaagatttgatatctactattaaactggagaacccagccgagagggtgcaccagatccaacaaatcctcgtcacccttccccgcgtggtcattgtggtcatgagatacctcttcgctttcctcaaccacctctcccagtatagcgacgagaacatgatggatccctacaacctggccatctgcttcgggcctaccctcatgcacatccctgatgggcaggaccctgtgtcctgccaggcacacatcaatgaagtcatcaaaaccatcatcatccatcatgaagccatcttccccagcccccgggagctagagggacctgtgtatgaaaaatgcatggctggaggggaagaatattgtgacagcccacacagcgagccaggggccatcgatgaagttgaccatgacaatggcactgagcctcataccagcgatgaagaagtggagcagatcgaggctattgccaagtttgactacatggggcggtccccgcgtgagctatccttcaagaagggggcctcgctgctcctgtaccaccgcgcctcggaggactggtgggagggccggcacaacggcgtggatggactcatcccccatcagtacatagttgtacaggacatggatgatgccttctccgacagcctgagccagaaggctgacagcgaggccagcagtgggccattgctggatgacaaggcctcttccaaaaacgacctccagtcccccacggagcacatctcggattacggctttgggggggtgatgggccgagtgcggttacgatctgatggagcagccatccccagacgccgaagcgggggcgacacacacagcccgccccggggcctgggccccagcatagacacaccaccccgggctgctgcctgccccagcagcccccacaaaatccccctcacccgggggaggatcgagagccctgagaagcggaggatggcgacgttcgggagcgctggcagcatcaactaccctgacaagaaggcgctctccgaagggcactcgatgaggtcgacctgcggttccaccaggcacagcagcctaggggaccacaagtccctggaggccgaggccctggcagaagacatcgagaagaccatgagcacggctctgcacgagttgcgggaactcgagaggcagaacacggtcaagcaggcgccagatgtggtgctggacaccctggagcccctgaagaacccgccaggccccgtcagctcggagcccgccagtccccttcacaccatcgtcatccgcgaccccgatgccgccatgcgccgcagcagcagctcctccaccgagatgatgaccaccttcaagccagccctgtccgcccgcctggctggcgcccagctccgcccgccccccatgcggcccgtgcggccggtggtccagcaccggtccagcagcagcagcagctcgggcgtgggcagcccggccgtgacgcccaccgagaagatgttccccaacagctcagcggacaagtcgggcaccatgtga

SEQ ID NO: 70 - protein, 1075 aa, Homo sapiens
MSSQTKFKKDKEIIAEYEAQIKEIRTQLVEQFKCLEQQSESRLQLLQDLQEFFRRKAEIELEYSRSLEKLAERFSSKIRSSREHQFKKDQYLLSPVNCWYLVLHQTRRESRDHATLNDIFMNNVIVRLSQISEDVIRLFKKSKEIGLQMHEELLKVTNELYTVMKTYHMYHAESISAESKLKEAEKQEEKQFNKSGDLSMNLLRHEDRPQRRSSVKKIEKMKEKRQAKYSENKLKCTKARNDYLLNLAATNAAISKYYIHDVSDLIDCCDLGFHASLARTFRTYLSAEYNLETSRHEGLDVIENAVDNLDSRSDKHTVMDMCNQVFCPPLKFEFQPHMGDEVCQVSAQQPVQTELLMRYHQLQSRLATLKIENEEVRKTLDATMQTLQDMLTVEDFDVSDAFQHSRSTESVKSAASETYMSKINIAKRRANQQETEMFYFTKFKEYVNGSNLITKLQAKHDLLKQTLGEGERAECGTTRGRRNARTRNQDSGQAIPLVVESCIRYINLYGLQQQGIFRVPGSQVEVNDIKNSFERGEDPLVDDQNERDINSVAGVLKLYFRGLENPLFPKERFQDLISTIKLENPAERVHQIQQILVTLPRVVIVVMRYLFAFLNHLSQYSDENMMDPYNLAICFGPTLMHIPDGQDPVSCQAHINEVIKTIIIHHEAIFPSPRELEGPVYEKCMAGGEEYCDSPHSEPGAIDEVDHDNGTEPHTSDEEVEQIEAIAKFDYMGRSPRELSFKKGASLLLYHRASEDWWEGRHNGVDGLIPHQYIVVQDMDDAFSDSLSQKADSEASSGPLLDDKASSKNDLQSPTEHISDYGFGGVMGRVRLRSDGAAIPRRRSGGDTHSPPRGLGPSIDTPPRAAACPSSPHKIPLTRGRIESPEKRRMATFGSAGSINYPDKKALSEGHSMRSTCGSTRHSSLGDHKSLEAEALAEDIEKTMSTALHELRELERQNTVKQAPDVVLDTLEPLKNPPGPVSSEPASPLHTIVIRDPDAAMRRSSSSSTEMMTTFKPALSARLAGAQLRPPPMRPVRPVVQHRSSSSSSSGVGSPAVTPTEKMFPNSSADKSGTM

SEQ ID NO: 71 - DNA, 2841 bp, Homo sapiens
atggccgctcacgggaagctgcggcgggagcgggggctgcaggctgagtatgagacgcaagtcaaagagatgcgctggcagctgagcgagcagctgcgctgcctggagctgcagggcgagctgcggcgggagttgctgcaggagctggcagagttcatgcggcgccgcgctgaggtggagctggaatactcccggggcctggaaaagctggccgagcgcttctccagccgtggaggccgcctggggagcagccgggagcaccaaagcttccggaaggagccgtccctcctgtcgcccttgcactgctgggcggtgctgctgcagcacacgcggcagcagagccgggagagcgcggccctgagtgaggtgctggccgggcccctggcccagcgcctgagtcacattgcagaggacgtggggcgcctggtcaagaagagcagggatctggagcagcagctgcaggatgagctcctggaggtggtctcagagctccagacggccaagaagacgtaccaggcatatcacatggagagcgtgaatgccgaggccaagctccgggaggccgagcggcaggaggagaagcgggcaggccggagtgtccccaccaccaccgctggtgccactgaggcagggcccctccgcaagagctccctcaagaagggagggaggctggtggagaagcggcaggccaagttcatggagcacaaactcaagtgcacaaaggcgcgcaacgagtacctgcttagcctggctagtgtcaacgctgctgtcagtaactactacctgcatgacgtcttggacctcatggactgctgtgacacagggttccacctggccctggggcaggtgctccggagctacacggccgctgagagccgcacccaagcctcccaagtgcagggcctgggcagcctggaagaagctgtggaggccctggatcctccaggggacaaagccaaggttctcgaggtgcatgctaccgtcttctgtcccccgctgcgctttgactaccacccccatgatggggatgaggtggctgagatctgcgttgaaatggagctgcgggacgagattctgcccagagcccagaacatccagagccgcctggaccgacagaccattgagacagaggaggtgaacaagactctgaaggcgacactgcaggccctgctggaggtggtggcctcggatgacggggatgtgcttgattccttccagaccagcccctccaccgagtccctcaagtccaccagctcagacccaggcagccggcaggcgggccggaggcgcggccagcagcaggagaccgaaaccttctacctcacgaagctccaggagtatctgagtggacggagcatcctcgccaagctgcaggccaagcacgagaagctgcaggaggcccttcagcgaggtgacaaggaggagcaggaggtgtcttggacccagtacacacagagaaaattccagaagagccgccagccccgccccagctcccagtataaccagagactctttgggggagacatggagaagtttatccagagctcaggccagcctgtgcccctggtggtggagagctgcattcgcttcatcaacctcaatggcctgcagcatgaaggcatcttccgggtatcgggtgcccagctccgggtctcagagatccgtgatgccttcgagagaggggaggacccactggtggagggctgcactgcccatgacctggactcggtggccggggtgctgaagctctacttccggagcctggagcccccactcttccccccagacctgttcggcgagctgctggcttcttcggagctggaggacacagcggagagggtggagcacgtgagccgcctgctgtggcggctgcccgcgccggtgctggtggttctgcgctacctcttcaccttcctcaaccacctggcccagtacagcgatgagaacatgatggacccctacaacctggccgtgtgcttcgggcccacgctgctaccggtgcccgctgggcaggacccggtggcgctgcagggccgggtgaaccagctggtgcagacgctcatagtgcagcccgatcgggtcttcccgcccctgacctcgctgcctggccccgtctacgagaagtgcatggcaccgccttccgccagctgcctgggggacgcccagctggagagcctgggggcggacaatgatccggagctggaagccgagatgcccgcacaggaggatgacctggagggggtcgtggaggctgtggcctgctttgcctacacgggccgcacagcccaggagctgagcttccggcggggggacgtactgcggctgcacgagagggcctcgagcgactggtggcggggggagcacaacggcatgcggggcctcatcccccacaagtatatcacgctgcccgccgggacggagaagcaggtggtgggcgcagggctgcagactgcaggggagtctgggagcagtcccgagggcctcctggcatcggagctggtccaccggccagagccatgcacctcacctgaggccatgggaccctctggacacagacgacgctgcttggtcccagcctccccagagcaacacgtggaggtggataaggctgtggcacagaacatggactctgtgtttaaggagctcttgggaaagacctctgtccgccagggccttgggccagcatctaccacctctcccagtcctgggccccgaagcccaaaggccccgcccagcagccgcctgggcaggaacaaaggcttctcccggggccctggggccccagcctcaccctcagcttcccacccccagggcctagacacgacccccaagccacactga

SEQ ID NO: 72 - protein, 946 aa, Homo sapiens
MAAHGKLRRERGLQAEYETQVKEMRWQLSEQLRCLELQGELRRELLQELAEFMRRRAEVELEYSRGLEKLAERFSSRGGRLGSSREHQSFRKEPSLLSPLHCWAVLLQHTRQQSRESAALSEVLAGPLAQRLSHIAEDVGRLVKKSRDLEQQLQDELLEVVSELQTAKKTYQAYHMESVNAEAKLREAERQEEKRAGRSVPTTTAGATEAGPLRKSSLKKGGRLVEKRQAKFMEHKLKCTKARNEYLLSLASVNAAVSNYYLHDVLDLMDCCDTGFHLALGQVLRSYTAAESRTQASQVQGLGSLEEAVEALDPPGDKAKVLEVHATVFCPPLRFDYHPHDGDEVAEICVEMELRDEILPRAQNIQSRLDRQTIETEEVNKTLKATLQALLEVVASDDGDVLDSFQTSPSTESLKSTSSDPGSRQAGRRRGQQQETETFYLTKLQEYLSGRSILAKLQAKHEKLQEALQRGDKEEQEVSWTQYTQRKFQKSRQPRPSSQYNQRLFGGDMEKFIQSSGQPVPLVVESCIRFINLNGLQHEGIFRVSGAQLRVSEIRDAFERGEDPLVEGCTAHDLDSVAGVLKLYFRSLEPPLFPPDLFGELLASSELEDTAERVEHVSRLLWRLPAPVLVVLRYLFTFLNHLAQYSDENMMDPYNLAVCFGPTLLPVPAGQDPVALQGRVNQLVQTLIVQPDRVFPPLTSLPGPVYEKCMAPPSASCLGDAQLESLGADNDPELEAEMPAQEDDLEGVVEAVACFAYTGRTAQELSFRRGDVLRLHERASSDWWRGEHNGMRGLIPHKYITLPAGTEKQVVGAGLQTAGESGSSPEGLLASELVHRPEPCTSPEAMGPSGHRRRCLVPASPEQHVEVDKAVAQNMDSVFKELLGKTSVRQGLGPASTTSPSPGPRSPKAPPSSRLGRNKGFSRGPGAPASPSASHPQGLDTTPKPH

SEQ ID NO: 73 - DNA, 1644 bp, Homo sapiens
atgagctggggcacggagctgtgggatcagttcgacagcttagacaagcatacacaatggggaattgacttcttggaaagatatgccaaatttgttaaagagaggatagaaattgaacagaactatgcgaaacaattgagaaatctggttaagaagtactgccccaaacgttcatccaaagatgaagagccacggtttacctcgtgtgtagccttttttaatatccttaatgagttaaatgactatgcaggacagcgagaagttgtagcagaagaaatggcgcacagagtgtatggtgaattaatgagatatgctcatgatctgaaaactgaaagaaaaatgcatctgcaagaaggacgaaaagctcaacaatatcttgacatgtgctggaaacagatggataatagtaaaaagaagtttgaaagagaatgtagagaggcagaaaaggcacaacagagttatgaaagattggataatgatactaatgcaaccaaggcagatgttgaaaaggccaaacagcagttgaatctgcgtacgcatatggccgatgaaaataaaaatgaatatgctgcacaattacaaaactttaatggagaacaacataaacatttttatgtagtgattcctcagatttacaagcaactacaagaaatggacgaacgaaggactattaaactcagtgagtgttacagaggatttgctgactcagaacgcaaagttattcccatcatttcaaaatgtttggaaggaatgattcttgcagcaaaatcagttgatgaaagaagagactctcaaatggtggtagactccttcaaatctggttttgaacctccaggagactttccatttgaagattacagtcaacatatatatagaaccatttctgatgggactatcagtgcatccaaacaggagagtgggaagatggatgccaaaaccacagtaggaaaggccaagggcaaattgtggctctttggaaagaagccaaagggcccagcactagaagatttcagtcatctgccaccagaacagagacgtaaaaaactacagcagcgcattgatgaacttaacagagaactacagaaagaatcagaccaaaaagatgcactcaacaaaatgaaagatgtatatgagaagaatccacaaatgggggatccagggagtttgcagcctaaattagcagagaccatgaataacattgaccgcctacgaatggaaatccataagaatgaggcttggctctctgaagtcgaaggcaaaacaggtgggagaggagacagaagacatagcagtgacataaatcatcttgtaacacagggacgagaaagtcctgagggaagttacactgatgatgcaaaccaggaagtccgtgggccaccccagcagcatggtcaccacaatgagtttgatgatgaatttgaggatgatgatcccttgcctgctattggacactgcaaagctatctacccttttgatggacataatgaaggtactctagcaatgaaagaaggtgaagttctctacattatagaggaggacaaaggtgacggatggacaagagctcggagacagaacggtgaagaaggctacgttcccacgtcatacatagatgtaactctagagaaaaacagtaaaggttcctga

SEQ ID NO: 74 - protein, 547 aa, Homo sapiens
MSWGTELWDQFDSLDKHTQWGIDFLERYAKFVKERIEIEQNYAKQLRNLVKKYCPKRSSKDEEPRFTSCVAFFNILNELNDYAGQREVVAEEMAHRVYGELMRYAHDLKTERKMHLQEGRKAQQYLDMCWKQMDNSKKKFERECREAEKAQQSYERLDNDTNATKADVEKAKQQLNLRTHMADENKNEYAAQLQNFNGEQHKHFYVVIPQIYKQLQEMDERRTIKLSECYRGFADSERKVIPIISKCLEGMILAAKSVDERRDSQMVVDSFKSGFEPPGDFPFEDYSQHIYRTISDGTISASKQESGKMDAKTTVGKAKGKLWLFGKKPKGPALEDFSHLPPEQRRKKLQQRIDELNRELQKESDQKDALNKMKDVYEKNPQMGDPGSLQPKLAETMNNIDRLRMEIHKNEAWLSEVEGKTGGRGDRRHSSDINHLVTQGRESPEGSYTDDANQEVRGPPQQHGHHNEFDDEFEDDDPLPAIGHCKAIYPFDGHNEGTLAMKEGEVLYIIEEDKGDGWTRARRQNGEEGYVPTSYIDVTLEKNSKGS

SEQ ID NO: 75 - protein, 10 aa, Homo sapiens
MGCIKSKRKD

SEQ ID NO: 76 - DNA, 1650 bp, Homo sapiens
atggcgtcggcctcctccgggccgtcgtcttcggtcggtttttcatcctttgatcccgcggtcccttcctgtaccttgtcctcagcatctggaatcaagagacccatggcatctgaggtgccttatgcctctggcatgcccatcaagaaaataggccatagaagtgttgattcctcaggagagacaacatataaaaagacaacctcatcagccttgaaaggtgccatccagttaggcattacccacactgtggggagcctgagtaccaaaccagagcgtgatgtcctcatgcaagatttctacgtggttgagagtatcttctttcccagtgaagggagcaacctgacccctgctcatcactacaatgactttcgtttcaagacctatgcacctgttgccttccgctacttccgggagctatttggtatccggcccgatgattacttgtattccctctgcagtgagccgctgattgaactctgtagctctggagctagtggttccctattctatgtgtccagcgacgatgagttcattattaagacagtccaacataaagaggcggaatttctgcagaagctgcttccaggatactacatgaacctcaaccagaaccctcggactttgctgcctaaattctatggactgtactgtgtgcaggcaggtggcaagaacattcggattgtggtgatgaacaatcttttaccaagatcggtaaaaatgcatatcaaatatgacctcaaaggctcaacctacaaacggcgggcttcccagaaagagcgagagaagcctcttcccacatttaaagacctagacttcttacaagacatccctgatggtctttttttggatgctgacatgtacaacgctctctgtaagaccctgcagcgtgactgtttggtgctgcagagcttcaagataatggattacagcctcttgatgtcaatccataatatagatcatgcacaacgagagcccttaagcagtgaaacacagtactcagttgatactcgaagaccggccccccaaaaggctctgtattccacagccatggaatccatccagggagaggctcgacggggtggtaccatggagactgatgaccatatgggtggcatccctgcccggaatagtaaaggggaaaggcttctgctttatattggcatcattgacattctacagtcttacaggtttgttaagaagttggagcactcttggaaagccctggtacatgacggagacactgtctcagtgcatcgcccaggcttctacgctgaacggttccagcgcttcatgtgcaacacagtatttaagaagattcccttgaagccttctccttccaaaaagtttcggtctggctcatctttctctcggcgagcaggctccagtggcaactcctgcattacttaccagccatcggtctctggggaacacaaggcacaagtgacaacaaaggcagaagtggagccaggcgttcaccttggtcgtcctgatgttttacctcagactccacctttggaggaaatcagtgagggctcgcctattcctgaccccagtttctcacctctagttggagagactttgcaaatgctaactacaagtacaaccttggaaaagcttgaagttgcagagtcagagttcacccattaa

SEQ ID NO: 77 - protein, 549 aa, Homo sapiens
MASASSGPSSSVGFSSFDPAVPSCTLSSASGIKRPMASEVPYASGMPIKKIGHRSVDSSGETTYKKTTSSALKGAIQLGITHTVGSLSTKPERDVLMQDFYVVESIFFPSEGSNLTPAHHYNDFRFKTYAPVAFRYFRELFGIRPDDYLYSLCSEPLIELCSSGASGSLFYVSSDDEFIIKTVQHKEAEFLQKLLPGYYMNLNQNPRTLLPKFYGLYCVQAGGKNIRIVVMNNLLPRSVKMHIKYDLKGSTYKRRASQKEREKPLPTFKDLDFLQDIPDGLFLDADMYNALCKTLQRDCLVLQSFKIMDYSLLMSIHNIDHAQREPLSSETQYSVDTRRPAPQKALYSTAMESIQGEARRGGTMETDDHMGGIPARNSKGERLLLYIGIIDILQSYRFVKKLEHSWKALVHDGDTVSVHRPGFYAERFQRFMCNTVFKKIPLKPSPSKKFRSGSSFSRRAGSSGNSCITYQPSVSGEHKAQVTTKAEVEPGVHLGRPDVLPQTPPLEEISEGSPIPDPSFSPLVGETLQMLTTSTTLEKLEVAESEFTH

SEQ ID NO: 78 - DNA, 1623 bp, Homo sapiens
atgtcttctgctgctgaaaatggagaggcagcacctggaaaacaaaatgaagaaaaaacctataaaaagactgcatcatctgctattaaaggtgctattcagctgggaataggatacacagtgggtaatctcacttccaagccagaacgagatgttcttatgcaagacttttatgtggtggaaagtgtgttcctacccagcgaagggagcaatctgaccccagcacatcactacccagactttagatttaagacatacgctccattagcattccgatatttcagagaactttttggtatcaagcctgatgattacttgtattccatctgcagtgaacctctaatagaactgtctaaccctggagccagtggatccttgttttttgtgaccagtgatgatgaatttatcatcaaaacagttcagcacaaagaagctgagtttcttcagaagctactgccaggctattacatgaatttaaaccagaatccaaggactcttttgccaaaattttacggactgtattgtatgcaatcaggaggcattaatatcaggattgtggtgatgaacaacgttttgccacgctccatgagaatgcactttacatatgacttgaaaggctcaacgtataagcgaagagcatcccgtaaagagagagagaaatccaaccccacatttaaggacttagatttcctgcaagacatgcacgaagggttgtattttgatacggaaacatacaacgcgcttatgaaaacacttcagagagactgccgggtgctagaaagcttcaagatcatggattatagccttctgttgggaattcatttcctggaccattccctcaaagagaaagaggaggagaccccacaaaatgtgcctgatgctaagcggactgggatgcagaaggttctctactcaacagccatggaatctatccagggtccagggaaatctggagatgggataatcacagagaacccagacacaatgggaggcattccagctaaaagccataggggagaaaaactacttttatttatgggcattattgacattctgcaatcatataggttaatgaagaagttagaacattcctggaaagctcttgtttatgatggggacactgtttctgttcatagaccaagcttttatgcagacagatttcttaagttcatgaattccagagttttcaagaaaattcaagctttgaaggcttcaccgtctaagaaacggtgcaattcaatcgccgccctaaaggccacttcacaggagattgtgtcctcaattagccaggaatggaaggatgagaagcgggatttgctgactgaaggacaaagttttagcagccttgatgaagaagccctgggatcccgacacaggccagacctggtccctagcactccatcactgtttgaagctgcttccttggcaaccacaatttcatcttcttccttatacgtcaatgagcactatccacacgacaggcctacactctattcaaacagcaaagggttaccttccagttcaacatttaccttggaagaggggaccatctacttgaccgctgagcccaacactctggaagtgcaggatgacaatgcttctgtgcttgacgtctatttataa

SEQ ID NO: 79 - protein, 540 aa, Homo sapiens
MSSAAENGEAAPGKQNEEKTYKKTASSAIKGAIQLGIGYTVGNLTSKPERDVLMQDFYVVESVFLPSEGSNLTPAHHYPDFRFKTYAPLAFRYFRELFGIKPDDYLYSICSEPLIELSNPGASGSLFFVTSDDEFIIKTVQHKEAEFLQKLLPGYYMNLNQNPRTLLPKFYGLYCMQSGGINIRIVVMNNVLPRSMRMHFTYDLKGSTYKRRASRKEREKSNPTFKDLDFLQDMHEGLYFDTETYNALMKTLQRDCRVLESFKIMDYSLLLGIHFLDHSLKEKEEETPQNVPDAKRTGMQKVLYSTAMESIQGPGKSGDGIITENPDTMGGIPAKSHRGEKLLLFMGIIDILQSYRLMKKLEHSWKALVYDGDTVSVHRPSFYADRFLKFMNSRVFKKIQALKASPSKKRCNSIAALKATSQEIVSSISQEWKDEKRDLLTEGQSFSSLDEEALGSRHRPDLVPSTPSLFEAASLATTISSSSLYVNEHYPHDRPTLYSNSKGLPSSSTFTLEEGTIYLTAEPNTLEVQDDNASVLDVYL

SEQ ID NO: 80 - DNA, 2007 bp, Homo sapiens
atggagctggaggtaccggacgaggcggagagcgctgaggcgggggccgtgccctcggaggcggcgtgggcggcagagagcggggcggcggcaggtttggctcagaagaaggcggccccaacagaggttctgtccatgacggcacagccgggccctggccatgggaagaagttgggccatcgaggtgtggacgcatccggcgaaaccacctacaagaagaccacctcctccaccctgaagggtgccatccagctgggcatcggctacaccgtgggccacctgagctccaagcccgaacgcgacgtgctcatgcaggacttctacgtggtggagagcatcttcttccccagcgaaggcagcaacctcacccccgcccaccacttccaggacttccgcttcaagacctatgcacctgtcgccttccgctacttccgggagctctttgggatccggccagatgattacttgtactccctgtgcaatgagccgctgatcgagctgtccaacccgggcgccagtggctccctcttctacgtcaccagcgacgacgagttcatcatcaagaccgtcatgcacaaggaggccgagttcctgcagaagctgctccctggctactacatgaacctcaaccagaacccgcggacgctgctgcccaagttctatgggctgtactgcgtgcagtcggggggcaagaacatccgcgtcgtggtcatgaacaacatcctgccccgcgtggtcaagatgcacctcaagttcgacctcaagggctccacctacaagcggcgcgccagcaagaaggagaaggagaagagcttccccacctacaaggacctggacttcatgcaggacatgcccgaggggctcctgctggacgccgacaccttcagcgccctggtcaagacgctgcagcgggactgcctggtcctggaaagtttcaagatcatggactacagcctgctgctgggcgtgcacaacatcgaccagcacgagcgcgagcggcaggcgcagggcgcccagagcacctcagatgagaagcggcctgtgggccagaaggcgctctactccacggccatggagtccatccagggtggcgccgcgcgcggggaggccatcgaatcggatgacacgatgggcgggatccccgctgtgaacggccgcggggagcggctgctgctgcacattggcatcatcgacatcctgcagtcctacaggttcatcaagaaactggagcacacctggaaggccctcgtccacgatggggacacggtgtccgtccaccgccccagcttctatgccgagcgctttttcaagttcatgagcaacacggtctttcggaagaactcctccctgaagtcctcgccctccaagaaggggcgcggcggagccttgctagctgtgaaaccgctggggcccaccgctgccttctcggccagccagatccctagcgagcgggaggaggcccagtacgacctgcggggggcccgcagctaccccacgctggaggacgaaggccggcccgacctcctgccctgcacgccaccttctttcgaagaagccactacagcctccattgccacgactctgtcatccacatccctctccattcctgagcggtccccctcggagacgtcggagcagccgcggtacaggcggcgcacacagtcgtctggacaggatggcaggccgcaggaggagccacccgcggaagaggatctgcagcagattacagtgcaggtggagcctgcgtgcagcgtggagattgtggtccccaaagaggaggacgcaggggtggaggcttccccggccggtgcctctgctgctgttgaagtagaaactgccagccaggcctcagacgaggagggcgcacctgccagccaggcctcggacgaggaggacgcgcccgccaccgacatctactttcccaccgatgagaggagctgggtgtactccccgctccactatagcgcccaggcccccccggcctccgacggcgagagcgacacataa

SEQ ID NO: 81 - protein, 668 aa, Homo sapiens
MELEVPDEAESAEAGAVPSEAAWAAESGAAAGLAQKKAAPTEVLSMTAQPGPGHGKKLGHRGVDASGETTYKKTTSSTLKGAIQLGIGYTVGHLSSKPERDVLMQDFYVVESIFFPSEGSNLTPAHHFQDFRFKTYAPVAFRYFRELFGIRPDDYLYSLCNEPLIELSNPGASGSLFYVTSDDEFIIKTVMHKEAEFLQKLLPGYYMNLNQNPRTLLPKFYGLYCVQSGGKNIRVVVMNNILPRVVKMHLKFDLKGSTYKRRASKKEKEKSFPTYKDLDFMQDMPEGLLLDADTFSALVKTLQRDCLVLESFKIMDYSLLLGVHNIDQHERERQAQGAQSTSDEKRPVGQKALYSTAMESIQGGAARGEAIESDDTMGGIPAVNGRGERLLLHIGIIDILQSYRFIKKLEHTWKALVHDGDTVSVHRPSFYAERFFKFMSNTVFRKNSSLKSSPSKKGRGGALLAVKPLGPTAAFSASQIPSEREEAQYDLRGARSYPTLEDEGRPDLLPCTPPSFEEATTASIATTLSSTSLSIPERSPSETSEQPRYRRRTQSSGQDGRPQEEPPAEEDLQQITVQVEPACSVEIVVPKEEDAGVEASPAGASAAVEVETASQASDEEGAPASQASDEEDAPATDIYFPTDERSWVYSPLHYSAQAPPASDGESDT

SEQ ID NO: 82 - RNA, 19 nt, Artificial Sequence
  SYNTHETIC RNA
gggaugugcuugauuccuu

SEQ ID NO: 83 - RNA, 19 nt, Artificial Sequence
  SYNTHETIC RNA
cuaccugcaugacgucuug

SEQ ID NO: 84 - RNA, 19 nt, Artificial Sequence
  SYNTHETIC RNA
agaagacguaccaggcaua

SEQ ID NO: 85 - RNA, 19 nt, Artificial Sequence
  SYNTHETIC RNA
cgcaagagcucccucaaga

SEQ ID NO: 86 - RNA, 19 nt, Artificial Sequence
  SYNTHETIC RNA
ugaacugucucucgaguau

SEQ ID NO: 87 - RNA, 19 nt, Artificial Sequence
  SYNTHETIC RNA
acuauugcauguaucgaaa

SEQ ID NO: 88 - RNA, 19 nt, Artificial Sequence
  SYNTHETIC RNA
aguuugaguuuaucgguga

SEQ ID NO: 89 - RNA, 19 nt, Artificial Sequence
  SYNTHETIC RNA
cguugaaacacgagguaua

SEQ ID NO: 90 - RNA, 19 nt, Artificial Sequence
  SYNTHETIC RNA
gcacuacuguacauaucaa

SEQ ID NO: 91 - RNA, 19 nt, Artificial Sequence
  SYNTHETIC RNA
caagggcuguaucgaauug

SEQ ID NO: 92 - RNA, 19 nt, Artificial Sequence
  SYNTHETIC RNA
gaacggauacggaugauug

SEQ ID NO: 93 - RNA, 19 nt, Artificial Sequence
  SYNTHETIC RNA
ggagaaacgucacuuugga

SEQ ID NO: 94 - RNA, 19 nt, Artificial Sequence
  SYNTHETIC RNA
acacacaaguuucgcaaau

SEQ ID NO: 95 - RNA, 19 nt, Artificial Sequence
  SYNTHETIC RNA
ugaacgagcuacaucauuu

SEQ ID NO: 96 - RNA, 19 nt, Artificial Sequence
  SYNTHETIC RNA
uagcauagguguuguugau

SEQ ID NO: 97 - RNA, 19 nt, Artificial Sequence
  SYNTHETIC RNA
ucauauacgucuagaggaa

SEQ ID NO: 98 - RNA, 19 nt, Artificial Sequence
  SYNTHETIC RNA
guauccaugguaacgaaua

SEQ ID NO: 99 - RNA, 19 nt, Artificial Sequence
  SYNTHETIC RNA
ucucaugacugucucaaau

SEQ ID NO: 100 - RNA, 19 nt, Artificial Sequence
  SYNTHETIC RNA
ggucaaaguaucacuacac

SEQ ID NO: 101 - RNA, 19 nt, Artificial Sequence
  SYNTHETIC RNA
cguagcaauuguagaguuu

SEQ ID NO: 102 - RNA, 19 nt, Artificial Sequence
  SYNTHETIC RNA
caaagaacucggagacguu

SEQ ID NO: 103 - RNA, 19 nt, Artificial Sequence
  SYNTHETIC RNA
caucaugaacggcgucaca

SEQ ID NO: 104 - RNA, 19 nt, Artificial Sequence
  SYNTHETIC RNA
guaugcgggugaaggccau

SEQ ID NO: 105 - RNA, 19 nt, Artificial Sequence
  SYNTHETIC RNA
auuacguguccgacggcua

SEQ ID NO: 106 - RNA, 19 nt, Artificial Sequence
  SYNTHETIC RNA
acaaagagauuauccauga

SEQ ID NO: 107 - RNA, 19 nt, Artificial Sequence
  SYNTHETIC RNA
gaagcaaugugguuaggaa

SEQ ID NO: 108 - RNA, 19 nt, Artificial Sequence
  SYNTHETIC RNA
gcaacgggacugaacauga

SEQ ID NO: 109 - RNA, 19 nt, Artificial Sequence
  SYNTHETIC RNA
cugcagaacuacugaauuc

SEQ ID NO: 110 - RNA, 19 nt, Artificial Sequence
  SYNTHETIC RNA
gcagagaacgaaugcggua

SEQ ID NO: 111 - RNA, 19 nt, Artificial Sequence
  SYNTHETIC RNA
aagaaauccaaccgaguua

SEQ ID NO: 112 - RNA, 19 nt, Artificial Sequence
  SYNTHETIC RNA
gaugaagccugguccgcaa

SEQ ID NO: 113 - RNA, 19 nt, Artificial Sequence
  SYNTHETIC RNA
uagcugagguuaacgggaa

SEQ ID NO: 114 - RNA, 19 nt, Artificial Sequence
  SYNTHETIC RNA
agaaagagguucagcgcuu

SEQ ID NO: 115 - RNA, 19 nt, Artificial Sequence
  SYNTHETIC RNA
gguuacaggaagugcgaga

SEQ ID NO: 116 - RNA, 19 nt, Artificial Sequence
  SYNTHETIC RNA
gaaccagcacggcgaggua

SEQ ID NO: 117 - RNA, 19 nt, Artificial Sequence
  SYNTHETIC RNA
gcuuagauguggagcgcug

SEQ ID NO: 118 - RNA, 19 nt, Artificial Sequence
  SYNTHETIC RNA
uggguaaaggcucgaaaua

SEQ ID NO: 119 - RNA, 19 nt, Artificial Sequence
  SYNTHETIC RNA
gcacuuuacacucgcuuaa

SEQ ID NO: 120 - RNA, 19 nt, Artificial Sequence
  SYNTHETIC RNA
uaaauaaggaagcucgaaa

SEQ ID NO: 121 - RNA, 19 nt, Artificial Sequence
  SYNTHETIC RNA
gagagaaagcugacaucga

SEQ ID NO: 122 - RNA, 19 nt, Artificial Sequence
  SYNTHETIC RNA
cgaggauccugaagcagua

SEQ ID NO: 123 - RNA, 19 nt, Artificial Sequence
  SYNTHETIC RNA
gguguugggugagcagauu

SEQ ID NO: 124 - RNA, 19 nt, Artificial Sequence
  SYNTHETIC RNA
gaagagugguguuguccug

SEQ ID NO: 125 - RNA, 19 nt, Artificial Sequence
  SYNTHETIC RNA
gaaaguggauggcccagcg

SEQ ID NO: 126 - RNA, 19 nt, Artificial Sequence
  SYNTHETIC RNA
gaagaagacuaugcgaaga

SEQ ID NO: 127 - RNA, 19 nt, Artificial Sequence
  SYNTHETIC RNA
gaaaacaccaucacaauaa

SEQ ID NO: 128 - RNA, 19 nt, Artificial Sequence
  SYNTHETIC RNA
gugagaacuucaagaaaga

SEQ ID NO: 129 - RNA, 19 nt, Artificial Sequence
  SYNTHETIC RNA
gaagugcgaccaccacauu

SEQ ID NO: 130 - RNA, 19 nt, Artificial Sequence
  SYNTHETIC RNA
cgaguguccucaagcgauu

SEQ ID NO: 131 - RNA, 19 nt, Artificial Sequence
  SYNTHETIC RNA
gugccaggcuuucgagaau

SEQ ID NO: 132 - RNA, 19 nt, Artificial Sequence
  SYNTHETIC RNA
ggccacaccuaccgagauu

SEQ ID NO: 133 - RNA, 19 nt, Artificial Sequence
  SYNTHETIC RNA
caggguggcugcacgauuu

SEQ ID NO: 134 - RNA, 19 nt, Artificial Sequence
  SYNTHETIC RNA
gagcggacgucauccagug

SEQ ID NO: 135 - RNA, 19 nt, Artificial Sequence
  SYNTHETIC RNA
gagcugagguagucgucca

SEQ ID NO: 136 - RNA, 19 nt, Artificial Sequence
  SYNTHETIC RNA
gggaaaggcagccggaauu

SEQ ID NO: 137 - RNA, 19 nt, Artificial Sequence
  SYNTHETIC RNA
ccaagaacauggccaagua

SEQ ID NO: 138 - RNA, 19 nt, Artificial Sequence
  SYNTHETIC RNA
cagacuacgcucauuauua

SEQ ID NO: 139 - RNA, 19 nt, Artificial Sequence
  SYNTHETIC RNA
ccacggagcaugacuguau

SEQ ID NO: 140 - RNA, 19 nt, Artificial Sequence
  SYNTHETIC RNA
gaacguggccgauucugua

SEQ ID NO: 141 - RNA, 19 nt, Artificial Sequence
  SYNTHETIC RNA
ccaguugucuaacggguuu

SEQ ID NO: 142 - RNA, 19 nt, Artificial Sequence
  SYNTHETIC RNA
accuaaagggcucggacua

SEQ ID NO: 143 - RNA, 19 nt, Artificial Sequence
  SYNTHETIC RNA
gcgaaagaguacaaacgag

SEQ ID NO: 144 - RNA, 19 nt, Artificial Sequence
  SYNTHETIC RNA
ggguagaauuuccgccaca

SEQ ID NO: 145 - RNA, 19 nt, Artificial Sequence
  SYNTHETIC RNA
ggacuuuacguucguuuaa

SEQ ID NO: 146 - RNA, 19 nt, Artificial Sequence
  SYNTHETIC RNA
gaaaugggcuuuaggaaua

SEQ ID NO: 147 - RNA, 19 nt, Artificial Sequence
  SYNTHETIC RNA
caacaugggaguaaucuuu

SEQ ID NO: 148 - RNA, 19 nt, Artificial Sequence
  SYNTHETIC RNA
cuagugauuugcugauuaa

SEQ ID NO: 149 - RNA, 19 nt, Artificial Sequence
  SYNTHETIC RNA
guauauaagcuaccagaaa

SEQ ID NO: 150 - RNA, 19 nt, Artificial Sequence
  SYNTHETIC RNA
cgagaaaggcccacaguau

SEQ ID NO: 151 - RNA, 19 nt, Artificial Sequence
  SYNTHETIC RNA
caagaaggccuaccauuug

SEQ ID NO: 152 - RNA, 19 nt, Artificial Sequence
  SYNTHETIC RNA
gaacagcagcuacauccau

SEQ ID NO: 153 - RNA, 19 nt, Artificial Sequence
  SYNTHETIC RNA
ugacagaggcagacaaggu

SEQ ID NO: 154 - RNA, 19 nt, Artificial Sequence
  SYNTHETIC RNA
caaauuauguggaggcgau

SEQ ID NO: 155 - RNA, 19 nt, Artificial Sequence
  SYNTHETIC RNA
cccuuaaugucccgagcaa

SEQ ID NO: 156 - RNA, 19 nt, Artificial Sequence
  SYNTHETIC RNA
ccucacugaugaacgauga

SEQ ID NO: 157 - RNA, 19 nt, Artificial Sequence
  SYNTHETIC RNA
cugaggugguuccgagcca

SEQ ID NO: 158 - RNA, 19 nt, Artificial Sequence
  SYNTHETIC RNA
ccaacuacguggagugugu

SEQ ID NO: 159 - RNA, 19 nt, Artificial Sequence
  SYNTHETIC RNA
acaaucagccggaaagaga

SEQ ID NO: 160 - RNA, 19 nt, Artificial Sequence
  SYNTHETIC RNA
ggacauggaacaggccuuu

SEQ ID NO: 161 - RNA, 19 nt, Artificial Sequence
  SYNTHETIC RNA
agacaaaagcucaguauga

SEQ ID NO: 162 - RNA, 19 nt, Artificial Sequence
  SYNTHETIC RNA
gaacgaauaucuccuaaca

SEQ ID NO: 163 - RNA, 19 nt, Artificial Sequence
  SYNTHETIC RNA
gcagauaagugaggauucu

SEQ ID NO: 164 - RNA, 19 nt, Artificial Sequence
  SYNTHETIC RNA
caugagggcuuagacauua

SEQ ID NO: 165 - RNA, 19 nt, Artificial Sequence
  SYNTEHTIC RNA
uuaacgaucugauuucuug

SEQ ID NO: 166 - RNA, 19 nt, Artificial Sequence
  SYNTHETIC RNA
ccacacgagcgaugacgaa

SEQ ID NO: 167 - RNA, 19 nt, Artificial Sequence
  SYNTHETIC RNA
gcacaauggcaucgacgga

SEQ ID NO: 168 - RNA, 19 nt, Artificial Sequence
  SYNTHETIC RNA
cugcuuuaccagcgggcuu

SEQ ID NO: 169 - RNA, 19 nt, Artificial Sequence
  SYNTHETIC RNA
gggucauguagccgauauu

SEQ ID NO: 170 - RNA, 19 nt, Artificial Sequence
  SYNTHETIC RNA
gaacugcucaugcguuauc

SEQ ID NO: 171 - RNA, 19 nt, Artificial Sequence
  SYNTHETIC RNA
ggaccuaucucucagcuga

SEQ ID NO: 172 - RNA, 19 nt, Artificial Sequence
  SYNTHETIC RNA
caacgcagcuauaagcaaa

SEQ ID NO: 173 - RNA, 19 nt, Artificial Sequence
  SYNTHETIC RNA
gaggagaagcaguucaaua

SEQ ID NO: 174 - RNA, 19 nt, Artificial Sequence
  SYNTHETIC RNA
aguaagaagcagugcguua

SEQ ID NO: 175 - RNA, 19 nt, Artificial Sequence
  SYNTHETIC RNA
caagauaagugggcggaaa

SEQ ID NO: 176 - RNA, 19 nt, Artificial Sequence
  SYNTHETIC RNA
gcauuagcagucuuagaaa

SEQ ID NO: 177 - RNA, 19 nt, Artificial Sequence
  SYNTHETIC RNA
gguggaauuuaauguauca

SEQ ID NO: 178 - RNA, 19 nt, Artificial Sequence
  SYNTHETIC RNA
acuccuaagccgcgacaga

SEQ ID NO: 179 - RNA, 19 nt, Artificial Sequence
  SYNTHETIC RNA
ggggauuaggggagugaga

SEQ ID NO: 180 - RNA, 19 nt, Artificial Sequence
  SYNTHETIC RNA
uauccaagcugucagaaua

SEQ ID NO: 181 - RNA, 19 nt, Artificial Sequence
  SYNTHETIC RNA
ccacagugucgcugcaaau

SEQ ID NO: 182 - RNA, 19 nt, Artificial Sequence
  SYNTHETIC RNA
uggagauucaggccgauua

SEQ ID NO: 183 - RNA, 19 nt, Artificial Sequence
  SYNTHETIC RNA
gagggagaaccacggccaa

SEQ ID NO: 184 - RNA, 19 nt, Artificial Sequence
  SYNTHETIC RNA
uccagaagcucguguccga

SEQ ID NO: 185 - RNA, 19 nt, Artificial Sequence
  SYNTHETIC RNA
gcccagucucuuugaguaa

SEQ ID NO: 186 - RNA, 19 nt, Artificial Sequence
  SYNTHETIC RNA
gaaauuuaagcaacggcua

SEQ ID NO: 187 - RNA, 19 nt, Artificial Sequence
  SYNTHETIC RNA
acuucaggccgcacgaaua

SEQ ID NO: 188 - RNA, 19 nt, Artificial Sequence
  SYNTHETIC RNA
ccauggauuuaucggugua

SEQ ID NO: 189 - RNA, 19 nt, Artificial Sequence
  SYNTHETIC RNA
aguucaaacaggcgcuuua

SEQ ID NO: 190 - RNA, 19 nt, Artificial Sequence
  SYNTHETIC RNA
guggauagcuuguuuagca

SEQ ID NO: 191 - RNA, 19 nt, Artificial Sequence
  SYNTHETIC RNA
gaaccggccaugcaaguaa

SEQ ID NO: 192 - RNA, 19 nt, Artificial Sequence
  SYNTHETIC RNA
uauccaguccuuaaaguaa

SEQ ID NO: 193 - RNA, 19 nt, Artificial Sequence
  SYNTHETIC RNA
auaguauacuggaguccua

SEQ ID NO: 194 - RNA, 19 nt, Artificial Sequence
  SYNTHETIC RNA
gaagaugcacgaucaguua

SEQ ID NO: 195 - RNA, 19 nt, Artificial Sequence
  SYNTHETIC RNA
guauucaucuucuggcuua

SEQ ID NO: 196 - RNA, 19 nt, Artificial Sequence
  SYNTHETIC RNA
gcagagaugaucaagguua

SEQ ID NO: 197 - RNA, 19 nt, Artificial Sequence
  SYNTHETIC RNA
gaacaacuuaguaggauaa

SEQ ID NO: 198 - RNA, 19 nt, Artificial Sequence
  SYNTHETIC RNA
ggaagugccuggauggaau

SEQ ID NO: 199 - RNA, 19 nt, Artificial Sequence
  SYNTHETIC RNA
aaagaauguuauagcgagu

SEQ ID NO: 200 - RNA, 19 nt, Artificial Sequence
  SYNTHETIC RNA
gauuugaacgcgauugcaa

SEQ ID NO: 201 - RNA, 19 nt, Artificial Sequence
  SYNTHETIC RNA
cagaaugaaggaacgauuu

SEQ ID NO: 202 - RNA, 19 nt, Artificial Sequence
  SYNTHETIC RNA
ggagaauaguaagcguaaa

SEQ ID NO: 203 - RNA, 19 nt, Artificial Sequence
  SYNTHETIC RNA
ccauuuacacggaguuuga

SEQ ID NO: 204 - RNA, 19 nt, Artificial Sequence
  SYNTHETIC RNA
gaaagaacgcaccgaagug

SEQ ID NO: 205 - RNA, 19 nt, Artificial Sequence
  SYNTHETIC RNA
caacaguccuucguacaga

SEQ ID NO: 206 - RNA, 19 nt, Artificial Sequence
  SYNTHETIC RNA
guaacacagggacgagaaa

SEQ ID NO: 207 - RNA, 19 nt, Artificial Sequence
  SYNTHETIC RNA
gaugaagagccacgguuua

SEQ ID NO: 208 - RNA, 19 nt, Artificial Sequence
  SYNTHETIC RNA
ggacaaaggugacggaugg

SEQ ID NO: 209 - RNA, 19 nt, Artificial Sequence
  SYNTHETIC RNA
cagaguguauggugaauua

SEQ ID NO: 210 - DNA, 4065 bp, Homo sapiens
atgtcgactggggacagttttgagactcgatttgaaaaaatggacaacctgctgcgggatcccaaatcggaagtgaattcggattgtttgctggatggattggatgctttggtatatgatttggattttcctgccttaagaaaaaacaaaaatattgacaactttttaagcagatataaagacacaataaataaaatcagagatttacgaatgaaagctgaagattatgaagtagtgaaggtgattggtagaggtgcatttggagaagttcaattggtaaggcataaatccaccaggaaggtatatgctatgaagcttctcagcaaatttgaaatgataaagagatctgattctgcttttttctgggaagaaagggacatcatggcttttgccaacagtccttgggttgttcagcttttttatgcattccaagatgatcgttatctctacatggtgatggaatacatgcctggtggagatcttgtaaacttaatgagcaactatgatgtgcctgaaaaatgggcacgattctatactgcagaagtagttcttgcattggatgcaatccattccatgggttttattcacagagatgtgaagcctgataacatgctgctggataaatctggacatttgaagttagcagattttggtacttgtatgaagatgaataaggaaggcatggtacgatgtgatacagcggttggaacacctgattatatttcccctgaagtattaaaatcccaaggtggtgatggttattatggaagagaatgtgactggtggtcggttggggtatttttatacgaaatgcttgtaggtgatacacctttttatgcagattctttggttggaacttacagtaaaattatgaaccataaaaattcacttacctttcctgatgataatgacatatcaaaagaagcaaaaaaccttatttgtgccttccttactgacagggaagtgaggttagggcgaaatggtgtagaagaaatcaaacgacatctcttcttcaaaaatgaccagtgggcttgggaaacgctccgagacactgtagcaccagttgtacccgatttaagtagtgacattgatactagtaattttgatgacttggaagaagataaaggagaggaagaaacattccctattcctaaagctttcgttggcaatcaactaccttttgtaggatttacatattatagcaatcgtagatacttatcttcagcaaatcctaatgataacagaactagctccaatgcagataaaagcttgcaggaaagtttgcaaaaaacaatctataagctggaagaacagctgcataatgaaatgcagttaaaagatgaaatggagcagaagtgcagaacctcaaacataaaactagacaagataatgaaagaattggatgaagagggaaatcaaagaagaaatctagaatctacagtgtctcagattgagaaggagaaaatgttgctacagcatagaattaatgagtaccaaagaaaagctgaacaggaaaatgagaagagaagaaatgtagaaaatgaagtttctacattaaaggatcagttggaagacttaaagaaagtcagtcagaattcacagcttgctaatgagaagctgtcccagttacaaaagcagctagaagaagccaatgacttacttaggacagaatcggacacagctgtaagattgaggaagagtcacacagagatgagcaagtcaattagtcagttagagtccctgaacagagagttgcaagagagaaatcgaattttagagaattctaagtcacaaacagacaaagattattaccagctgcaagctatattagaagctgaacgaagagacagaggtcatgattctgagatgattggagaccttcaagctcgaattacatctttacaagaggaggtgaagcatctcaaacataatctcgaaaaagtggaaggagaaagaaaagaggctcaagacatgcttaatcactcagaaaaggaaaagaataatttagagatagatttaaactacaaacttaaatcattacaacaacggttagaacaagaggtaaatgaacacaaagtaaccaaagctcgtttaactgacaaacatcaatctattgaagaggcaaagtctgtggcaatgtgtgagatggaaaaaaagctgaaagaagaaagagaagctcgagagaaggctgaaaatcgggttgttcagattgagaaacagtgttccatgctagacgttgatctgaagcaatctcagcagaaactagaacatttgactggaaataaagaaaggatggaggatgaagttaagaatctaaccctgcaactggagcaggaatcaaataagcggctgttgttacaaaatgaattgaagactcaagcatttgaggcagacaatttaaaaggtttagaaaagcagatgaaacaggaaataaatactttattggaagcaaagagattattagaatttgagttagctcagcttacgaaacagtatagaggaaatgaaggacagatgcgggagctacaagatcagcttgaagctgagcaatatttctcgacactttataaaacccaggtaaaggaacttaaagaagaaattgaagaaaaaaacagagaaaatttaaagaaaatacaggaactacaaaatgaaaaagaaactcttgctactcagttggatctagcagaaacaaaagctgagtctgagcagttggcgcgaggccttctggaagaacagtattttgaattgacgcaagaaagcaagaaagctgcttcaagaaatagacaagagattacagataaagatcacactgttagtcggcttgaagaagcaaacagcatgctaaccaaagatattgaaatattaagaagagagaatgaagagctaacagagaaaatgaagaaggcagaggaagaatataaactggagaaggaggaggagatcagtaatcttaaggctgcctttgaaaagaatatcaacactgaacgaacccttaaaacacaggctgttaacaaattggcagaaataatgaatcgaaaagattttaaaattgatagaaagaaagctaatacacaagatttgagaaagaaagaaaaggaaaatcgaaagctgcaactggaactcaaccaagaaagagagaaattcaaccagatggtagtgaaacatcagaaggaactgaatgacatgcaagcgcaattggtagaagaatgtgcacataggaatgagcttcagatgcagttggccagcaaagagagtgatattgagcaattgcgtgctaaacttttggacctctcggattctacaagtgttgctagttttcctagtgctgatgaaactgatggtaacctcccagagtcaagaattgaaggttggctttcagtaccaaatagaggaaatatcaaacgatatggctggaagaaacagtatgttgtggtaagcagcaaaaaaattttgttctataatgacgaacaagataaggagcaatccaatccatctatggtattggacatagataaactgtttcacgttagacctgtaacccaaggagatgtgtatagagctgaaactgaagaaattcctaaaatattccagatactatatgcaaatgaaggtgaatgtagaaaagatgtagagatggaaccagtacaacaagctgaaaaaactaatttccaaaatcacaaaggccatgagtttattcctacactctaccactttcctgccaattgtgatgcctgtgccaaacctctctggcatgtttttaagccaccccctgccctagagtgtcgaagatgccatgttaagtgccacagagatcacttagataagaaagaggacttaatttgtccatgtaaagtaagttatgatgtaacatcagcaagagatatgctgctgttagcatgttctcaggatgaacaaaaaaaatgggtaactcatttagtaaagaaaatccctaagaatccaccatctggttttgttcgtgcttcccctcgaacgctttctacaagatccactgcaaatcagtctttccggaaagtggtcaaaaatacatctggaaaaactagttaa

SEQ ID NO: 211 - protein, 1354 aa, Homo sapiens
MSTGDSFETRFEKMDNLLRDPKSEVNSDCLLDGLDALVYDLDFPALRKNKNIDNFLSRYKDTINKIRDLRMKAEDYEVVKVIGRGAFGEVQLVRHKSTRKVYAMKLLSKFEMIKRSDSAFFWEERDIMAFANSPWVVQLFYAFQDDRYLYMVMEYMPGGDLVNLMSNYDVPEKWARFYTAEVVLALDAIHSMGFIHRDVKPDNMLLDKSGHLKLADFGTCMKMNKEGMVRCDTAVGTPDYISPEVLKSQGGDGYYGRECDWWSVGVFLYEMLVGDTPFYADSLVGTYSKIMNHKNSLTFPDDNDISKEAKNLICAFLTDREVRLGRNGVEEIKRHLFFKNDQWAWETLRDTVAPVVPDLSSDIDTSNFDDLEEDKGEEETFPIPKAFVGNQLPFVGFTYYSNRRYLSSANPNDNRTSSNADKSLQESLQKTIYKLEEQLHNEMQLKDEMEQKCRTSNIKLDKIMKELDEEGNQRRNLESTVSQIEKEKMLLQHRINEYQRKAEQENEKRRNVENEVSTLKDQLEDLKKVSQNSQLANEKLSQLQKQLEEANDLLRTESDTAVRLRKSHTEMSKSISQLESLNRELQERNRILENSKSQTDKDYYQLQAILEAERRDRGHDSEMIGDLQARITSLQEEVKHLKHNLEKVEGERKEAQDMLNHSEKEKNNLEIDLNYKLKSLQQRLEQEVNEHKVTKARLTDKHQSIEEAKSVAMCEMEKKLKEEREAREKAENRVVQIEKQCSMLDVDLKQSQQKLEHLTGNKERMEDEVKNLTLQLEQESNKRLLLQNELKTQAFEADNLKGLEKQMKQEINTLLEAKRLLEFELAQLTKQYRGNEGQMRELQDQLEAEQYFSTLYKTQVKELKEEIEEKNRENLKKIQELQNEKETLATQLDLAETKAESEQLARGLLEEQYFELTQESKKAASRNRQEITDKDHTVSRLEEANSMLTKDIEILRRENEELTEKMKKAEEEYKLEKEEEISNLKAAFEKNINTERTLKTQAVNKLAEIMNRKDFKIDRKKANTQDLRKKEKENRKLQLELNQEREKFNQMVVKHQKELNDMQAQLVEECAHRNELQMQLASKESDIEQLRAKLLDLSDSTSVASFPSADETDGNLPESRIEGWLSVPNRGNIKRYGWKKQYVVVSSKKILFYNDEQDKEQSNPSMVLDIDKLFHVRPVTQGDVYRAETEEIPKIFQILYANEGECRKDVEMEPVQQAEKTNFQNHKGHEFIPTLYHFPANCDACAKPLWHVFKPPPALECRRCHVKCHRDHLDKKEDLICPCKVSYDVTSARDMLLLACSQDEQKKWVTHLVKKIPKNPPSGFVRASPRTLSTRSTANQSFRKVVKNTSGKTS

SEQ ID NO: 212 - DNA, 4167 bp, Homo sapiens
atgagccggcccccgccgacggggaaaatgcccggcgcccccgagaccgcgccgggggacggggcaggcgcgagccgccagaggaagctggaggcgctgatccgagaccctcgctcccccatcaacgtggagagcttgctggatggcttaaattccttggtccttgatttagattttcctgctttgaggaaaaacaagaacatagataatttcttaaatagatatgagaaaattgtgaaaaaaatcagaggtctacagatgaaggcagaagactatgatgttgtaaaagttattggaagaggtgcttttggtgaagtgcagttggttcgtcacaaggcatcgcagaaggtttatgctatgaagcttcttagtaagtttgaaatgataaaaagatcagattctgcctttttttgggaagaaagagatattatggcctttgccaatagcccctgggtggttcagcttttttatgcctttcaagatgataggtatctgtacatggtaatggagtacatgcctggtggagaccttgtaaaccttatgagtaattatgatgtgcctgaaaaatgggccaaattttacactgctgaagttgttcttgctctggatgcaatacactccatgggtttaatacacagagatgtgaagcctgacaacatgctcttggataaacatggacatctaaaattagcagattttggcacgtgtatgaagatggatgaaacaggcatggtacattgtgatacagcagttggaacaccggattatatatcacctgaggttctgaaatcacaagggggtgatggtttctatgggcgagaatgtgattggtggtctgtaggtgttttcctttatgagatgctagtgggggatactccattttatgcggattcacttgtaggaacatatagcaaaattatggatcataagaattcactgtgtttccctgaagatgcagaaatttccaaacatgcaaagaatctcatctgtgctttcttaacagatagggaggtacgacttgggagaaatggggtggaagaaatcagacagcatcctttctttaagaatgatcagtggcattgggataacataagagaaacggcagctcctgtagtacctgaactcagcagtgacatagacagcagcaatttcgatgacattgaagatgacaaaggagatgtagaaaccttcccaattcctaaagcttttgttggaaatcagctgcctttcatcggatttacctactatagagaaaatttattattaagtgactctccatcttgtagagaaactgattccatacaatcaaggaaaaatgaagaaagtcaagagattcagaaaaaactgtatacattagaagaacatcttagcaatgagatgcaagccaaagaggaactggaacagaagtgcaaatctgttaatactcgcctagaaaaaacagcaaaggagctagaagaggagattaccttacggaaaagtgtggaatcagcattaagacagttagaaagagaaaaggcgcttcttcagcacaaaaatgcagaatatcagaggaaagctgatcatgaagcagacaaaaaacgaaatttggaaaatgatgttaacagcttaaaagatcaacttgaagatttgaaaaaaagaaatcaaaactctcaaatatccactgagaaagtgaatcaactccagagacaactggatgaaaccaatgctttactgcgaacagagtctgatactgcagcccggttaaggaaaacccaggcagaaagttcaaaacagattcagcagctggaatctaacaatagagatctacaagataaaaactgcctgctggagactgccaagttaaaacttgaaaaggaatttatcaatcttcagtcagctctagaatctgaaaggagggatcgaacccatggatcagagataattaatgatttacaaggtagaatatgtggcctagaagaagatttaaagaacggcaaaatcttactagcgaaagtagaactggagaagagacaacttcaggagagatttactgatttggaaaaggaaaaaagcaacatggaaatagatatgacataccaactaaaagttatacagcagagcctagaacaagaagaagctgaacataaggccacaaaggcacgactagcagataaaaataagatctatgagtccatcgaagaagccaaatcagaagccatgaaagaaatggagaagaagctcttggaggaaagaactttaaaacagaaagtggagaacctattgctagaagctgagaaaagatgttctctattagactgtgacctcaaacagtcacagcagaaaataaatgagctccttaaacagaaagatgtgctaaatgaggatgttagaaacctgacattaaaaatagagcaagaaactcagaagcgctgccttacacaaaatgacctgaagatgcaaacacaacaggttaacacactaaaaatgtcagaaaagcagttaaagcaagaaaataaccatctcatggaaatgaaaatgaacttggaaaaacaaaatgctgaacttcgaaaagaacgtcaggatgcagatgggcaaatgaaagagctccaggatcagctcgaagcagaacagtatttctcaaccctttataaaacacaagttagggagcttaaagaagaatgtgaagaaaagaccaaacttggtaaagaattgcagcagaagaaacaggaattacaggatgaacgggactctttggctgcccaactggagatcaccttgaccaaagcagattctgagcaactggctcgttcaattgctgaagaacaatattctgatttggaaaaagagaagatcatgaaagagctggagatcaaagagatgatggctagacacaaacaggaacttacggaaaaagatgctacaattgcttctcttgaggaaactaataggacactaactagtgatgttgccaatcttgcaaatgagaaagaagaattaaataacaaattgaaagatgttcaagagcaactgtcaagattgaaagatgaagaaataagcgcagcagctattaaagcacagtttgagaagcagctattaacagaaagaacactcaaaactcaagctgtgaataagttggctgagatcatgaatcgaaaagaacctgtcaagcgtggtaatgacacagatgtgcggagaaaagagaaggagaatagaaagctacatatggagcttaaatctgaacgtgagaaattgacccagcagatgatcaagtatcagaaagaactgaatgaaatgcaggcacaaatagctgaagagagccagattcgaattgaactgcagatgacattggacagtaaagacagtgacattgagcagctgcggtcacaactccaagccttgcatattggtctggatagttccagtataggcagtggaccaggggatgctgaggcagatgatgggtttccagaatcaagattagaaggatggctttcattgcctgtacgaaacaacactaagaaatttggatgggttaaaaagtatgtgattgtaagcagtaagaagattcttttctatgacagtgaacaagataaagaacaatccaatccttacatggttttagatatagacaagttatttcatgtccgaccagttacacagacagatgtgtatagagcagatgctaaagaaattccaaggatattccagattctgtatgccaatgaaggagaaagtaagaaggaacaagaatttccagtggagccagttggagaaaaatctaattatatttgccacaagggacatgagtttattcctactctttatcatttcccaaccaactgtgaggcttgtatgaagcccctgtggcacatgtttaagcctcctcctgctttggagtgccgccgttgccatattaagtgtcataaagatcatatggacaaaaaggaggagattatagcaccttgcaaagtatattatgatatttcaacggcaaagaatctgttattactagcaaattctacagaagagcagcagaagtgggttagtcggttggtgaaaaagatacctaaaaagcccccagctccagacccttttgcccgatcatctcctagaacttcaatgaagatacagcaaaaccagtctattagacggccaagtcgacagcttgccccaaacaaacctagctaa

SEQ ID NO: 213 - protein, 1388 aa, Homo sapiens
MSRPPPTGKMPGAPETAPGDGAGASRQRKLEALIRDPRSPINVESLLDGLNSLVLDLDFPALRKNKNIDNFLNRYEKIVKKIRGLQMKAEDYDVVKVIGRGAFGEVQLVRHKASQKVYAMKLLSKFEMIKRSDSAFFWEERDIMAFANSPWVVQLFYAFQDDRYLYMVMEYMPGGDLVNLMSNYDVPEKWAKFYTAEVVLALDAIHSMGLIHRDVKPDNMLLDKHGHLKLADFGTCMKMDETGMVHCDTAVGTPDYISPEVLKSQGGDGFYGRECDWWSVGVFLYEMLVGDTPFYADSLVGTYSKIMDHKNSLCFPEDAEISKHAKNLICAFLTDREVRLGRNGVEEIRQHPFFKNDQWHWDNIRETAAPVVPELSSDIDSSNFDDIEDDKGDVETFPIPKAFVGNQLPFIGFTYYRENLLLSDSPSCRETDSIQSRKNEESQEIQKKLYTLEEHLSNEMQAKEELEQKCKSVNTRLEKTAKELEEEITLRKSVESALRQLEREKALLQHKNAEYQRKADHEADKKRNLENDVNSLKDQLEDLKKRNQNSQISTEKVNQLQRQLDETNALLRTESDTAARLRKTQAESSKQIQQLESNNRDLQDKNCLLETAKLKLEKEFINLQSALESERRDRTHGSEIINDLQGRICGLEEDLKNGKILLAKVELEKRQLQERFTDLEKEKSNMEIDMTYQLKVIQQSLEQEEAEHKATKARLADKNKIYESIEEAKSEAMKEMEKKLLEERTLKQKVENLLLEAEKRCSLLDCDLKQSQQKINELLKQKDVLNEDVRNLTLKIEQETQKRCLTQNDLKMQTQQVNTLKMSEKQLKQENNHLMEMKMNLEKQNAELRKERQDADGQMKELQDQLEAEQYFSTLYKTQVRELKEECEEKTKLGKELQQKKQELQDERDSLAAQLEITLTKADSEQLARSIAEEQYSDLEKEKIMKELEIKEMMARHKQELTEKDATIASLEETNRTLTSDVANLANEKEELNNKLKDVQEQLSRLKDEEISAAAIKAQFEKQLLTERTLKTQAVNKLAEIMNRKEPVKRGNDTDVRRKEKENRKLHMELKSEREKLTQQMIKYQKELNEMQAQIAEESQIRIELQMTLDSKDSDIEQLRSQLQALHIGLDSSSIGSGPGDAEADDGFPESRLEGWLSLPVRNNTKKFGWVKKYVIVSSKKILFYDSEQDKEQSNPYMVLDIDKLFHVRPVTQTDVYRADAKEIPRIFQILYANEGESKKEQEFPVEPVGEKSNYICHKGHEFIPTLYHFPTNCEACMKPLWHMFKPPPALECRRCHIKCHKDHMDKKEEIIAPCKVYYDISTAKNLLLLANSTEEQQKWVSRLVKKIPKKPPAPDPFARSSPRTSMKIQQNQSIRRPSRQLAPNKPS

SEQ ID NO: 214 - DNA, 3615 bp, Homo sapiens
atgtccttcttcaatttccgtaagatcttcaagttggggagcgagaagaagaagaagcagtacgaacacgtgaagagggacctgaaccccgaagacttttgggagattataggagaactgggcgacggagcctttgggaaagtgtacaaggcccagaataaagagaccagtgttttagctgctgcaaaagtgattgacactaaatctgaagaagaacttgaagattacatggtagagattgacatattagcatcttgtgatcacccaaatatagtcaagcttctagatgccttctattatgagaacaatctttggatcctcattgaattttgtgcaggtggagcagtagatgctgtgatgcttgaacttgagagaccattaactgagtcccaaatacaagtagtttgcaagcagactttagatgcattgaactacttacatgataataagatcatccacagagatctgaaggctggcaacattctctttaccttagatggagatatcaaattggcggattttggagtatcagctaaaaacacgaggacaattcaaagaagagattcctttattggtacaccatattggatggctcctgaagtagtcatgtgtgaaacatctaaggacagaccctatgactacaaagctgatgtttggtccctgggtatcactttaatagaaatggctgagatagaaccacctcatcatgaattaaatccaatgcgagtgctgctaaaaatagcaaaatctgagccacctacattagcacagccatccagatggtcttcaaattttaaggactttctaaagaaatgcttagaaaagaatgtggatgccaggtggactacatctcagctgctgcagcatccctttgttactgttgattccaacaaacccatccgagaattgattgcagaggcgaaggctgaagtaacagaagaagttgaagatggcaaagaggaagatgaagaggaggaaacagaaaattctctgccaatacctgcaagtaagcgtgcatcttctgaccttagtatcgccagctctgaagaagataaactttcacaaaatgcttgtattttggagtctgtctcagaaaaaacagaacgtagtaactctgaagataaactcaacagcaaaattcttaatgaaaaacccaccactgatgaacctgaaaaggctgtggaggatattaatgaacatattaccgatgctcagttagaagcaatgactgaactccatgacagaacagcagtaatcaaggagaatgaaagagagaagaggcccaagcttgaaaatctgcctgacacagaagaccaagaaactgtggacattaattcagtcagtgaaggaaaagagaataatataatgataaccttagaaacaaatattgaacataatctaaaatctgaggaagaaaaggatcaggaaaagcaacagatgtttgaaaataagcttataaaatctgaagaaattaaagatactattttgcaaacagtagatttagtttctcaagagactggagaaaaagaggcaaatattcaggcagttgatagtgaagttgggcttacaaaggaagacacccaagagaaattgggggaagacgacaaaactcaaaaagatgtgatcagcaatacaagtgatgtgataggaacatgtgaggcagcagatgtggctcagaaagtggatgaagacagtgctgaggatacgcagagtaatgatgggaaagaagtggtcgaagtaggccagaaattaattaataagcccatggtgggtcctgaggctggtggtactaaggaagttcctattaaagaaatagttgaaatgaatgaaatagaagaaggtaaaaataaggaacaagcaataaacagttcagagaacataatggacatcaatgaggaaccaggaacaactgaaggtgaagaaatcactgagtcaagtagcactgaagaaatggaggtcagaagtgtggtggctgatactgaccaaaaggctttaggaagtgaagttcaggatgcttctaaagtcactactcagatagataaagagaaaaaagaaattccagtgtcaattaaaaaagagcctgaagttactgtagtttcacagcccactgaacctcagcctgttctaatacccagtattaatatcaactctgacagtggagaaaataaagaagaaataggttctttatcaaaaactgaaactattctgccaccagaatctgagaatccaaaggaaaatgataatgattcaggcactggttccactgctgatactagcagtattgacttgaatttatccatctctagctttctaagtaaaactaaagacagtggatcgatatctttacaagaaacaagaagacaaaagaaaacattgaagaaaacacgcaaatttattgttgatggtgtagaagtgagtgtaacaacatcaaagatagttacagatagtgattccaaaactgaagaattgcggtttcttagacgtcaggaacttcgggaattaagatttcttcagaaagaagagcaaagagcccaacaacagctcaatagcaaactacagcaacaacgagaacaaattttccggcgctttgagcaggaaatgatgagtaaaaagcgacaatatgaccaggaaattgagaatctagaaaaacagcagaaacagactatcgaacgcctggaacaagagcacacaaatcgcttgcgagatgaagccaaacgcatcaaaggagaacaagagaaagagttgtccaaatttcagaatatgctgaagaaccgaaagaaggaggaacaagagtttgttcagaaacaacagcaagaattagatggctctctgaaaaagatcatccagcagcagaaggcagagttagctaatattgagagagagtgcctgaataacaagcaacagctcatgagagctcgagaagctgcaatttgggagctcgaagaacgacacttacaagaaaaacaccagctgctcaaacagcagcttaaagatcagtatttcatgcaaagacatcagctacttaagcgccacgagaaggaaacagagcaaatgcagcgttacaatcaaagacttattgaggaattgaaaaacagacagactcaagaaagagcaagactgcccaagattcagcgcagtgaagccaagactcgaatggccatgtttaagaagagtttgagaattaactcaacagccacaccagatcaggaccgtgataaaattaaacagtttgctgcacaagaagaaaagaggcagaaaaatgagagaatggctcagcatcagaaacatgagaatcaaatgcgagatcttcagttgcagtgtgaagccaatgtccgcgaactgcatcagctgcagaatgaaaaatgccacttgttggttgagcatgagactcagaaactgaaggagttagatgaggaacatagccaagaattaaaggagtggagagagaaattgagacctaggaaaaagacactggaagaagagtttgccaggaaactacaggaacaggaagtattctttaaaatgactggggagtctgaatgccttaacccatcaacacagagccggatttccaaattttatcctattcccagcttgcattccaccggatcataa

SEQ ID NO: 215 - protein, 1204 aa, Homo sapiens
MSFFNFRKIFKLGSEKKKKQYEHVKRDLNPEDFWEIIGELGDGAFGKVYKAQNKETSVLAAAKVIDTKSEEELEDYMVEIDILASCDHPNIVKLLDAFYYENNLWILIEFCAGGAVDAVMLELERPLTESQIQVVCKQTLDALNYLHDNKIIHRDLKAGNILFTLDGDIKLADFGVSAKNTRTIQRRDSFIGTPYWMAPEVVMCETSKDRPYDYKADVWSLGITLIEMAEIEPPHHELNPMRVLLKIAKSEPPTLAQPSRWSSNFKDFLKKCLEKNVDARWTTSQLLQHPFVTVDSNKPIRELIAEAKAEVTEEVEDGKEEDEEEETENSLPIPASKRASSDLSIASSEEDKLSQNACILESVSEKTERSNSEDKLNSKILNEKPTTDEPEKAVEDINEHITDAQLEAMTELHDRTAVIKENEREKRPKLENLPDTEDQETVDINSVSEGKENNIMITLETNIEHNLKSEEEKDQEKQQMFENKLIKSEEIKDTILQTVDLVSQETGEKEANIQAVDSEVGLTKEDTQEKLGEDDKTQKDVISNTSDVIGTCEAADVAQKVDEDSAEDTQSNDGKEVVEVGQKLINKPMVGPEAGGTKEVPIKEIVEMNEIEEGKNKEQAINSSENIMDINEEPGTTEGEEITESSSTEEMEVRSVVADTDQKALGSEVQDASKVTTQIDKEKKEIPVSIKKEPEVTVVSQPTEPQPVLIPSININSDSGENKEEIGSLSKTETILPPESENPKENDNDSGTGSTADTSSIDLNLSISSFLSKTKDSGSISLQETRRQKKTLKKTRKFIVDGVEVSVTTSKIVTDSDSKTEELRFLRRQELRELRFLQKEEQRAQQQLNSKLQQQREQIFRRFEQEMMSKKRQYDQEIENLEKQQKQTIERLEQEHTNRLRDEAKRIKGEQEKELSKFQNMLKNRKKEEQEFVQKQQQELDGSLKKIIQQQKAELANIERECLNNKQQLMRAREAAIWELEERHLQEKHQLLKQQLKDQYFMQRHQLLKRHEKETEQMQRYNQRLIEELKNRQTQERARLPKIQRSEAKTRMAMFKKSLRINSTATPDQDRDKIKQFAAQEEKRQKNERMAQHQKHENQMRDLQLQCEANVRELHQLQNEKCHLLVEHETQKLKELDEEHSQELKEWREKLRPRKKTLEEEFARKLQEQEVFFKMTGESECLNPSTQSRISKFYPIPSLHSTGS

SEQ ID NO: 216 - DNA, 2907 bp, Homo sapiens
atggcttttgccaatttccgccgcatcctgcgcctgtctaccttcgagaagagaaagtcccgcgaatatgagcacgtccgccgcgacctggaccccaacgaggtgtgggagatcgtgggcgagctgggcgacggcgccttcggcaaggtttacaaggccaagaataaggagacgggtgctttggctgcggccaaagtcattgaaaccaagagtgaggaggagctggaggactacatcgtggagattgagatcctggccacctgcgaccacccctacattgtgaagctcctgggagcctactatcacgacgggaagctgtggatcatgattgagttctgtccagggggagccgtggacgccatcatgctggagctggacagaggcctcacggagccccagatacaggtggtttgccgccagatgctagaagccctcaacttcctgcacagcaagaggatcatccaccgagatctgaaagctggcaacgtgctgatgaccctcgagggagacatcaggctggctgactttggtgtgtctgccaagaatctgaagactctacagaaacgagattccttcatcggcacgccttactggatggcccccgaggtggtcatgtgtgagaccatgaaagacacgccctacgactacaaagccgacatctggtccctgggcatcacgctgattgagatggcccagatcgagccgccacaccacgagctcaaccccatgcgggtcctgctaaagatcgccaagtcggaccctcccacgctgctcacgccctctaagtggtctgtagagttccgtgacttcctgaagatagccctggataagaacccagaaacccgacccagtgccgcgcagctgctggagcatcccttcgtcagcagcatcaccagtaacaaggctctgcgggagctggtggctgaggccaaggccgaggtgatggaagagatcgaagacggccgggatgagggggaagaggaggacgccgtggatgccgcctccaccctggagaaccatactcagaactcctctgaggtgagtccgccaagcctcaatgctgacaagcctctcgaggagtcaccttccaccccgctggcacccagccagtctcaggacagtgtgaatgagccctgcagccagccctctggggacagatccctccaaaccaccagtcccccagtcgtggcccctggaaatgagaacggcctggcagtgcctgtgcccctgcggaagtcccgacccgtgtcaatggatgccagaattcaggtagcccaggagaagcaagttgctgagcagggtggggacctcagcccagcagccaacagatctcaaaaggccagccagagccggcccaacagcagcgccctggagaccttgggtggggagaagctggccaatggcagcctggagccacctgcccaggcagctccagggccttccaagagggactcggactgcagcagcctctgcacctctgagagcatggactatggtaccaatctctccactgacctgtcgctgaacaaagagatgggctctctgtccatcaaggacccgaaactgtacaaaaaaaccctcaagcggacacgcaaatttgtggtggatggtgtggaggtgagcatcaccacctccaagatcatcagcgaagatgagaagaaggatgaggagatgagatttctcaggcgccaggaactccgagagcttcggctgctccagaaagaagagcatcggaaccagacccagctgagtaacaagcatgagctgcagctggagcaaatgcataaacgttttgaacaggaaatcaacgccaagaagaagttctttgacacggaattagagaacctggagcgtcagcaaaagcagcaagtggagaagatggagcaagaccatgccgtgcgccgccgggaggaggccaggcggatccgcctggagcaggatcgggactacaccaggttccaagagcagctcaaactgatgaagaaagaggtgaagaacgaggtggagaagctcccccgacagcagcggaaggaaagcatgaagcagaagatggaggagcacacgcagaaaaagcagcttcttgaccgggactttgtagccaagcagaaggaggacctggagctggccatgaagaggctcaccaccgacaacaggcgggagatctgtgacaaggagcgcgagtgcctcatgaagaagcaggagctccttcgagaccgggaagcagccctgtgggagatggaagagcaccagctgcaggagaggcaccagctggtgaagcagcagctcaaagaccagtacttcctccagcggcacgagctgctgcgcaagcatgagaaggagcgggagcagatgcagcgctacaaccagcgcatgatagagcagctgaaggtgcggcagcaacaggaaaaggcgcggctgcccaagatccagaggagtgagggcaagacgcgcatggccatgtacaagaagagcctccacatcaacggcgggggcagcgcagctgagcagcgtgagaagatcaagcagttctcccagcaggaggagaagaggcagaagtcggagcggctgcagcaacagcagaaacacgagaaccagatgcgggacatgctggcgcagtgtgagagcaacatgagcgagctgcagcagctgcagaatgaaaagtgccacctcctggtagagcacgaaacccagaaactgaaggccctggatgagagccataaccagaacctgaaggaatggcgggacaagcttcggccgcgcaagaaggctctggaagaggatctgaaccagaagaagcgggagcaggagatgttcttcaagctgagcgaggaggcggagtgcccaaacccctccaccccaagcaaggccgccaagttcttcccctacagttctgcggatgcttcttaa

SEQ ID NO: 217 - protein, 968 aa, Homo sapiens
MAFANFRRILRLSTFEKRKSREYEHVRRDLDPNEVWEIVGELGDGAFGKVYKAKNKETGALAAAKVIETKSEEELEDYIVEIEILATCDHPYIVKLLGAYYHDGKLWIMIEFCPGGAVDAIMLELDRGLTEPQIQVVCRQMLEALNFLHSKRIIHRDLKAGNVLMTLEGDIRLADFGVSAKNLKTLQKRDSFIGTPYWMAPEVVMCETMKDTPYDYKADIWSLGITLIEMAQIEPPHHELNPMRVLLKIAKSDPPTLLTPSKWSVEFRDFLKIALDKNPETRPSAAQLLEHPFVSSITSNKALRELVAEAKAEVMEEIEDGRDEGEEEDAVDAASTLENHTQNSSEVSPPSLNADKPLEESPSTPLAPSQSQDSVNEPCSQPSGDRSLQTTSPPVVAPGNENGLAVPVPLRKSRPVSMDARIQVAQEKQVAEQGGDLSPAANRSQKASQSRPNSSALETLGGEKLANGSLEPPAQAAPGPSKRDSDCSSLCTSESMDYGTNLSTDLSLNKEMGSLSIKDPKLYKKTLKRTRKFVVDGVEVSITTSKIISEDEKKDEEMRFLRRQELRELRLLQKEEHRNQTQLSNKHELQLEQMHKRFEQEINAKKKFFDTELENLERQQKQQVEKMEQDHAVRRREEARRIRLEQDRDYTRFQEQLKLMKKEVKNEVEKLPRQQRKESMKQKMEEHTQKKQLLDRDFVAKQKEDLELAMKRLTTDNRREICDKERECLMKKQELLRDREAALWEMEEHQLQERHQLVKQQLKDQYFLQRHELLRKHEKEREQMQRYNQRMIEQLKVRQQQEKARLPKIQRSEGKTRMAMYKKSLHINGGGSAAEQREKIKQFSQQEEKRQKSERLQQQQKHENQMRDMLAQCESNMSELQQLQNEKCHLLVEHETQKLKALDESHNQNLKEWRDKLRPRKKALEEDLNQKKREQEMFFKLSEEAECPNPSTPSKAAKFFPYSSADAS

SEQ ID NO: 218 - DNA, 20 bp, Artificial Sequence
  SYNTHETIC PRIMER
gctgcttctcattgtctcgg

SEQ ID NO: 219 - DNA, 20 bp, Artificial Sequence
  SYNTHETIC PRIMER
gccaggagaatgaggtggtc

SEQ ID NO: 220 - DNA, 20 bp, Artificial Sequence
  SYNTHETIC PRIMER
cctgctgcttatcgtggctg

SEQ ID NO: 221 - DNA, 20 bp, Artificial Sequence
  SYNTHETIC PRIMER
gccaggagaatgaggtggtc

(19) United States
(12) Patent Application Publication
(10) Pub. No.: US 2024/0167037 A1
(43) Pub. Date: May 23, 2024

(54) CANCER THERAPY

(71) Applicant: NATIONAL UNIVERSITY CORPORATION KOBE UNIVERSITY, Kobe-shi (JP)

(72) Inventors: Kazuya TSUJITA, Kobe-shi (JP); Toshiki ITOH, Kobe-shi (JP); Kiyoko FUKAMI, Hachioji (JP); Reiko SATOW, Hachioji (JP)

(73) Assignee: NATIONAL UNIVERSITY CORPORATION KOBE UNIVERSITY, Kobe-shi (JP)

(21) Appl. No.: 18/550,777

(22) PCT Filed: Mar. 17, 2022
(86) PCT No.: PCT/IB2022/052457

§ 371 Date: Sep. 15, 2023

Related U.S. Application Data
(60) Provisional application No. 63/162,214, filed on Mar. 17, 2021.

(51) Int. Cl. C12N 15/113; A61K 9/00; A61K 9/20; A61K 9/48; A61K 31/7088; A61K 38/45; A61K 38/46; A61K 47/02; A61K 47/06; A61K 47/12; A61K 48/00; A61P 35/00; C12N 15/115

(52) U.S. Cl. CPC C12N 15/113; A61K 9/0019; A61K 9/0073; A61K 9/2013; A61K 9/2018; A61K 9/2027; A61K 9/2054; A61K 9/2059; A61K 9/4858; A61K 9/4866; A61K 31/7088; A61K 38/45; A61K 38/465; A61K 47/02; A61K 47/06; A61K 47/12; A61K 48/005; A61P 35/00; C12N 15/115; C12Y 207/01068; C12N 2310/14; C12N 2310/141; C12N 2310/531

(57) ABSTRACT

The disclosure provides for agents, compositions of the agent for the use in treating cancer by contacting a cancer cell with an agent to increase the tension of a plasma membrane of the cancer cell, thereby preventing or reducing cell migration and/or proliferation of the cancer cell and treating the cancer.

### RELATED APPLICATIONS

This application claims priority under 35 U.S.C. § 119(e) to U.S. Provisional Patent Application No. 63/162,214 filed Mar. 17, 2021, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Mar. 17, 2022, is named 521_003WO1_SL.txt and is 45,200 bytes in size.

### BACKGROUND OF THE INVENTION

Although metastasis is the major cause of cancer-related deaths, our understanding of key determinants underlying tumor dissemination is limited. From a biophysical perspective, it has long been proposed that changes in cell mechanics are integral to metastatic dissemination given that cancer cells must undergo extensive deformation to migrate through tissues and enter blood vessels. Recent advances in nanotechnology have unveiled ‘mechanical signatures’ of malignant cells, as showed by the strong correlation between decreasing cell stiffness and increasing invasive and metastatic efficiency. This suggests that heritable changes in cell mechanics, which should be caused by malignant progression, are critical to the metastatic process. Conversely, epithelial cells may already have strategies to maintain mechanical homeostasis, which could function as an endogenous tumor suppressor. However, the key cell-intrinsic physical parameters for the transition to the malignant phenotype remain unknown, limiting our understanding of the link between oncogenic signaling and dysregulation of cell mechanics, and of how mechanical changes are transduced (mechanotransduction) to regulate cancer cell motility.

Cell motility is fundamental to metastatic dissemination. Recent studies using three-dimensional (3D) environments have indicated that malignant cells exhibit two different invasive migration modes, termed mesenchymal and amoeboid migration. Mesenchymal migration, which shows a spindle-like shape, depends on PM protrusions pushed forward by Arp 2/3 complex-dependent branched actin polymerization. In contrast, amoeboid migration, which is characterized by a rounded morphology, is highly heterogeneous and displays both actin-based protrusions and contractility-driven membrane blebs. Importantly, these two migration modes are interconvertible: cancer cells can actively switch between these modes of movement and even exhibit mixed phenotypes of both. Such plasticity and complexity is considered a major obstacle in developing therapeutic strategies to limit tumor invasion and dissemination.

The PM reversibly associates with the actin cortex via linker proteins, such as ezrin, radixin, and moesin (ERM) family proteins, whereby cell membrane mechanics are intrinsically dependent on the degree of membrane-cortex adhesion (MCA). Indeed, it has emerged that PM tension, which is primarily determined by this composite structure, plays an essential role in cell shape changes and motility. Given that tense membranes resist cell membrane deformations, PM tension is assumed to disfavor the formation of any membrane protrusions and ultimately, cell motility.

Accordingly, there is a need for compounds and methods of use that increase membrane tension, thereby suppressing metastasis of malignant cells. The present disclosure satisfies these needs.

### SUMMARY OF THE INVENTION

In the present disclosure, optical tweezers, genetic interference, cancer genome data, mechanical perturbations, and in vivo studies are used to identify homeostatic PM tension as a mechanical suppressor of cancer cell dissemination by counteracting mechanosensitive BAR proteins. This data demonstrates that reduced PM tension is a mechanical hallmark of malignant cells, regardless of whether they display mesenchymal or amoeboid motility modes. Maintaining high PM tension is sufficient to suppress such 3D migration modes, tumor invasion, and metastasis while having, in principle, no effect on non-tumorigenic cells. This work paves the way for new precise therapeutic strategies for the treatment of metastatic cancers by targeting the cell membrane mechanics of cancer cells.

Accordingly, the disclosure provides for method of treating cancer comprising contacting a cancer cell with an agent to increase tension of a plasma membrane of the cancer cell, thereby treating the cancer cell.

In some embodiments, the agent causes the tension of the plasma membrane to increase to and/or is maintained at about 100-200 pN/μm or greater. This may be accomplished in many ways, such as, but not limited to, increasing an internal pressure of the cell by manipulating a permeability function of the cell (increasing or decreasing the permeability of the plasma membrane), or increasing or decreasing an amount of a component of the plasma membrane. Such components of the plasma membrane are one or more of a lipid, a phospholipid, a glycolipid, a protein, a glycoprotein, and cholesterol. In some embodiments, the phospholipid is phosphatidylinositol 4,5-bisphosphate (PIP2), and the amount of PIP2 in the plasma membrane is increased.

In some embodiments, the agent used to increase the tension of the plasma membrane causes an increase in a membrane-actin cortex attachment (MCA) wherein the increase in the MCA is compared to a cell that is not contacted by the agent. In some embodiments, the agent used to increase the MCA is an expression vector or construct comprising one or more of phosphatidylinositol 4-phosphate 5-kinase (PIP5K) genes, wherein the PIP5K gene encodes one or more of PIP5K1A, PIP5K1B, and PIP5K1C to produce phosphatidylinositol 4,5-bisphosphate (PIP2).

In some embodiments, the agent increases the tension of the plasma membrane by causing an increase in expression of an ezrin, radixin, and moesin (ERM) protein comprising one or more of EZR, RDX, and MSN. In some embodiments, certain genes may be used to activate the ERM proteins such as an upstream kinase of an ERM protein. In some embodiments, the kinase is one or more of RHOA, ROCK1, ROCK2, SLK, and STK10 that directly or indirectly phosphorylates an ERM protein. In some embodiments, the agent is one or more expression vectors that comprise one or more ERM proteins and/or one or more upstream kinases

In other embodiments, the agent inhibits or reduces expression of one or more proteins comprising a Bin, Amphiphysin, and Rvs (BAR) domain. Preferably, the BAR domain proteins comprise a gene product of one or more of MTSS1L/ABBA, FNBP1L/Toca-1, TRIP10/CIP4, ARHGAP4, ARHGAP10/GARF2, ARHGAP17/RICH1, ARHGAP26/GRAF1, ARHGAP29, ARHGAP42/GRAF, ARHGAP44/RICH2, ARHGAP45/HMHA1, ARHGEF37, ARHGEF38, IRSp53/BAIAP2, BAIAP2L1/IRTKS, DNMBP/Tuba, FCHSD1, FCHSD2, FER; FES, FNBP1/FBP17, GAS7, GMIP, MTSS1/MIM, OPHN1, PACSIN1, PACSIN2, PACSIN3, SH3BP1, SRGAP1, SRGAP2, and SRGAP3.

In some embodiments, the agent is an antibody, aptamer, short interfering RNA (siRNA), micro-RNA (miRNA), short hairpin RNA (shRNA), a nanobody, an affimer, DNA, a CRISPR/Cas9 system, or a chemical compound.

In some embodiments, the agent is one or more of siRNA, miRNA, and shRNA that binds to one or more of a messenger ribonucleic acid of MTSS1L/ABBA, FNBP1/FBP17, TRIP10/CIP4, ARHGAP4, ARHGAP10/GARF2, ARHGAP17/RICH1, ARHGAP26/GRAF1, ARHGAP29, ARHGAP42/GRAF, ARHGAP44/RICH2, ARHAGAP45/HMHA1, ARHGEF37, ARHGEF38, IRSp53/BAIAP2, BAIAP2L1/IRTKS, DNMBP/Tuba, FCHSD1, FCHSD2, FER; FES, FNBP1L/Toca-1, GAS7, GMIP, MTSS1/MIM, OPHN1, PACSIN1, PACSIN2, PACSIN3, SH3BP1, SRGAP1, SRGAP2, and SRGAP3. In some embodiments, the one or more of siRNA, miRNA, and shRNA binds to a messenger ribonucleic acid of MTSS1L/ABBA, FNBP1/FBP17, and TRIP10/CIP4.

In some embodiments, the agent is an expression vector or construct configured to express an ezrin fusion protein comprising a conserved myristylation sequence of Lyn fused with ezrin, wherein the ezrin comprises a phosphomimic activating mutation (T567E). In other embodiments, the agent is an expression vector or construct configured to express one or more kinases selected from ROCK1, ROCK2, SLK, STK10, and RHOA that directly or indirectly phosphorylate an ERM protein, thereby activating the ERM protein.

In some embodiments, the agent is formulated as a composition comprising a pharmaceutically acceptable carrier.

The disclosure also provides for a method of inhibiting migration and/or proliferation of a cell comprising reducing expression of one or more proteins comprising a BAR domain or increasing expression of an ezrin, radixin, and moesin (ERM) protein, thereby inhibiting the migration and/or proliferation of a cell. In some embodiments, an increase in ERM phosphorylation causes the inhibition of migration and/or proliferation of the cell.

In another embodiment, the disclosure provides for a method of increasing or decreasing the rate of cell division of eukaryotic cells in a cell culture comprising contacting the eukaryotic cell with an agent to cause a change in the tension of a plasma membrane of the cell, thereby increasing or decreasing the rate of cell division compared to a rate of cell division of a Eukaryotic cell not contacted with the agent.

In some embodiments, the agent causing the increase in the rate of cell division decreases expression of one or more ezrin, radixin, and moesin (ERM) proteins or decreases phosphorylation of one or more ERM proteins, thereby causing an increase in the cell division. In another embodiment, the agent is an expression vector or construct configured to express an ezrin fusion protein comprising a conserved myristylation sequence of Lyn fused with ezrin, wherein the ezrin comprises a phosphomimic activating mutation (T567E).

In other embodiments, the agent causing the increasing in the rate of cell division increases expression of one or more BAR domain proteins. In other embodiments, the agent causing the decrease in in cell division increases expression of one or more ezrin, radixin, and moesin (ERM) proteins or phosphorylation of one or more ERM proteins, or decreases the expression of one or more BAR domain proteins. Preferably, the agent is an antisense RNA, a siRNA, shRNA, or miRNA or an antibody.

These and other features and advantages of this invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Plasma membrane (PM) tension is higher in non-invasive cells than in their metastatic counterparts. a Scatter plot comparing the tether force of the indicated cells. n=35 (MCF10A), n=24 (MDCK II), n=31 (IAR-2), n=23 (AU565), n=23 (MCF7), n=24 (MDA-MB-231 with ruffling), n=14 (MDA-MB-231 with blebbing), n=21 (Hs578T with ruffling), n=13 (Hs578T with blebbing), n=19 (PC-3 with ruffling), n=14 (PC-3 with blebbing), n=24 (PANC-1) cells pooled from three independent experiments. Mean SD. b Quantification of confocal images of the indicated cells stained with anti-phosphorylated ERM (pERM) antibodies and phalloidin. Membrane/cytoplasm intensity ratio of pERM and F-actin of n=30 (MCF10A), n=26 (Au565), n=24 (MDA-MB-231 with ruffling), n=20 (MDA-MB-231 with blebbing), n=22 (Hs578T with ruffling), and n=18 (Hs578T with blebbing) cells pooled from three independent experiments. Mean±SD. **P=0.0073; ***P=0.0014. c Quantification of confocal images of MCF10A or MDA-MB-231 cells stained with anti-pERM antibodies, phalloidin, and wheat germ agglutinin (WGA) in a 3D collagen matrix (3D). Plasma membrane (PM) was labeled with WGA. Membrane/cytoplasm intensity ratio of pERM and F-actin of n=24 (MCF10A), n=16 (AU565), n=15 (MDA-MB-231, elongated), n=12 (MDA-MB-231, rounded with actin-based protrusion), n=17 (MDA-MB-231, rounded with blebs), and n=17 (Hs578T) cells pooled from three independent experiments. Mean±SD. Significance tested using one-way ANOVA with Tukey's multiple comparisons test (a) and the two-tailed Mann-Whitney test (b, c). n.s., not significant; ****P<0.0001. All scale bars, 10 μm.

FIG. 2. Decreased PM tension transforms epithelial cells into a mesenchymal migratory phenotype in both 2D and 3D environments. a Scatter plots comparing the estimated PM tension of MCF10A cells treated with the indicated RNAi. n=60 (si-Control), n=40 (si-RHOA), n=28 (si-ERM), and n=25 (si-SLK+STK10) cells pooled from three independent experiments. Mean±SD. b Quantification of confocal images of MCF10A cells treated with RNAi, stained with anti-pERM antibodies and phalloidin. (Membrane/cytoplasm intensity ratio of pERM and F-actin of n=29 (si-Control), n=26 (si-RHOA), n=19 (si-ERM), and n=28 (si-SLK+STK10) cells pooled from three independent experiments. Mean±SD. c Phase-contrast images of MCF10A cells treated with the indicated RNAi grown in 3D on-top culture. Images are representative of three independent experiments with similar results. Scale bar, 20 μm. d Quantification of siRNA-treated MCF10A cells migrated through 8 μm pores. n=9 fields from three independent experiments. Mean±SD. **P=0.0011. e Quantification of 3D migration phenotypes of n=155 (si-Control), n=126 (si-RHOA), n=128 (si-ERM), and n=123 (si-SLK+STK10) cells from three independent experiments. f Quantification of confocal images of the indicated RNAi-treated MCF10A cells stained with anti-pERM antibodies and phalloidin in 3D. Membrane/cytoplasm intensity ratio of pERM and F-actin of n=20 (si-Control), n=14 (si-RHOA), n=15 (si-ERM), and n=18 (si-SLK+STK10) cells pooled from three independent experiments. Mean±SD. Significance tested using the two-tailed Mann-Whitney test (a, b, e), two-tailed Student's t-test (c,), and chi-square test (d). ****P<0.0001.

FIG. 3. Correlation between decreased PM tension and malignant progression. a Quantification of confocal images of MCF10A or Snail-expressing cells stained with anti-pERM antibodies and phalloidin. Membrane/cytoplasm intensity ratio of pERM and F-actin of n=23 (MCF10A) and n=25 (Snail-expressing cells) cells pooled from three independent experiments. Mean±SD. b Scatter plots comparing the estimated PM tension of the indicated cells. n=38 (MCF10A), n=33 (Snail-expressing cells), and n=36 (Slug-expressing cells) cells pooled from three independent experiments. Mean±SD. c Phase-contrast images of MCF10A cells treated with the indicated RNAi grown in 3D on-top culture. Images are representative of three independent experiments with similar results. Scale bar, 20 μm. d Quantification of confocal images of MCF10A or Snail-expressing cells stained with anti-pERM antibodies and phalloidin in 3D. The yellow arrowhead indicates actin-based protrusion. Scale bars, 10 μm. Membrane/cytoplasm intensity ratio of pERM and F-actin of n=21 (MCF10A) and n=21 (Snail-expressing cells) cells pooled from three independent experiments. Mean±SD. e Genetic alterations of RHOA, SLK, and STK10 across 14 carcinoma types in The Cancer Genome Atlas (TCGA) data (6586 samples). f Kaplan-Meier plots showing the overall survival of breast, lung, and gastric cancer patients, which were stratified according to the mRNA expression of SLK+STK10. Significance tested using the two-tailed Mann-Whitney test (a, b, c) and two-tailed log-rank test (e). ****P<0.0001.

FIG. 4. Increasing PM tension is sufficient for the suppression of 3D migration and metastasis. a Upper, schematic outline of membrane-anchoring active ezrin. Lower, scatter plots comparing the estimated PM tension of the indicated cells. n=26 (parental), n=29 (ezrin), and n=31 (MA-ezrin) cells pooled from three independent experiments. b Quantification of the protrusions of n=207 (Parental), n=224 (ezrin), and n=214 (MA-ezrin) cells from three independent experiments. c Quantification of the migration or invasion rates of the indicated cells. n=9 fields from three independent experiments stained with anti-HA antibodies and phalloidin. d Quantification of the 3D migration phenotypes of n=176 (Parental), n=187 (ezrin), and n=175 (MA-ezrin) cells from three independent experiments. e Trajectories of cell centroids of the indicated cells tracked in (d) for 8 h. Right, the average speed of one cell over the course of 8 h. n=34 (ezrin) and n=44 (MA-ezrin) cells pooled from three independent experiments. f Quantification of representative hematoxylin and eosin (H&E)-stained sections of the primary tumor and surrounding tissue of mice injected with the indicated cells. Tumor invasive area at the tumor rim was quantified. n=9 areas for three tumors per group. g Quantification of spontaneous lung metastasis by quantitative PCR. n=6 mice (parental), n=3 (ezrin), and n=6 mice (MA-ezrin). **P=0.0152; ***P=0.0119. h Quantification of whole images of the lungs and H&E staining of lung sections (bottom) after tail vein injection of the indicated cells. n=8 mice per group. ***P=0.0002. All data were expressed as mean SD. Significance tested using the two-tailed Mann-Whitney test (a, c, g, h, i), two-tailed Student's t-test (c), and chi-square test (b, d). n.s., not significant; ****P<0.0001.

FIG. 5. Homeostatic PM tension suppresses cancer cell migration by counteracting BAR proteins. a Fraction of MCF10A spheroids with invasive structures grown in 3D on-top culture treated with the indicated RNAi. Control siRNA alone and siRNAs targeting BAR proteins that reduce invasive structures induced by ERM deletion. Data are mean of two independent experiments with at least 50 cells per experiment. b Fraction of the invasive structures of the indicated cells in 3D on top culture in captured images. Data are mean±SD of three independent experiments with at least 200 cells per experiment. Scale bar, 20 μm. **P=0.002; ***P=0.0007. c Quantification of phase-contrast images of MDA-MB-231 cells treated with the indicated RNAi of 3D migration phenotypes of n=153 (si-Control), n=150 (si-MTSS1L), and n=155 (si-Toca proteins) cells from three independent experiments. d Trajectories of cell centroids of the indicated cells tracked in c for 8 h. Right, the average speed of one cell over the course of 8 h. n=35 (si-Control), n=43 (si-MTSS1L), and n=46 (si-Toca proteins) cells pooled from three independent experiments. Mean±SD. e Quantification of protrusions of the indicated cells in 3D. n=151 (si-Control), n=132 (si-MTSS1L), and n=138 (si-Toca proteins) from three independent experiments. f Quantification of confocal images of indicted cells expressing GFP-FBP17 puncta of n=26 (si-Control), n=22 (si-ERM), n=22 (si-SLK+STK10), and n=22 (Snail-expressing cells) cells pooled from three independent experiments. g Quantification of confocal images of the indicated cells stained with phalloidin and WGA in 3D showing GFP-FBP17 puncta of n=20 (ezrin) and n=20 (MA-ezrin) cells pooled from three independent experiments. All data, except for a, were expressed as mean SD. Significance tested using the two-tailed Student's t-test (b, f, g), two-tailed Mann-Whitney test (d), and chi-square test (c, e). ****P<0.0001.

FIG. 6. Proposed model describing how homeostatic PM tension acts as the mechanical suppressor of cancer cell dissemination. a Proposed model to describe how cancer progression is linked to the disruption of homeostatic PM tension, leading to cancer cell dissemination via BAR proteins. b Homeostatic PM tension sustained by membrane-to-cortex attachment (MCA) can maintain a non-motile state by suppressing the assembly of BAR proteins, key regulators of both actin- and bleb-based protrusions.

FIG. 7. Analysis of the tether force and PM tension. a Schematic illustration of the measurements of tether force (Fₜₑₜₕₑᵣ) with optical tweezers. k is the stiffness of the trap and Δx is the displacement of the bead from the trap center. PM tension can be estimated with the formula as described. B is the bending stiffness of the membrane. See the Methods section for details. b Schematic illustration of PM tension regulation by membrane-to-cortex attachment (MCA) via ERM proteins. ERM proteins are activated by RHOA through ERM kinases, including ROCK1/2, SLK, and STK10. c Mean fluorescence intensity of the line scan across the PM on the lateral side from confocal images of indicated cells stained with anti-pERM antibodies, phalloidin, and wheat germ agglutinin (WGA). n=10 (MCF10A), n=10 (AU565), n=10 (MDA-MB-231), and n=10 (HS578T) cells from three independent experiments. d Quantification of protrusions of the indicated cells in a 3D collagen matrix (3D). n=144 (MCF10A), n=107 (AU565), n=175 (MDA-MB-231), and n=126 (Hs578T) cells from two independent experiments. Confocal images were of AU565 or Hs578T cells stained with anti-pERM antibodies, phalloidin, and WGA in a 3D collagen matrix. Chi-square test. n.s., not significant; ****P<0.0001.

FIG. 8. A decrease in PM tension induces a mesenchymal migratory phenotype in epithelial cells. a Confirmation of the downregulated expression of target proteins by RNAi analysis using western blotting. Images are representative of two independent experiments with similar results. b Scatter plots comparing the tether force of MCF10A cells treated with the indicated RNAi. n=60 (si-Control), n=40 (si-RHOA), n=28 (si-ERM), and n=25 (si-SLK+STK10) cells pooled from three independent experiments. Mean±SD. See also FIG. 2a. c Western blot of endogenous phospho-myosin light chain (pS19MLC), MLC, E-cadherin, vimentin, and β-actin levels in the indicated cells. Images are representative of two independent experiments with similar results. d Trajectories of cell centroids of the indicated cells tracked for 6 h in 2D. n=21 (si-Control), n=22 (si-RHOA), n=17 (si-ERM), and n=16 (si-SLK+STK10) cells from three independent experiments. Right, scatter plots comparing their aspect ratios of n=81 (si-Control), n=98 (si-RHOA), n=81 (si-ERM), and n=85 (si-SLK+STK10) cells pooled from three independent experiments. Mean±SD. e Quantification of AU565 or MCF7 cells that invaded through Matrigel. n=6 fields from two independent experiments. Mean±SD. f Proliferation rates of MCF10A cells treated with the indicated RNAi. Data are from the mean±SD of three independent experiments. Statistical comparison to the appropriate control was performed using two-tailed Mann-Whitney test (b, d) and two-tailed Student's t-test (e). ****P<0.0001.

FIG. 9. Downregulation of MCA regulators in cancer patients. a Western blot of endogenous E-cadherin, vimentin, and β-actin levels in the indicated cells. Images are representative of two independent experiments with similar results. b Scatter plots comparing the estimated PM tension of the indicated cells. n=15 (MDCK II cells, Dox [−]) and n=16 (MDCK II cells expressing RasV12, Dox [+]) cells pooled from three independent experiments. Mean SD. Two-tailed Mann-Whitney test. ****P<0.0001. c Genetic alterations of the indicated genes across 14 human tumor types in TCGA data (6586 samples). d Genetic alterations of the indicated genes across 961 cancer cells in the Cancer Cell Line Encyclopedia (CCLE) data.

FIG. 10. Increasing PM tension suppresses 3D migration. a Proliferation rates of MDA-MB-231 cells are expressed as indicated. Data are presented as the mean±SD from three independent experiments. b Quantification of drug-treated cells that invaded through Matrigel. n=6 fields from two independent experiments. Mean±SD. n.s., not significant; ****P<0.0001. c Scatter plots comparing the estimated PM tension of MDA-MB-231 cells treated with MβCD. n=20 (Mock, water) and n=21 (MβCD) cells pooled from three independent experiments. Mean±SD. ****P<0.0001. d Primary tumor growth after injection of the indicated cells into the mammary fat pad. n=12 mice (MDA-MB-231 parental), n=4 (ezrin) and n=6 mice (MA-ezrin). Mean±SD. The mean tumor volumes are shown in the graph. **P=0.0095; ***P=0.0001. Statistical analysis using two-tailed Student's t-test (b, c) and two-tailed Mann-Whitney test (d).

FIG. 11. Homeostatic PM tension inhibits cancer cell migration by suppressing BAR protein assembly. a, b Quantification of the indicated RNAi-treated MDA-MB-231 cells using phase-contrast images in 3D on-top culture. (a) or MCF10A cells (b) that invaded through Matrigel. n=9 fields from three independent experiments. Mean±SD. c Quantification of GFP-FBP17 or GFP-MTSS1L puncta at the PM. n=20 (GFP-FBP17) and n=20 (GFP-MTSS1L) cells pooled from three independent experiments. d Quantification of GFP-MTSS1L puncta at the PM of n=20 (si-Control), n=20 (si-ERM), and n=20 (Snail-expressing cells) cells pooled from three independent experiments. Mean±SD. e Quantification of GFP-FBP17 puncta at the PM of n=20 (Mock, water) and n=20 (MβCD) cells pooled from three independent experiments. Mean±SD. f Quantification of confocal images of GFP-FBP17 or GFP-MTSS1L in MDA-MB-231 cells before and after mechanical stretching (20%). n=20 (GFP-FBP17) or n=20 (GFP-MTSS1L) cells pooled from two independent experiments. Mean±SD. Statistical analysis using the two-tailed Student's t-test (a, c, e), one-way ANOVA with Tukey's multiple comparisons test (b), and two-tailed Mann-Whitney test (d, f). ****P<0.0001.

### DETAILED DESCRIPTION OF THE INVENTION

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five substituents on the ring.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number 1” to “number 2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number 10”, it implies a continuous range that includes whole numbers and fractional numbers less than number 10, as discussed above. Similarly, if the variable disclosed is a number greater than “number 10”, it implies a continuous range that includes whole numbers and fractional numbers greater than number 10. These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The compounds and compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation, or limitations not specifically disclosed herein.

As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” in the context of a peptide indicates that a peptide comprises a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93% or 94%, or even 95%, 96%, 97% 98% or 99%, sequence identity to the reference sequence over a specified comparison window. In certain embodiments, optimal alignment is conducted using the homology alignment algorithm of Needleman and Wunsch (Needleman and Wunsch, JMB, 48, 443 (1970)). An indication that two peptide sequences are substantially identical is that one peptide is immunologically reactive with antibodies raised against the second peptide. Thus, a peptide is substantially identical to a second peptide, for example, where the two peptides differ only by a conservative substitution. Thus, the invention also provides nucleic acid molecules and peptides that are substantially identical to the nucleic acid molecules and peptides presented herein.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

A gene is “targeted” by a siRNA according to the present invention when, for example, the siRNA molecule selectively decreases or inhibits the expression of the gene. The phrase “selectively decrease or inhibit” as used herein encompasses siRNAs that affect expression of a gene. Alternatively, a siRNA targets a gene when (one strand of) the siRNA hybridizes under stringent conditions to the gene transcript, i.e., its mRNA. Hybridizing “under stringent conditions” means annealing to the target mRNA region under standard conditions, e.g., high temperature and/or low salt content which tend to disfavor hybridization. A suitable protocol (involving 0.1*SSC, 68° C. for 2 hours) is described in Maniatis et al., Molecular Cloning: A Laboratory Manual, Cold Spring Harbor Laboratory, 1982, at pages 387-389.

Nucleic acid sequences cited herein are written in a 5′ to 3′ direction unless indicated otherwise. The term “nucleic acid” refers to either DNA or RNA or a modified form thereof comprising the purine or pyrimidine bases present in DNA (adenine “A”, cytosine “C”, guanine “G”, thymine “T”) or in RNA (adenine “A”, cytosine “C”, guanine “G”, uracil “U”). Interfering RNAs provided herein may comprise “T” bases, for example at 3′ ends, even though “T” bases do not naturally occur in RNA. In some cases, these bases may appear as “dT” to differentiate deoxyribonucleotides present in a chain of ribonucleotides.

As used herein, an “expression vector” is meant a vector that permits the expression of a polynucleotide inside a cell. Expression of a polynucleotide includes transcriptional and/or posttranscriptional events.

The term “gene” as used herein refers to any and all discrete coding regions of a host genome, or regions that code for a functional RNA only (e.g., tRNA, rRNA, regulatory RNAs such as ribozymes etc.) as well as associated non-coding regions and optionally regulatory regions. In certain embodiments, the term “gene” includes within its scope the open reading frame encoding specific polypeptides, introns, and adjacent 5′ and 3′ non-coding nucleotide sequences involved in the regulation of expression. In this regard, the gene may further comprise control signals such as promoters, enhancers, termination and/or polyadenylation signals that are naturally associated with a given gene, or heterologous control signals. The gene sequences may be eDNA or genomic DNA or a fragment thereof. The gene may be introduced into an appropriate vector for extrachromosomal maintenance or for integration into the host.

The term “mRNA” means a messenger RNA, which is a “transcript” produced in a cell using DNA as a template, which itself encodes a protein. mRNA is typically comprised of a 5′-UTR, a protein encoding (i.e., coding) region and a 3′-UTR. mRNA has a limited half-life in cells, which is determined, in part, by stability elements, particularly within the 3′-UTR but also in the 5′-UTR and protein encoding region.

By “operably connected” or “operably linked” and the like is meant a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the nucleic acid sequences being linked are typically contiguous and, where necessary to join two protein coding regions, contiguous and in reading frame. A coding sequence is “operably linked to” another coding sequence when RNA polymerase will transcribe the two coding sequences into a single mRNA, which is then translated into a single polypeptide having amino acids derived from both coding sequences. The coding sequences need not be contiguous to one another so long as the expressed sequences are ultimately processed to produce the desired protein. “Operably connecting” a promoter to a transcribable polynucleotide is meant placing the transcribable polynucleotide (e.g., protein encoding polynucleotide or other transcript) under the regulatory control of a promoter, which then controls the transcription and optionally translation of that polynucleotide. In the construction of heterologous promoter/structural gene combinations, it is generally preferred to position a promoter or variant thereof at a distance from the transcription start site of the transcribable polynucleotide, which is approximately the same as the distance between that promoter and the gene it controls in its natural setting; i.e.: the gene from which the promoter is derived. As is known in the art, some variation in this distance can be accommodated without loss of function. Similarly, the preferred positioning of a regulatory sequence element (e.g., an operator, enhancer etc.) with respect to a transcribable polynucleotide to be placed under its control is defined by the positioning of the element in its natural setting; i.e., the genes from which it is derived.

By “promoter” is meant a region of DNA, generally upstream (5′) of a coding region, which controls at least in part the initiation and level of transcription. Reference herein to a “promoter” is to be taken in its broadest context and includes the transcriptional regulatory sequences of a classical genomic gene, including a TATA box and CCAAT box sequences, as well as additional regulatory elements (i.e., activating sequences, enhancers and silencers) that alter gene expression in response to developmental and/or environmental stimuli, or in a tissue-specific or cell-type-specific manner. A promoter is usually, but not necessarily, positioned upstream or 5′, of a structural gene, the expression of which it regulates. Furthermore, the regulatory elements comprising a promoter are usually positioned within 2 kb of the start site of transcription of the gene. Promoters according to the invention may contain additional specific regulatory elements, located more distal to the start site to further enhance expression in a cell, and/or to alter the timing or inducibility of expression of a structural gene to which it is operably connected. The term “promoter” also includes within its scope inducible, repressible and constitutive promoters as well as minimal promoters. Minimal promoters typically refer to minimal expression control elements that are capable of initiating transcription of a selected DNA sequence to which they are operably linked. In some examples, a minimal promoter is not capable of initiating transcription in the absence of additional regulatory elements (e.g., enhancers or other cis-acting regulatory elements) above basal levels. A minimal promoter frequently consists of a TATA box or TATA-like box. Numerous minimal promoter sequences are known in the literature. For example, minimal promoters may be selected from a wide variety of known sequences, including promoter regions from fos, CMV, SV40 and IL-2, among many others. Illustrative examples are provided which use a minimal CMV promoter or a minimal IL2 gene promoter (−72 to +45 with respect to the start site; Siebenlist, 1986).

### Embodiments of the Invention

This disclosure provides for methods for treating cancer comprising increasing the tension of the plasma membrane of the cancer cell, thereby preventing migration and proliferation of the cancer cell. Preferably, the tension of the plasma membrane is increased to and/or maintained at about 100-200 pN/μm.

The disclosure also provides for methods of increasing or decreasing the rate of cell division, the rate of cell motility, and the rate of cell proliferation.

In some embodiments of the disclosure, increasing the tension of the plasma membrane may comprise manipulating a permeability function of the cancer cell. For example, certain agents (e.g., drugs, proteins, nucleic acids) may contact the cancer cell and cause a change in membrane permeability, leading to a subsequent influx of efflux of solutes from the cancer cell. The influx or efflux of solutes may increase internal pressure, thereby causing an increase in plasma membrane tension. In other aspects, the agent may contact a cancer cell and cause an increase or decrease of one or more cellular components that regulate internal pressure (e.g., ion transporter proteins, small molecule transporter proteins, water-channel proteins, sugar synthesis proteins, and transport proteins (e.g., glucose transporter), etc.).

In some embodiments, the membrane permeability may be affected to cause an influx of water molecules into the cancer cell.

In other embodiments of the disclosure, increasing the tension of the plasma membrane comprises increasing or decreasing an amount of a component of the plasma membrane such, as but not limited to, a lipid, a phospholipid, a glycolipid, a protein, a glycoprotein, and cholesterol.

In some embodiments, an increase or decrease in the tension of the plasma membrane may be modulated, changed, effected, by strengthening or weakening the membrane-actin cortex attachment (MCA). Accordingly, an agent of the disclosure may target various genes and/or their protein products that regulate the MCA.

In one embodiment, the agent promotes activation of various membrane-actin linker proteins that result in enhanced or increased MCA. Enhancement or increased MCA may be compared to control cells that are not contacted by the agent or are defective in one or more MCA interactions. In some embodiments, using optical tweezers, increased MCA can be directly confirmed by measuring the force applied to the membrane tether that is formed. It is known that MCA is dependent on the linker proteins (especially ERM proteins) that connect the membrane to actin, hence analysis of ERM activity (in this case phosphorylation state) can measure indirectly whether MCA is enhanced compared to the control cell.

One molecule that may enhance or increase the MCA is phospholipid Phosphatidylinositol 4,5-bisphosphate (PIP2). PIP2 is a component of the plasma membrane and can activate the membrane-actin linker proteins such as the ERM proteins. Accordingly, in some embodiments, the MCA is increased or strengthened by increasing expression of one or more proteins that are known to synthesize the formation of PIP2. For example, phosphatidylinositol 4-phosphate 5-kinase (PIP5K) increase PIP2 synthesis. (See, for example, Ben-Aissa et al., Cell Biology, Volume 287, Issue 20, P16311-16323, May 2012). In some embodiments, an increase in expression of one or more PIP5k proteins such as PIP5K1A (SEQ ID NO: 77), PIP5K1B (SEQ ID NO: 79), and PIP5K1C (SEQ ID NO: 81) cause an increase in MCA and an increase in plasma membrane tension by increasing the amount of PIP2 that subsequently activates the ERM proteins. In other embodiments, an expression vector comprising one more PIP5K genes may be introduced into a cell to increase the MCA. These genes include PIP5K1A (SEQ ID NO: 76), PIP5K1B (SEQ ID NO: 78), and PIP5K1C (SEQ ID NO: 80)

In some embodiments, the expression level and/or activity of one or more ERM proteins are modulated to increase the plasma membrane tension. This may be achieved, for example, by increasing expression of certain proteins that are known to activate the ERM proteins (e.g., via phosphorylation) either directly or indirectly, thus increasing the amount of active ERM protein, thereby increasing the amount of MCA and plasma membrane tension. In some embodiments, certain upstream kinases of the ERM proteins are used to increase the activity of the ERM proteins via phosphorylation of ERM. In some embodiments, the kinases that phosphorylate and increase the activity of the ERM proteins are one or more of ROCK1 (DNA sequence SEQ ID NO: 210; amino acid sequence: SEQ ID NO: 211), ROCK2 (DNA sequence SEQ ID NO: 212; amino acid sequence: SEQ ID NO: 213), SLK (DNA sequence SEQ ID NO: 214; amino acid sequence: SEQ ID NO: 215), STK10 (DNA sequence SEQ ID NO: 216; amino acid sequence: SEQ ID NO: 217), and RHOA (DNA sequence SEQ ID NO: 7; amino acid sequence: SEQ ID NO: 8).

In some embodiments of the disclosure, increasing tension of the plasma membrane is affected by contacting the cancer cell, or a cell suspected of being cancerous, with an agent that is internalized into the cancer cell to cause an increase in tension of the plasma membrane.

In some embodiments, the agent may comprise one or more of an antibody, antibody fragment, antibody mimetic, aptamer, siRNA, micro-RNA, shRNA, a nanobody, DNA, or a chemical compound. The agent may inhibit expression of a certain gene or genes, or may inactivate, sequester, degrade, or otherwise reduce or inhibit the protein products of said genes.

The term “antibody” as used herein refers to a polypeptide (or set of polypeptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CHI, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen, which is sometimes referred to herein as the antigen binding domain. The constant regions of the antibodies may mediate the binding of the immunoglobulin to host tissues or factors, including various cells of the immune system (e.g., effector cells) and the first component (C1q) of the classical complement system. The term “antibody” includes, but is not limited to, monoclonal antibodies, human antibodies, humanized antibodies, camelised antibodies, chimeric antibodies, bispecific or multispecific antibodies and anti-idiotypic (anti-Id) antibodies (including, e.g., anti-Id antibodies to antibodies described herein), single chain variable fragments, and single domain antibodies. The antibodies can be of any isotype/class (e.g., IgG, IgE, IgM, IgD, IgA and IgY) or subclass (e.g., IgG1, IgG2, IgG3, IgG4, IgA1 and IgA2). Both the light and heavy chains are divided into regions of structural and functional homology. The terms “constant” and “variable” are used functionally. In this regard, it will be appreciated that the variable domains of both the light (V_{L}) and heavy (V_{H}) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminus is a variable region and at the C-terminus is a constant region; the CH3 and CL domains actually comprise the carboxy-terminus of the heavy and light chain, respectively.

In certain embodiments, the antibody moieties may comprise one or more of a single chain variable fragment (scFv), single domain antibody, a bispecific antibody, or multispecific antibody.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}. ScFv molecules are known in the art and their production is described, for example, in U.S. Pat. Nos. 4,946,778, and 5,641,870.

The term “bispecific antibody” refers to an antibody that shows specificities to two different types of antigens. The term “multispecific antibody” as used herein refers to a molecule that binds to two or more different epitopes on one antigen or on two or more different antigens. Recognition of each antigen is generally accomplished with an “antigen binding domain”. The multispecific antibody may include one polypeptide chain that comprises a plurality, e.g., two or more, e.g., two, antigen binding domains. In some embodiments, the multispecific antibody may include two, three, four or more polypeptide chains that together comprise a plurality, e.g., two or more, e.g., two, antigen binding domains. Examples of the production and isolation of bispecific and multispecific antibodies are described in, for example, PCT Pat. Pubs. WO2014031174 and WO2009080252.

The term “single domain antibodies” refers to the variable regions of either the heavy (VH) or light (VL) chain of an antibody. Single domain antibodies are described, for example in U.S. Pat. Pub. No. 20060002935A1.

In certain embodiments of the disclosure, the antibody mimetic comprises or consists of an affibody, an Affilin, an affimer, an affitin, an alphabody, an anticalin, and avimer, a DARPin, a Fynomer, a Kunitz domain peptide, or a monobody.

As used herein, the term “antibody mimetic” is intended to describe an organic compound that specifically binds a target sequence and has a structure distinct from a naturally occurring antibody. Antibody mimetics may comprise a protein, a nucleic acid, or a small molecule. The target sequence to which an antibody mimetic of the disclosure specifically binds may be an antigen. Antibody mimetics may provide superior properties over antibodies including, but not limited to, superior solubility, tissue penetration, stability towards heat and enzymes (e.g., resistance to enzymatic degradation), and lower production costs. Exemplary antibody mimetics include, but are not limited to, an affibody, an Affilin, an affimer, an affitin, an alphabody, an anticalin, and avimer (also known as avidity multimer), a DARPin (Designed Ankyrin Repeat Protein), a Fynomer, a Kunitz domain peptide, and a monobody.

Affibody molecules of the disclosure comprise a protein scaffold comprising or consisting of one or more alpha helix without any disulfide bridges. Preferably, affibody molecules of the disclosure comprise or consist of three alpha helices. For example, an affibody molecule of the disclosure may comprise an immunoglobulin binding domain. An affibody molecule of the disclosure may comprise, for example, the Z domain of protein A.

Affilin molecules of the disclosure comprise a protein scaffold produced by modification of exposed amino acids of, for example, either gamma-B crystallin or ubiquitin. Affilin molecules functionally mimic an antibody's affinity to antigen, but do not structurally mimic an antibody. In any protein scaffold used to make an affilin, those amino acids that are accessible to solvent or possible binding partners in a properly folded protein molecule are considered exposed amino acids. Any one or more of these exposed amino acids may be modified to specifically bind to a target sequence or antigen.

Affimer molecules of the disclosure comprise a protein scaffold comprising a highly stable protein engineered to display peptide loops that provide a high affinity binding site for a specific target sequence. Exemplary affimer molecules of the disclosure comprise a protein scaffold based upon a cystatin protein or tertiary structure thereof. Exemplary affimer molecules of the disclosure may share a common tertiary structure of comprising an alpha-helix lying on top of an anti-parallel beta-sheet.

In some embodiments, the agent may affect the stability of RNA. As used herein, stability of RNA refers to any modulation in the stability of ERM protein RNA, Bar domain protein RNA, or other RNA of a gene disclosed herein by an agent disclosed herein. More specifically, RNA modifications are changes to the chemical composition of ribonucleic acid (RNA) molecules post-synthesis that have the potential to alter function or stability.

RNA modifications that increase stability may include capping i.e., the addition of a methylated guanine nucleotide cap to the 5′ end of mRNAs, cleavage and polyadenylation i.e., cleavage of the 3′ end of the RNA and then the addition of about 250 adenine residues to form a poly(A) tail. Thus, in some embodiments, the agents may modulate RNA stability by directly or indirectly modulating any of the processes involved in capping, cleavage and/or polyadenylation.

In other embodiments, the agents may modulate RNA stability by directly or indirectly modulating its degradation. More specifically, RNA degradation is mediated by three major classes of intracellular RNA-degrading enzymes (ribonucleases or RNases): endonucleases that cut RNA internally, 5′ exonucleases that hydrolyze RNA from the 5′ end, and 3′ exonucleases that degrade RNA from the 3′ end. The specificity of RNA degradation mechanisms is frequently conferred by cofactors such as helicases, polymerases and chaperones.

The ATP-dependent RNA helicases are a large protein family that participates in almost all pathways of RNA processing and degradation. The eukaryotic exosome complex exhibit both 3′ exonuclease and endonuclease activity and function together with helicase family members Mtr4 and Ski2, in the RNA degradation process.

Still further, in some additional or alternative embodiments, the agents may either increase or decrease the translation of the RNAs of, for example, the ERM protein genes and/or the BAR domain protein genes disclosed herein.

In some embodiments, the agents may be a nucleic acid molecule. More particularly, the nucleic acid molecule may be a molecule comprising at least one of a single stranded DNA (ssDNA), a single stranded RNA (ssRNA), a double stranded DNA (dsDNA), a double stranded RNA (dsRNA), nucleic acid molecule having at least one modified nucleotide/s and any combinations thereof.

More specifically, in certain embodiments an agent reduces the amount or levels of ERM and/or Bar domain protein RNA (either by reducing the stability, increasing the degradation and/or reducing synthesis thereof), and/or inhibiting or reducing the activity of Erm or BAR domain RNA, may be a nucleic acid molecule that may comprise at least one of a short hairpin RNA (shRNA), a small interfering RNA (siRNA), microRNA (miRNA), antisense oligonucleotide (ASO), locked nucleic acid (LNA), as well as other nucleic acids derivatives.

Interfering RNA (which may be interchangeably referred to as RNAi or an interfering RNA sequence) refers to double-stranded RNA that is capable of silencing, reducing, or inhibiting expression of a target gene by any mechanism of action now known or yet to be disclosed. For example, RNAi may act by mediating the degradation of mRNAs which are complementary to the sequence of the RNAi when the RNAi is in the same cell as the target gene. As used herein, RNAi may refer to double-stranded RNA formed by two complementary RNA strands or by a single, self-complementary strand. RNAi may be substantially or completely complementary to the target mRNA or may comprise one or more mismatches upon alignment to the target mRNA. The sequence of the interfering RNA may correspond to the full-length target mRNA, or any subsequence thereof.

Generally speaking, RNAi is a multistep process. In a first step, there is cleavage of large dsRNAs into 21-23 ribonucleotides-long double-stranded effector molecules called “small interfering RNAs” or “short interfering RNAs” (siRNAs). These siRNAs duplexes then associate with an endonuclease-containing complex, known as RNA-induced silencing complex (RISC). The RISC specifically recognizes and cleaves the endogenous mRNAs/RNAs containing a sequence complementary to one of the siRNA strands. One of the strands of the double-stranded siRNA molecule (the “guide” strand) comprises a nucleotide sequence that is complementary to a nucleotide sequence of the target gene, or a portion thereof, and the second strand of the double-stranded siRNA molecule (the passenger” strand) comprises a nucleotide sequence substantially similar to the nucleotide sequence of the target gene, or a portion thereof.

In more particular embodiments, siRNAs comprise a duplex, or double-stranded region, of about 18-25 nucleotides long. Often, siRNAs contain from about two to four unpaired nucleotides at the 3′ end of each strand. At least a portion of one strand of the duplex or double-stranded region of a siRNA is substantially homologous to or substantially complementary to a target sequence within the gene product (i.e., RNA) molecule as herein defined. The strand complementary to a target RNA molecule is the “antisense guide strand”, the strand homologous to the target RNA molecule is the “sense passenger strand” (which is also complementary to the siRNA antisense guide strand). siRNAs may also be contained within structured such as miRNA and shRNA which has additional sequences such as loops, linking sequences as well as stems and other folded structures.

As noted above, RNAi includes small-interfering RNA, which, herein, may interchangeably be referred to as siRNA. siRNA is described for example in U.S. Pat. Nos. 9,328,347; 9,328,348; 9,289,514; 9,289,505; and 9,273,312, the content of each of which is incorporated by reference herein in its entirety. A siRNA may be any interfering RNA with a duplex length of about 15-60, 15-50, or 15-40 nucleotides in length, more typically about 15-30, 15-25, or 18-23 nucleotides in length. Each complementary sequence of the double-stranded siRNA may be 15-60, 15-50, 15-40, 15-30, 15-25, or 18-23 nucleotides in length, but other noncomplementary sequences may be present. For example, siRNA duplexes may comprise 3′ overhangs of 1 to 4 or more nucleotides and/or 5′ phosphate termini comprising 1 to 4 or more nucleotides. A siRNA may be synthesized in any of a number of conformations. One of ordinary skill in the art would recognize the type of siRNA conformation to be used for a particular purpose. Examples of siRNA conformations include, but need not be limited to, a double-stranded polynucleotide molecule assembled from two separate stranded molecules, wherein one strand is the sense strand and the other is the complementary antisense strand; a double-stranded polynucleotide molecule assembled from a single-stranded molecule, where the sense and antisense regions are linked by a nucleic acid-based or non-nucleic acid-based linker; a double-stranded polynucleotide molecule with a hairpin secondary structure having complementary sense and antisense regions; or a circular single-stranded polynucleotide molecule with two or more loop structures and a stem having self-complementary sense and antisense regions. In the case of the circular polynucleotide, the polynucleotide may be processed either in vivo or in vitro to generate an active double-stranded siRNA molecule.

SiRNA can be chemically synthesized, may be encoded by a plasmid and transcribed, or may be vectored by a virus engineered to express the siRNA. A siRNA may be a single stranded molecule with complementary sequences that self-hybridize into duplexes with hairpin loops. siRNA can also be generated by cleavage of parent dsRNA through the use of an appropriate enzyme such as E. coli RNase III or Dicer (Yang et al., Proc. Natl. Acad. Sci. USA 99, 9942-9947 (2002); Calegari et al., Proc. Natl. Acad. Sci. USA 99, 14236-14240 (2002); Byrom et al, Ambion TechNotes 10, 4-6 (2003); Kawasaki et al, Nucleic Acids Res 31, 981-987 (2003); and Knight et al., Science 293, 2269-2271 (2001). A parent dsRNA may be any double stranded RNA duplex from which a siRNA may be produced, such as a full or partial mRNA transcript.

A mismatch motif may be any portion of a siRNA sequence that is not 100% complementary to its target sequence. A siRNA may have zero, one, two, or three or more mismatch regions. The mismatch regions may be contiguous or may be separated by any number of complementary nucleotides. The mismatch motifs or regions may comprise a single nucleotide or may comprise two or more consecutive nucleotides.

SiRNA molecules can be provided in several forms including, e.g., as one or more isolated siRNA duplexes, as longer double-stranded RNA (dsRNA), or as siRNA or dsRNA transcribed from a transcriptional cassette in a DNA plasmid. The siRNA sequences may have overhangs (as 3′ or 5′ overhangs as described in Elbashir et al, Genes Dev 15, 188 (2001), the content of each of which is incorporated by reference herein in its entirety) or may lack overhangs (i.e., have blunt ends).

One or more DNA plasmids encoding one or more siRNA templates may be used to provide siRNA. siRNA can be transcribed as sequences that automatically fold into duplexes with hairpin loops from DNA templates in plasmids having RNA polymerase Ill transcriptional units, for example, based on the naturally occurring transcription units for small nuclear RNA U6 or human RNase P, RNAse H1 (Brummelkamp et al, Science 296, 550 (2002); Donze et al, Nucleic Acids Res 30, e46 (2002); Paddison et al, Genes Dev 16, 948 (2002)). Typically, a transcriptional unit or cassette will contain an RNA transcript promoter sequence, such as an H1-RNA or a U6 promoter, operably linked to a template for transcription of a desired siRNA sequence and a termination sequence, comprised of 2-3 uridine residues and a polythymidine (T5) sequence (polyadenylation signal). The selected promoter can provide for constitutive or inducible transcription. Compositions and methods for DNA-directed transcription of RNA interference molecules are described in detail in U.S. Pat. No. 6,573,099, incorporated by reference herein in its entirety. The transcriptional unit is incorporated into a plasmid or DNA vector from which the interfering RNA is transcribed. Plasmids suitable for in vivo delivery of genetic material for therapeutic purposes are described in detail in U.S. Pat. Nos. 5,962,428 and 5,910,488, the content of each of which is incorporated by reference herein in its entirety. The selected plasmid can provide for transient or stable delivery of a nucleic acid to a target cell. It will be apparent to those of skill in the art that plasmids originally designed to express desired gene sequences can be modified to contain a transcriptional unit cassette for transcription of siRNA.

Methods for isolating RNA, synthesizing RNA, hybridizing nucleic acids, making and screening cDNA libraries, and performing PCR are well known in the art (see, e.g., Gubler and Hoffman, Gene 25, 263-269 (1983); Sambrook and Russell, Molecular Cloning: A Laboratory Manual, Cold Spring Harbor Laboratory Press, Cold Spring Harbor N.Y., (2001), the content of each of which is incorporated by reference herein in its entirety) as are PCR methods (see, U.S. Pat. Nos. 4,683,195 and 4,683,202; PCR Protocols: A Guide to Methods and Applications, Innis et al, eds, (1990).

A siRNA molecule may be chemically synthesized. In one example of chemical synthesis, a single-stranded nucleic acid that includes the siRNA duplex sequence can be synthesized using any of a variety of techniques known in the art, such as those described in Usman et al, J Am Chem Soc, 109, 7845 (1987); Scaringe et al, Nucl Acids Res, 18, 5433 (1990); Wincott et al, Nucl Acids Res, 23, 2677-2684 (1995); and Wincott et al, Methods Mol Bio 74, 59 (1997), the content of each of which is incorporated by reference herein in its entirety. Synthesis of the single-stranded nucleic acid makes use of common nucleic acid protecting and coupling groups, such as dimethoxytrityl at the 5′-end and phosphoramidites at the 3′-end. As a non-limiting example, small scale syntheses can be conducted on an Applied Biosystems synthesizer (Thermo Fisher Scientific, Waltham, Mass.) using a 0.2 micromolar scale protocol with a 2.5 min coupling step for 2′-O-methylated nucleotides. Alternatively, syntheses at the 0.2 micromolar scale can be performed on a 96-well plate synthesizer from Thermo Fisher Scientific. However, larger or smaller scale synthesis are also encompassed by the invention, including any method of synthesis now known or yet to be disclosed. Suitable reagents for synthesis of siRNA single-stranded molecules, methods for RNA deprotection, and methods for RNA purification are known to those of skill in the art.

In certain embodiments, siRNA can be synthesized via a tandem synthesis technique, wherein both strands are synthesized as a single continuous fragment or strand separated by a linker that is subsequently cleaved to provide separate fragments or strands that hybridize to form a siRNA duplex. Linkers may be any linker, including a polynucleotide linker or a non-nucleotide linker. The tandem synthesis of siRNA can be readily adapted to both multiwell/multiplate synthesis platforms as well as large scale synthesis platforms employing batch reactors, synthesis columns, and the like. In some embodiments, siRNA can be assembled from two distinct single-stranded molecules, wherein one strand includes the sense strand and the other includes the antisense strand of the siRNA. For example, each strand can be synthesized separately and joined together by hybridization or ligation following synthesis and/or deprotection. Either the sense or the antisense strand may contain additional nucleotides that are not complementary to one another and do not form a double stranded siRNA. In certain instances, siRNA molecules can be synthesized as a single continuous fragment, where the self-complementary sense and antisense regions hybridize to form a siRNA duplex having hairpin secondary structure.

A siRNA molecule may comprise a duplex having two complementary strands that form a double-stranded region with least one modified nucleotide in the double-stranded region. The modified nucleotide may be on one strand or both. If the modified nucleotide is present on both strands, it may be in the same or different positions on each strand. A modified siRNA may be less immunostimulatory than a corresponding unmodified siRNA sequence but retains the capability of silencing the expression of a target sequence.

Examples of modified nucleotides suitable for use in the present invention include, but are not limited to, ribonucleotides having a 2′-O-methyl (2′OMe), 2′-deoxy-2′-fluoro (2′F), 2′-deoxy, 5-C-methyl, 2′-O-(2-methoxyethyl) (MOE), 4′-thio, 2′-amino, or 2′-C-allyl group. Modified nucleotides having a conformation such as those described in the art, for example in Sanger, Principles of Nucleic Acid Structure, Springer-Verlag Ed. (1984), incorporated by reference herein in its entirety, are also suitable for use in siRNA molecules. Other modified nucleotides include, without limitation: locked nucleic acid (LNA) nucleotides, G-clamp nucleotides, or nucleotide base analogs. LNA nucleotides include but need not be limited to 2′-O, 4′-C-methylene-(D-ribofuranosyl)nucleotides), 2′-O-(2-methoxyethyl) (MOE) nucleotides, 2′-methyl-thio-ethyl nucleotides, 2′-deoxy-2′-fluoro (2′F) nucleotides, 2′-deoxy-2′-chloro (2C1) nucleotides, and 2′-azido nucleotides. A G-clamp nucleotide refers to a modified cytosine analog wherein the modifications confer the ability to hydrogen bond both Watson-Crick and Hoogsteen faces of a complementary guanine nucleotide within a duplex (Lin et al, J Am Chem Soc, 120, 8531-8532 (1998) incorporated by reference herein in its entirety). Nucleotide base analogs include for example, C-phenyl, C-naphthyl, other aromatic derivatives, inosine, azole carboxamides, and nitroazole derivatives such as 3-nitropyrrole, 4-nitroindole, 5-nitroindole, and 6-nitroindole (Loakes, Nucl Acids Res, 29, 2437-2447 (2001) incorporated by reference herein in its entirety).

A siRNA molecule may comprise one or more non-nucleotides in one or both strands of the siRNA. A non-nucleotide may be any subunit, functional group, or other molecular entity capable of being incorporated into a nucleic acid chain in the place of one or more nucleotide units that is not or does not comprise a commonly recognized nucleotide base such as adenosine, guanine, cytosine, uracil, or thymine, such as a sugar or phosphate.

Chemical modification of siRNA may comprise attaching a conjugate to a siRNA molecule. The conjugate can be attached at the 5′- and/or the 3′-end of the sense and/or the antisense strand of the siRNA via a covalent attachment such as a nucleic acid or non-nucleic acid linker. The conjugate can be attached to the siRNA through a carbamate group or other linking group (see, e.g., U.S. Patent Publication Nos. 2005/0074771, 2005/0043219, and 2005/0158727, the content of each of which is incorporated by reference herein in its entirety). A conjugate may be added to siRNA for any of a number of purposes. For example, the conjugate may be a molecular entity that facilitates the delivery of siRNA into a cell or may be a molecule that comprises a drug or label. Examples of conjugate molecules suitable for attachment to siRNA of the present invention include, without limitation, steroids such as cholesterol, glycols such as polyethylene glycol (PEG), human serum albumin (HSA), fatty acids, carotenoids, terpenes, bile acids, folates (e.g., folic acid, folate analogs and derivatives thereof), sugars (e.g., galactose, galactosamine, N-acetyl galactosamine, glucose, mannose, fructose, fucose, etc.), phospholipids, peptides, ligands for cellular receptors capable of mediating cellular uptake, and combinations thereof (see, e.g., U.S. Patent Publication Nos. 2003/0130186, 2004/0110296, and 2004/0249178; U.S. Pat. No. 6,753,423; the content of each of which is incorporated by reference herein in its entirety). Other examples include the lipophilic moiety, vitamin, polymer, peptide, protein, nucleic acid, small molecule, oligosaccharide, carbohydrate cluster, intercalator, minor groove binder, cleaving agent, and cross-linking agent conjugate molecules described in U.S. Patent Publication Nos. 2005/0119470 and 2005/0107325, the content of each of which is incorporated by reference herein in its entirety. Other examples include the 2′-O-alkyl amine, 2′-O-alkoxyalkyl amine, polyamine, C5-cationic modified pyrimidine, cationic peptide, guanidinium group, amidininium group, cationic amino acid conjugate molecules described in U.S. Patent Publication No. 2005/0153337, incorporated by reference herein in its entirety. Additional examples of conjugate molecules include a hydrophobic group, a membrane active compound, a cell penetrating compound, a cell targeting signal, an interaction modifier, or a steric stabilizer as described in U.S. Patent Publication No. 2004/0167090, incorporated by reference herein in its entirety. Further examples include the conjugate molecules described in U.S. Patent Publication No. 2005/0239739, incorporated by reference herein in its entirety

In other embodiments, strands of a double-stranded interfering RNA (e.g., siRNA) may be connected to form a hairpin or stem-loop structure (e.g., shRNA). Thus, as mentioned above the agent also may be a short hairpin RNA (shRNA).

According to other embodiments, an agent may comprise a micro-RNA (miRNA). miRNAs are small RNAs made from genes encoding primary transcripts of various sizes. They have been identified in both animals and plants. The primary transcript (termed the “pri-miRNA”) is processed through various nucleolytic steps to a shorter precursor miRNA, or “pre-miRNA.” The pre-miRNA is present in a folded form so that the final (mature) miRNA is present in a duplex, the two strands being referred to as the miRNA. The pre-miRNA is a substrate for a form of dicer that removes the miRNA duplex from the precursor, after which, similarly to siRNAs, the duplex can be taken into the RISC complex. Unlike, siRNAs, miRNAs bind to transcript sequences with only partial complementarity and usually repress translation without affecting steady-state RNA levels. Both miRNAs and siRNAs are processed by Dicer and associate with components of the RNA-induced silencing complex (RISC). (See, for example, Michaels et al., Nature Communications, 19:818, 2019)

An agent may comprise a nucleic acid agent that may comprise at least one shRNA molecule. In more particular embodiments, such shRNA may comprise a nucleic acid sequence complementary at least in part to an EMR protein RNA or BAR domain protein RNA, or to any fragment/s or variant/s thereof. The term “shRNA”, as used herein, refers to an RNA agent having a stem-loop structure, comprising a first and second region of complementary sequence. The degree of complementarity and orientation of the regions being sufficient such that base pairing occurs between the regions. The first and second regions being joined by a loop region, the loop resulting from a lack of base pairing between nucleotides (or nucleotide analogs) within the loop region. Some of the nucleotides in the loop can be involved in base-pair interactions with other nucleotides in the loop.

In some specific embodiments, the shRNA may comprise a sequence complementary to the target ERM protein RNA and/or BAR domain protein RNA, having a length of between about 5 to 50 nucleotides, specifically, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 45, 46, 37, 38, 39, 40, 41, 42, 43, 44, 45, 46, 47, 48, 49, 50 or more nucleotides. In more specific embodiments, the complementary sequence may be in a length ranging between 9 to 29 nucleotides.

In some embodiments, target genes such as those of the ERM proteins and BAR domain proteins may be edited to reduce or suppress expression of said protein using clustered regularly interspaced short palindromic repeats (CRISPR) and CRISPR-associated (Cas) proteins as described, for example, in U.S. Pat. Nos. 10,266,850, 10,227,611, 10,000,772, 10,113,167, and U.S. Pat. Pub. No. 20190134227.

In general, the term “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR system may comprise, for example, CRISPR/Cas nuclease or CRISPR/Cas nuclease system includes a non-coding RNA molecule (guide) RNA, which sequence-specifically binds to DNA, and a Cas protein (e.g., Cas9), with nuclease functionality (e.g., two nuclease domains). In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes.

In some embodiments, a Cas nuclease and guide RNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. In some embodiments, the target site is selected based on its location immediately 5′ of a protospacer adjacent motif (PAM) sequence, such as typically NGG, or NAG. In this respect, the gRNA is targeted to the desired sequence by modifying the first 20 nucleotides of the guide RNA to correspond to the target DNA sequence.

In some embodiments, one or more vectors driving expression of one or more elements of the CRISPR system are introduced into the cell such that expression of the elements of the CRISPR system direct formation of the CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. In some embodiments, CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron). In some embodiments, the CRISPR enzyme, guide sequence, tracr mate sequence, and tracr sequence are operably linked to and expressed from the same promoter.

In some embodiments, a vector comprises one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors. In some embodiments, a vector comprises an insertion site upstream of a tracr mate sequence, and optionally downstream of a regulatory element operably linked to the tracr mate sequence, such that following insertion of a guide sequence into the insertion site and upon expression the guide sequence directs sequence-specific binding of the CRISPR complex to a target sequence in a eukaryotic cell. In some embodiments, a vector comprises two or more insertion sites, each insertion site being located between two tracr mate sequences so as to allow insertion of a guide sequence at each site. In such an arrangement, the two or more guide sequences may comprise two or more copies of a single guide sequence, two or more different guide sequences, or combinations of these. When multiple different guide sequences are used, a single expression construct may be used to target CRISPR activity to multiple different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to the cell.

In some embodiments, a vector comprises a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2. In some embodiments, the unmodified CRISPR enzyme has DNA cleavage activity, such as Cas9.

In some embodiments the CRISPR enzyme is Cas9 and may be Cas9 from S. pyogenes or S. pneumoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce non-homologous end joining.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of the CRISPR complex to a target sequence may be assessed by any suitable assay. For example, the components of the CRISPR system sufficient to form the CRISPR complex, including the guide sequence to be tested, may be provided to the cell having the corresponding target sequence, such as by transfection with vectors encoding the components of the CRISPR sequence, followed by an assessment of preferential cleavage within the target sequence. Similarly, cleavage of a target polynucleotide sequence may be evaluated in a test tube by providing the target sequence, components of the CRISPR complex, including the guide sequence to be tested and a control guide sequence different from the test guide sequence, and comparing binding or rate of cleavage at the target sequence between the test and control guide sequence reactions.

A guide sequence may be selected to target any target sequence. In some embodiments, the target sequence is a sequence within a genome of a cell. Exemplary target sequences include those that are unique in the target genome. In some embodiments, a guide sequence is selected to reduce the degree of secondary structure within the guide sequence. Secondary structure may be determined by any suitable polynucleotide folding algorithm.

In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a CRISPR complex at a target sequence, wherein the CRISPR complex comprises the tracr mate sequence hybridized to the tracr sequence. In general, degree of complementarity is with reference to the optimal alignment of the tracr mate sequence and tracr sequence, along the length of the shorter of the two sequences.

Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. In some aspects, loop forming sequences for use in hairpin structures are four nucleotides in length and have the sequence GAAA. However, longer or shorter loop sequences may be used, as well as alternative sequences. In some embodiments, the sequences include a nucleotide triplet (for example, AAA), and an additional nucleotide (for example C or G). Examples of loop forming sequences include CAAA and AAAG. In some embodiments, the transcript or transcribed polynucleotide sequence has at least two or more hairpins. In some embodiments, the transcript has two, three, four or five hairpins. In a further embodiment, the transcript has at most five hairpins. In some embodiments, the single transcript further includes a transcription termination sequence, such as a polyT sequence, for example six T nucleotides.

In some embodiments, the agent inhibits or reduces expression of one or more proteins comprising a BAR domain. The BAR (Bin/Amphiphysin/Rvs) domain is found in over 35 proteins encoded in the human genome. These proteins function in diverse cellular processes such as endocytosis (i.e., endophilins, sorting nexins, and amphiphysin) and actin reorganization (i.e., RhoGAPs and RhoGEFs). The functional BAR dimer is a banana shaped bundle of six helices with positive charges on the tips and along its concave surface that may mediate phospholipid binding. The curvature of the concave surface would fit a rounded membrane with a diameter of ˜220 angstroms. For example, the BAR domain of amphiphysin is approximately 210 amino acids in length and consists of a coiled coil composed of three extended a helices. Two monomers dimerize to form the functional banana shaped bundle of six helices. Positive charges are clustered at the tips of the banana like structure and along its concave surface. These positive charges are thought to mediate binding to phospholipids where the curvature of the concave surface would fit a rounded membrane with a diameter of ˜220 angstroms. While all BAR domains may bind curved lipids, a subset of BAR domains may induce membrane curvature. It is therefore predicted that BAR domains may function during vesiculation to either target proteins to curved membranes or to physically assist in inducing membrane curvature.

Exemplary BAR domain genes and their proteins include MTSS1L/ABBA, FNBP1/FBP17, TRIP10/CIP4, ARHGAP10/GARF2, ARHGAP17/RICH1, ARHGAP26/GRAF1, ARHGAP29, ARHGAP42/GRAF, ARHGAP44/RICH2, ARHGAP45/HMHA1, ARHGEF37, ARHGEF38, IRSp53/BAIAP2, BAIAP2L1/IRTKS, DNMBP/Tuba, FCHSD2, FER; FES, FCHSD1, GAS7, GMIP, MTSS1/MIM, OPHN1, PACSIN1, PACSIN2, PACSIN3, SH3BP1, SRGAP1, SRGAP2, SRGAP3, and ARHGAP4.

In some embodiments, the BAR domain genes are 90%, 95%, 96%, 97%, 98%, 99%, or 100% identical to MTSS1L/ABBA (SEQ ID NO: 11), FNBP1/FBP17 (SEQ ID NO: 13), TRIP10/CIP4 (SEQ ID NO: 15), ARHGAP10/GARF2 (SEQ ID NO: 17), ARHGAP17/RICH1 (SEQ ID NO: 19), ARHGAP26/GRAF1 (SEQ ID NO: 21), ARHGAP29 (SEQ ID NO: 23), ARHGAP42/GRAF (SEQ ID NO: 25), ARHGAP44/RICH2 (SEQ ID NO: 27), ARHGAP45/HMHA1 (SEQ ID NO: 29), ARHGEF37 (SEQ ID NO: 31), ARHGEF38 (SEQ ID NO: 33), IRSp53/BAIAP2 (SEQ ID NO: 35), BAIAP2L1/IRTKS (SEQ ID NO: 37), DNMBP/Tuba (SEQ ID NO: 39), FCHSD2 (SEQ ID NO: 41), FER (SEQ ID NO: 43); FES (SEQ ID NO: 45), FCHSD1 (SEQ ID NO: 47), GAS7 (SEQ ID NO: 49), GMIP (SEQ ID NO: 51), MTSS1/MIM (SEQ ID NO: 53), OPHN1 (SEQ ID NO: 55), PACSIN1 (SEQ ID NO: 57), PACSIN2 (SEQ ID NO: 59), PACSIN3 (SEQ ID NO: 61), SH3BP1 (SEQ ID NO: 63), SRGAP1 (SEQ ID NO: 65), SRGAP2 (SEQ ID NO: 67), SRGAP3 (SEQ ID NO: 69), ARHGAP4 (SEQ ID NO: 71), and FNBPL1 (SEQ ID NO: 73).

In some embodiments, the BAR domain proteins are 90%, 95%, 96%, 97%, 98%, 99%, or 100% identical to MTSS1L/ABBA (SEQ ID NO: 12), FNBP1/FBP17 (SEQ ID NO: 14), TRIP10/CIP4 (SEQ ID NO: 16), ARHGAP10/GARF2 (SEQ ID NO: 18), ARHGAP17/RICH1 (SEQ ID NO: 20), ARHGAP26/GRAF1 (SEQ ID NO: 22), ARHGAP29 (SEQ ID NO: 24), ARHGAP42/GRAF (SEQ ID NO: 26), ARHGAP44/RICH2 (SEQ ID NO: 28), ARHGAP45/HMHA1 (SEQ ID NO: 30), ARHGEF37 (SEQ ID NO: 32), ARHGEF38 (SEQ ID NO: 34), IRSp53/BAIAP2 (SEQ ID NO: 36), BAIAP2L1/IRTKS (SEQ ID NO: 38), DNMBP/Tuba (SEQ ID NO: 40), FCHSD2 (SEQ ID NO: 42), FER (SEQ ID NO: 44); FES (SEQ ID NO: 46), FCHSD1 (SEQ ID NO: 48), GAS7 (SEQ ID NO: 50), GMIP (SEQ ID NO: 52), MTSS1/MIM (SEQ ID NO: 54), OPHN1 (SEQ ID NO: 56), PACSIN1 (SEQ ID NO: 58), PACSIN2 (SEQ ID NO: 60), PACSIN3 (SEQ ID NO: 62), SH3BP1 (SEQ ID NO: 64), SRGAP1 (SEQ ID NO: 66), SRGAP2 (SEQ ID NO: 68), SRGAP3 (SEQ ID NO: 70), ARHGAP4 (SEQ ID NO: 72) and FNBP1L (SEQ ID NO: 74).

Accordingly, in some embodiments, the agent is an siRNA, shRNA, miRNA that binds to one or more mRNAs of ARHGAP4, ARHGAP10/GARF2, ARHGAP17/RICH1, ARHGAP26/GRAF1, ARHGAP29, ARHGAP42/GRAF, ARHGAP44/RICH2, ARHGAP45/HMHA1, ARHGEF37, ARHGEF38, IRSp53/BAIAP2, BAIAP2L1/IRTKS, DNMBP/Tuba, FCHSD1, FCHSD2, FER; FES, FNBP1/FBP17, FNBP1L/Toca-1, GAS7, GMIP, MTSS1/MIM, MTSS1L/ABBA, OPHN1, PACSIN1, PACSIN2, PACSIN3, SH3BP1, SRGAP1, SRGAP2, SRGAP3, and TRIP10/CIP4.

In some embodiments, the siRNA that binds to one or more mRNAs of a BAR domain protein comprise any one of SEQ ID NO: 82-85 that bind to the mRNA of ARHGAP4, any one of SEQ ID NO: 86-89 that bind to an mRNA of ARHGAP10, any one of SEQ ID NO: 90-93 that bind to an mRNA of ARHGAP26, any one of SEQ ID NO: 94-97 that bind to an mRNA of ARHGAP29, any one of SEQ ID NO: 98-101 that bind to an mRNA of ARHGAP42, any one of SEQ ID NO: 102-105 that bind to an mRNA of BAIAP2, any one of SEQ ID NO: 106-109 that bind to an mRNA of BAIAP2L1, any one of SEQ ID NO: 110-113 that bind to an mRNA of DNMBP, any one of SEQ ID NO: 114-117 that bind to and mRNA of FCHSD1, any one of SEQ ID NO: 118-121 that bind to and mRNA of FCHSD2, any one of SEQ ID NO: 122-125 that bind to and mRNA of FES, any one of SEQ ID NO: 126-129 that bind to and mRNA of GAS7, any one of SEQ ID NO: 130-133 that bind to and mRNA of GMIP, any one of SEQ ID NO: 134-137 that bind to and mRNA of HMHA1, any one of SEQ ID NO: 138-141 that bind to and mRNA of MTSS1, any one of SEQ ID NO: 142-145 that bind to and mRNA of MTSS1L, any one of SEQ ID NO: 146-149 that bind to and mRNA of OPHN1, any one of SEQ ID NO: 150-153 that bind to and mRNA of PACSIN1, any one of SEQ ID NO: 154-157 that bind to and mRNA of PACSIN2, any one of SEQ ID NO: 158-161 that bind to and mRNA of PACSIN3, any one of SEQ ID NO: 162-165 that bind to and mRNA of SRGAP1, any one of SEQ ID NO: 166-169 that bind to and mRNA of SRGAP2, any one of SEQ ID NO: 170-173 that bind to and mRNA of SRGAP3, any one of SEQ ID NO: 174-177 that bind to the mRNA of ARHGAP17, any one of SEQ ID NO: 178-181 that bind to the mRNA of ARHGAP44, any one of SEQ ID NO: 182-185 that bind to the mRNA of SH3BP1, any one of SEQ ID NO: 186-189 that bind to the mRNA of ARHGEF37, any one of SEQ ID NO: 190-193 that bind to the mRNA of ARHGEF38, any one of SEQ ID NO: 194-197 that bind to the mRNA of FER, any one of SEQ ID NO: 198-201 that bind to the mRNA of FNBP1, any one of SEQ ID NO: 202-205 that bind to the mRNA of TRIP10, or any one of SEQ ID NO: 206-209 that bind to the mRNA of FNBP1L.

In some embodiments, the agent reduces or knocks down the bar domain genes MTSS1L/ABBA, FNBP1/FBP17, and TRIP10/CIP4 or their protein products.

In other embodiments, the agent may cause an increase in ERM protein expression such as one or more of EZR, RDX, and MSN, or an upstream activating protein such as a kinase or other activating protein. In some embodiments, the ERM genes are 90%, 95%, 96%, 97%, 98%, 99%, or 100% identical to EZR (SEQ ID NO: 1), RDX (SEQ ID NO: 3), and MSN (SEQ ID NO: 5). In some embodiments, the ERM genes are 90%, 95%, 96%, 97%, 98%, 99%, or 100% identical to EZR (SEQ ID NO: 2), RDX (SEQ ID NO: 4), and MSN (SEQ ID NO: 6). In one embodiment, the activating protein is transforming protein RhoA (SEQ ID NO: 8) having a DNA sequence according to SEQ ID NO: 7. In other embodiments, the activating protein is one or more of ROCK1 (SEQ ID NO: 211), ROCK 2 (SEQ ID NO: 213), SKL (SEQ ID NO: 215), and STK10 (SEQ ID NO: 217) or a gene encoding said protein, namely ROCK1 (SEQ ID NO: 210), ROCK2 (SEQ ID NO: 212), SKL (SEQ ID NO: 214), and STK10 (SEQ ID NO: 216). In other embodiments, the activating protein is an expression vector comprising one more PIP5K genes may be introduced into a cell to activate an ERM protein. These genes include PIP5K1A (SEQ ID NO: 76), PIP5K1B (SEQ ID NO: 78), and PIP5K1C (SEQ ID NO: 80) that encode PIP5K1A protein (SEQ ID NO: 77), PIP5K1B protein (SEQ ID NO: 79), and PIP5K1C protein (SEQ ID NO: 81), respectively.

In some embodiments, DNA comprising a coding region for a fusion protein, such as those that anchor an ERM protein to the plasma membrane, may be introduced into the cell where the DNA is subsequently expressed, and the fusion protein cause an increase in the tension of the plasma membrane. In one embodiment, the DNA is an expression vector or construct configured to express an MA-ezrin fusion protein comprising a conserved myristylation sequence of Lyn fused with ezrin, wherein the ezrin comprises a phosphomimic activating mutation (T567E). The DNA sequence of MA-ezrin fusion protein gene is SEQ ID NO: 9 and the amino acid sequence of the MA-ezrin protein is SEQ ID NO: 10. In some embodiments, the agent is an expression vector comprising the DNA sequence of the MA-ezrin fusion protein SEQ ID NO: 9.

In other embodiments of the disclosure, a method of inhibiting migration and/or proliferation of a cell comprises reducing expression of one or more proteins comprising a BAR domain, thereby inhibiting the migration and/or proliferation of a cell. Methods of reducing BAR domain protein proteins are discussed throughout the disclosure.

Agents may be delivered to and internalized into the cell using methods that are well known in the art. For example, in some embodiments, an agent such as a siRNA, shRNA, or miRNA, may be encapsulated in, bound to or adsorbed on a cationic lipid, a liposome, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, a cationic nanoemulsion or combinations thereof.

In some embodiments, the agent, and in particular, siRNA, shRNA, miRNA, or other polynucleotide molecule, may enter the cell via liposomes. Various amphiphilic lipids can form bilayers in an aqueous environment to encapsulate an RNA-containing aqueous core as a liposome. These lipids can have an anionic, cationic or zwitterionic hydrophilic head group. Formation of liposomes from anionic phospholipids dates back to the 1960s, and cationic liposome-forming lipids have been studied since the 1990s. Some phospholipids are anionic whereas other are zwitterionic. Suitable classes of phospholipid include, but are not limited to, phosphatidylethanolamines, phosphatidylcholines, phosphatidylserines, and phosphatidylglycerols. Useful cationic lipids include, but are not limited to, dioleoyl trimethylammonium propane (DOTAP), 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropanne (DLinDMA), 1,2-dilinolenyloxy-N,N-dimethyl-3-aminopropane (DLenDMA). Zwitterionic lipids include, but are not limited to, acyl zwitterionic lipids and ether zwitterionic lipids. Examples of useful zwitterionic lipids are DPPC, DOPC and dodecylphosphocholine. The lipids can be saturated or unsaturated.

Liposomes can be formed from a single lipid or from a mixture of lipids. A mixture may comprise (i) a mixture of anionic lipids (ii) a mixture of cationic lipids (iii) a mixture of zwitterionic lipids (iv) a mixture of anionic lipids and cationic lipids (v) a mixture of anionic lipids and zwitterionic lipids (vi) a mixture of zwitterionic lipids and cationic lipids or (vii) a mixture of anionic lipids, cationic lipids and zwitterionic lipids. Similarly, a mixture may comprise both saturated and unsaturated lipids. For example, a mixture may comprise DSPC (zwitterionic, saturated), DlinDMA (cationic, unsaturated), and/or DMPG (anionic, saturated). Where a mixture of lipids is used, not all of the component lipids in the mixture need to be amphiphilic e.g., one or more amphiphilic lipids can be mixed with cholesterol.

The hydrophilic portion of a lipid can be PEGylated (i.e., modified by covalent attachment of a polyethylene glycol). This modification can increase stability and prevent non-specific adsorption of the liposomes. For instance, lipids can be conjugated to PEG using techniques such as those disclosed in Heyes et au. (2005) J Controlled Release 107:276-87.

Liposomes are usually divided into three groups: multilamellar vesicles (MLV); small unilamellar vesicles (SUV); and large unilamellar vesicles (LUV). MLVs have multiple bilayers in each vesicle, forming several separate aqueous compartments. SUVs and LUVs have a single bilayer encapsulating an aqueous core; SUVs typically have a diameter 50 nm, and LUVs have a diameter>50 un. Liposomes useful with of the invention are ideally LUVs with a diameter in the range of 50-220 nm. For a composition comprising a population of LUVs with different diameters: (i) at least 80% by number should have diameters in the range of 20-220 nm, (ii) the average diameter (Zav, by intensity) of the population is ideally in the range of 40-200 nm, and/or (iii) the diameters should have a polydispersity index<0.2.

Techniques for preparing suitable liposomes are well known in the art e.g., see Liposomes: Methods and Protocols, Volume 1: Pharmaceutical Nanocarriers: Methods and Protocols. (ed. Weissig). Humana Press, 2009. ISBN 160327359X; Liposome Technology, volumes I, II & III. (ed. Gregoriadis). Informa Healthcare, 2006; and Functional Polymer Colloids and Microparticles volume 4 (Microspheres, microcapsules & liposomes). (eds. Arshady & Guyot). Citus Books, 2002.

In other embodiments, the agent, such as an siRNA, shRNA, miRNA, or other polynucleotide molecules, may enter the cell as part of a microparticle. various polymers can form microparticles to encapsulate or adsorb the agent. The use of a substantially non-toxic polymer means that a recipient can safely receive the particles, and the use of a biodegradable polymer means that the particles can be metabolized after delivery to avoid long-term persistence. Useful polymers are also sterilisable, to assist in preparing pharmaceutical grade formulations.

Suitable non-toxic and biodegradable polymers include, but are not limited to, poly(α-hydroxyacids), polyhydroxy butyric acids, polylactones (including polycaprolactones), polydioxanones, polyvalerolactone, polyorthoesters, polyanhydrides, polycyanoacrylates, tyrosine-derived polycarbonates, polyvinyl-pyrrolidinones or polyester-amides, and combinations thereof.

In some embodiments, the microparticles are formed from poly(α-hydroxy acids), such as a poly(lactides) (“PLA”), copolymers of lactide and glycolide such as a poly(D,L-lactide-co-glycolide) (“PLG”), and copolymers of D,L-lactide and caprolactone, Useful PLG polymers include those having a lactide/glycolide molar ratio ranging, for example, from 20:80 to 80:20 e.g., 25:75, 40:60, 45:55, 55:45, 60:40, 75:25. Useful PLG polymers include those having a molecular weight between, for example, 5,000-200,000 Da e.g., between 10,000-100,000, 20,000-70,000, 40,000-50,000 Da. Microparticles ideally have a diameter in the range of 0.02 μm to 8 μm. For a composition comprising a population of microparticles with different diameters at least 80% by number should have diameters in the range of 0.03-7 μm.

Techniques for preparing suitable microparticles are well known in the art e.g., see Functional Polymer Colloids and Microparticles volume 4 (Microspheres, microcapsules & liposomes). (eds. Arshady & Guyot). Citus Books, 2002: Polymers in Drug Delivery. (eds. Uchegbu & Schatzlein). CRC Press, 2006. (Chapter 7) and Microparticulate Systems for the Delivery of Proteins and Vaccines. (eds. Cohen & Bernstein). CRC Press, 1996. To facilitate adsorption of the agent, a microparticle may include a cationic surfactant and/or lipid e.g., as disclosed in O'Hagan et al. (2001) J Virology 75:9037-9043; and Singh et al. (2003) Pharmaceutical Research 20: 247-251. An alternative way of making polymeric microparticles is by molding and curing e.g., as disclosed in WO2009/132206.

In some embodiments, an RNAi molecule e.g., siRNA or other polynucleotide molecule can be adsorbed to the microparticles, and adsorption is facilitated by including cationic materials (e.g., cationic lipids) in the microparticle.

In some embodiments, the liposome or microparticle may include a targeting moiety such as an antibody, antibody fragment, antibody mimetic, aptamer, or ligand to target the liposome or microparticle to a specific cell e.g., by targeting specific receptor on the cell surface, thereby causing internalization of the liposome or microparticle.

In some embodiments, the agent (e.g., an siRNA molecule, shRNA molecule, etc.) may be introduced into cells by way of receptor-mediated endocytosis such as described in U.S. Pat. No. 6,090,619.

In some embodiments, a transfection reagent (e.g., cationic lipids, such as Lipofectamine) is typically used to facilitate transfection of the cell with the siRNA, shRNA, miRNA in the form of, for example, dsRNA.

In other embodiments, certain vectors may be used to deliver an agent to a target cell. These vectors include, for example, bacteriophage, plasmids, phagemides, viruses, integratable DNA fragments, episomal plasmids/viruses, and other vehicles or system, which enable the transfer of nucleic acid molecules into a desired target host cell, or enable the integration of a specific nucleic acid molecule in a particular location and in some further embodiments, leads to expression of said transduced nucleic acid molecule in the target cell. Non limiting examples for systems that may be used by the invention for specific targeted transfer of nucleic acid molecules may include the Clustered Regularly Interspaced Short Palindromic Repeat (CRISPR/Cas) system, the transcription activator effector nuclease (TALEN), the zinc finger protein (ZEN) systems and any equivalent system.

Vectors are typically self-replicating DNA or RNA constructs containing the desired nucleic acid sequences, and operably linked genetic control elements that are recognized in a suitable host cell and effect the transcription and translation of the desired gene. Generally, the genetic control elements can include a prokaryotic promoter system or a eukaryotic promoter expression control system. Such system typically includes a transcriptional promoter, transcription enhancers to elevate the level of RNA expression. Vectors usually contain an origin of replication that allows the vector to replicate independently of the host cell. However, there are vectors which are deliberately designed to be replication defective, enabling delivery into the cell and integration into the host genome, be it at random or at predesigned sites.

Accordingly, the ten control and regulatory elements includes promoters, terminators and other expression control elements. Such regulatory elements are described in Molecular Cell Biology Editors: H. Lodish et al., 7^{th} edition 2013 (or 8^{th} edition 2016). For instance, any of a wide variety of expression control sequences that control the expression of a DNA sequence when operatively linked to it may be used in these vectors to express DNA sequences encoding any desired RNA or protein using the method of this invention.

A vector or delivery vehicle may additionally include appropriate restriction sites, antibiotic resistance, fluorescence tags or other markers for positive selection (such as G418 resistance) or negative selection (such as the Herpes viral TK) of vector-containing cells. Plasmids are the most commonly used form of vectors but other forms of vectors which serve an equivalent function and which are, or become, known in the art are suitable for use herein. See, e.g., Ausubel et al., Current Protocols in Molecular Biology (2016) Wiley online library.

In some embodiments the delivery vehicle according to the present disclosure may be at least one viral vector, such as, lentivirus, adenovirus or AAV. Examples of adenoviral vectors and methods for gene transfer are described in PCT publications WO 00/12738 and WO 00/09675 and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

In some embodiments, an expression construct comprise one or more elements in any order selected from the group consisting of: (i) a multiple cloning site for introducing a sequence of nucleotides, which site is suitably cleavable enzymatically or other wise biochemically to provide a linearized vector into which PCR amplification products are cloneable directly (e.g., an EclHK1 site); (ii) a reporter gene; (iii) a promoter and/or enhancer for regulating expression of a transcribable polynucleotide (e.g., the polynucleotide that encodes the polypeptide); (iv) a polyadenylation sequence: (v) a selectable marker gene; and (vi) an origin of replication.

In certain embodiments, the expression constructs are in the form of vectors or sets of vectors, particularly but not exclusively plasmids, with applications in the study or measurement or monitoring of gene expression (e.g., promoter or enhancer activity). The vectors are suitably in the form of prokaryotic or eukaryotic vectors. Many other vectors could also be used such as for example viruses, artificial chromosomes, and other non-plasmid vectors.

The disclosure also provides for methods of modulating the rate of cell division of a Eukaryotic cell in culture. Certain cells may have a difficult time undergoing cell division when grown in culture while other cells only exhibit a slow rate of cell division. In other instances, it may be beneficial to slow the rate of cell division of some type of cells. The disclosure provides for methods of modulating the rate of cell division to increase the rate of cell division or decrease the rate of cell division depending on the needs of the user of the cell culture.

The disclosure also provides methods of increasing or decreasing a rate of cell division of eukaryotic cells in a cell culture comprising contacting the eukaryotic cell with an agent to cause a change in the tension of a plasma membrane of the cell, thereby increasing or decreasing the rate of cell division compared to a rate of cell division of a eukaryotic cell not contacted with the agent.

In some embodiments, the agent causing the decrease in the rate of cell division increases expression of one or more of ezrin, radixin, and moesin (ERM) proteins, or causes an increase in phosphorylation of the ERM proteins. In some embodiments, agent causing the increase in expression of the ERM proteins comprises an expression vector comprising one or more of ezrin (SEQ ID NO: 1), radixin (SEQ ID NO: 3), and moesin genes (SEQ ID NO: 5). In some embodiments, agent causing the increase in phosphorylation of the ERM proteins comprises an expression vector comprising one or more of ROCK1 (SEQ ID NO: 210), ROCK2 (SEQ ID NO: 212), SLK (SEQ ID NO: 214), STK10 (SEQ ID NO: 16), and RHOA (SEQ ID NO: 7) genes. The agent may comprise several expression vectors comprising one or more ERM proteins and/or ERM kinase as described above.

In one embodiment, the decrease in the rate of cell division is caused by an expression vector configured to express an ezrin fusion protein comprising a conserved myristylation sequence of Lyn fused with ezrin, wherein the ezrin comprises a phosphomimic activating mutation (T567E). In some embodiments, the ezrin fusion protein has the DNA sequence and amino acid sequence of SEQ ID NO: 8 and SEQ ID NO: 9, respectively.

In another embodiment, the agent causing the increase in the rate of cell division increases expression of one or more BAR domain proteins.

In some embodiments, the agent causing a change (e.g., increase) in the rate of cell division decreases expression or phosphorylation of one or more of ezrin, radixin, and moesin (ERM) proteins. For example, the agent can be a compound that inhibits one or more upstream kinases of an ERM protein. In one example, the agent is an inhibitor of ROCK1 and/or ROCK2 such as Y-27632 or ROCKi-IV as discussed, for example in Ohata et al., Cancer Res; 72(19) Oct. 1, 2012. Other inhibitors of ERM kinases are known in the art. In other embodiments, the agent is an antisense RNA, a siRNA, shRNA, or miRNA or an antibody.

The disclosure also provides for methods of modulating a characteristic of a cell comprising contacting the cell with an agent to change a tension of a plasma membrane of the cell, wherein the change in the tension of the plasma membrane modulates the characteristic of the cell.

In some embodiments, the characteristic of the cell is controlled by a membrane-actin cortex attachment (MCA), and the agent increases or decreases the MCA compared to a control cell.

In some embodiments, the characteristic of the cell is an increase or decrease in cell motility and invasion of surrounding tissue. In other embodiments, the characteristic of the cell is an increase or decrease in cell proliferation and tumor formation. Agents that may be used to modulate a characteristic of a cell are discussed throughout the specification.

### Results and Discussion

Malignancy is associated with changes in cell mechanics that contribute to the extensive cell deformation required for metastatic dissemination. We hypothesized that the cell-intrinsic physical factors that maintain epithelial cell mechanics could function as tumor suppressors. Here we show, using optical tweezers, genetic interference, mechanical perturbations, and in vivo studies, that epithelial cells maintain higher plasma membrane (PM) tension than their metastatic counterparts, and that high PM tension potently inhibits cancer cell migration and invasion by counteracting membrane-curvature sensing/generating BAR family proteins. This tensional homeostasis is achieved by membrane-cortex attachments (MCA) regulated by ERM proteins, whose disruption spontaneously transforms epithelial cells into a mesenchymal migratory phenotype powered by BAR proteins. Consistently, induction of epithelial-mesenchymal transition (EMT) by forced expression of EMT-transcription factors leads to a decrease in PM tension. In metastatic cells, increasing PM tension by manipulating MCA is sufficient to suppress both mesenchymal and amoeboid 3D migration and tumor invasion by compromising membrane-mediated mechanosignaling by BAR proteins, thereby uncovering a previously undescribed mechanical tumor suppressor mechanism.

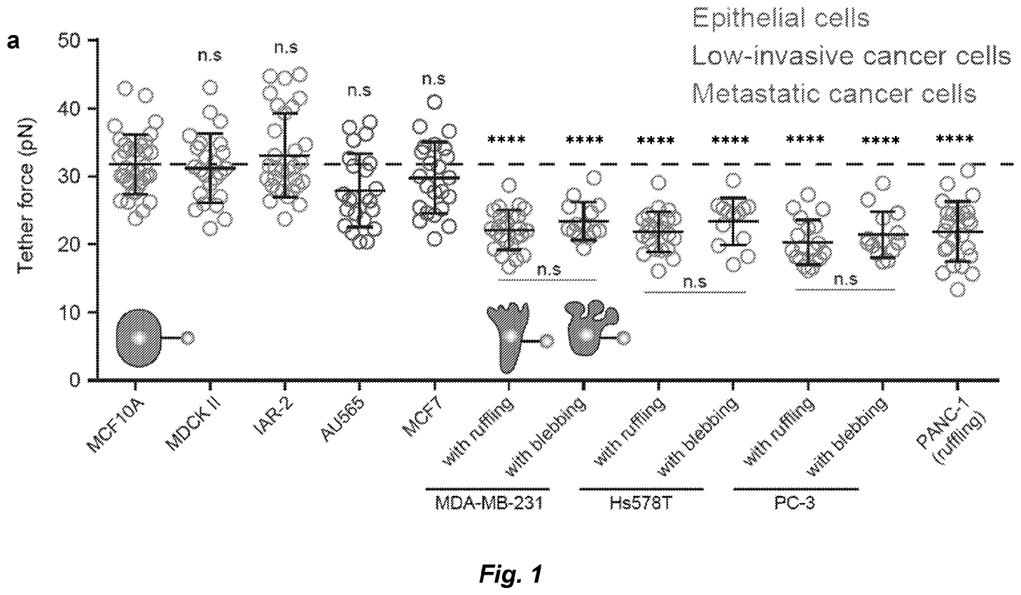

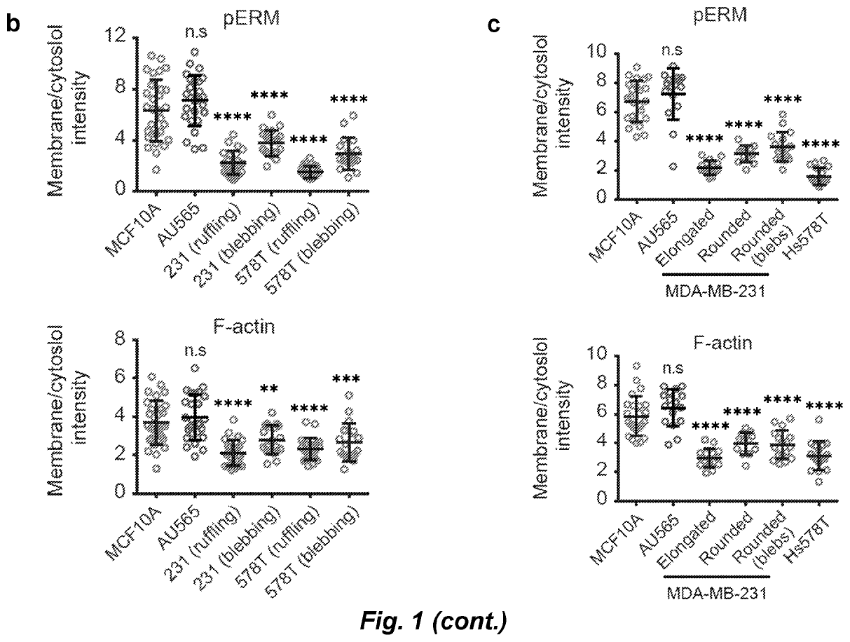

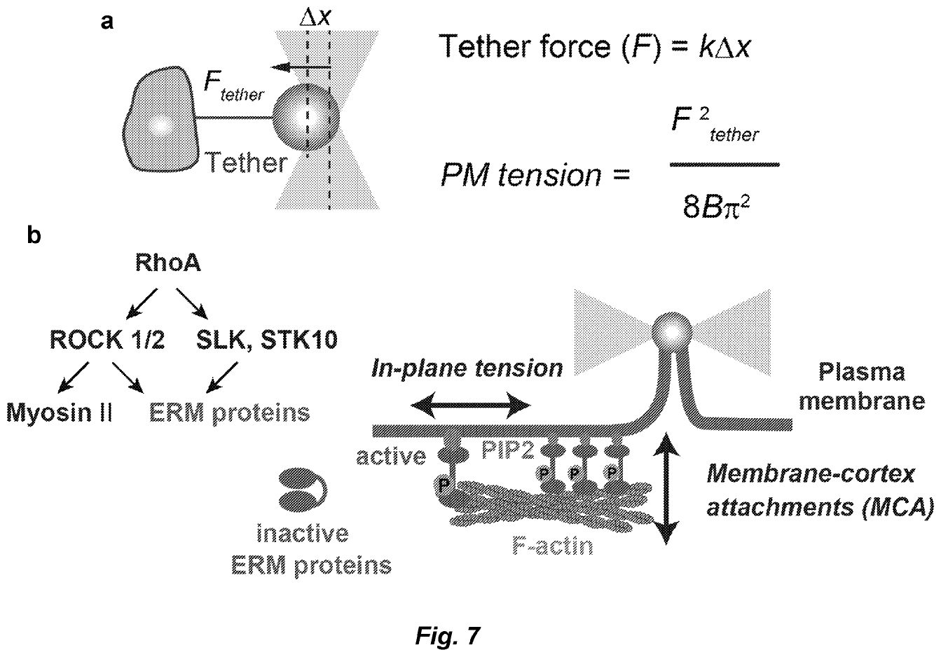

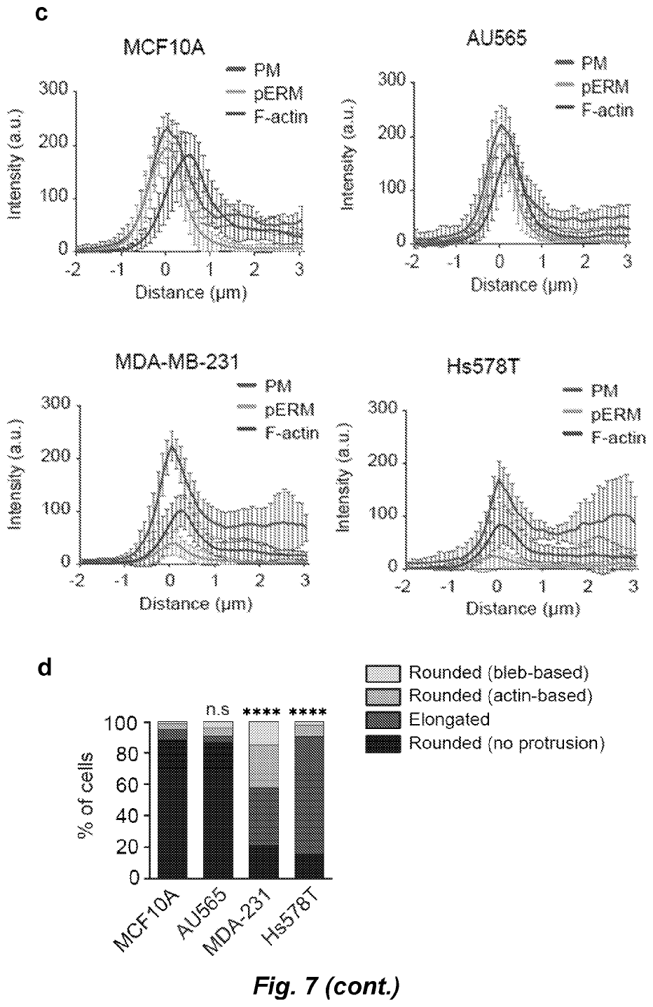

Epithelial cells have higher PM tension than their malignant counterparts. To determine whether there is a difference in PM tension between epithelial cells and their metastatic counterparts, we used optical tweezers to analyze the membrane tether force, which is proportional to PM tension (FIG. 7a). We primarily used human non-invasive mammary epithelial cells (MCF10A) and metastatic breast cancer cells (MDA-MB-231), as they are a commonly used model of malignant transition. To avoid cell-extrinsic effects, such as cell-cell contact, we measured the tether force at the single-cell level. We found that the tether force of MCF10A cells was largely comparable to that of the low-invasive human breast cancer cells (AU565 and MCF7; FIG. 1a). Unlike these low-invasive cells, metastatic breast cancer cells, such as MDA-MB-231 and Hs578T cells, formed both prominent membrane ruffles and blebs when cultured on uncoated glass substrates. Interestingly, we found that there was no difference in the tether force between ruffling and blebbing cells, and their tether force was significantly lower than that of their low-motility counterparts (FIG. 1a). Similar results were obtained in aggressive prostate cancer cells (PC-3) and pancreatic cancer cells (PANC-1; FIG. 1a). PM tension was approximately two-fold lower in metastatic cells than in low-motility cells (Table 1). The ability to maintain higher PM tension than malignant cells appear to be a common characteristic of epithelial cells across species and tissues, as the tether force of normal epithelial cells, such as canine kidney MDCK II cells and rat liver IAR-2 cells, was similar to that of MCF10A cells (FIG. 1a, Table 1).

TABLE 1
Mean values of tether force and PM tension in FIG. 1a.

| Cell Type | Tether Force (pN) | PM Tension (pN/μm) |
|---|---|---|
| MCF-10A | 31.76 | 91.89 |
| MDCK II | 31.2 | 90.42 |
| IAR-2 | 33.1 | 102.6 |
| AU565 | 27.92 | 73.09 |
| MCF7 | 29.79 | 82.78 |
| MDA-MB-231 (ruffling) | 22.15 | 45.19 |
| MDA-MB-231 (blebbing) | 23.45 | 50.45 |
| Hs578T (ruffling) | 21.89 | 44.05 |
| Hs578T (blebbing) | 23.36 | 50.43 |
| PC-3 (ruffling) | 20.32 | 38.33 |
| PC-3 (ruffling) | 21.44 | 42.61 |
| PANC-1 (ruffling) | 21.88 | 45.08 |

PM tension has contributions from the in-plane tension of the lipid bilayer and MCA (FIG. 7b). Given that PM tension is thought to be largely dependent on MCA, we focused on ERM proteins and F-actin beneath the PM. ERM proteins are activated by the phosphorylation of conserved threonine residues. We observed that MCA10A and AU565 cells had intense phosphorylated ERM (pERM) signals globally decorating the PM, where they partially co-localized with F-actin (FIGS. 1b and 7c). In contrast, MDA-MB-231 and Hs578T cells had globally lower levels of membrane-associated pERM and F-actin, regardless of whether they exhibited membrane ruffling or blebbing, and some pERM levels were restricted to the cell rear and possibly shrinking blebs (FIGS. 1b and 7c). Consistent with these 2D observations, confocal and reconstituted 3D images showed that MCF10A and AU565 cells embedded in a collagen matrix (3D) always exhibited a rounded shape with uniform and intense pERM and F-actin levels beneath the PM (FIG. 1c and FIG. 7d). MDA-MB-231 cells exhibited both actin-based elongated mesenchymal and actin- and bleb-based rounded amoeboid migration phenotypes in 3D (FIG. 7d). In both cases, membrane-proximal pERM and F-actin displayed overall decreased levels (FIG. 1c). Similarly, Hs578T cells, which predominantly exhibit an actin-based elongated phenotype in 3D (FIG. 7d), gave a consistent phenotype (FIG. 1c). These results suggest that the observed differences in PM tension between non-motile and invasive cells are primarily due to ERM-mediated changes in MCA and that such mechanical disruptions correlate with an invasive phenotype irrespective of the type of protrusions and motile modes.

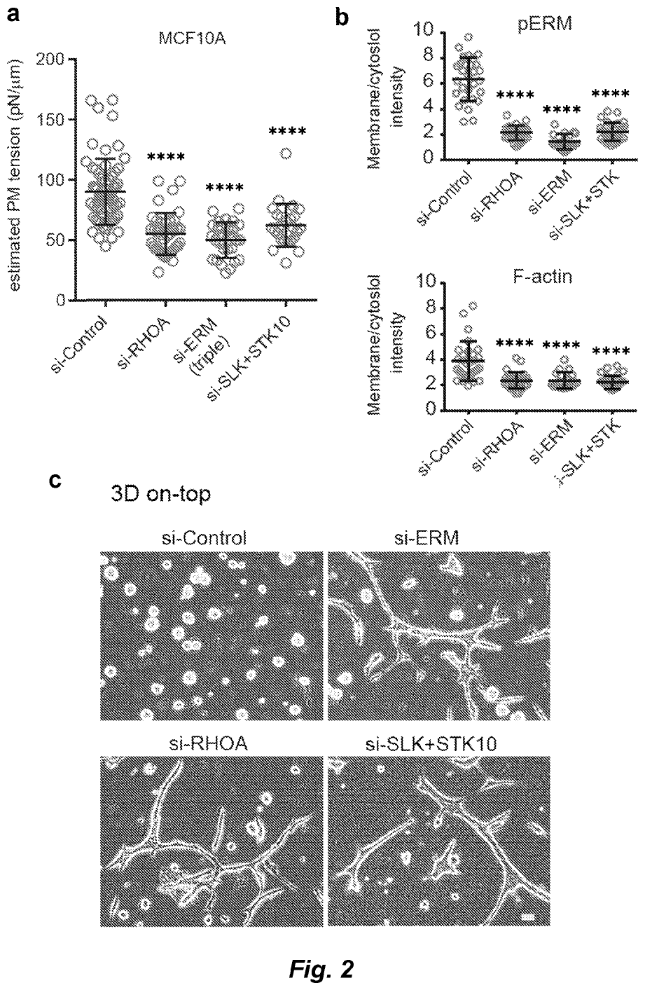

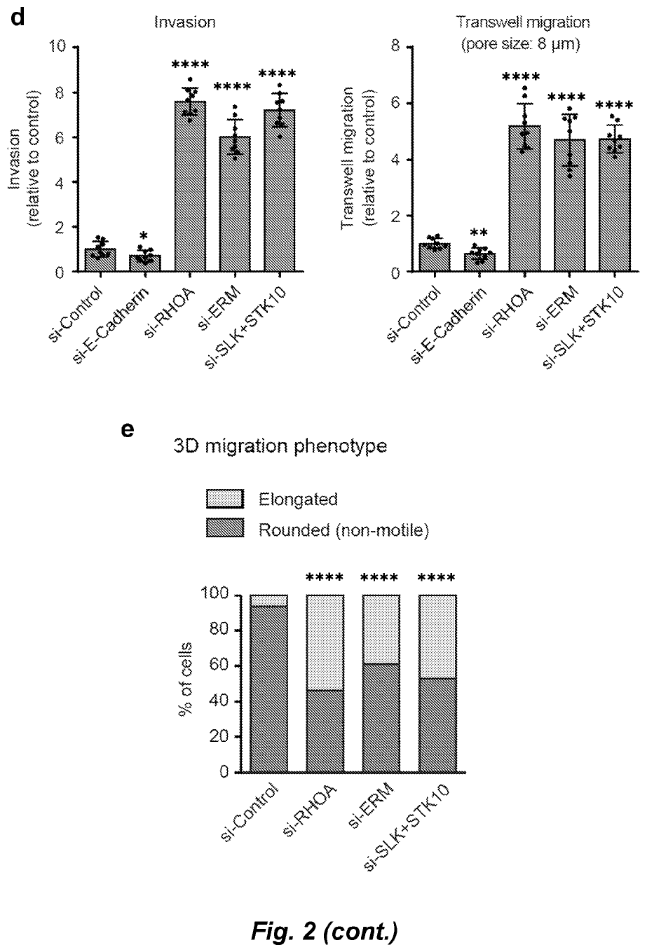

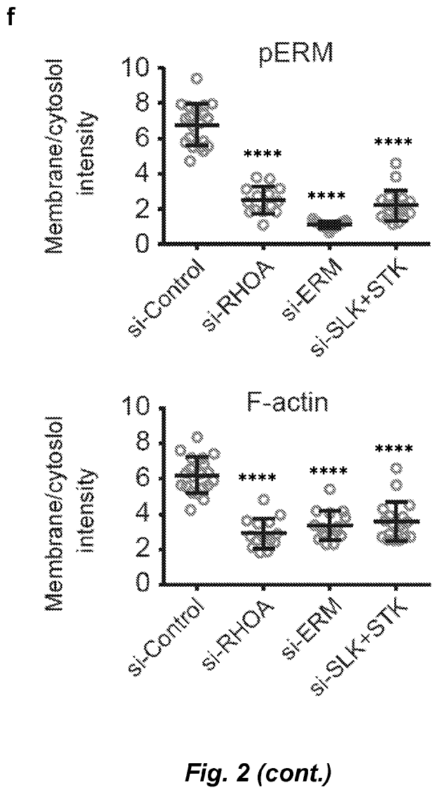

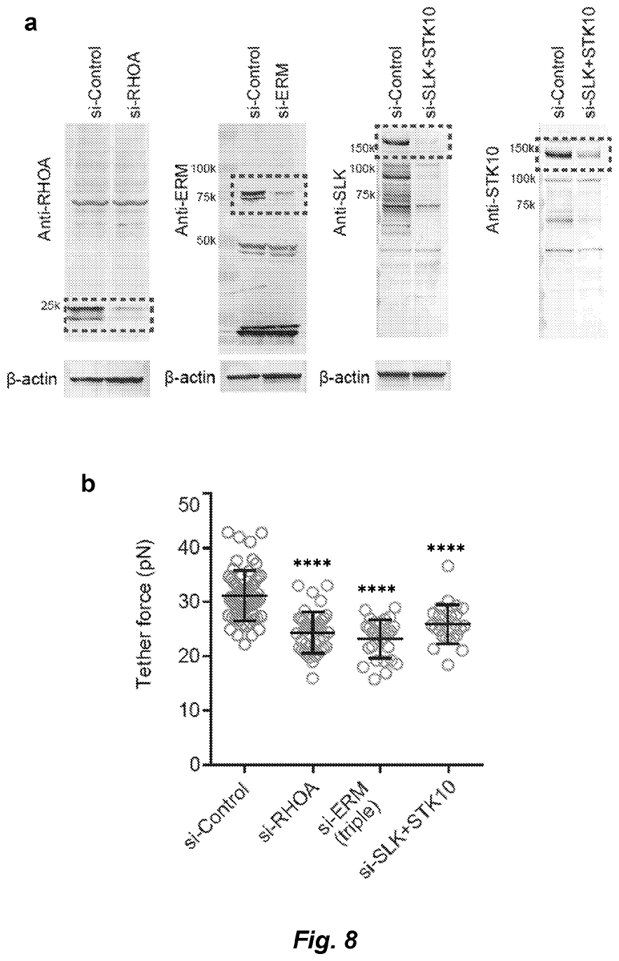

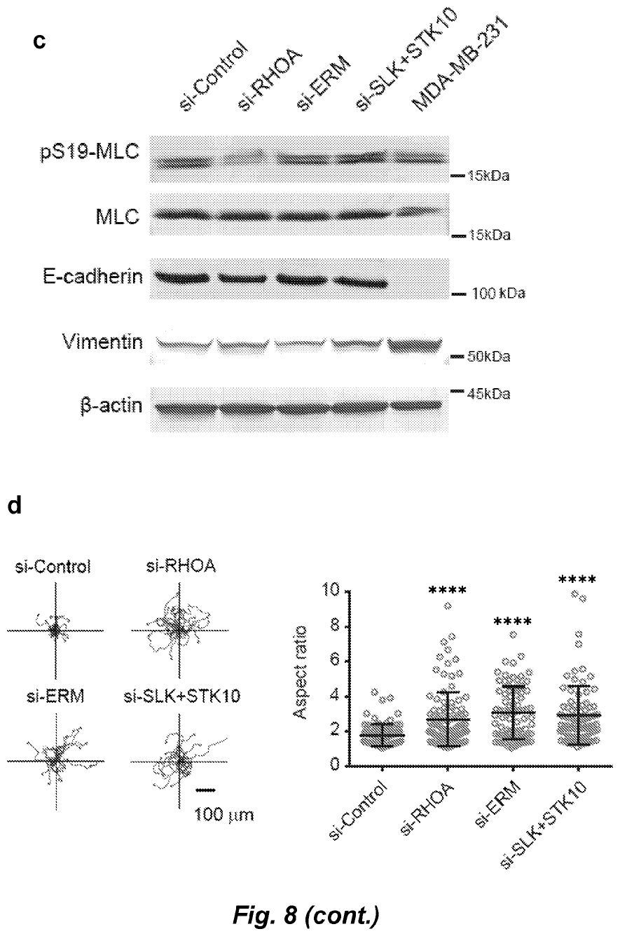

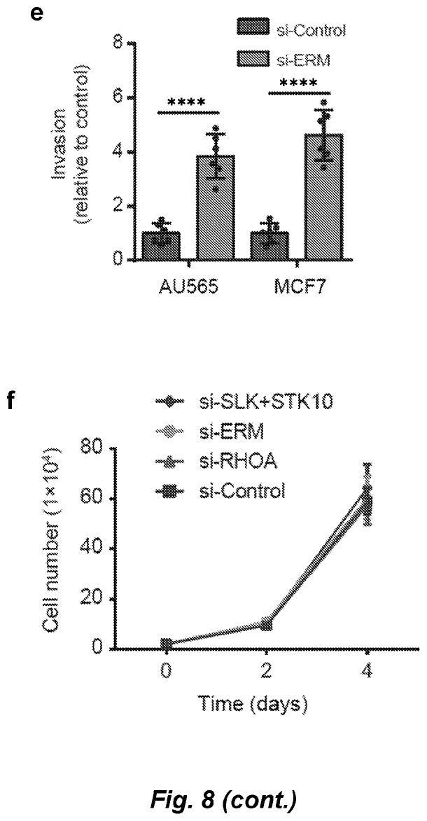

Epithelial cells spontaneously convert to a mesenchymal migratory phenotype when PM tension is reduced. The above findings prompted us to hypothesize that reducing PM tension may be sufficient for epithelial cells to acquire invasive behavior. To test this, we manipulated MCA by simultaneously knocking down three members of ERM (ezrin, radixin, and moesin) or their specific kinases (SLK and STK10 [also known as LOK]) (FIG. 8a). We also focused on RHOA, as it is known to act as an upstream regulator of these ERM kinases (FIG. 7b). Paradoxically, despite its key role in cell migration, RHOA has been reported to play an inhibitory role in invasion and metastasis. Notably, knockdown of these proteins in MCF10A cells led to a significant decrease in tether force (FIG. 8b), which corresponded to a decrease in PM tension (FIG. 2a). Of note, phosphorylated myosin light chain (pS19MLC), a marker for myosin II activation, was not affected in ERM and SLK+STK10 depleted cells (FIG. 8c), indicating that the observed differences in PM tension were specifically due to changes in MCA rather than those in actomyosin contractility. These knocked-down cells exhibited overall decreased levels of membrane-associated pERM and F-actin, concomitant with cell spreading with prominent actin-based protrusions, such as lamellipodia and membrane ruffling in 2D (FIG. 2b, FIG. 8d). We next evaluated the effects of decreased PM tension in a 3D on-top culture system with a mixture of collagen and Matrigel commonly used in cancer research. As expected, control cells formed perfectly rounded spheroids (FIG. 2c). Strikingly, over 60% of the low-tension cells displayed an elongated invasive phenotype, exhibiting cell dissemination into the surrounding matrix (FIG. 2c). These cells consistently displayed a marked increase in motility behaviors, including invasion and confined migration through narrow gaps (Fig. d). The observed invasive phenotype seemed to be independent of the classical EMT program, as these low-tension cells retained epithelial characteristics (FIG. 8c), and E-cadherin depletion resulted in a slight but significant inhibition of invasion and migration (Fig. d). The deletion of ERM proteins in low-invasive breast cancer cells also led to a drastic increase in invasion (FIG. 8e). Decreased MCA did not affect the proliferation of MCF10A cells (FIG. 8f).

We further investigated the effect of PM tension reduction on single-cell motility behavior in 3D. Time-lapse movies showed that approximately 95% of control RNAi-treated cells displayed a rounded shape with a non-motile phenotype in a single-cell state when embedded in a collagen matrix (FIG. 2e). In contrast, knockdown of the MCA regulators all led to conversion to the migration phenotype in a mesenchymal fashion: approximately 50% of the cells displayed an elongated migration phenotype with dynamic cell-shape changes showing membrane protrusion and retraction (FIG. 2e). No amoeboid-like movement characterized by a rounded shape was observed in these knocked-down cells. In 3D, such an elongated phenotype was closely associated with lower levels of pERM and F-actin beneath the PM (FIG. 2f) as observed in metastatic cells. These data indicate that non-motile epithelial cells can spontaneously convert to a mesenchymal-like motility phenotype when PM tension is reduced.

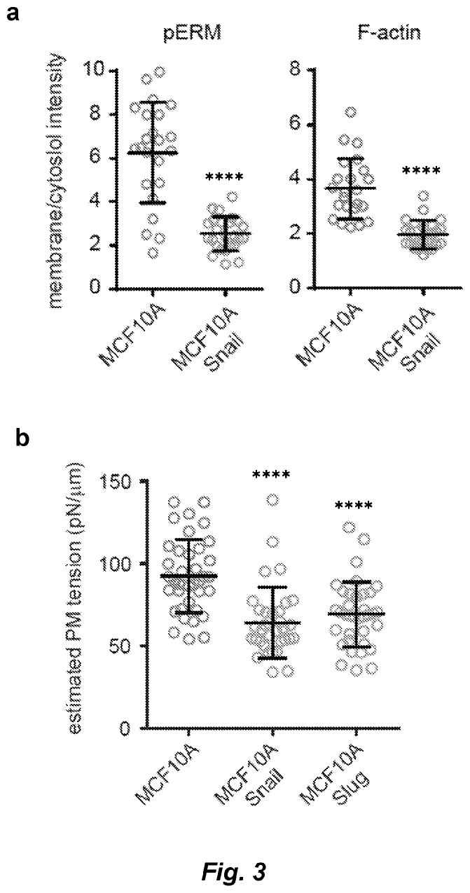

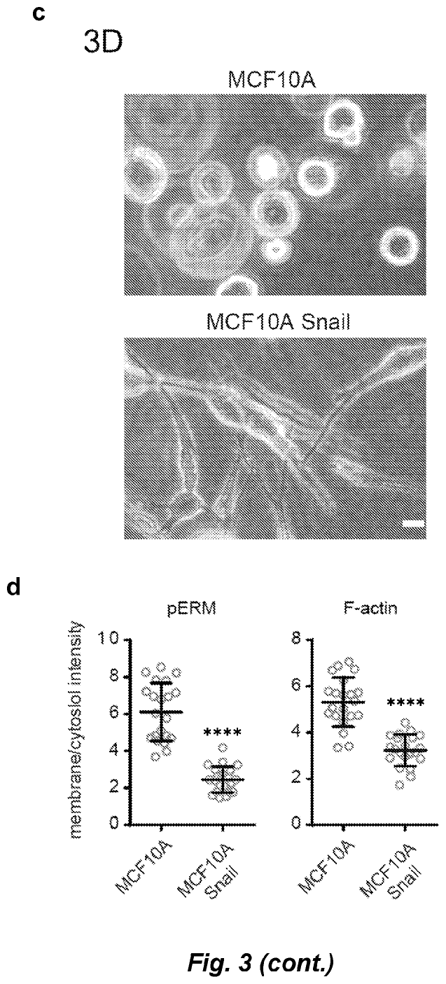

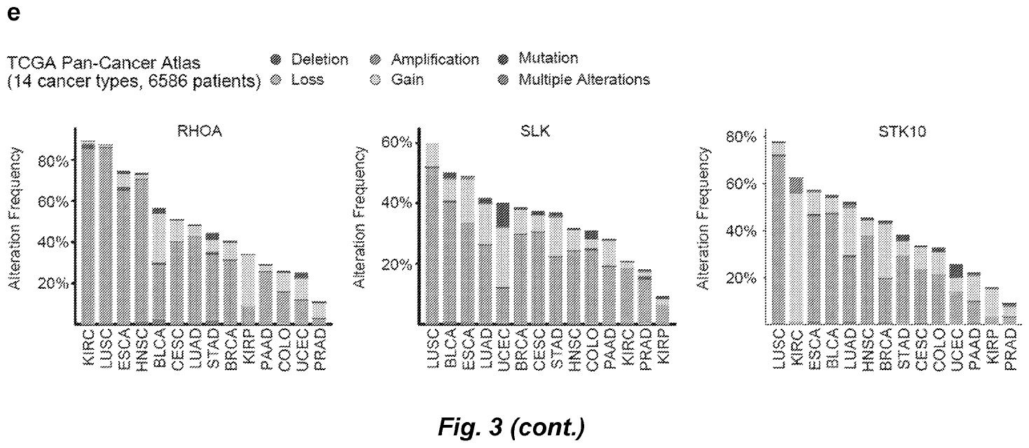

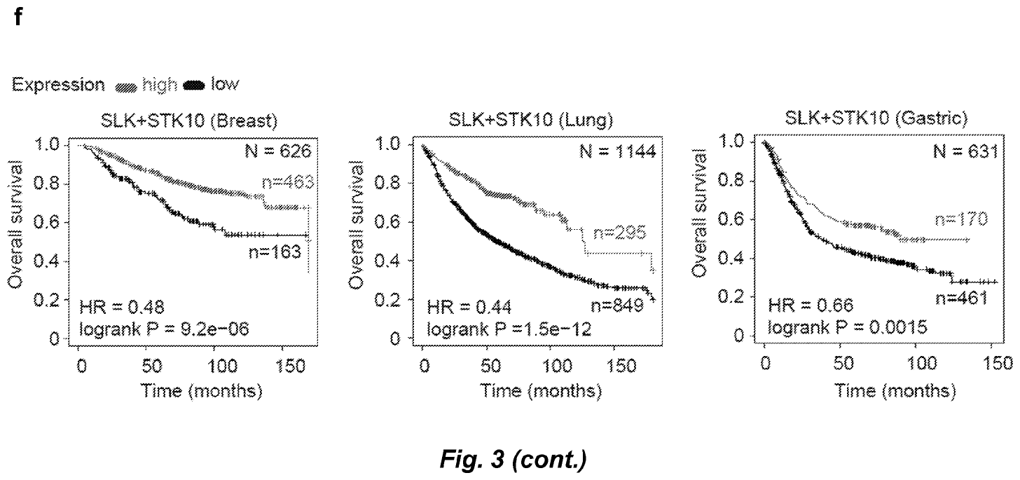

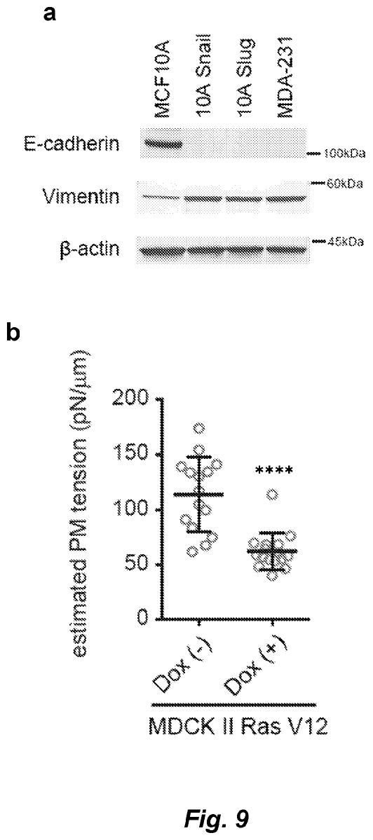

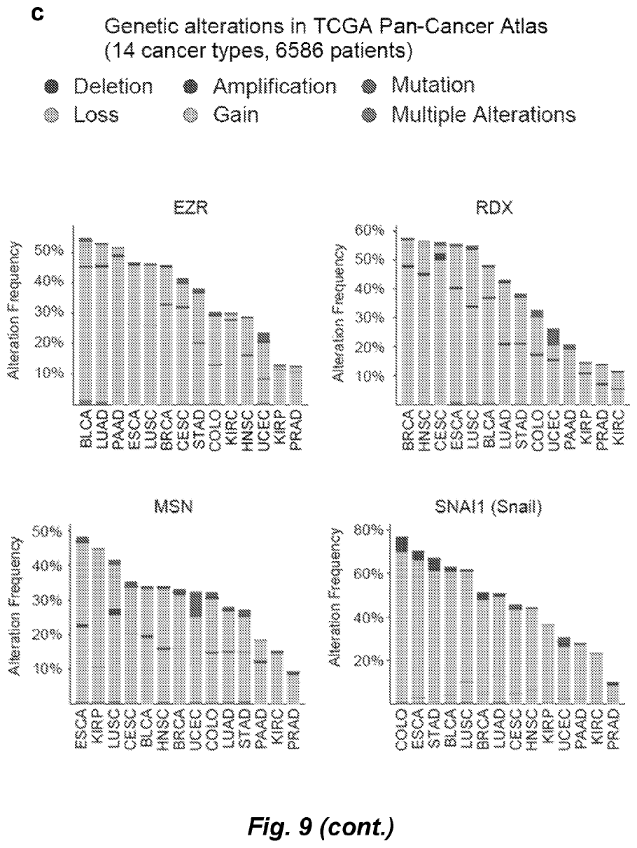

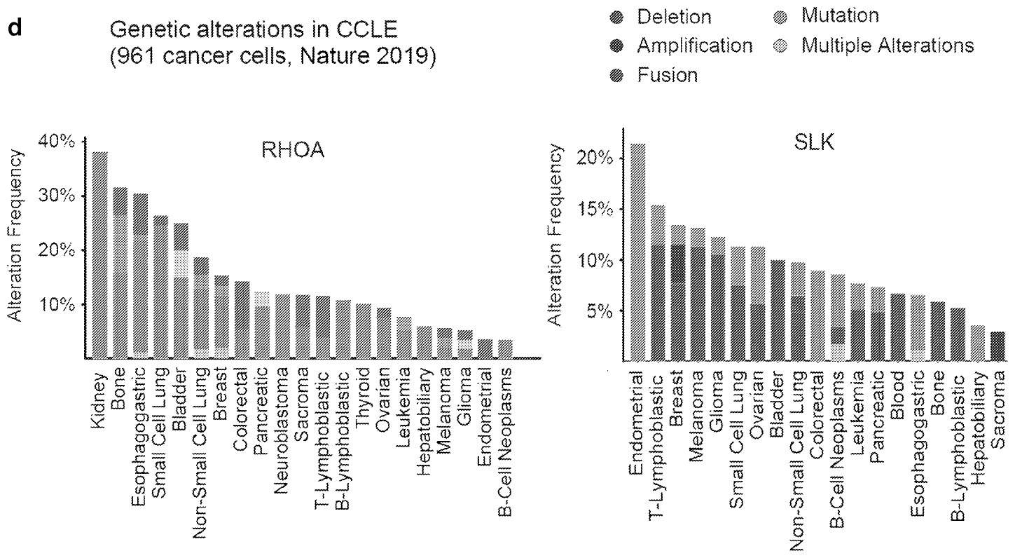

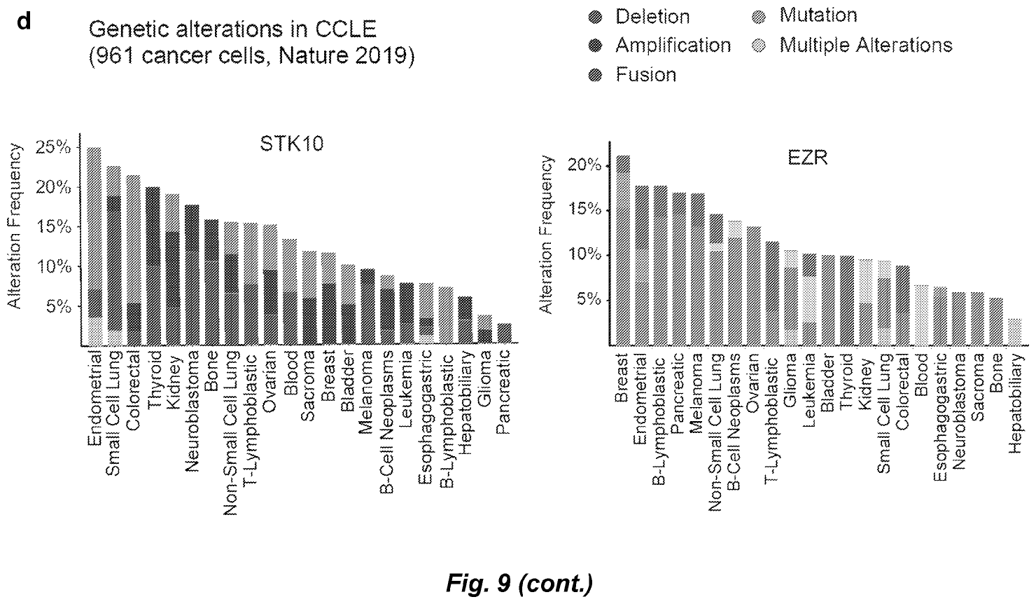

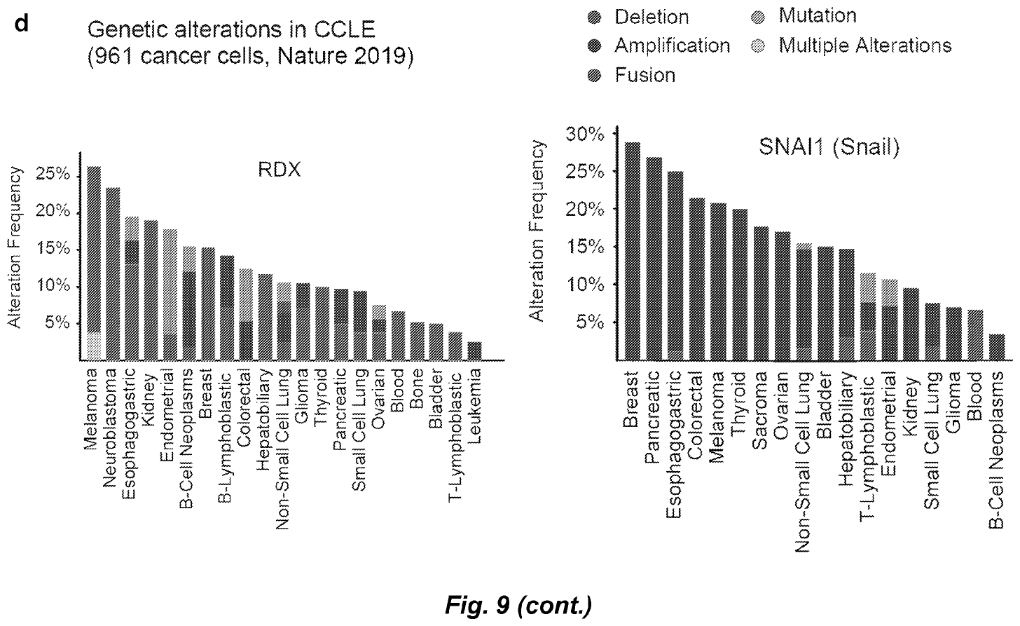

Correlation between reduced PM tension and malignant progression. We noted that the elongated cell-shape changes induced by decreased PM tension were strikingly similar to those induced by EMT. Therefore, we assumed that the disruption of tensional homeostasis in the PM might be commonly associated with malignant transformation. To test this hypothesis, we established MCF10A cell lines stably overexpressing Snail or Slug, transcription factors that strongly induce EMT. As expected, these cells displayed EMT characteristics, including actin-based protrusion formation (FIG. 3a, arrowhead) and altered expression of E-cadherin and vimentin (FIG. 9a). Interestingly, Snail expression resulted in a significant reduction in membrane-associated pERM and F-actin levels (FIG. 3a). Moreover, MCF10A cells overexpressing Snail or Slug exhibited lower PM tension than that in the parental cells (FIG. 3b). A similar result was obtained in MDCK II cells inducibly expressing a K-Ras-activating mutant (G12V) (FIG. 9b), which is a known key driver of cancer progression and metastasis. In 3D, EMT-induced elongated morphology was closely associated with altered pERM and actin staining patterns, as in 2D (FIGS. 3c and d), suggesting a close correlation between changes in PM tension and the acquisition of an invasive phenotype.

To further clarify whether PM tension reduction is a common feature involved in cancer progression, we analyzed cancer genomes using pan-cancer data from The Cancer Genome Atlas (TCGA). Surprisingly, a comprehensive analysis of 14 major carcinomas across 6586 patients revealed that epithelial tumors frequently harbor putative heterozygous deletions of RHOA, SLK, STK10, and ERM (FIG. 3e and FIG. 9c). Similar trends were observed in 961 cancer cell lines in the Cancer Cell Line Encyclopedia (CCLE; FIG. 9d). Moreover, a meta-analysis revealed significant associations between the increased expression of ERM kinases and increased patient survival in breast, lung, and gastric cancers (FIG. 3f). These data suggest that the disruption of homeostatic PM tension is a common mechanical characteristic of malignant cells and may be correlated with cancer progression.

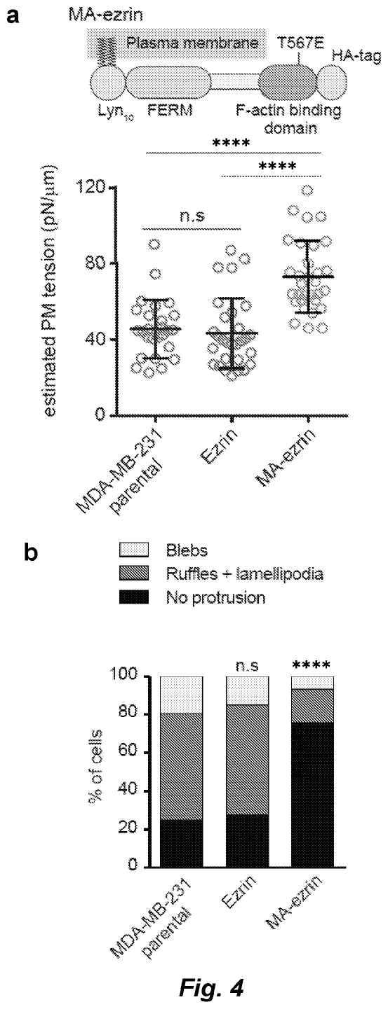

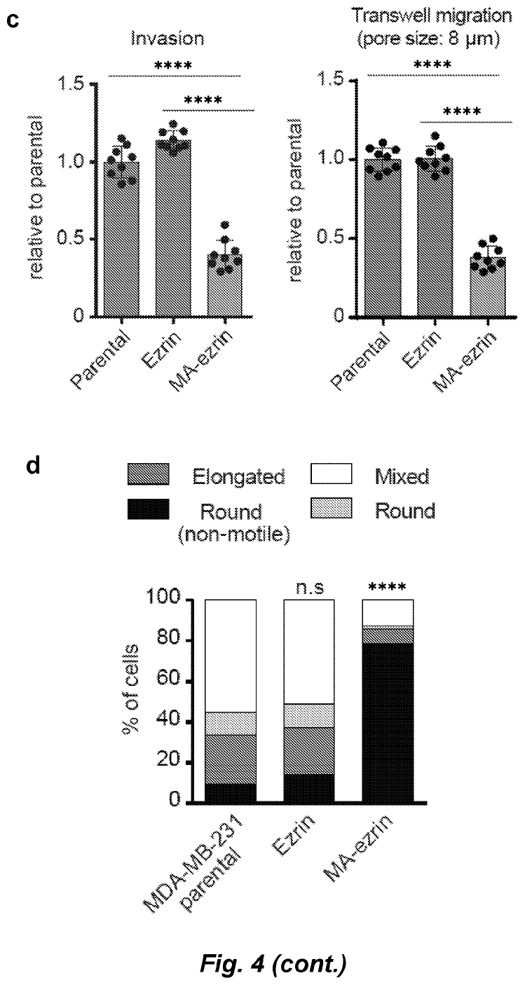

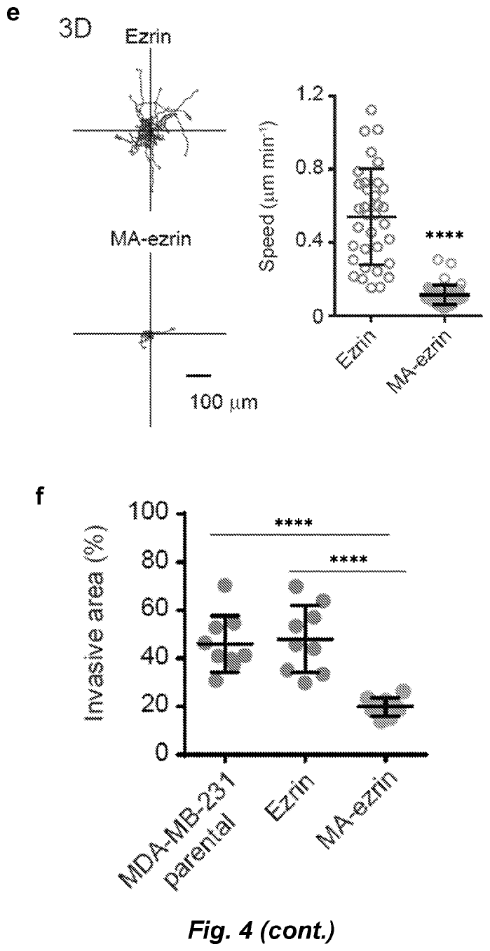

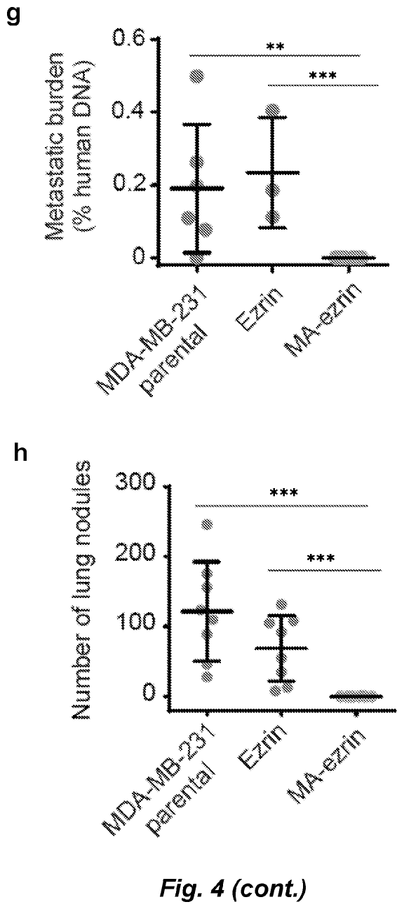

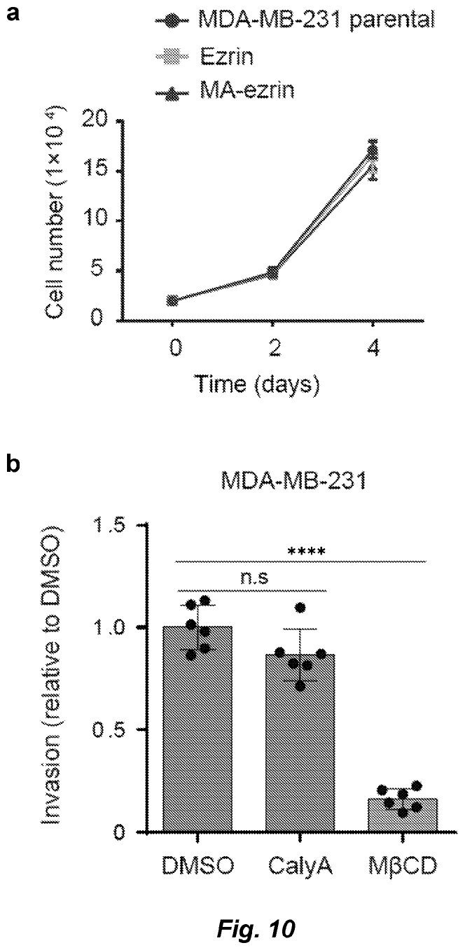

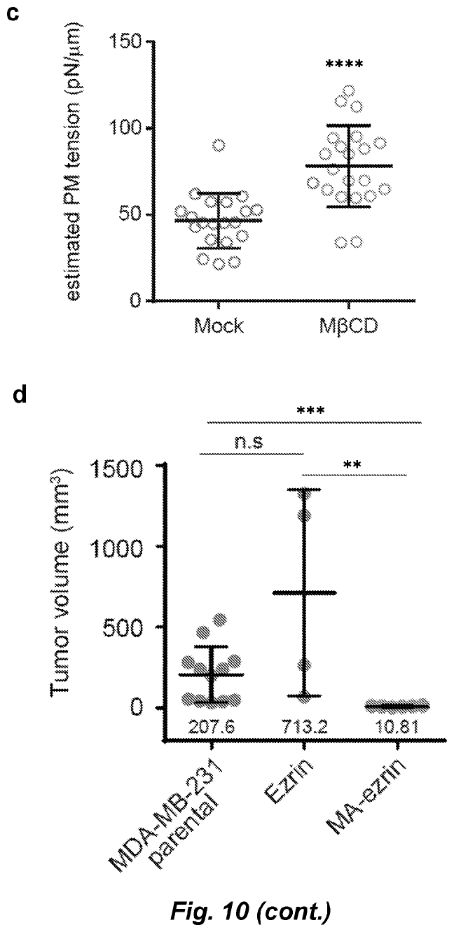

Increasing PM tension is sufficient to suppress 3D migration, tumor invasion, and metastasis. We hypothesized that if PM tension reduction is key to acquiring migration and invasiveness, increasing MCA might be sufficient to suppress cancer cell dissemination. Therefore, we attempted to increase PM tension by directly manipulating MCA. Given that ERM proteins globally dissociate from the PM in aggressive cancer cells, we reasoned that membrane-targeted active ezrin (MA-ezrin) could rescue decreased PM tension. To test this, we engineered an MA-ezrin construct in which the conserved myristoylation sequence of Lyn was fused with ezrin, followed by the introduction of a phosphomimetic-activating mutation (T567E), thereby maintaining its active state throughout the PM (FIG. 4a). Notably, its expression in MDA-MB-231 cells led to a significant increase in PM tension (FIG. 4a). MA-ezrin was globally localized to the PM, resulting in the suppression of prominent actin- and bleb-based membrane protrusions (FIGS. 4b and b). These high-tension cells exhibited normal cell proliferation ability in vitro (FIG. 10a), but a marked decrease in invasion and migration (FIG. 4c).

ERM activity has also been reported to be associated with cortical stiffness, at least in mitotic cell rounding. We found that treatment with calyculin A, which is commonly used to increase cortical stiffness or tension by increasing myosin II activity, has no effect on the invasive ability (FIG. 10b), reflecting the plasticity of cancer cell migration due to changes in contractility. Furthermore, treating cells with methyl-β-cyclodextrin (MβCD), a cholesterol-removing compound that increases PM tension (FIG. 10c) but decreases cortical stiffness, significantly reduced invasion (FIG. 10b), ruling out potential effects of cortical stiffness on inhibiting cancer cell migration.

Next, we tested whether an increase in PM tension affected 3D migration. 3D reconstituted confocal images showed that while ezrin-expressing cells displayed different protrusion phenotypes, as in parental cells, MA-ezrin-expressing cells had rounded shapes with no protrusion formation, reminiscent of epithelial cell characteristics. Indeed, time-lapse movies showed that parental and ezrin-expressing MDA-MB-231 cells exhibited elongated mesenchymal motility (approximately 20%), rounded amoeboid motility, and phenotypic switching between the two modes (mixed phenotype; 50%) (FIGS. 4d and e), as previously reported. In contrast, the majority of MA-ezrin-expressing cells displayed rounded shapes with non-migratory behavior (80%) and markedly reduced migration velocity (over 4-fold slower) (FIGS. 4d and e, Fig). These data demonstrate that increasing PM tension is sufficient to suppress both two different modes of 3D migration.

To further investigate whether maintaining high PM tension plays an active role in suppressing cancer cell dissemination, we employed an orthotopic mouse model of human breast tumor formation, local invasion, and spontaneous metastasis. When injected into the mammary fat pad, MA-ezrin-expressing MDA-MB-231 cells showed reduced tumorigenic ability and produced significantly smaller tumors (FIG. 10d), although they proliferated at rates comparable to that of parental cells in vitro (Table 2; FIG. 10a).

TABLE 2
Tumor formation after injection of the indicated
cells into the mammary fat pad.

| Cell | Tumor Formation |
|---|---|
| Parental | 12/12 |
| Ezrin | 4/4 |
| MA-Ezrin | 6/13 |

In addition, in contrast to control tumors, which had a prominent invasive behavior with numerous cancer cells invading into the surrounding adipose tissue, MA-ezrin-expressing tumors displayed a clear borderline between the region of tumor cells and the adjacent tissue, showing a significant reduction in invasiveness (FIG. 4f). Moreover, MA-ezrin cells exhibited a marked reduction in spontaneous (FIG. 4g) and experimental (tail vein injection) lung metastasis (Fig. h). Taken together, these data indicate that PM tension sustained by MCA acts as a potent tumor suppressor that inhibits tumorigenesis, invasion, and metastasis.

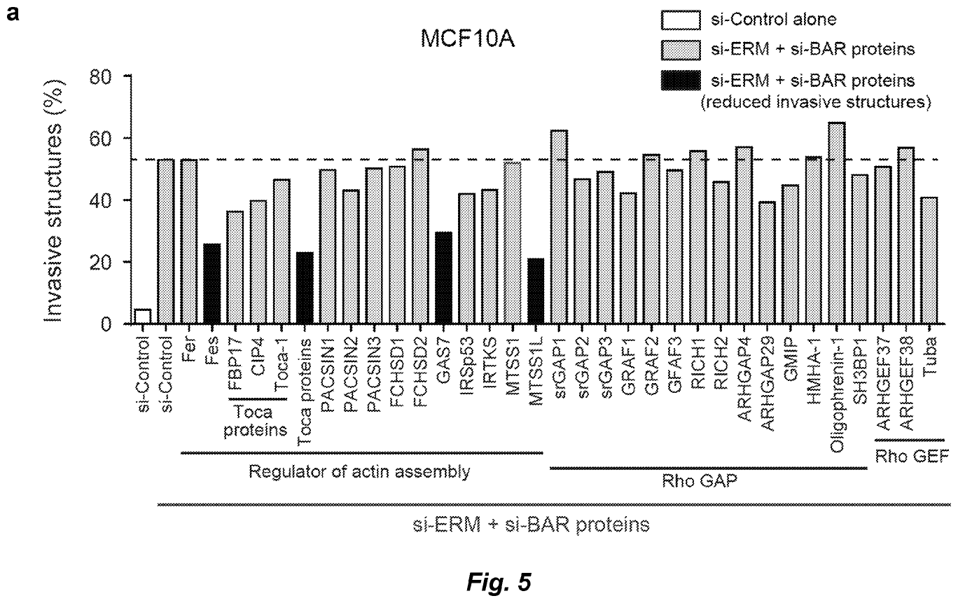

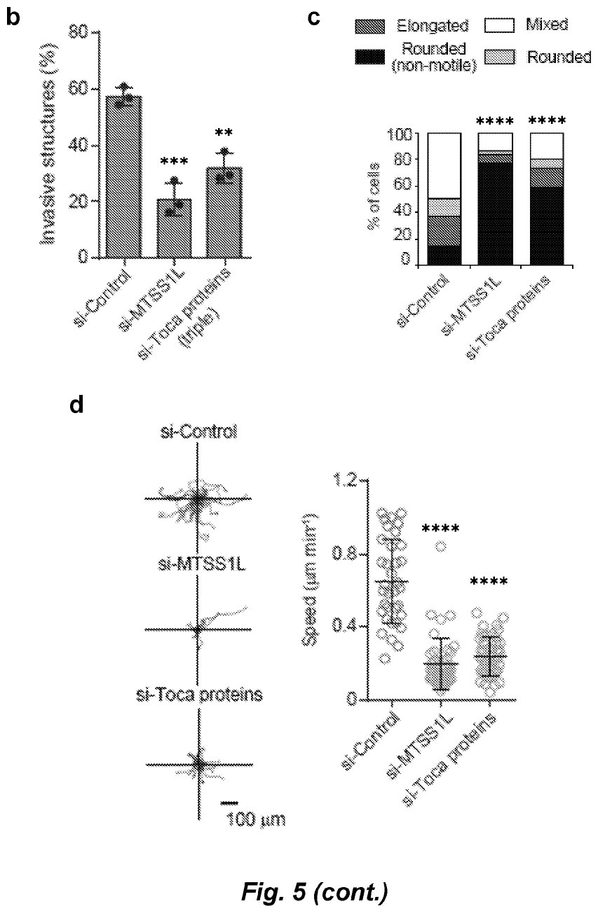

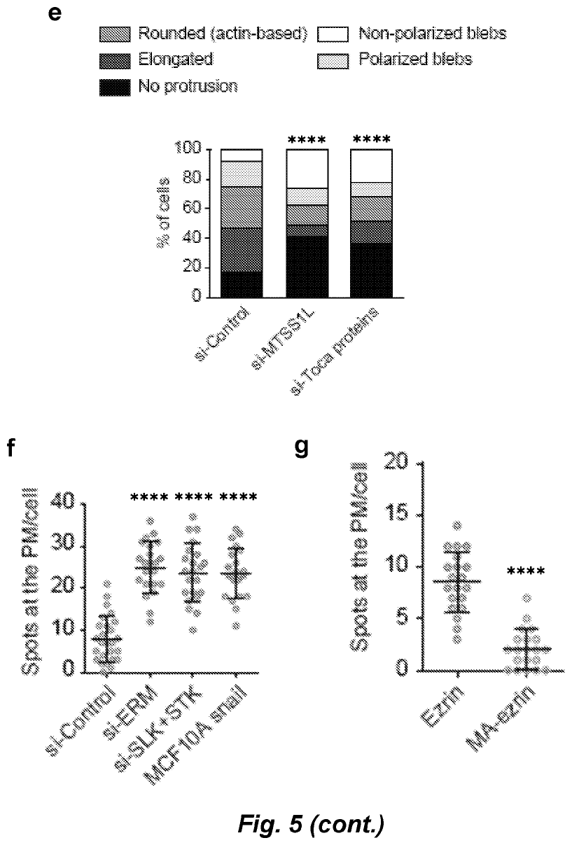

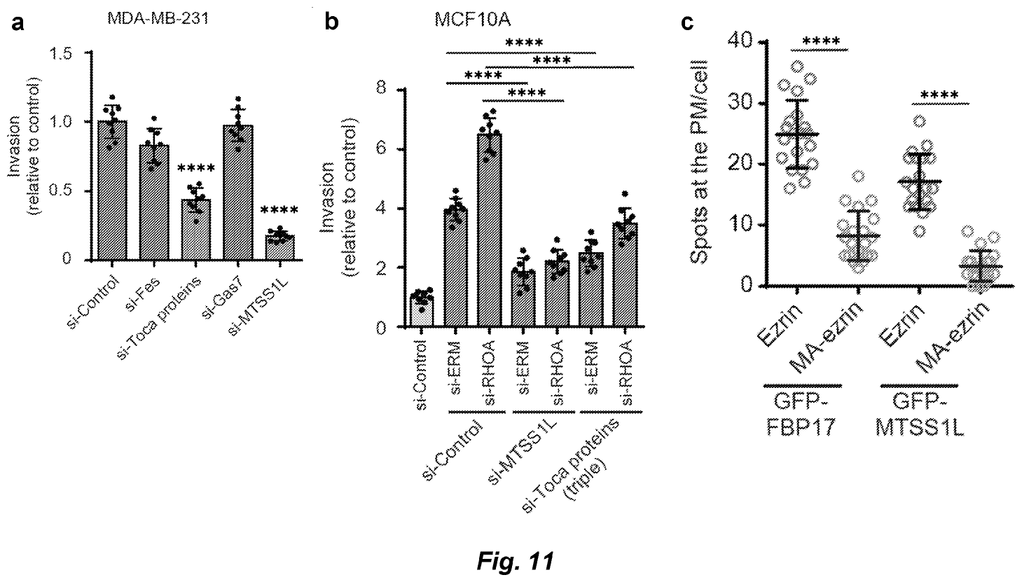

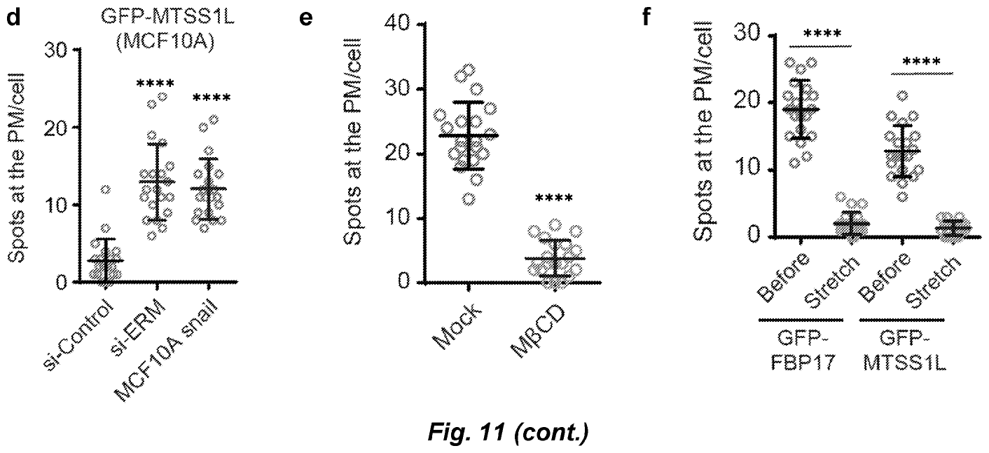

Homeostatic PM tension suppresses cancer cell motility by counteracting BAR proteins. Next, we examined the mechanisms by which PM tension inhibited cancer cell motility. We previously demonstrated that FBP17, a BAR domain-containing protein regulating membrane curvature, acts as a sensor of PM tension involved in actin-based directed migration. Consistently, a mathematical model indicated that the polymerizing ability of BAR proteins is intrinsically dependent on membrane tension, suggesting a universal tension-sensing mechanism through BAR proteins. Thus, we performed an siRNA screen for BAR proteins upon ERM knockdown to examine whether BAR proteins play a critical role in the low PM tension-induced invasive phenotype in 3D on-top culture (FIG. 5a). We identified several BAR proteins, including MTSS1L (also known as ABBA) (FIG. 5a). We also noted that knockdown of Toca proteins, such as FBP17 and CIP4, modestly reduced the elongated invasive behavior induced by ERM depletion, suggesting their functional redundancy as previously reported. In fact, their simultaneous depletion led to increased suppression of the invasive phenotype (FBP17+CIP4+Toca-1, triple KD) (FIG. 5a). Of the identified proteins, MTSS1L and Toca proteins were also required for invasion of MDA-MB-231 cells (FIG. 11a) or MCF10A cells induced by the depletion of ERM proteins or RHOA (FIG. 11b). Thus, in subsequent analyses, we focused on these proteins. MTSS1L and Toca proteins are implicated in lamellipodia formation or membrane ruffling through the activation of Arp2/3 complex-dependent actin nucleation, suggesting that these proteins may play a role in low-tension-mediated actin-based protrusions. Indeed, we found that knocking down these BAR proteins suppressed an elongated invasive phenotype driven by Snail overexpression (FIG. 5b). We further examined the role of BAR proteins in 3D cancer cell motility. Time-lapse movies showed that the depletion of MTSS1L or Toca proteins suppressed both mesenchymal and amoeboid motility; the majority of cells exhibited a non-motile phenotype with rounded shapes and 3-fold slower migration speeds (FIGS. 5c and d). Consistently, the deletion of MTSS1L or Toca proteins resulted in not only the inhibition of actin-based protrusions, but also the formation of non-polarized membrane blebs (FIG. 5e).

These data suggest that low tension-mediated mechanosignaling by BAR proteins plays a pivotal role in cancer cell motility, whose mechanisms are normally suppressed by homeostatic PM tension in non-motile epithelial cells. Indeed, GFP-FBP17 mainly exhibited cytoplasmic distribution in MCF10A cells; however, the knockdown of ERM, their kinases, or Snail expression, resulted in the accumulation of GFP-FBP17 at the PM (FIG. 5f). In contrast, in MDA-MB-231 cells expressing ezrin, GFP-FBP17 spontaneously accumulated in both actin- and bleb-based membrane protrusions in 2D (FIG. 11 c) and 3D environments (FIG. 5g). However, no prominent accumulation of GFP-FBP17 was observed in high-tension MA-ezrin-expressing cells (FIG. 5g and FIG. 11c). Similar results were obtained with GFP-MTSS1L (FIGS. 11c and d). Moreover, increasing PM tension by MBCD treatment was also sufficient to prevent the recruitment of FBP17 to the PM (FIGS. 11f and e). To further investigate whether PM tension directly controls the assembly of BAR proteins, we employed a cell-stretching device to increase tension by modulating the in-plane membrane tension. Mechanical stretching of the PM led to rapid disassembly of FBP17 or MTSS1L, accompanied by the disappearance of the leading edges. These data indicate that homeostatic PM tension acts as a mechanical tumor suppressor that inhibits cancer cell dissemination by counteracting membrane-mediated mechanotransduction by BAR proteins.

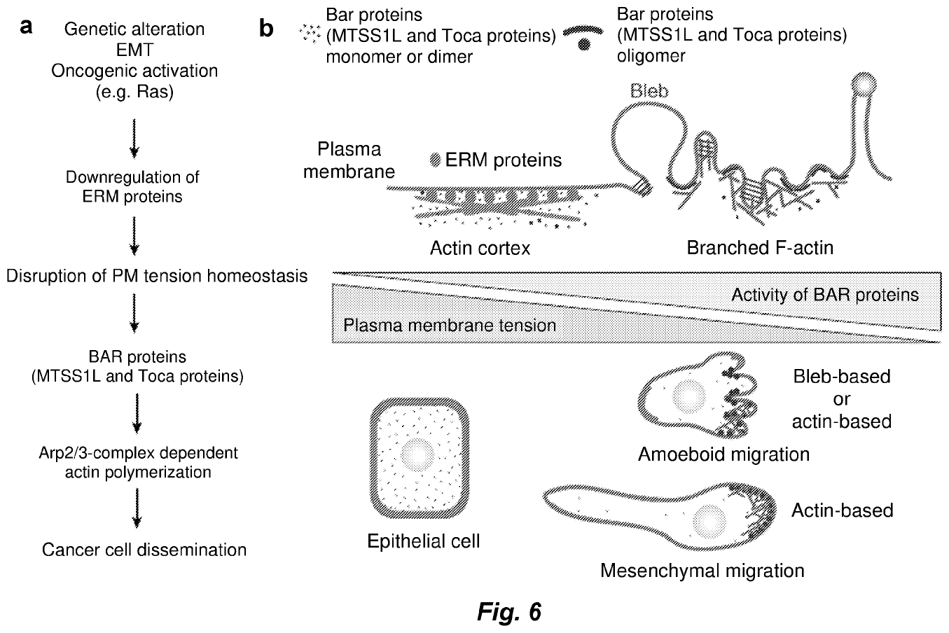

It has long been considered that cell mechanics are inherently associated with invasion and metastasis. However, how such mechanical changes influence tumor dissemination ability at the cellular and molecular levels has remained elusive owing to a lack of understanding of the key physical parameters underlying the malignant phenotype. Here, we show that metastatic cells exhibit significantly lower PM tension than epithelial cells, and that this mechanical property is closely associated not only with membrane protrusion, but also with tumor invasion and metastasis. Our data further indicate that this decreased MCA-based tension is translated into Arp2/3 complex dependent actin polymerization via self-assembly of BAR proteins, such as MTSS1L and Toca proteins, promoting cancer cell migration and invasion (FIG. 6a, b). Recent studies have shown that a variety of BAR proteins play an active role in invasion and metastasis of various cancers. An important aspect of our findings is that invasive behaviors, irrespective of the type of protrusions and motile modes, can be phenotypically normalized by simply increasing MCA-based PM tension. This is consistent with recent studies, showing that membrane-actin detachment is key to both actin- and bleb-based protrusions. An in vivo study reported that the loss of moesin (the sole Drosophila ERM protein) alone is sufficient to induce invasion in Drosophila. Furthermore, analysis of clinical samples showed that ERM proteins commonly exhibit cytoplasmic distribution in malignant tumors, including breast, lung, and head and neck squamous cell carcinoma. Therefore, our study, together with these observations, supports a general role for MCA-based PM tension as a mechanical tumor suppressor.

A limitation of our study is that we cannot exclude the possibility that MA-ezrin expression could have effects other than PM tension, as ERM proteins are known to be involved in the regulation of a variety of signaling molecules. A recent study reports the development of a synthetic molecular tool (iMC-linker) to manipulate MCA by simply connecting the cell membrane to the actin cortex. Interestingly, two recent complementary studies using this tool and constitutively active ezrin show that stem cell spreading, correlated with cell differentiation, is inhibited by increased MCA-based tension. Future studies using this tool will be needed to support the importance of ERM-mediated MCA in inhibiting cancer cell motility.

Our data indicate that ERM-mediated MCA is responsible for maintaining homeostatic PM tension in non-invasive cells. However, changes in the actin structure itself are also considered to affect PM tension. Migrating cells are characterized by less stabilized F-actin and significantly enhanced actin filament turnover rates primarily mediated by cofilin, which contributes to dynamic protrusion formation by providing G-actin monomers. Such accelerated depolymerization may also lead to changes in PM tension, thereby synergistically promoting cancer cell migration. This raises the question of how changes in PM tension led to two different protrusion formations and subsequent migration modes. In epithelial cells, an overall reduction in MCA appeared to induce only slow mesenchymal-like motility. This indicates that a local increase in MCA, especially at the cell rear, may be required for fast migration modes, such as amoeboid migration. In addition, experimental studies and mathematical considerations suggest that decreased PM tension and increased cortical tension favor bleb formation and thus bleb-based migration, in accordance with our tether force data and previous cortical tension measurements. Therefore, a decrease in PM tension may be a prerequisite for both types of protrusion formation, and cortical tension is important for their switching and subsequent migration modes, reflecting why maintaining high PM tension efficiently suppresses both migration modes. Future studies examining the mechanical relationship between these two forces in migratory behaviors, particularly in 3D environments, will advance our understanding of how cell mechanics control cancer cell migration.

Recent studies have shown that the disruption of cell-cell adhesion in epithelial cells does not necessarily lead to single-cell dissemination. Our study suggests that homeostatic PM tension is a cell-autonomous characteristic of non-motile cells, which may partially explain the above phenomenon. In addition, cancer cells are known to exploit collective migration characterized by multicellular coordination through cell-cell adhesion for dissemination. Such collective processes are mechanically mediated by the coordination between the cell adhesion forces and actomyosin contractility. It will be interesting to investigate how PM tension integrates with these forces to control collective migration and whether manipulating PM tension could suppress this type of movement.

An unexpected finding was that homeostatic PM tension may also play an active role in hindering tumor formation and growth. Interestingly, it has been suggested that changes in cell surface mechanics appear to be correlated with cancer stemness. Moreover, a recent study suggests a direct link between membrane protrusions and cancer progression. Our data indicate that such membrane fluctuations driven by EMT or oncogenic transformation can be explained by decreased PM tension, suggesting that maintaining high PM tension may also function as an effective mechanism to suppress tumorigenicity. It will be interesting to further explore the link between PM tension and cancer progression, especially in the context of cancer stemness.

Our data showed that epithelial cells have a PM tension of ˜100 pN/μm that is comparable with the membrane tension at which BAR domain self-assembly is inhibited (100-200 pN/μm), as determined by reconstitution experiments and mathematical models, suggesting a threshold tension for BAR protein assembly. We propose that PM tension above a critical threshold inhibits the self-organization of BAR proteins, thereby suppressing branched actin assembly and subsequent actin-based migration. This inhibitory mechanism could also partially explain why increased PM tension suppresses tumor development, as some BAR proteins are reported to play an active role in tumorigenesis. Our unexpected finding was that BAR proteins are also required for bleb-based movement. Our knockdown experiments suggested that BAR proteins are not required for the formation of membrane blebs but are involved in their polarization. This may be relevant to a recent study that reported that local membrane invaginations driven by BAR proteins enable local membrane protrusions essential for directed bleb-based migration. Importantly, because PM tension regulated by MCA should serve as a local parameter, local membrane undulations upon its detachment may recruit curvature-sensitive BAR proteins, driving both polarized actin-based and bleb-based motility. Overall, these observations suggest that the low PM tension-BAR protein axis may serve as a general form of membrane-mediated mechanosignaling that drives cancer cell migration, re-emphasizing PM tension as a promising target for limiting tumor dissemination.

Abnormal changes in the cell membrane shape, including the formation of microvesicles/exosomes and micropinocytosis, are hallmarks of cancer. It is tempting to speculate that these functions could be directly controlled by PM tension. Our findings provide a foundation for future investigations into whether MCA manipulation can be exploited for therapeutic interventions aimed at normalizing cell membrane mechanics.

### Pharmaceutical Formulations

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard or soft-shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.50% to about 60%, about 10% to about 250%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze-drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The compounds described herein can be effective anti-tumor agents and have higher potency and/or reduced toxicity as compared to BHPI. Preferably, compounds of the invention are more potent and less toxic than BHPI, and/or avoid a potential site of catabolic metabolism encountered with BHPI, i.e., have a different metabolic profile than BHPI. Furthermore, the compounds described herein cause less severe ataxia than BHPI and other known compounds.

The invention provides therapeutic methods of treating cancer in a vertebrate such as a mammal, which involve administering to a mammal having cancer an effective amount of a compound or composition described herein. A mammal includes a primate, human, rodent, canine, feline, bovine, ovine, equine, swine, caprine, bovine and the like. Cancer refers to any of the various type of malignant neoplasm, which are in general characterized by an undesirable cellular proliferation, e.g., unregulated growth, lack of differentiation, local tissue invasion, and metastasis. Cancers that can be treated by a compound described herein include, for example, breast cancer, cervical carcinoma, colon cancer, endometrial cancer, leukemia, lung cancer, melanoma, pancreatic cancer, prostate cancer, ovarian cancer, or uterine cancer, and in particular, any cancer that is ERα positive.

The ability of a compound of the invention to treat cancer may be determined by using assays well known to the art. For example, the design of treatment protocols, toxicity evaluation, data analysis, quantification of tumor cell kills, and the biological significance of the use of transplantable tumor screens are known. In addition, ability of a compound to treat cancer may be determined using the Tests as described below.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Materials and Methods

Cell culture. Human non-tumorigenic mammary epithelial cells (MCF10A), human breast cancer cells (MCF7, AU565, MDA-MB-231, and Hs578T), and human pancreatic (PANC-1) cancer cells were purchased from the American Type Culture Collection (ATCC). Normal rat liver (IAR-2) epithelial cells and human prostate (PC-3) cancer cells were obtained from the Japanese Collection of Research Bioresources (JCRB) Cell Bank. Normal canine kidney (MDCK II) epithelial cells were kindly provided by M. Murata (University of Tokyo). MDCK II cells carrying doxycycline inducible RasV12 were previously described. MCF10A cells were cultured in DMEM/F12 (Invitrogen) supplemented with 5% horse serum (Gibco), EGF (20 ng/ml; R&D Systems), insulin (10 μg/ml; Sigma-Aldrich), cholera toxin (80 μg/ml; Wako), and hydrocortisone (0.5 μg/ml; Sigma-Aldrich). AU565 cells were cultured in RPMI-1640 (Nacalai Tesque) supplemented with 10% FBS (Sigma-Aldrich). Other cell lines were cultured in DMEM (Nacalai Tesque) supplemented with 10% FBS (Sigma-Aldrich). All cell lines were cultured at 37° C. in 5% CO₂. All cell lines were regularly tested for mycoplasma contamination using a PCR-based mycoplasma detection kit (Venor GeM Classic; MB minerva biolabs).

For 3D culture, cells were incorporated into 3D collagen lattices (type-I bovine collagen; final concentration, 1.67 mg/ml; Advanced BioMatrix). For 3D on-top culture, cells were grown in a mix of Matrigel:collagen, as previously described (https://brugge.med.harvard.edu/Protocols). We used growth factor reduced Matrigel (BD Biosciences).

Tether force measurement with optical tweezers and the estimation of PM tension. Tether force measurements were performed with an optical tweezers system (NanoTracker™ 2, JPK Instruments) equipped with an infrared (IR) laser source (3 W, 1064 nm) on an Olympus IX-73 inverted microscope with a 60×(numerical aperture=1.2) water immersion objective (Olympus). Silica beads (1.5 μm diameter, Polysciences) were incubated with concanavalin A (Sigma-Aldrich) at 1 mg/ml for 1 h. Cells were plated onto glass-bottom dishes (WPI). Coated beads were added to the cell culture medium supplemented with 25 mM HEPES (pH 7.5), and experiments were performed at 37° C. Tether force (F) can be calculated using Hooke's law: F=kΔx, where k is the stiffness of the trap and Δx is the displacement of the bead from the trap center. Trap stiffness (k, typically ˜0.15 pN/nm) was calibrated for each experiment by a power spectrum analysis. A single bead was captured by the optical trap, kept in contact with the plasma membrane for 500 milliseconds, then pulled away to form a membrane tether (5 μm length) by moving a piezo stage under computer control at 1 μm/s. The displacement of the bead in the trap center (Δx) was determined by a quadrant photodiode detector with high precision (the resolution less than 1 nm). Data analysis was performed using the JPK data processing software. To compare PM tension between non-motile epithelial cells and malignant cells, we measured the tethering force on the lateral sides of cells. We noted that cells with low PM tension, like malignant cells, frequently formed double tethers, which exhibited a two-fold increase in the tether force. This may be due to their low tension. Therefore, we carefully confirmed whether one tether was formed and excluded data from double tethers. We also noted that the membrane tether tended to extend longer in metastatic cells (>15 μm), as opposed to that in epithelial cells whose tethers were easy to break.

PM tension can be estimated with the following formula: T=Fₜₑₜₕₑᵣ²/8Bπ², where T is the apparent plasma membrane tension (PM tension), Fₜₑₜₕₑᵣ is the tether force measured by optical tweezers, and B is the bending stiffness of the membrane. Given that B is known to be relatively invariable between the cell types tested (from 1-3×10⁻¹⁹ Nm), we used B (1.4×10⁻¹⁹ N m) to calculate PM tension.

Materials. Methyl-β-cyclodextrin (MCD) (Sigma-Aldrich) and calyculin A (Cell Signaling Technology) were used at a final concentration of 4 mM and 0.5 nM, respectively. The following antibodies were used: anti-ERM (rabbit polyclonal, 1:1000 for immunoblotting; #3142, Cell Signaling Technology [CST]); anti-phospho-ERM (rabbit monoclonal, 1:100 for immunostaining; #3726, CST); anti-RHOA (mouse monoclonal, 1:1000 for immunoblotting; #sc-418, Santa Cruz Biotechnology); anti-pS19 MLC (Phospho-myosin light chain 2 [Ser19]) (mouse monoclonal, 1:1000 for immunoblotting; #3675, CST); anti-MLC (rabbit polyclonal, 1:1000 for immunoblotting; #3672, CST); anti-E-cadherin (rabbit monoclonal, 1:1000 for immunoblotting; #3915, CST); anti-vimentin (rabbit monoclonal, 1:1000 for immunoblotting; #5741, CST); anti-MTSS1L (rabbit polyclonal, 1:200 for immunoblotting; #NBP2-57037, Novus Biologicals); anti-FBP17 (FNBP1) (rabbit polyclonal, 1:1000 for immunoblotting); anti-CIP4 (TRIP10) (mouse monoclonal, 1:1000 for immunoblotting; #612556, BD Transduction Laboratories); anti-HA-Tag (rabbit monoclonal [C29F4], 1:100 for immunostaining; #3724, CST), and anti-f-actin (rabbit polyclonal 1:2000 for immunoblotting; #PM053, MBL). Alexa-Fluor-488-conjugated secondary antibody (1:500 for immunostaining; rabbit, #A11034; mouse, #A11029) was obtained from Thermo Scientific.

Human Snail and Slug were subcloned into pMXs-IRES-Puro retroviral vector (Cell Biolabs, Inc; modified by introducing an HA tag to the C-terminus). Lyn₁₀-ezrinT567E (MA-ezrin) was constructed by the fusion of the PM targeting signal (MGCIKSKRKD (SEQ ID NO: 75), a myristoylation motif derived from Lyn tyrosine kinase) to the N-terminus of human ezrin, followed by the generation of the T567E mutation using PCR primers and the sequence was confirmed. Ezrin and MA-ezrin constructs were subcloned into the pQCXIN-HA retroviral vector (Clontech; modified by introducing an HA tag to the C-terminus) with a neomycin-resistant gene. Human MTSS1L was subcloned into the pEGFP C-1 vector. For retrovirus infection, cells were plated in a 6-well plate and incubated with viruses in the presence of 4 μg/ml polybrene (Sigma-Aldrich). Infected cells were selected with G418 (0.8 mg/ml) or puromycin (1.5 μg/ml). Transgene expression was assessed by western analysis and confocal microscopy.

Short interfering RNA (siRNA) and transfection. For knockdown experiments, Dharmacon SMARTpool-ON-TARGETplus siRNAs (a mixture of four different siRNAs; Thermo Scientific) against human genes were used: EZR/ezrin (L-017370-00); RDX/radixin (L-011762-00); MSN/moesin (L-011732-00); RHOA (L-003860-00); SLK (L-003850-00); STK10 (L-004168-00); ARHGAP4 (L-003628-00); ARHGAP10/GARF2 (L-009382-01); ARHGAP17/RICH1 (L-008335-02); ARHGAP26/GRAF1 (L-008426-00); ARHGAP29 (L-008277-00); ARHGAP42/GRAF3 (L-026507-01); ARHGAP44/RICH2 (L-009238-01); ARHAGP45/HMHA1 (L-023893-00); ARHGEF37 (L-032927-01); ARHGEF38 (L-020676-00); IRSp53/BAIAP2 (L-012206-02); BAIAP2L1/IRTKS (L-018664-02); DNMBP/Tuba (L-026304-01); FCHSD1 (L-015107-02); FCHSD2 (L-021240-01); FER (L-003129-00); FES (L-003130-00); FNBP1/FBP17 (L-026214-02); FNBP1L/Toca-1 (L-020718-01); GAS7 (L-011492-00); GIMP (L-021160-01); MTSS1/MIM (L-018506-00); MTSS1L/ABBA (L-022582-01); OPHN1/Oligophrenin 1 (L-009444-00); PACSIN1 (L-007735-00); PACSIN2 (L-019666-02); PACSIN3 (L-015343-00); SH3BP1 (L-009546-01); SRGAP1 (L-026974-00); SRGAP2 (L-021531-02); SRGAP3 (L-014175-00); TRIP10/CIP4 (L-012685-00). ON-TARGETplus Non-Targeting siRNA Pool (D-001810-10) was used as a control siRNA. RNAs (25 nM) were transfected into cells with Lipofectamine RNAi MAX (Invitrogen). Analyses were performed 72 h after transfection. We confirmed the knockdown of key proteins (ERM proteins, RHOA, SLK, STK10, MTSS1L, FBP17, and CIP4) by western analysis. Plasmid transfections were performed using FuGENE HD (Roche) according to the manufacturer's protocol. Transfected cells were examined after 24 h.

In vitro invasion and migration assays. For invasion and migration assays, we used BioCoat Matrigel Invasion Chambers (Corning) and 8.0 μm PET trans-migration inserts (Corning), respectively. In the invasion assay, 1×10⁵ cells were suspended in serum-free medium and seeded on top of the chamber's membrane, in which medium containing serum was placed at the bottom. For MβCD or calyculin A treatment, drugs were added in both sides. Low-invasive epithelial (MCF10A, AU565, and MCF7) cells and MDA-MB-231 cells were incubated for 36 h and 24 h, respectively, and then fixed in 4% formaldehyde. Invaded cells were imaged using a BZ-X700 microscope (Keyence) with 10× magnification and counted. In the migration assay, 5×10⁴ cells were seeded on top of the PET membrane, incubated for 24 h, and analyzed as described for the invasion assay. For the quantification of invasive structures grown in 3D on-top culture (FIGS. 5a and b), the elongated mesenchymal-like and rounded morphologies were defined as invasive and non-invasive phenotypes, respectively, and quantified. No amoeboid-like movement characterized by a rounded shape was observed in ERM-knocked-down cells or Snail-expressing cells.

Confocal microscopy, live-cell imaging, and image analysis. For immunofluorescence analysis, cells were fixed with 4% formaldehyde in phosphate-buffered saline (PBS) for 10 min and permeabilized with 0.2% Triton X-100 for 10 min in PBS. Cells were blocked with 5% goat serum (Sigma-Aldrich) in PBS for 1 h and were incubated with primary antibodies for at least 3 h. Then, cells were incubated with secondary antibodies for 1 h. For membrane visualization, Alexa Fluor™ 350 conjugated wheat germ agglutinin (WGA) (Thermo Scientific) was incubated with fixed cells for 30 min before permeabilization. For visualization of F-actin, Alexa Fluor™ 568 Phalloidin (Thermo Scientific) was incubated with fixed cells for 30 min. Fluorescence images were captured using a confocal microscopy system (FluoView 1000-D; Olympus) equipped with 405-, 473-, and 568-nm diode lasers through an objective lens (60× oil immersion objective, NA=1.35). For 3D images, Z stack images of consecutive optical planes spaced by 0.5 μm were acquired for the whole cell. Maximum intensity z-projections were reconstructed using Image J and Imaris 8.0.2. All other confocal images were displayed as a single plane. In the 3D data shown in FIGS. 1-3, the plane image near the middle of 3D stacks, where the membrane region can be clearly distinguishable, was selected as the representative image. For live imaging with phase-contrast microscopy, cells were mixed with a collagen matrix and plated in 8 well plates (IWAKI). The images were taken using the BZ-X700 microscope (Keyence) with 20× magnification at 37° C. and 5% CO₂. Single cells were manually tracked using Manual Tracking Tool ImageJ software plugin. Migration plots and cell velocities were obtained with Chemotaxis and Migration Tool (Ibidi). To evaluate the cell morphodynamics in 3D, cells were classified as mesenchymal (elongated; aspect ratio>4) or amoeboid phenotypes (rounded with actin or bleb-based protrusions; aspect ratio<4), as previously described. The aspect ratio was determined as a ratio of the major cell axis length to minor cell axis length, which was calculated automatically by Image J. We observed that elongated cells typically have an aspect ratio of 5-8, whereas that of amoeboid cells is 2-3. To calculate the membrane/cytoplasm intensity ratio, the entire membrane region was segmented using the threshold images of the WGA channel and the membrane regions were manually selected using the brush selection tool with a size of 5 pixels (approximately 500 nm in width). The mean intensity of pERM or F-actin all along the membrane regions was then calculated and divided by their mean intensities throughout the cytosol (avoiding the nucleus). To quantify 3D images, a single plane image (corresponding to the image near the middle of 3D stacks) with clearly distinguishable membrane regions by WGA staining, as shown in FIG. 1d, was used for quantitative analysis as in 2D. Additionally, two images that shifted up and down in the z-direction from the selected middle image by about 1.5 μm (elongated cells) to 4 μm (rounded cells) were quantified to account for the variation in fluorescence intensity due to the thickness of 3D stacks. The average membrane/cytoplasm intensity ratio of all three images was used as one sample. To quantify the accumulation of BAR proteins at the PM, the number of BAR protein spots that merged with the membrane region defined by the membrane marker was quantified.

Western blotting. Cell lysates were extracted using Laemmli buffer. The samples were electrophoresed in SDS-PAGE gels, transferred to a polyvinylidene difluoride membrane, blocked with 5% BSA or nonfat dry milk in TBS containing 0.1% Tween 20, incubated with primary antibodies, and then incubated secondary antibodies.

Proliferation assay. 2×10⁴ cells were seeded in duplicate for each time-point in 6-well plates and counted after 2 and 4 days using a Countess Automated cell counter (Thermo Fisher).

Analysis of clinical data sets. TCGA and CCLE datasets were analyzed in the 2020 version of cBioPortal (www.cbioportal.org/). Clinical data sets of cancer patients from the 2020 version of KMplotter (www.kmplot.com) were analyzed using each probe (SLK: 206875_s_at and STK10: 228394_at) and the auto-selection best cutoff. The significance of survival differences between groups was assessed by the log-rank test.

Animal studies. For tumor formation, local invasion, and spontaneous metastasis assays, 1×10⁷ cells were resuspended in PBS (0.1 mL) and injected into the mammary fat pad of 6-week-old female BALB/c nu/nu mice. Mice were maintained in a temperature (23±1° C., 55±5% humidity) with a 12/12 hour light/dark cycle. The mice were killed 7 weeks after injection, and the tumors and lungs were collected. The tumor volume was calculated according to the formula V=½(A×B²), where A and B represent the largest and smallest dimensions of the tumor, respectively. Resected tumors were fixed in 4% paraformaldehyde, embedded in paraffin, and stained with hematoxylin and eosin (H&E). For invasiveness analysis, the borders of the tumor to the adipose tissue were manually defined, and 37,829 μm² quantification boxes were located. In each box, the tumor invasive areas were calculated using Image J. To quantify spontaneous metastasis, the ratio of human/mouse DNA was assessed using human-specific quantitative PCR (qPCR), as previously described. Briefly, qPCR was performed using PowerTrack SYBR Green Master Mix (Thermo Fisher Scientific) and 100 ng of lung genomic DNA with human- and mouse-specific PTGER2 primer pairs. Primers used for qPCR are included in Table 1. A standard curve was generated using genomic DNA extracted from MDA-MB-231 cells and xenograft-naive mouse lung. qPCR was performed using the StepOnePlus Real-Time PCR System. For experimental lung metastasis assays, 2×10⁶ cells were resuspended in 0.1 ml PBS and injected intravenously into 6-week-old female BALB/c nu/nu mice, and lung metastasis was assessed after 8 weeks. Lung was fixed and stained using H&E. All sections were examined under the BZ-X700 microscope or the BZ-8000 microscope (Keyence). All animal experiments were reviewed by the Institutional Ethics Committee and performed in compliance with the Guidelines for Laboratory Animal Research of the Tokyo University of Pharmacy and Life Sciences (Tokyo, Japan).

TABLE 3
List of primers for qPCR analysis

| Gene | Forward Primer | Common reverse primer |
|---|---|---|
| Human-specific PTGER2 | GCTGCTTCTCATTGTCTCGG | GCCAGGAGAATGAGGTGGTC |
| | (SEQ ID NO: 218) | (SEQ ID NO: 219) |
| Mouse-specific PTGER2 | CCTGCTGCTTATCGTGGCTG | GCCAGGAGAATGAGGTGGTC |
| | (SEQ ID NO: 220) | (SEQ ID NO: 221) |

Mechanical stretch of cells. Cells were grown on silicon chambers (STB-CH-04, STREX) coated with fibronectin (0.05 mg/ml; Sigma-Aldrich) for 24 h. The chambers were set on the stretching device (STB-100, STREX), and stretched uniaxially for 5 min (20% stretch).

Statistics. Statistical analyses were carried out with GraphPad Prism 6 and Excel. D'Agostino Pearson omnibus and Kolmogorov-Smirnov (with the Dallal-Wilkinson-Lillie for P-value) tests were used to test datasets for Gaussian distribution. Statistical significance was determined using: two-tailed Student's t-test or non-parametric Mann-Whitney U-test for two groups; one-way ANOVA with Tukey's test for multiple comparisons. Phenotype distributions were compared using a chi-square test. The sample sizes, number of repeats for each experiment, and specific tests are stated in the figure legends.

Data availability. Source data are provided with this paper. TCGA and CCLE datasets are available from the cBioPortal (www.cbioportal.org/). Clinical data sets of cancer patients are available from KMplot (www.kmplot.com).

### Example 2. Pharmaceutical Dosage Forms

The following formulations illustrate representative pharmaceutical dosage forms that may be used for the therapeutic or prophylactic administration of a compound of a formula described herein, a compound specifically disclosed herein, or a pharmaceutically acceptable salt or solvate thereof (hereinafter referred to as ‘Compound X’):

| (i) Tablet 1 | mg/tablet |
|---|---|
| ‘Compound X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| ‘Compound X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Compound X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| ‘Compound X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/mL) | mg/mL |
|---|---|
| ‘Compound X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| ‘Compound X’ | 20 |
| Oleic acid | 10 |
| Trichloromonofluoromethane | 5,000 |
| Dichlorodifluoromethane | 10,000 |
| Dichlorotetrafluoroethane | 5,000 |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Compound X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference in particular, Tsujita et al., Nat Commun 12(1), 5930 (2021). No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A method of treating cancer comprising contacting a cancer cell with an agent to increase tension of a plasma membrane of the cancer cell, thereby treating the cancer cell.

2. The method of claim 1 wherein the tension of the plasma membrane is increased to and/or maintained at about 100-200 pN/μm or greater.

3. The method of claim 1 wherein the increasing tension of the plasma membrane comprises increasing an internal pressure of the cancer cell.

4. The method of claim 3 wherein the increasing of the internal pressure of the cancer cell comprises manipulating a permeability function of the cancer cell.

5. The method of claim 1 wherein the increasing tension of the plasma membrane comprises increasing or decreasing an amount of a component of the plasma membrane.

6. The method of claim 5 wherein the component of the plasma membrane is one or more of a lipid, a phospholipid, a glycolipid, a protein, a glycoprotein, and cholesterol.

7. The method of claim 1 wherein the agent is internalized into the cancer cell.

8. The method of claim 1 wherein the agent to increase the tension of the plasma membrane causes an increase in membrane-actin cortex attachment (MCA) wherein the increase in the MCA is compared to a cell that is not contacted by the agent.

9. The method of claim 8 wherein the agent is an expression vector comprising a phosphatidylinositol 4-phosphate 5-kinase (PIP5K) gene, wherein the PIP5K gene is one or more of PIP5K1A, PIP5K1B, and PIP5K1C, wherein a product of the one or more PIP5K gene increases an amount of phosphatidylinositol 4,5-bisphosphate (PIP2) in the plasma membrane.

10. The method of claim 1 wherein the agent causes an increase in phosphorylation of an ezrin, radixin, and moesin (ERM) protein comprising one or more of EZR, RDX, and MSN.

11. The method of claim 10 wherein the increase in phosphorylation of the ERM protein comprises increasing the expression or activation of a kinase protein to phosphorylate the ERM protein.

12. The method of claim 10 wherein the kinase protein is one or more of is one or more of RHOA, ROCK1, ROCK2, SLK, and STK10.

13. The method of claim 1 wherein the agent inhibits or reduces expression of one or more proteins comprising a Bin, Amphiphysin, and Rvs (BAR) domain.

14. The method of claim 13 wherein the one or more proteins comprise a gene product of one or more of MTSS1L/ABBA, FNBP1L/Toca-1, TRIP10/CIP4, ARHGAP4, ARHGAP10/GARF2, ARHGAP17/RICH1, ARHGAP26/GRAF1, ARHGAP29, ARHGAP42/GRAF, ARHGAP44/RICH2, ARHGAP45/HMHA1, ARHGEF37, ARHGEF38, IRSp53/BAIAP2, BAIAP2L1/IRTKS, DNMBP/Tuba, FCHSD1, FCHSD2, FER; FES, FNBP1/FBP17, GAS7, GMIP, MTSS1/MIM, OPHN1, PACSIN1, PACSIN2, PACSIN3, SH3BP1, SRGAP1, SRGAP2, and SRGAP3.

15. The method of claim 13 wherein the agent is an antibody, aptamer, short interfering RNA (siRNA), micro-RNA (miRNA), short hairpin RNA (shRNA), a nanobody, an affimer, DNA, a CRISPR/Cas9 system, or a chemical compound.

16. The method of claim 15 wherein the agent is one or more of siRNA, miRNA, and shRNA that binds to one or more of a messenger ribonucleic acid of MTSS1L/ABBA, FNBP1/FBP17, TRIP10/CIP4, ARHGAP4, ARHGAP10/GARF2, ARHGAP17/RICH1, ARHGAP26/GRAF1, ARHGAP29, ARHGAP42/GRAF, ARHGAP44/RICH2, ARHGAP45/HMHA1, ARHGEF37, ARHGEF38, IRSp53/BAIAP2, BAIAP2L1/IRTKS, DNMBP/Tuba, FCHSD1, FCHSD2, FER; FES, FNBP1L/Toca-1, GAS7, GMIP, MTSS1/MIM, OPHN1, PACSIN1, PACSIN2, PACSIN3, SH3BP1, SRGAP1, SRGAP2, and SRGAP3.

17. The method of claim 16 wherein the one or more of siRNA, miRNA, and shRNA binds to a messenger ribonucleic acid of one or more of MTSS1L/ABBA, FNBP1/FBP17, and TRIP10/CIP4.

18. The method of claim 15 wherein the DNA is an expression vector configured to express an ezrin fusion protein comprising a conserved myristylation sequence of Lyn fused with ezrin, wherein the ezrin comprises a phosphomimic activating mutation (T567E).

19. The method of claim 1 wherein the agent is formulated as a composition comprising a pharmaceutically acceptable carrier.

20. A method of inhibiting migration and/or proliferation of a cell comprising reducing expression of one or more proteins comprising a BAR domain or increasing expression or phosphorylation of an ezrin, radixin, and moesin (ERM) protein, thereby inhibiting the migration and/or proliferation of a cell.

21. A method of increasing or decreasing cell division of eukaryotic cells in a cell culture comprising:
contacting the eukaryotic cell with an agent to cause a change in the tension of a plasma membrane of the cell, thereby increasing or decreasing the rate of cell division compared to a rate of cell division of a eukaryotic cell not contacted with the agent.

22. The method of claim 21 wherein the agent causing the decrease in cell division increases expression of one or more of ezrin, radixin, and moesin (ERM) proteins, or causes an increase in phosphorylation of the ERM proteins.

23. The method of claim 22 wherein the agent causing the increase in phosphorylation of the ERM proteins comprises an expression vector comprising one or more of ROCK1, ROCK2, SLK, STK10, and RHOA.

24. The method of claim 21 wherein the agent causing the decrease in cell division is an expression vector configured to express an ezrin fusion protein comprising a conserved myristylation sequence of Lyn fused with ezrin, wherein the ezrin comprises a phosphomimic activating mutation (T567E).

25. The method of claim 21 wherein the agent causing the increase in cell division increases expression of one or more BAR domain proteins.

26. The method of claim 21 wherein the agent causing the increase in cell division decreases expression of one or more of ezrin, radixin, and moesin (ERM) proteins.

27. The method of claim 21 wherein the agent is an antisense RNA, a siRNA, shRNA, or miRNA or an antibody.

28. A method of modulating a characteristic of a cell comprising contacting the cell with an agent to change a tension of a plasma membrane of the cell, wherein the change in the tension of the plasma membrane modulates the characteristic of the cell.

29. The method of claim 28 wherein the characteristic of the cell is controlled by a membrane-actin cortex attachment (MCA), and the agent increases or decreases the MCA compared to a control cell.

30. The method of claim 29 wherein the characteristic of the cell is an increase or decrease in cell motility and invasion of surrounding tissue.

31. The method of claim 29 wherein the characteristic of the cell is an increase or decrease in cell proliferation and tumor formation.
